(19) United States
(12) Patent Application Publication
(10) Pub. No.: US 2025/0255797 A1
(43) Pub. Date: Aug. 14, 2025

(54) Recombinant Nucleic Acids Encoding Cosmetic Protein(s) For Aesthetic Applications

(71) Applicant: Krystal Biotech, Inc., Pittsburgh, PA (US)

(72) Inventors: Suma Krishnan, Pittsburgh, PA (US); Trevor Parry, Pittsburgh, PA (US); Pooja Agarwal, Pittsburgh, PA (US)

(21) Appl. No.: 18/896,155

(22) Filed: Sep. 25, 2024

Related U.S. Application Data
(63) Continuation of application No. 16/915,695, filed on Jun. 29, 2020, now Pat. No. 12,128,122.
Application No. 16/915,695 is a continuation of application No. 16/395,896, filed on Apr. 26, 2019, now Pat. No. 10,786,438.
(60) Provisional application No. 62/663,476, filed on Apr. 27, 2018.

(51) Int. Cl. A61K 8/65; A61K 8/64; A61Q 19/08

(52) U.S. Cl. CPC A61K 8/65; A61K 8/64; A61Q 19/08; A61K 2800/86; A61K 2800/91; C12N 2710/16621; C12N 2710/16631

(57) ABSTRACT

The present disclosure provides recombinant nucleic acids comprising one or more polynucleotides encoding one or more cosmetic proteins (e.g., one or more human collagen proteins); viruses comprising the recombinant nucleic acids; compositions (e.g., cosmetic formulations) comprising the recombinant nucleic acids and/or viruses; methods of their use; and articles of manufacture or kits thereof.

### CROSS REFERENCE TO RELATED APPLICATIONS

This application is a continuation of U.S. patent application Ser. No. 16/915,695, filed Jun. 29, 2020, which is a continuation of U.S. patent application Ser. No. 16/395,896, filed Apr. 26, 2019, now issued as U.S. Pat. No. 10,786,438, which claims the priority benefit of U.S. Provisional Application Ser. No. 62/663,476, filed Apr. 27, 2018, each of which is incorporated herein by reference in its entirety.

### SUBMISSION OF SEQUENCE LISTING ON ASCII TEXT FILE

The Sequence Listing associated with this application is filed in electronic format and is hereby incorporated by reference into the specification in its entirety. The name of the XML file containing the Sequence Listing is 2407502.xml. The size of the XML file is 258,534 bytes and the XML file was created on Sep. 19, 2024.

### FIELD OF THE INVENTION

The present disclosure relates, in part, to recombinant nucleic acids comprising one or more polynucleotides encoding one or more cosmetic proteins (e.g., one or more human collagen proteins); to viruses comprising the recombinant nucleic acids; to compositions (e.g., cosmetic formulations) comprising the recombinant nucleic acids and/or viruses; to methods of their use; and to articles of manufacture or kits thereof.

### BACKGROUND

Skin, like all organs in the human body, undergoes sequential and often cumulative alterations with the passage of time. Aging of the skin occurs as the result of numerous factors, including inherent changes within the skin, the effects of gravity and facial muscles acting on the skin, soft tissue loss or shift, and loss of tissue elasticity. Interestingly, the “aged” phenotype of skin may be accelerated by environmental factors, most notably, chronic exposure to ultraviolet irradiation (e.g., from the sun). Clinically, the aged phenotype of skin may be described as wrinkled, sagging, and/or generally less elastic and resilient than its youthful counterpart, although variations within this phenotype exist between natural, chronological aging and photoaging.

The dermal extracellular matrix (ECM) comprises the bulk of skin and confers both strength and resiliency. Collagen, a major component of the connective tissue providing support to the skin, decreases as a person ages. In aged skin, collagen fibrils display high levels of degradation and fragmentation, and are replenished by dermal fibroblasts at diminishing rates. These degraded and fragmented collagen bundles become looser and lose strength (disrupting the structural organization of the dermal ECM), and inextricably leads to an “aged” manifestation of the skin.

Numerous skincare products have been developed for improving the appearance of human skin. Wrinkles and skin folds are commonly treated with dermal and subdermal injections of aesthetic facial fillers; however, such a superficial approach does not address the structural changes underlying skin aging, in particular, the damage or loss of collagen. Thus, there exists a clear need for alternative strategies to supplement, strengthen, or replace dermal ECM components (e.g., human collagen), in individuals desiring to combat or reverse the physiological effects of skin aging.

All references cited herein, including patent applications, patent publications, non-patent literature, and NCBI/UniProtKB/Swiss-Prot Accession numbers are herein incorporated by reference in their entirety, as if each individual reference were specifically and individually indicated to be incorporated by reference.

### BRIEF SUMMARY

In order to meet these and other needs, provided herein are recombinant nucleic acids (e.g., recombinant herpes viral genomes) encoding one or more cosmetic proteins for use in viruses (e.g., herpes viruses), compositions, formulations, medicaments, and/or methods for aesthetic/cosmetic applications (e.g., treating wrinkles). The present inventors have shown that the recombinant, attenuated viruses described herein were capable of 1) effectively transducing human epidermal/dermal cells, and 2) successfully expressing the encoded exogenous human collagen (mRNA and protein), where the protein could then localize to the appropriate region in skin-equivalent organotypic cultures (see e.g., Example 2). Moreover, the present inventors have shown that the viruses described herein may be successfully administered either topically or intradermally without significant host cell cytotoxicity, allowing for the human collagen expressed from these viruses to localize to the appropriate region of the dermal ECM after in vivo administration without observable damage to the skin (see e.g., Examples 3 and 7). In addition, the present inventors have shown that multiple different HSV backbones can be used to construct viruses expressing human collagens (see e.g., Example 2), that multiple strategies can be employed to successfully express more than one human collagen protein from a single recombinant genome (see e.g., Example 5), and that candidate viruses can successfully express human collagen proteins in multiple relevant in vitro and in vivo models of chronological or UV-induced skin aging (see e.g., Examples 6 and 7). Furthermore, the present inventors have shown that the viruses described herein can be successfully engineered to express other cosmetic proteins (e.g., human laminins) both in vitro and in vivo, where these proteins localize to the appropriate region of the dermal ECM (see e.g., Example 8). Without wishing to be bound by theory, the data described herein provides strong evidence that the recombinant nucleic acids and/or viruses of the present disclosure may constitute a novel means for delivering cosmetic proteins (e.g., human collagen proteins, such as human Collagen 1 and human Collagen 3), and in particular, to supplement or replace natural human dermal ECM proteins in aesthetic applications (e.g., to reduce the appearance of age or photo-induced wrinkles).

Accordingly, certain aspects of the present disclosure relate to a recombinant herpes virus genome comprising a first polynucleotide encoding a first polypeptide comprising a first cosmetic protein. In some embodiments, the recombinant herpes virus genome comprises two or more copies of the first polynucleotide. In some embodiments, the recombinant herpes virus genome is replication competent. In some embodiments, the recombinant herpes virus genome is replication defective. In some embodiments that may be combined with any of the preceding embodiments, the recombinant herpes virus genome is selected from a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any derivatives thereof. In some embodiments that may be combined with any of the preceding embodiments, the recombinant herpes virus genome is a recombinant herpes simplex virus genome. In some embodiments, the recombinant herpes simplex virus genome is a recombinant type 1 herpes simplex virus (HSV-1) genome, a recombinant type 2 herpes simplex virus (HSV-2) genome, or any derivatives thereof.

In some embodiments, the recombinant herpes simplex virus genome is a recombinant type 1 herpes simplex virus (HSV-1) genome. In some embodiments that may be combined with any of the preceding embodiments, the recombinant herpes simplex virus genome comprises an inactivating mutation. In some embodiments that may be combined with any of the preceding embodiments, the inactivating mutation is in a herpes simplex virus gene. In some embodiments, the inactivating mutation is a deletion of the coding sequence of the herpes simplex virus gene. In some embodiments, the herpes simplex virus gene is selected from Infected Cell Protein (ICP) 0, ICP4, ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivating mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivating mutation in the UL41 gene. In some embodiments that may be combined with any of the preceding embodiments, the recombinant herpes simplex virus genome comprises an inactivating mutation in one or both copies of the ICP0 gene. In some embodiments that may be combined with any of the preceding embodiments, the recombinant herpes simplex virus genome comprises an inactivating mutation in the ICP27 gene. In some embodiments that may be combined with any of the preceding embodiments, the recombinant herpes simplex virus genome comprises an inactivating mutation in the UL55 gene. In some embodiments that may be combined with any of the preceding embodiments, the recombinant herpes simplex virus genome comprises an inactivating mutation in the Joint region. In some embodiments, the recombinant herpes simplex virus genome comprises a deletion of the Joint region. In some embodiments that may be combined with any of the preceding embodiments, the recombinant herpes simplex virus genome comprises the first polynucleotide within one or both of the ICP4 viral gene loci.

In some embodiments that may be combined with any of the preceding embodiments, the first cosmetic protein is selected from a first collagen protein, a first fibronectin protein, a first elastin protein, a first lumican protein, a first vitronectin protein, a first vitronectin receptor protein, a first laminin protein, a first neuromodulator protein, and a first fibrillin protein. In some embodiments that may be combined with any of the preceding embodiments, the first cosmetic protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21 and 53-64. In some embodiments, the first cosmetic protein is a structural extracellular matrix protein (e.g., a collagen protein, an elastin protein, a fibronectin protein, a laminin protein, a fibrillin protein, etc.). In some embodiments, the first cosmetic protein is a collagen protein, an elastin protein, a fibronectin protein, or a laminin protein (e.g., a human collagen protein, a human elastin protein, a human fibronectin protein, or a human laminin protein). In some embodiments that may be combined with any of the preceding embodiments, the first collagen protein is a human collagen protein. In some embodiments that may be combined with any of the preceding embodiments, the first collagen protein is selected from a Collagen alpha-1(I) chain polypeptide (COL1-1), Collagen alpha-2(I) chain polypeptide (COL1-2), a Collagen alpha-1(II) chain polypeptide (COL2), a Collagen alpha-1(III) chain polypeptide (COL3), a Collagen alpha-1(IV) chain polypeptide (COL4-1), a Collagen alpha-2(IV) chain polypeptide (COL4-2), a Collagen alpha-3(IV) chain polypeptide (COL4-3), a Collagen alpha-4(IV) chain polypeptide (COL4-4), a Collagen alpha-5(IV) chain polypeptide (COL4-5), a Collagen alpha-6(IV) chain polypeptide (COL4-6), a Collagen alpha-1(V) chain polypeptide (COL5-1), a Collagen alpha-2(V) chain polypeptide (COL5-2), a Collagen alpha-3(V) chain polypeptide (COL5-3), a Collagen alpha-1(VI) chain polypeptide (COL6-1), a Collagen alpha-2(VI) chain polypeptide (COL6-2), a Collagen alpha-3(VI) chain polypeptide (COL6-3), a Collagen alpha-4(VI) chain polypeptide (COL6-4), a Collagen alpha-5(VI) chain polypeptide (COL6-5), a Collagen alpha-6(VI) chain polypeptide (COL6-6), a Collagen alpha-1(VIII) chain polypeptide (COL8), a Collagen alpha-1(IX) chain polypeptide (COL9-1), a Collagen alpha-2(IX) chain polypeptide (COL9-2), a Collagen alpha-3(IX) chain polypeptide (COL9-3), a Collagen alpha-1(X) chain polypeptide (COL10), a Collagen alpha-1(XI) chain polypeptide (COL11-1), a Collagen alpha-2(XI) chain polypeptide (COL11-2), a Collagen alpha-1(XII) chain polypeptide (COL12), a Collagen alpha-1(XIII) chain polypeptide (COL13), a Collagen alpha-1(XIV) chain polypeptide (COL14), a Collagen alpha-1(XV) chain polypeptide (COL15), a Collagen alpha-1(XVI) chain polypeptide (COL16), a Collagen alpha-1(XVII) chain polypeptide (COL17), a Collagen alpha-1(XVIII) chain polypeptide (COL18), a Collagen alpha-1(XIX) chain polypeptide (COL19), a Collagen alpha-1(XX) chain polypeptide (COL20), a Collagen alpha-1(XXI) chain polypeptide (COL21), a Collagen alpha-1(XXII) chain polypeptide (COL22), a Collagen alpha-1(XXIII) chain polypeptide (COL23), a Collagen alpha-1(XXIV) chain polypeptide (COL24), a Collagen alpha-1(XXV) chain polypeptide (COL25), a Collagen alpha-1(XXVI) chain polypeptide (COL26), a Collagen alpha-1(XXVII) chain polypeptide (COL27), and a Collagen alpha-1(XXVIII) chain polypeptide (COL28). In some embodiments that may be combined with any of the preceding embodiments, the first collagen protein is selected from COL1-1, COL1-2, COL3, COL4-1, COL4-2, COL6-1, and COL17. In some embodiments that may be combined with any of the preceding embodiments, the first collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments that may be combined with any of the preceding embodiments, the first collagen protein is COL3. In some embodiments that may be combined with any of the preceding embodiments, the first collagen protein is human COL3. In some embodiments that may be combined with any of the preceding embodiments, the first collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 17. In some embodiments that may be combined with any of the preceding embodiments, the first cosmetic protein is not a Collagen alpha-1(VII) chain polypeptide (COL7).

In some embodiments, the first polypeptide consists essentially of the first cosmetic protein. In some embodiments, the first polypeptide consists of the first cosmetic protein. In some embodiments, the first polypeptide comprises: (a) the first cosmetic protein; (b) a further cosmetic protein; and (c) a linker polypeptide linking (a) to (b). In some embodiments, the further cosmetic protein is selected from a collagen protein, a fibronectin protein, a elastin protein, a lumican protein, a vitronectin protein, a vitronectin receptor protein, a laminin protein, a neuromodulator protein, and a fibrillin protein. In some embodiments, the further cosmetic protein is a structural extracellular matrix protein (e.g., a collagen protein, an elastin protein, a fibronectin protein, a laminin protein, a fibrillin protein, etc.). In some embodiments, the further cosmetic protein is a collagen protein, an elastin protein, a fibronectin protein, or a laminin protein (e.g., a human collagen protein, a human elastin protein, a human fibronectin protein, or a human laminin protein). In some embodiments, the further collagen protein (e.g., a further human collagen protein) is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the further collagen protein (e.g., a further human collagen protein) is selected from COL1-1, COL1-2, COL3, COL4-1, COL4-2, COL6-1, COL7, and COL17. In some embodiments, the first cosmetic protein and the further cosmetic protein are different. In some embodiments, the first cosmetic protein is COL1-1 (e.g., human COL1-1) and the further cosmetic protein is COL1-2 (e.g., human COL1-2). In some embodiments, the linker polypeptide is a cleavable linker polypeptide. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected SEQ ID NOS: 28-31.

In some embodiments that may be combined with any of the preceding embodiments, the first polynucleotide encodes a polycistronic mRNA comprising: (a) a first open reading frame (ORF) encoding the first polypeptide; (b) a second ORF encoding an additional cosmetic protein; and (c) an internal ribosomal entry site (IRES) separating (a) and (b). In some embodiments, the additional cosmetic protein is selected from a collagen protein, a fibronectin protein, a elastin protein, a lumican protein, a vitronectin protein, a vitronectin receptor protein, a laminin protein, a neuromodulator protein, and a fibrillin protein. In some embodiments, the additional cosmetic protein is a structural extracellular matrix protein (e.g., a collagen protein, an elastin protein, a fibronectin protein, a laminin protein, a fibrillin protein, etc.). In some embodiments, the additional cosmetic protein is a collagen protein, an elastin protein, a fibronectin protein, or a laminin protein (e.g., a human collagen protein, a human elastin protein, a human fibronectin protein, or a human laminin protein). In some embodiments, the additional collagen protein (e.g., an additional human collagen protein) is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the additional collagen protein (e.g., an additional human collagen protein) is selected from COL1-1, COL1-2, COL3, COL4-1, COL4-2, COL6-1, COL7, and COL17. In some embodiments, the first cosmetic protein and the additional cosmetic protein are different. In some embodiments, the first cosmetic protein is COL1-1 (e.g., human COL1-1) and the additional cosmetic protein is COL1-2 (e.g., human COL1-2). In some embodiments, the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23.

In some embodiments that may be combined with any of the preceding embodiments, the recombinant herpes virus genome further comprises a second polynucleotide encoding a second cosmetic protein. In some embodiments, the second cosmetic protein is selected from a collagen protein, a fibronectin protein, a elastin protein, a lumican protein, a vitronectin protein, a vitronectin receptor protein, a laminin protein, a neuromodulator protein, and a fibrillin protein. In some embodiments, the second cosmetic protein is a structural extracellular matrix protein (e.g., a collagen protein, an elastin protein, a fibronectin protein, a laminin protein, a fibrillin protein, etc.). In some embodiments, the second cosmetic protein is a collagen protein, an elastin protein, a fibronectin protein, or a laminin protein (e.g., a human collagen protein, a human elastin protein, a human fibronectin protein, or a human laminin protein). In some embodiments, the second collagen protein (e.g., a second human collagen protein) is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the second collagen protein (e.g., a second human collagen protein) is selected from COL1-1, COL1-2, COL3, COL4-1, COL4-2, COL6-1, COL7, and COL17. In some embodiments, the first and second cosmetic proteins are different. In some embodiments, the first cosmetic protein is COL1-1 (e.g., human COL1-1) and the second cosmetic protein is COL1-2 (e.g., human COL1-2). In some embodiments, the first cosmetic protein is COL1-1 (e.g., human COL1-1) and the second cosmetic protein is COL3 (e.g., human COL3).

In some embodiments that may be combined with any of the preceding embodiments, the recombinant herpes virus genome has reduced cytotoxicity when introduced into a target cell, as compared to a corresponding wild-type herpes virus genome. In some embodiments, the target cell is a cell of the epidermis and/or dermis. In some embodiments, the target cell is a human cell. In some embodiments, the target cell is a fibroblast.

Other aspects of the present disclosure relate to a herpes virus comprising any of the recombinant herpes virus genomes described herein. In some embodiments, the herpes virus is replication competent. In some embodiments, the herpes virus is replication defective. In some embodiments, the herpes virus is attenuated. In some embodiments that may be combined with any of the preceding embodiments, the herpes virus has reduced cytotoxicity as compared to a corresponding wild-type herpes virus. In some embodiments that may be combined with any of the preceding embodiments, the herpes virus is selected from a herpes simplex virus, a varicella zoster virus, a human cytomegalovirus, a herpesvirus 6A, a herpesvirus 6B, a herpesvirus 7, and a Kaposi's sarcoma-associated herpesvirus. In some embodiments that may be combined with any of the preceding embodiments, the herpes virus is a herpes simplex virus. In some embodiments, the herpes simplex virus is a type 1 herpes simplex virus (HSV-1), a type 2 herpes simplex virus (HSV-2), or any derivatives thereof. In some embodiments, the herpes simplex virus is a type 1 herpes simplex virus (HSV-1).

Other aspects of the present disclosure relate to a composition comprising: (a) any of the recombinant herpes virus genomes described herein and/or any of the herpes viruses described herein; and (b) an excipient. In some embodiments, the composition is sterile. In some embodiments that may be combined with any of the preceding embodiments, the composition is suitable for topical, transdermal, subcutaneous, intradermal, oral, intranasal, intratracheal, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, or epicutaneous administration. In some embodiments that may be combined with any of the preceding embodiments, the composition is suitable for intradermal administration. In some embodiments that may be combined with any of the preceding embodiments, the composition is suitable for superficial injection. In some embodiments that may be combined with any of the preceding embodiments, the composition is a cosmetic composition. In some embodiments that may be combined with any of the preceding embodiments, the composition is a skin care product.

Other aspects of the present disclosure relate to the use of any of the recombinant herpes virus genomes described herein and/or any of the herpes viruses described herein as a medicament (e.g., for an aesthetic indication).

Other aspects of the present disclosure relate to the use of any of the recombinant herpes virus genomes described herein and/or any of the herpes viruses described herein as a therapy (e.g., as an aesthetic or cosmetic therapy).

Other aspects of the present disclosure relate to the use of any of the recombinant herpes virus genomes described herein and/or any of the herpes viruses described herein in the manufacture of a medicament useful for treating one or more signs or symptoms of dermatological aging.

Other aspects of the present disclosure relate to a method of enhancing, increasing, augmenting, and/or supplementing the levels of one or more dermal extracellular matrix proteins in a subject, the method comprising administering to the subject an effective amount of any of the herpes viruses described herein and/or any of the compositions described herein.

Other aspects of the present disclosure relate to a method of enhancing, increasing, augmenting, and/or supplementing the levels of one or more collagen proteins in a subject, the method comprising administering to the subject an effective amount of any of the herpes viruses described herein and/or any of the compositions described herein. In some embodiments, the one or more collagen proteins are collagen 3. In some embodiments, the levels of endogenous collagen 3 are reduced as a result of chronological or photo-aging.

Other aspects of the present disclosure relate to a method of enhancing, increasing, augmenting, and/or supplementing the soft tissue of a subject, the method comprising administering to the subject an effective amount of any of the herpes viruses described herein and/or any of the compositions described herein. In some embodiments, the composition is injected into the soft tissue of the subject.

Other aspects of the present disclosure relate to a method of improving skin condition, quality, and/or appearance in a subject in need thereof, the method comprising administering to the subject an effective amount of any of the herpes viruses described herein and/or any of the compositions described herein. In some embodiments, the composition is administered to one or more sites of sun damage or other UV exposure, rough texture, skin sagging, wrinkles, or any combinations thereof.

Other aspects of the present disclosure relate to a method of reducing the appearance of one or more superficial depressions in the skin of a subject in need thereof, the method comprising administering to the subject an effective amount of any of the herpes viruses described herein and/or any of the compositions described herein. In some embodiments, the one or more superficial depressions in the skin are selected from the group consisting of nasolabial folds, crows' feet, frown lines, worry lines, scars, glabellar lines, brow ptosis, tear troughs, nasojugal lines, bunny lines, cheek/mid-face ptosis, marionette lines, poppy dimpling, smile lines, laugh lines, chin creases, neck lines, platysma bands, and any combinations thereof.

Other aspects of the present disclosure relate to a method of increasing and/or improving at least one of texture, smoothness, elasticity, or tension of the skin of a subject in need thereof, the method comprising administering to the subject an effective amount of any of the herpes viruses described herein and/or any of the compositions described herein.

In some embodiments that may be combined with any of the preceding embodiments, the skin of the subject is aging skin. In some embodiments that may be combined with any of the preceding embodiments, the skin of the subject has been damaged due to exposure to ultraviolet light. In some embodiments that may be combined with any of the preceding embodiments, the skin of the subject is wrinkled.

Other aspects of the present disclosure relate to a method of diminishing one or more dermatological signs of aging in a subject in need thereof, the method comprising administering to the subject an effective amount of any of the herpes viruses described herein and/or any of the compositions described herein. In some embodiments, the diminishing of one or more dermatological signs of aging is indicated by the: (a) treatment, reduction, and/or prevention of fine lines and/or wrinkles; (b) reduction of skin pore size; (c) improvement in skin thickness, plumpness, and/or tautness; (d) improvement in skin smoothness, suppleness, and/or softness; (e) improvement in skin tone, radiance, and/or clarity; (f) improvement in procollagen and/or collagen production; (g) improvement in skin texture and or promotion of retexturization; (h) improvement in appearance of skin contours; (i) restoration of skin luster and/or brightness; (j) improvement of skin appearance decreased by aging and/or menopause; (k) improvement in skin moisturization; (l) increase in skin elasticity and/or resiliency; (m) treatment, reduction, and/or prevention or skin sagging; (n) improvement in skin firmness; (o) reduction of pigment spots, mottled skin, and/or scars (such as acne scars); (p) improvement of optical properties of skin by light diffraction or reflection; or (q) any combinations thereof.

In some embodiments that may be combined with any of the preceding embodiments, the subject is a human. In some embodiments that may be combined with any of the preceding embodiments, the herpes virus or composition is administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, peri-articularly, locally, or via inhalation to the subject. In some embodiments that may be combined with any of the preceding embodiments, the herpes virus or composition is administered intradermally to the subject. In some embodiments that may be combined with any of the preceding embodiments, the herpes virus or composition is administered by superficial injection.

Other aspects of the present disclosure relate to a composition comprising: a herpes simplex virus (HSV) comprising a recombinant nucleic acid, wherein the recombinant nucleic acid comprises a first polynucleotide encoding a first polypeptide comprising a first human collagen protein, and an excipient. In some embodiments, the recombinant nucleic acid comprises two or more copies of the first polynucleotide. In some embodiments that may be combined with any of the preceding embodiments, the HSV is replication-defective. In some embodiments that may be combined with any of the preceding embodiments, the HSV is replication-competent. In some embodiments that may be combined with any of the preceding embodiments, the HSV is a herpes simplex type 1 virus, a herpes simplex type 2 virus, or any derivatives thereof.

In some embodiments, the recombinant nucleic acid is a herpes simplex virus amplicon. In some embodiments, the herpes simplex virus amplicon is an HSV-1 amplicon or an HSV-1 hybrid amplicon. In some embodiments, the HSV-1 hybrid amplicon is an HSV/AAV hybrid amplicon, an HSV/EBV hybrid amplicon, and HSV/EBV/RV hybrid amplicon, or an HSV/Sleeping Beauty hybrid amplicon.

In some embodiments, the recombinant nucleic acid is a recombinant herpes simplex virus genome. In some embodiments, the recombinant herpes simplex virus genome is a recombinant HSV-1 genome, a recombinant HSV-2 genome, or any derivatives thereof. In some embodiments that may be combined with any of the preceding embodiments, the recombinant herpes simplex virus genome comprises an inactivating mutation in a herpes simplex virus gene. In some embodiments, the herpes simplex virus gene is selected from the group consisting of Infected Cell Protein (ICP) 0, ICP4, ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivation mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivation mutation in the UL41 gene. In some embodiments that may be combined with any of the preceding embodiments, the recombinant herpes simplex virus genome comprises an inactivation mutation in the ICP0 gene. In some embodiments that may be combined with any of the preceding embodiments, the recombinant herpes simplex virus genome comprises an inactivation mutation in the ICP27 gene. In some embodiments that may be combined with any of the preceding embodiments, the inactivating mutation is a deletion of the coding sequence of the gene(s).

In some embodiments that may be combined with any of the preceding embodiments, the recombinant herpes simplex virus genome comprises the first polynucleotide within a viral gene locus. In some embodiments that may be combined with any of the preceding embodiments, the recombinant herpes simplex virus genome comprises the first polynucleotide within one or both copies of the ICP4 viral gene loci. In some embodiments that may be combined with any of the preceding embodiments, the recombinant herpes simplex virus genome comprises the first polynucleotide within the ICP22 viral gene locus. In some embodiments that may be combined with any of the preceding embodiments, the recombinant herpes simplex virus genome comprises the first polynucleotide within the UL41 viral gene locus. In some embodiments that may be combined with any of the preceding embodiments, the HSV has reduced cytotoxicity as compared to a wild-type herpes simplex virus.

In some embodiments that may be combined with any of the preceding embodiments, the first human collagen protein is selected from Collagen alpha-1(I) chain polypeptide (COL1-1), Collagen alpha-2(I) chain polypeptide (COL1-2), a Collagen alpha-1(II) chain polypeptide (COL2), a Collagen alpha-1(III) chain polypeptide (COL3), a Collagen alpha-1(IV) chain polypeptide (COL4-1), a Collagen alpha-2(IV) chain polypeptide (COL4-2), a Collagen alpha-3(IV) chain polypeptide (COL4-3), a Collagen alpha-4(IV) chain polypeptide (COL4-4), a Collagen alpha-5(IV) chain polypeptide (COL4-5), a Collagen alpha-6(IV) chain polypeptide (COL4-6), a Collagen alpha-1(V) chain polypeptide (COL5-1), a Collagen alpha-2(V) chain polypeptide (COL5-2), a Collagen alpha-3(V) chain polypeptide (COL5-3), a Collagen alpha-1(VI) chain polypeptide (COL6-1), a Collagen alpha-2(VI) chain polypeptide (COL6-2), a Collagen alpha-3(VI) chain polypeptide (COL6-3), a Collagen alpha-4(VI) chain polypeptide (COL6-4), a Collagen alpha-5(VI) chain polypeptide (COL6-5), a Collagen alpha-6(VI) chain polypeptide (COL6-6), a Collagen alpha-1(VII) chain polypeptide (COL7), a Collagen alpha-1(VIII) chain polypeptide (COL8), a Collagen alpha-1(IX) chain polypeptide (COL9-1), a Collagen alpha-2(IX) chain polypeptide (COL9-2), a Collagen alpha-3(IX) chain polypeptide (COL9-3), a Collagen alpha-1(X) chain polypeptide (COL10), a Collagen alpha-1(XI) chain polypeptide (COL11-1), a Collagen alpha-2(XI) chain polypeptide (COL11-2), a Collagen alpha-1(XII) chain polypeptide (COL12), a Collagen alpha-1(XIII) chain polypeptide (COL13), a Collagen alpha-1(XIV) chain polypeptide (COL14), a Collagen alpha-1(XV) chain polypeptide (COL15), a Collagen alpha-1(XVI) chain polypeptide (COL16), a Collagen alpha-1(XVII) chain polypeptide (COL17), a Collagen alpha-1(XVIII) chain polypeptide (COL18), a Collagen alpha-1(XIX) chain polypeptide (COL19), a Collagen alpha-1(XX) chain polypeptide (COL20), a Collagen alpha-1(XXI) chain polypeptide (COL21), a Collagen alpha-1(XXII) chain polypeptide (COL22), a Collagen alpha-1(XXIII) chain polypeptide (COL23), a Collagen alpha-1(XXIV) chain polypeptide (COL24), a Collagen alpha-1(XXV) chain polypeptide (COL25), a Collagen alpha-1(XXVI) chain polypeptide (COL26), a Collagen alpha-1(XXVII) chain polypeptide (COL27), and a Collagen alpha-1(XXVIII) chain polypeptide (COL28). In some embodiments that may be combined with any of the preceding embodiments, the first human collagen protein is selected from COL1-1, COL1-2, COL3, COL4-1, COL6-1, COL7, and COL17. In some embodiments that may be combined with any of the preceding embodiments, the nucleic acid sequence encoding the first human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments that may be combined with any of the preceding embodiments, the first human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments that may be combined with any of the preceding embodiments, the first human collagen protein is not COL7.

In some embodiments that may be combined with any of the preceding embodiments, the first polypeptide comprises: (a) the first human collagen protein; (b) a further human collagen protein; and (c) a linker polypeptide linking (a) to (b). In some embodiments, the linker polypeptide is a cleavable linker polypeptide. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 28-31. In some embodiments, the further human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the further human collagen protein is selected from COL1-1, COL1-2, COL3, COL4-1, COL6-1, COL7, and COL17. In some embodiments, the nucleic acid sequence encoding the further human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the further human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first human collagen protein and the further human collagen protein are different.

In some embodiments that may be combined with any of the preceding embodiments, the first polynucleotide encodes a polycistronic mRNA comprising: (a) a first open reading frame (ORF) encoding the first polypeptide; (b) a second ORF encoding an additional human collagen protein; and (c) an internal ribosomal entry site (IRES) separating (a) and (b). In some embodiments, the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23. In some embodiments, the additional human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the additional human collagen protein is selected from COL1-1, COL1-2, COL3, COL4-1, COL6-1, COL7, and COL17. In some embodiments, the nucleic acid sequence encoding the additional human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the additional human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first human collagen protein and the additional human collagen protein are different.

In some embodiments that may be combined with any of the preceding embodiments, the recombinant nucleic acid further comprises a second polynucleotide encoding a second human collagen protein. In some embodiments, the recombinant nucleic acid comprises two or more copies of the second polynucleotide. In some embodiments, the second human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the second human collagen protein is selected from COL1-1, COL1-2, COL3, COL4-1, COL6-1, COL7, and COL17. In some embodiments, the nucleic acid sequence encoding the second human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the second human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first and second human collagen proteins are different.

In some embodiments that may be combined with any of the preceding embodiments, the recombinant nucleic acid is a recombinant herpes simplex virus genome, and wherein the recombinant herpes simplex virus genome comprises the second polynucleotide within a viral gene locus. In some embodiments, the recombinant herpes simplex virus genome comprises the second polynucleotide within one or both copies of the ICP4 viral gene loci. In some embodiments, the recombinant herpes simplex virus genome comprises the second polynucleotide within the ICP22 viral gene locus. In some embodiments, the recombinant herpes simplex virus genome comprises the second polynucleotide within the UL41 viral gene locus. In some embodiments, the recombinant herpes simplex virus genome comprises the first polynucleotide within one or both copies of the ICP4 viral gene loci and the second polynucleotide within the ICP22 viral gene locus. In some embodiments, the recombinant herpes simplex virus genome comprises the first polynucleotide within one or both copies of the ICP4 viral gene loci and the second polynucleotide within the UL41 viral gene locus.

In some embodiments that may be combined with any of the preceding embodiments, the excipient is adapted for cutaneous (systemic or topical), transdermal, subcutaneous, and/or intradermal administration. In some embodiments that may be combined with any of the preceding embodiments, the excipient comprises a hydroxypropyl methylcellulose gel. In some embodiments that may be combined with any of the preceding embodiments, the excipient is adapted for intradermal administration. In some embodiments that may be combined with any of the preceding embodiments, the excipient comprises a phosphate buffer. In some embodiments that may be combined with any of the preceding embodiments, the excipient comprises glycerol. In some embodiments that may be combined with any of the preceding embodiments, the excipient comprises a lipid carrier. In some embodiments that may be combined with any of the preceding embodiments, the excipient comprises a nanoparticle carrier.

In some embodiments that may be combined with any of the preceding embodiments, the composition is a cosmetic composition. In some embodiments, the cosmetic composition is a skin care product.

Other aspects of the present disclosure relate to a kit comprising any of the compositions described herein and instructions for administering the composition.

Other aspects of the present disclosure relate to a method of enhancing, increasing, augmenting, and/or supplementing the levels of one or more human collagen proteins in a subject, the method comprising administering to the subject an effective amount of any of the compositions described herein.

Other aspects of the present disclosure relate to a method of enhancing, increasing, augmenting, and/or supplementing soft tissue of a subject, the method comprising administering to the subject an effective amount of any of the compositions described herein. In some embodiments, the composition is injected into a soft tissue of the subject.

Other aspects of the present disclosure relate to a method of improving skin quality, condition and/or appearance in a subject in need thereof, the method comprising administering to the subject an effective amount of any of the compositions described herein. In some embodiments, the condition is selected from sun damage, aging, UV exposure, rough texture, skin sagging, wrinkles, and any combinations thereof.

Other aspects of the present disclosure relate to a method of reducing the appearance of one or more superficial depressions in the skin of a subject in need thereof, the method comprising administering to the subject an effective amount of any of the compositions described herein. In some embodiments, administration of the composition reduces the appearance of the one or more superficial depressions in the skin of the subject for at least about three months, at least about six months, at least about nine months, or at least about 12 months. In some embodiments, the appearance of the one or more superficial depressions in the skin of the subject is reduced after administration of the composition, as compared to the appearance of the one or more superficial depression in the skin of the subject prior to administration of the composition.

Other aspects of the present disclosure relate to a method of increasing and/or improving at least one of texture, smoothness, elasticity, or tension of the skin of a subject in need thereof, the method comprising administering to the subject an effective amount of any of the compositions described herein. In some embodiments, the skin of the subject maintains at least one of an increased and/or improved texture, smoothness, elasticity, or tension for at least about three months, at least about six months, at least about nine months, or at least about 12 months after administration of the composition. In some embodiments, at least one of texture, smoothness, elasticity, or tension of the skin of the subject is increased and/or improved after administration of the composition, as compared to the texture, smoothness, elasticity, or tension of the skin of the subject prior to administration of the composition.

In some embodiments that may be combined with any of the preceding embodiments, the skin of the subject is aging skin. In some embodiments that may be combined with any of the preceding embodiments, the skin of the subject has been damaged due to exposure to ultraviolet light. In some embodiments that may be combined with any of the preceding embodiments, the skin of the subject is wrinkled.

Other aspects of the present disclosure relate to a method of diminishing one or more dermatological signs of aging in a subject in need thereof, the method comprising administering to the subject an effective amount of any of the compositions described herein. In some embodiments, the diminishing of one or more dermatological signs of aging is selected from: (a) treatment, reduction, and/or prevention of fine lines and/or wrinkles; (b) reduction of skin pore size; (c) improvement in skin thickness, plumpness, and/or tautness; (d) improvement in skin smoothness, suppleness, and/or softness; (e) improvement in skin tone, radiance, and/or clarity; (f) improvement in procollagen and/or collagen production; (g) improvement in skin texture and or promotion of retexturization; (h) improvement in appearance of skin contours; (i) restoration of skin luster and/or brightness; (j) improvement of skin appearance decreased by aging and/or menopause; (k) improvement in skin moisturization; (l) increase in skin elasticity and/or resiliency; (m) treatment, reduction, and/or prevention or skin sagging; (n) improvement in skin firmness; (o) reduction of pigment spots, mottled skin, and/or acne scars; (p) improvement of optical properties of skin by light diffraction or reflection; and (q) any combinations thereof. In some embodiments, the one or more dermatological signs of aging in the subject is diminished after administration of the composition, as compared to the one or more dermatological signs of aging in the subject prior to administration of the composition.

In some embodiments that may be combined with any of the preceding embodiments, the subject is a human. In some embodiments that may be combined with any of the preceding embodiments, the composition is administered cutaneously (systemically or topically), transdermally, subcutaneously, or intradermally to the subject. In some embodiments, the composition is administered by superficial injection. In some embodiments, the composition is administered intradermally to the subject. In some embodiments, the composition is administered once to the subject. In some embodiments, the composition is administered at least twice to the subject. In some embodiments, at least about 15, at least about 30, at least about 60, at least about 90, or at least about 120 days passes between administrations. In some embodiments that may be combined with any of the preceding embodiments, the composition is administered to one or more affected and/or unaffected areas of the subject. In some embodiments that may be combined with any of the preceding embodiments, the skin of the is abraded prior to administration.

Other aspects of the present disclosure relate to a recombinant nucleic acid comprising a first polynucleotide encoding a first polypeptide comprising a first human collagen protein, wherein the recombinant nucleic acid is a recombinant herpes simplex virus genome. In some embodiments, the recombinant nucleic acid comprises two or more copies of the first polynucleotide. In some embodiments, the recombinant herpes simplex virus genome is a recombinant HSV-1 genome, a recombinant HSV-2 genome, or any derivatives thereof.

In some embodiments that may be combined with any of the preceding embodiments, the recombinant herpes simplex virus genome comprises an inactivating mutation in a herpes simplex virus gene. In some embodiments, the herpes simplex virus gene is selected from the group consisting of Infected Cell Protein (ICP) 0, ICP4, ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivation mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivation mutation in the UL41 gene. In some embodiments that may be combined with any of the preceding embodiments, the recombinant herpes simplex virus genome comprises an inactivation mutation in the ICP0 gene. In some embodiments that may be combined with any of the preceding embodiments, the recombinant herpes simplex virus genome comprises an inactivation mutation in the ICP27 gene. In some embodiments that may be combined with any of the preceding embodiments, the inactivating mutation is a deletion of the coding sequence of the gene(s).

In some embodiments that may be combined with any of the preceding embodiments, the recombinant herpes simplex virus genome comprises the first polynucleotide within a viral gene locus. In some embodiments that may be combined with any of the preceding embodiments, the recombinant herpes simplex virus genome comprises the first polynucleotide within one or both copies of the ICP4 viral gene loci. In some embodiments that may be combined with any of the preceding embodiments, the recombinant herpes simplex virus genome comprises the first polynucleotide within the ICP22 viral gene locus. In some embodiments that may be combined with any of the preceding embodiments, the recombinant herpes simplex virus genome comprises the first polynucleotide within the UL41 viral gene locus. In some embodiments that may be combined with any of the preceding embodiments, the HSV has reduced cytotoxicity as compared to a wild-type herpes simplex virus.

In some embodiments that may be combined with any of the preceding embodiments, the first human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL2. In some embodiments that may be combined with any of the preceding embodiments, the first human collagen protein is selected from COL1-1, COL1-2, COL3, COL4-1, COL6-1, COL7, and COL17. In some embodiments that may be combined with any of the preceding embodiments, the nucleic acid sequence encoding the first human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments that may be combined with any of the preceding embodiments, the first human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments that may be combined with any of the preceding embodiments, the first human collagen protein is not COL7.

In some embodiments that may be combined with any of the preceding embodiments, the first polypeptide comprises: (a) the first human collagen protein; (b) a further human collagen protein; and (c) a linker polypeptide linking (a) to (b). In some embodiments, the linker polypeptide is a cleavable linker polypeptide. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 28-31. In some embodiments, the further human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the further human collagen protein is selected from COL1-1, COL1-2, COL3, COL4-1, COL6-1, COL7, and COL17. In some embodiments, the nucleic acid sequence encoding the further human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the further human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first human collagen protein and the further human collagen protein are different.

In some embodiments that may be combined with any of the preceding embodiments, the first polynucleotide encodes a polycistronic mRNA comprising: (a) a first open reading frame (ORF) encoding the first polypeptide; (b) a second ORF encoding an additional human collagen protein; and (c) an internal ribosomal entry site (IRES) separating (a) and (b). In some embodiments, the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23. In some embodiments, the additional human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the additional human collagen protein is selected from COL1-1, COL1-2, COL3, COL4-1, COL6-1, COL7, and COL17. In some embodiments, the nucleic acid sequence encoding the additional human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the additional human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first human collagen protein and the additional human collagen protein are different.

In some embodiments that may be combined with any of the preceding embodiments, the recombinant nucleic acid further comprises a second polynucleotide encoding a second human collagen protein. In some embodiments, the recombinant nucleic acid comprises two or more copies of the second polynucleotide. In some embodiments, the second human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28. In some embodiments, the second human collagen protein is selected from COL1-1, COL1-2, COL3, COL4-1, COL6-1, COL7, and COL17. In some embodiments, the nucleic acid sequence encoding the second human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, the second human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, the first and second human collagen proteins are different.

In some embodiments that may be combined with any of the preceding embodiments, the recombinant herpes simplex virus genome comprises the second polynucleotide within a viral gene locus. In some embodiments, the recombinant herpes simplex virus genome comprises the second polynucleotide within one or both copies of the ICP4 viral gene loci. In some embodiments, the recombinant herpes simplex virus genome comprises the second polynucleotide within the ICP22 viral gene locus. In some embodiments, the recombinant herpes simplex virus genome comprises the second polynucleotide within the UL41 viral gene locus. In some embodiments, the recombinant herpes simplex virus genome comprises the first polynucleotide within one or both copies of the ICP4 viral gene loci and the second polynucleotide within the ICP22 viral gene locus. In some embodiments, the recombinant herpes simplex virus genome comprises the first polynucleotide within one or both copies of the ICP4 viral gene loci and the second polynucleotide within the UL41 viral gene locus.

Other aspects of the present disclosure relate to a host cell comprising any of the recombinant nucleic acids described herein. In some embodiments, the host cell is a eukaryotic cell. In some embodiments, the host cell is a mammalian cell. In some embodiments, the host cell is a human cell or a non-human primate cell. In some embodiments, the host cell is a Vero cell. In some embodiments, the host cell is a complementing host cell.

Other aspects of the present disclosure relate to a method of collecting a herpes simplex virus, the method comprising: (a) contacting a complementing host cell with any of the recombinant nucleic acids described herein; and (b) collecting the herpes simplex virus generated by the complementing host cell.

Other aspects of the present disclosure relate to a method of collecting a herpes simplex virus, the method comprising; (a) culturing a host cell comprising any of the recombinant nucleic acids described herein; and (b) collecting the herpes simplex virus generated by the host cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-N show schematics of wild-type and modified herpes simplex virus genomes. FIG. 1A shows a wild-type herpes simplex virus genome. FIG. 1B shows a modified herpes simplex virus genome comprising deletions of the coding sequences of ICP4 (both copies) and ICP22, with a polynucleotide containing the coding sequence of a first human collagen polypeptide operably linked to a heterologous promoter integrated at each of the ICP4 loci. FIG. 1C shows a modified herpes simplex virus genome comprising deletions of the coding sequence of ICP4 (both copies), with a polynucleotide containing the coding sequence of a first human collagen polypeptide operably linked to a heterologous promoter integrated at each of the ICP4 loci. FIG. 1D shows a modified herpes simplex virus genome comprising deletions of the coding sequences of ICP4 (both copies) and ICP22, with a polynucleotide containing 1) the coding sequence of a first human collagen polypeptide operably linked to a first heterologous promoter, and 2) the coding sequence of a second human collagen polypeptide operably linked to a second heterologous promoter, integrated at each of the ICP4 loci. Both the first and second human collagen polypeptides are encoded on the same strand of DNA. FIG. 1E shows a modified herpes simplex virus genome comprising deletions of the coding sequence of ICP4 (both copies), with a polynucleotide containing 1) the coding sequence of a first human collagen polypeptide operably linked to a first heterologous promoter, and 2) the coding sequence of a second human collagen polypeptide operably linked to a second heterologous promoter, integrated at each of the ICP4 loci. Both the first and second human collagen polypeptides are encoded on the same strand of DNA. FIG. 1F shows a modified herpes simplex virus genome comprising deletions of the coding sequences of ICP4 (both copies) and ICP22, with a polynucleotide containing 1) the coding sequence of a first human collagen polypeptide operably linked to a first heterologous promoter, and 2) the coding sequence of a second human collagen polypeptide operably linked to a second heterologous promoter, integrated at each of the ICP4 loci. The first and second human collagen polypeptides are encoded on opposite strands of DNA. FIG. 1G shows a modified herpes simplex virus genome comprising deletions of the coding sequence of ICP4 (both copies), with a polynucleotide containing 1) the coding sequence of a first human collagen polypeptide operably linked to a first heterologous promoter, and 2) the coding sequence of a second human collagen polypeptide operably linked to a second heterologous promoter, integrated at each of the ICP4 loci. The first and second human collagen polypeptides are encoded on opposite strands of DNA. FIG. 1H shows a modified herpes simplex virus genome comprising deletions of the coding sequences of ICP4 (both copies) and ICP22, with a polynucleotide encoding a polycistronic mRNA operably linked to a heterologous promoter integrated at each of the ICP4 loci. The polycistronic mRNA contains the coding sequence of a first human collagen polypeptide and a second human collagen polypeptide separated by an internal ribosomal entry site (IRES). FIG. 1I shows a modified herpes simplex virus genome comprising deletions of the coding sequence of ICP4 (both copies), with a polynucleotide encoding a polycistronic mRNA operably linked to a heterologous promoter integrated at each of the ICP4 loci. The polycistronic mRNA contains the coding sequence of a first human collagen polypeptide and a second human collagen polypeptide separated by an internal ribosomal entry site (IRES). FIG. 1J shows a modified herpes simplex virus genome comprising deletions of the coding sequences of ICP4 (both copies) and ICP22, with a polynucleotide containing the coding sequence of a chimeric polypeptide operably linked to a heterologous promoter integrated at each of the ICP4 loci. The chimeric polypeptide comprises the amino acid sequence of a first human collagen polypeptide and second human collagen polypeptide separated by a cleavable linker. FIG. 1K shows a modified herpes simplex virus genome comprising deletions of the coding sequence of ICP4 (both copies), with a polynucleotide containing the coding sequence of a chimeric polypeptide operably linked to a heterologous promoter integrated at each of the ICP4 loci. The chimeric polypeptide comprises the amino acid sequence of a first human collagen polypeptide and second human collagen polypeptide separated by a cleavable linker. FIG. 1L shows a modified herpes simplex virus genome comprising deletions of the coding sequences of ICP4 (both copies) and ICP22, with a first polynucleotide containing the coding sequence of a first human collagen polypeptide operably linked to a heterologous promoter integrated at each of the ICP4 loci, and a second polynucleotide containing the coding sequence of a second human collagen polypeptide operably linked to a heterologous promoter integrated at the ICP22 locus. FIG. 1M shows a modified herpes simplex virus genome comprising deletions of the coding sequences of ICP4 (both copies), ICP22, and UL41, with a first polynucleotide containing the coding sequence of a first human collagen polypeptide operably linked to a heterologous promoter integrated at each of the ICP4 loci, and a second polynucleotide containing the coding sequence of a second human collagen polypeptide operably linked to a heterologous promoter integrated at the UL41 locus. FIG. 1N shows a modified herpes simplex virus genome comprising deletions of the coding sequences of ICP4 (both copies) and UL41, with a first polynucleotide containing the coding sequence of a first human collagen polypeptide operably linked to a heterologous promoter integrated at each of the ICP4 loci, and a second polynucleotide containing the coding sequence of a second human collagen polypeptide operably linked to a heterologous promoter integrated at the UL41 locus.

FIGS. 2A-B show schematics of replication-defective herpes simplex type-1 viruses carrying human collagen 7 (COL7) expression cassettes. FIG. 2A shows a schematic of the virus “KCA211”. FIG. 2B shows a schematic of the virus “SAR-COL7”.

FIGS. 3A-B show human COL7 expression in HaCaT cells infected with KCA211 or SAR-COL7 at the indicated MOIs. FIG. 3A shows human COL7 expression in HaCaT cells infected with KCA211 or SAR-COL7 at the indicated MOIs, as assessed by qPCR. Data is shown as fold change relative to SAR-COL7, after normalization to GAPDH. FIG. 3B shows human COL7 expression in uninfected HaCaT cells, or HaCaT cells infected with KCA211 or SAR-COL7 at the indicated MOIs, as assessed by western blot analysis.

FIGS. 4A-B show immunofluorescence images of human COL7 expression in mock infected primary human cells isolated from a healthy patient (Normal), and mock or SAR-COL7 infected primary human cells isolated from a patient suffering from recessive dystrophic epidermolysis bullosa (RDEB). FIG. 4A shows human COL7 expression in mock infected wild-type and RDEB primary human keratinocytes, or in RDEB primary human keratinocytes infected with SAR-COL7 at the indicated multiplicity of infections (MOIs). FIG. 4B shows human COL7 expression in mock infected wild-type and RDEB primary human fibroblasts, or in RDEB primary human fibroblasts infected with SAR-COL7 at the indicated MOIs.

FIGS. 5A-B show quantitative PCR analysis of human COL7 expression in mock infected primary human cells isolated from a healthy patient, and mock or SAR-COL7 infected primary human cells isolated from a patient suffering from recessive dystrophic epidermolysis bullosa (EB). FIG. 5A shows human COL7 expression in mock infected wild-type (N-HDK) and RDEB (EB-HDK) primary human keratinocytes, or in RDEB primary human keratinocytes infected with SAR-COL7 at the indicated MOIs. COL7 expression is shown as the relative fold change over mock infected wild-type primary human keratinocytes. FIG. 5B shows human COL7 expression in mock infected wild-type (N-HDF) and RDEB (EB-HDF) primary human fibroblasts, or in RDEB primary human fibroblasts infected with SAR-COL7 at the indicated MOIs. COL7 expression is shown as the relative fold change over mock infected wild-type primary human fibroblasts.

FIGS. 6A-B show cellular adhesion of uninfected (control) or SAR-COL7 infected RDEB primary human keratinocytes to untreated (plastic) or treated wells of a microwell plate. FIG. 6A shows cellular adhesion to untreated wells (plastic), or wells treated with increasing concentrations of rat tail Collagen 1. FIG. 6B shows cellular adhesion to untreated wells (plastic), or wells treated with increasing concentrations of human plasma fibronectin.

FIG. 7 show representative immunofluorescence images of human COL7 expression and deposition at the basement membrane zone (BMZ) at day 5 in organotypic cultures constructed with SAR-COL7 infected RDEB primary human keratinocytes and fibroblasts. Both keratinocytes and fibroblasts were infected in situ at the indicated MOI after culture construction.

FIGS. 8A-D show human COL7A1 transcript and genome levels observed in uninfected mouse skin (control), or in mouse skin after topical or intradermal delivery of SAR-COL7, as assessed by qPCR. Error bars represent SEM. FIG. 8A shows human COL7A1 transcripts levels/100 ng total RNA in mouse skin at day 3 after infection. FIG. 8B shows copy number of human COL7A1 DNA/100 ng total DNA in mouse skin at day 3 after infection. FIG. 8C shows human COL7A1 transcripts levels/100 ng total RNA in mouse skin at day 6 after infection. FIG. 8D shows copy number of human COL7A1 DNA/100 ng total DNA in mouse skin at day 6 after infection.

FIGS. 9A-B show representative immunofluorescence images of human COL7 expression in mouse skin after delivery of SAR-COL7. FIG. 9A shows a representative immunofluorescent image of human COL7 expression in mouse skin after intradermal delivery of SAR-COL7. FIG. 9B shows a representative immunofluorescent image of human COL7 expression in mouse skin after topical delivery of SAR-COL7.

FIGS. 10A-B show human COL7A1 transcript and genome levels observed in BALB/c mouse skin after intradermal delivery of vehicle, SAR-COL7, or KCA211, as assessed by qPCR. FIG. 10A shows human COL7A1 transcripts levels/100 ng total RNA in BALB/c mouse skin. FIG. 10B shows copy number of human COL7A1 DNA/100 ng total DNA in BALB/c mouse skin.

FIGS. 11A-B show human COL7A1 transcript and genome levels observed at each injection site in hypomorph mouse skin after high-dose intradermal delivery of HSV-GFP (GFP ctrl) or SAR-COL7, as assessed by qPCR. Each bar represents a single sample at the indicated time point. FIG. 11A shows human COL7A1 transcripts levels/100 ng total RNA in hypomorph mouse skin. FIG. 11B shows copy number of human COL7A1 DNA/100 ng total DNA in hypomorph mouse skin.

FIGS. 12A-B show representative immunofluorescence images of human COL7 expression in hypomorph mouse skin after high-dose intradermal delivery of HSV-GFP (GFP Control) or SAR-COL7. FIG. 12A shows control (GFP) and SAR-COL7 immunofluorescence imaging from hypomorph mouse 1 (harvested at day 3) at 10 and 20× magnification. FIG. 12B shows SAR-COL7 immunofluorescence imaging from hypomorph mouse 2 and hypomorph mouse 3 (harvested at day 7). The figure represents a tiled image of 16 fields acquired with a 10× lens, capturing the entire skin section.

FIG. 13 shows H&E stained samples from hypomorph mouse 1, 2, and 3 (harvested at days 3 and 3). The samples were taken from untreated hypomorph mouse skin, and hypomorph mouse skin after intradermal delivery of HSV-GFP or SAR-COL7.

FIGS. 14A-B show representative electron micrograph images of human COL7 expression in hypomorph mouse skin after intradermal delivery of SAR-COL7. The lamina densa is the dark band indicated through the middle of the images; the black dots are the stained NC domains of human COL71 the blue arrows indicate the formation of anchoring fibrils. FIG. 14A shows electron micrograph images of infected hypomorph mouse skin stained with an antibody specific to the NC2 domain of human COL7 (LH24). FIG. 14B shows electron micrograph images of infected hypomorph mouse skin stained with an antibody specific to the NC1 domain of human COL7 (NP185).

FIGS. 15A-B show human COL7A1 transcript and genome levels observed at each injection site in hypomorph mouse skin after low-dose intradermal delivery of SAR-COL7, as assessed by qPCR. Each bar represents a single sample at the indicated time point. FIG. 15A shows human COL7A1 transcripts levels/100 ng total RNA in hypomorph mouse skin. FIG. 15B shows copy number of human COL7A1 DNA/100 ng total DNA in hypomorph mouse skin.

FIG. 16 shows representative immunofluorescence images of human COL7 expression in hypomorph mouse skin (from mouse 1) after low-dose intradermal delivery of SAR-COL7.

FIGS. 17A-C show human COL1A1 and COL1A2 nucleic acid and protein analyses in Vero cells infected with the indicated clones of HSV encoding COL1A1 alone (inserted into the ICP4 loci) or COL1A1 and COL1A2 (inserted into the ICP4 and ICP22 loci, respectively). FIG. 17A shows the levels of human COL1A1 transcripts present in Vero cells 5 days after infection with the indicated HSV clones, as determined by qRT-PCR analysis. Data is presented for two replicates±SEM. FIG. 17B shows the levels of human COL1A2 transcripts present in Vero cells 5 days after infection with the indicated HSV clones, as determined by qRT-PCR analysis. Data is presented for two replicates±SEM. FIG. 17C shows western blot analysis of human COL1A1 and COL1A2 protein expression in Vero cells 5 days after infection with the indicated COL1A1/COL1A2 positive clones, as determined by qRT-PCR. Uninfected (mock) Vero cells were used as a negative control; GAPDH was used as a loading control.

FIG. 18 shows western blot analysis of human COL1A1 and COL1A2 protein expression in Vero cells 5 days after infection with an HSV isolate encoding a COL1A1-IRES-COL1A2 sequence (IRES-Isolate 6) inserted into the ICP4 loci. Infection with an isolate that does not contain the IRES construct (no insertion) was used as a negative control; GAPDH was used as a loading control.

FIGS. 19A-B show human COL3 nucleic acid and protein analyses in immortalized human keratinocytes (HaCaTs) infected with C3vec01. FIG. 19A shows the levels of human COL3A1 transcripts present in immortalized human keratinocytes (HKs) after infection with C3vec01 at the indicated MOIs. Uninfected (mock) and HSV-mCherry-infected (mCherry) cells were used as negative controls. Data is presented for two replicates±SEM. FIG. 19B shows representative immunofluorescence images of human COL3 protein expression in immortalized human keratinocytes 48 hours after infection with C3vec01 at the indicated MOIs. Uninfected (mock) cells were used as negative controls.

FIGS. 20A-B show human COL3 nucleic acid and protein analyses in immortalized human dermal fibroblasts (HDFs) infected with C3vec01. FIG. 20A shows the levels of human COL3A1 transcripts present in immortalized human dermal fibroblasts (HDFs) after infection with C3vec01 at the indicated MOIs. Uninfected (mock) and HSV-mCherry-infected (mCherry) cells were used as negative controls. Data is presented for two replicates±SEM. FIG. 20B shows representative immunofluorescence images of human COL3 protein expression in immortalized human dermal fibroblasts 48 hours after infection with C3vec01 at the indicated MOIs. Uninfected (mock) cells were used as negative controls.

FIGS. 21A-D show human COL3 nucleic acid and protein analyses in aged primary human fibroblasts (HDFs), sourced from two different vendors, infected with C3vec01 at the indicated MOIs. FIG. 21A shows the levels of human COL3A1 transcripts present in primary HDFs harvested from either a 65-year-old female patient or a 73-year-old male patient (vendor 1) after infection with C3vec01 at the indicated MOIs. Uninfected (mock) cells were used as a negative control. Data is presented for two replicates±SEM. FIG. 21B shows western blot analysis of human COL3A1 protein expression in primary HDFs harvested from a 73-year-old male patient (vendor 1) after infection with C3vec01 at the indicated MOIs. Uninfected (mock) cells were used as a negative control; recombinant human COL3A1 (rCOL3A1) was used as a positive control; GAPDH was used as a loading control. FIG. 21C shows the levels of human COL3A1 transcripts present in primary HDFs harvested from either a 75-year-old female patient or a 73-year-old male patient (vendor 2) after infection with C3vec01 at the indicated MOIs. Uninfected (mock) cells were used as a negative control. Data is presented for two replicates±SEM. FIG. 21D shows western blot analysis of human COL3A1 protein expression in primary HDFs harvested from a 75-year-old female patient (vendor 2) after infection with C3vec01 at the indicated MOIs. Uninfected (mock) cells were used as a negative control; recombinant human COL3A1 (rCOL3A1) was used as a positive control; GAPDH was used as a loading control.

FIGS. 22A-B show human COL3 nucleic acid and protein analyses in immortalized human dermal fibroblasts (HDFs) upon UV exposure. FIG. 22A shows the concentration of COL3 secreted into the supernatant of cultured HDFs 24 hours after exposure to various dosages and times of UV light, as assessed by ELISA. Supernatant collected from non-UV exposed (-UV) HDFs cultured in parallel was used as a control. FIG. 22B shows the levels of human COL3A1 transcripts present in UV-exposed immortalized human dermal fibroblasts (HDFs) after infection with C3vec01 at the indicated MOIs. Uninfected (mock) and HSV-mCherry-infected (mCherry) cells were used as negative controls. Data is presented for two replicates±SEM.

FIGS. 23A-C show COL3 nucleic acid and protein analyses of skin biopsies taken from control- or C3vec01-treated young (6-8-week-old) and old (˜13-months-old) C57BL/6 mice 48 hours after intradermal application. FIG. 23A shows the levels of human COL3A1 DNA present in skin biopsies taken from young and old mice 48 hours after being intradermally administered either C3vec01 or vehicle control, as assessed by qPCR analysis. FIG. 23B shows the levels of human COL3A1 transcripts present in skin biopsies taken from young and old mice 48 hours after being intradermally administered either C3vec01 or vehicle control, as assessed by qRT-PCR analysis. For each condition in the qPCR and qRT-PCR analysis, data is presented as the average of four tissue samples (two replicates/tissue sample)±SEM. FIG. 23C shows representative immunofluorescence images of human COL3 expression in skin biopsies taken from young and old mice 48 hours after being intradermally administered C3vec01. A young mouse intradermally administered vehicle alone was used as a negative control. DAPI staining was used to visualize nuclei.

FIGS. 24A-B show expression of wild-type (WT) human LamB3 in Vero cells infected with the indicated viral isolates. FIG. 24A shows expression of wild-type human LAMB3 in infected Vero cells, as assessed by qPCR analysis. FIG. 24B shows expression of wild-type human LamB3 protein in infected Vero cells, as assessed by western blot.

FIG. 25 shows expression of wild-type (WT) or codon-optimized (CO) human LamB3 protein in Vero cells infected with the indicated viral isolates, as assessed by western blot. Uninfected Vero cells were used as a negative control.

FIG. 26 shows expression of wild-type (WT) or codon-optimized (CO) human LamB3 protein in primary human keratinocytes infected with the indicated viral isolates, as assessed by western blot. Uninfected primary keratinocytes were used as a negative control.

FIGS. 27A-C show expression of wild-type (WT) and codon-optimized (CO) human LamC2 in Vero cells infected with the indicated viral isolates. FIG. 27A shows expression of wild-type human LAMC2 in infected Vero cells, as assessed by qPCR analysis. FIG. 27B shows expression of codon-optimized human LAMC2 in infected Vero cells, as assessed by qPCR analysis. FIG. 27C shows expression of wild-type and codon-optimized human LamC2 protein in infected Vero cells, as assessed by western blot. The boxed viral isolate “LGA” expressing codon-optimized LamC2 was selected for additional experimentation.

FIGS. 28A-C show human LAMC2 expressed from viral isolate “LGA” in immortalized primary human keratinocytes infected at the indicated multiplicities of infection (MOIs). FIG. 28A shows the viral genome copy number in primary immortalized human keratinocytes after infection with viral isolate “LGA” at the indicated MOIs. FIG. 28B shows the transcript level of codon-optimized LAMC2 expressed in primary immortalized human keratinocytes after infection with viral isolate “LGA” at the indicated MOIs. FIG. 28C shows expression of human LamC2 protein in primary immortalized human keratinocytes after infection with viral isolate “LGA” at the indicated MOIs, as assessed by western blot.

FIGS. 29A-D show LAMC2 nucleic acid and protein analysis of skin biopsies taken from control (vehicle)- or HSV isolate “LGA”-treated mice 72 hours after intradermal application. FIG. 29A shows a schematic of the intradermal injection sites on the treated animals. FIG. 29B shows the levels of human LAMC2 DNA present in skin biopsies taken from mice 72 hours after being intradermally administered either HSV isolate LGA or vehicle control, as assessed by qPCR analysis. FIG. 29C shows the levels of human LAMC2 transcripts present in skin biopsies taken from mice 72 hours after being intradermally administered either HSV isolate LGA or vehicle control, as assessed by qRT-PCR analysis. For each condition in the qPCR and qRT-PCR analysis, data is presented as the average of two replicates±SEM. FIG. 29D shows representative immunofluorescence images of human LAMC2 expression in skin biopsies taken from mice 72 hours after being intradermally administered HSV isolate LGA. A site that was intradermally administered vehicle alone was used as a negative control. DAPI staining was used to visualize nuclei; pKal staining was used to visualize mouse laminin-332.

### DETAILED DESCRIPTION

In some embodiments, the present disclosure relates to recombinant nucleic acids (e.g., recombinant herpes viral genomes) encoding one or more cosmetic proteins, and to uses of these recombinant nucleic acids in viruses (e.g., in a herpes virus), compositions, formulations, medicaments, and/or methods for delivering one or more cosmetic proteins to the skin, such as onto, into, and/or through the skin (e.g., to the dermal ECM). In some embodiments, the present disclosure relates to recombinant nucleic acids (e.g., recombinant herpes viral genomes) encoding one or more cosmetic proteins, and to uses of these recombinant nucleic acids in viruses (e.g., in a herpes virus), compositions, formulations, medicaments, and/or methods in order to increase, augment, and/or supplement one or more dermal ECM proteins (e.g., one or more collagen proteins). In some embodiments, the present disclosure relates to recombinant nucleic acids (e.g., recombinant herpes viral genomes) encoding one or more cosmetic proteins, and to uses of these recombinant nucleic acids in viruses (e.g., in a herpes virus), compositions, formulations, medicaments, and/or methods in the aesthetic context (e.g., to reduce one or more dermatological signs of aging). In some embodiments, the present disclosure relates to compositions comprising a recombinant herpes viral vector and methods comprising the delivery of the recombinant herpes viral vector onto, into, and/or through the skin of a mammal, wherein the recombinant herpes viral vector comprises a promoter operable in a mammalian cell and a heterologous nucleic acid which is expressed to achieve a cosmetic effect in mammalian skin. The heterologous nucleic acid may be delivered to a mammalian target skin cell of a mammal, comprising contacting the epidermis, dermis, or subcutaneous tissue of the mammal with the composition comprising the recombinant herpes viral vector, under conditions whereby the recombinant herpes viral vector is transported onto, into, and/or through the epidermis, dermis or subcutaneous tissue and introduced into the target skin cell, where it is expressed Without wishing to be bound by theory, it is believed that administering one or more of the recombinant nucleic acids, viruses, and/or formulations described herein to an individual will allow for increased production of functional dermal ECM proteins (e.g., human collagen) in the individual. Furthermore, without wishing to be bound by theory, it is believed that increasing, augmenting, and/or supplementing the levels of cosmetic proteins in an individual by administering one or more of the recombinant nucleic acids, viruses, and/or formulations described herein will lead to at least one of: 1) the enhancement, augmentation, and/or supplementation of soft tissue; 2) the improvement of skin quality, condition, and/or appearance; 3) the reduction of one or more superficial depressions in the skin (e.g., wrinkles); 4) the improvement of texture, smoothness, elasticity, and/or tension of the skin; and/or 5) the reduction of one or more dermatological signs of aging. Ultimately, without wishing to be bound by theory, it is believed that the recombinant nucleic acids, viruses, compositions, and methods described herein provide a novel strategy for delivering functional cosmetic proteins in aesthetic settings.

The following description sets forth exemplary methods, parameters, and the like. It should be recognized, however, that such a description is not intended as a limitation on the scope of the present disclosure but is instead provided as a description of exemplary embodiments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. Definitions

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a” “an” and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c”, “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operatively linked” or “operably linked” means that the DNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus or other vector that, upon introduction into an appropriate host cell, results in expression of the nucleic acids. Appropriate expression vectors are well known to those of skill in the art and include those that are replicable in eukaryotic cells, and those that remain episomal or those which integrate into the host cell genome.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, and/or translation of the polynucleotide.

As used herein, the term “transgene” refers to a polynucleotide that is capable of being transcribed into RNA and translated and/or expressed under appropriate conditions, after being introduced into a cell. In some aspects, it confers a desired property to a cell into which it was introduced, or otherwise leads to a desired cosmetic, therapeutic, or diagnostic outcome.

As used herein, the terms “polypeptide,” “protein,” and “peptide” are used interchangeably and may refer to a polymer of two or more amino acids.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject to provide an effective dose of the active ingredient(s) employed.

As used herein, “cutaneous administration” or “cutaneously administering” refers to the delivery of a composition to a subject by contacting, directly or otherwise, a formulation comprising the composition to all (“systemic”) or a portion (“topical”) of the skin of a subject. The term encompasses several routes of administration including, but not limited to, topical and transdermal. Topical administration may be used as a means to deliver a composition to the epidermis or dermis of a subject, or to specific strata thereof.

As used herein, “treatment” refers to clinical intervention designed to alter the natural course of the individual or cell being treated during the course of clinical pathology. Desirable effects of treatment include decreasing the rate of disease/disorder/defect progression, ameliorating or palliating the disease/disorder/defect state, and remission or improved prognosis. For example, an individual is successfully “treated” if one or more symptoms associated with dermatological aging are reduced, mitigated, or eliminated, including the reduction or elimination of wrinkles.

As used herein, the term “delaying progression of” a disease/disorder/defect refers to deferring, hindering, slowing, retarding, stabilizing, and/or postponing development of the disease/disorder/defect (e.g., skin wrinkles). This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease/disorder/defect.

### III. Recombinant Nucleic Acids

Certain aspects of the present disclosure relate to recombinant nucleic acids (e.g., isolated recombinant nucleic acids) comprising one or more polynucleotides (e.g., one or more, two or more, three or more, four or more, five or more, ten or more, etc.) encoding a cosmetic protein. Any suitable cosmetic protein described herein or known in the art may be encoded by the polynucleotides of the present disclosure, including, for example, collagen proteins, fibronectins, elastins, lumicans, vitronectins/vitronectin receptors, laminins, neuromodulators, fibrillins, additional dermal ECM proteins, etc. In some embodiments, the cosmetic protein is a structural extracellular matrix protein (e.g., a collagen, elastin, fibronectin, laminin, fibrillin, etc.). In some embodiments, the cosmetic protein is a collagen, elastin, fibronectin, or laminin protein (e.g., a human collagen, elastin, fibronectin, or laminin protein).

In some embodiments, the present disclosure relates to recombinant nucleic acids (e.g., isolated recombinant nucleic acids) comprising one or more polynucleotides (e.g., one or more, two or more, three or more, four or more, five or more, ten or more, etc.) encoding a collagen protein. In some embodiments, the collagen protein is a human collagen protein. In some embodiments, the present disclosure relates to recombinant nucleic acids comprising one or more polynucleotides encoding a homotrimeric collagen (e.g., a homotrimeric human collagen, such as human Collagen 3 (e.g., comprising three COL3A1 (COL3) polypeptides) or human Collagen 7 (e.g., comprising three COL7A1 (COL7) polypeptides). In some embodiments, the present disclosure relates to recombinant nucleic acids comprising one or more polynucleotides encoding a heterotrimeric collagen (e.g., a heterotrimeric human collagen, such as human Collagen 1 (e.g., comprising two COL1A1 (COL1-1) polypeptides and one COL1A2 (COL1-2) polypeptide) or human Collagen 4 (e.g., comprising two COL4A1 (COL4-1) polypeptides and one COL4A2 (COL4-2) polypeptide). In some embodiments, the present disclosure relates to recombinant nucleic acids comprising one or more polynucleotides encoding a homotrimeric collagen and a heterotrimeric collagen (e.g., a recombinant nucleic acid comprising one or more polynucleotides encoding a human Collagen 1 and a human Collagen 3). In some embodiments, the present disclosure relates to recombinant nucleic acids comprising one or more polynucleotides encoding human Collagen 1. In some embodiments, the present disclosure relates to recombinant nucleic acids comprising one or more polynucleotides encoding human Collagen 3.

In some embodiments, the present disclosure relates to recombinant nucleic acids comprising a first polynucleotide encoding a first polypeptide comprising a first cosmetic protein (e.g., a first human collagen protein). In some embodiments, the first polypeptide consists essentially of or consists of the first cosmetic protein (e.g., consists essentially of or consists of a first human collagen protein). In some embodiments, the present disclosure relates to recombinant nucleic acids comprising a first polynucleotide encoding a first polypeptide comprising: a first cosmetic protein (e.g., a first human collagen protein), a linker polypeptide, and a further cosmetic protein (e.g., a further human collagen protein). In some embodiments, the first and further cosmetic proteins (e.g., the first and further human collagen proteins) are the same. In some embodiments, the first and further cosmetic proteins (e.g., the first and further human collagen proteins) are different. In some embodiments, the linker polypeptide is a cleavable linker polypeptide.

In some embodiments, the present disclosure relates to recombinant nucleic acids comprising a first polynucleotide encoding a first polypeptide comprising a first cosmetic protein (e.g., a first human collagen protein), wherein the first polynucleotide encodes a polycistronic mRNA comprising: a first open reading frame (ORF) encoding the first polypeptide, an internal ribosomal entry site (IRES), and a second ORF encoding an additional cosmetic protein (e.g., an additional human collagen protein). In some embodiments, the first and additional cosmetic proteins (e.g., the first and additional human collagen proteins) are the same. In some embodiments, the first and additional cosmetic proteins (e.g., the first and additional human collagen proteins) are different.

In some embodiments, the present disclosure relates to recombinant nucleic acids comprising a first polynucleotide encoding a first polypeptide comprising a first cosmetic protein (e.g., a first human collagen protein), and a second polynucleotide encoding a second cosmetic protein (e.g., a second human collagen protein). In some embodiments, the first and second cosmetic proteins (e.g., the first and second human collagen proteins) are the same. In some embodiments, the first and second cosmetic proteins (e.g., the first and second human collagen proteins) are different.

In some embodiments, the recombinant nucleic acid is a vector. In some embodiments, the recombinant nucleic acid is a viral vector. In some embodiments, the recombinant nucleic acid is a herpes viral vector. In some embodiments, the recombinant nucleic acid is a herpes simplex virus amplicon. In some embodiments, the recombinant nucleic acid is a recombinant herpes virus genome. In some embodiments, the recombinant nucleic acid is a recombinant herpes simplex virus genome. In some embodiments, the recombinant herpes simplex virus genome is a recombinant type 1 herpes simplex virus (HSV-1) genome.

### Polynucleotides Encoding Cosmetic Proteins

### Polynucleotides Encoding Collagen Proteins

In some embodiments, the present disclosure relates to a recombinant nucleic acid comprising one or more polynucleotides comprising the coding sequence of a collagen gene. The coding sequence of any collagen gene (including any isoform thereof) from any suitable species known in the art may be encoded by a polynucleotide of the present disclosure, including, for example, human collagen genes (see e.g., NCBI Gene IDs: 1277, 1278, 1281, 1282, 1284, 1291, 1294, 1308, etc.), mouse collagen genes (see, e.g., NCBI Gene IDs: 12842, 12843, 12825, 12826, 12827, 12833, 12836, 12821, etc.), chimpanzee collagen genes (see e.g., NCBI Gene IDs: 104001053, 455117, 459815, 452689, 452661, 450204, 101056895, 101058306, etc.), rat collagen genes (see e.g., NCBI Gene IDs: 29393, 84352, 84032, 290905, 306628, 294337, 301012, 294027, etc.), rabbit collagen genes (see e.g., NCBI Gene IDs: 100347598, 100008997, 100009177, 100358256, 100358522, 100343947, 100356561, 100339335, etc.) etc. Methods of identifying collagen gene homologs/orthologs from additional species are known to one of ordinary skill in the art, including, for example, using a nucleic acid sequence alignment program such as the BLAST® blastn suite. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the collagen genes (and/or coding sequences thereof) described herein or known in the art.

In some embodiments, a polynucleotide of the present disclosure comprises a codon-optimized variant of the coding sequence of any of the collagen genes described herein or known in the art. In some embodiments, use of a codon-optimized variant of the coding sequence of a collagen gene increases stability and/or yield of heterologous expression (RNA and/or protein) of the encoded collagen protein in a target cell (such as a cell of the epidermis and/or dermis), as compared to the stability and/or yield of heterologous expression of a corresponding, non-codon-optimized, wild-type sequence. Any suitable method known in the art for performing codon optimization of a sequence for expression in one or more target cells (e.g., one or more human cells) may be used, including, for example, by the methods described by Fath et al. (PLoS One. 2011 Mar. 3; 6(3): e17596).

In some embodiments, the present disclosure relates to one or more polynucleotides (i.e., one or more first polynucleotides and/or one or more second polynucleotides) comprising the coding sequence of a human collagen gene. Any suitable human collagen gene (including any isoform thereof) known in the art may be encoded by a nucleic acid of the present disclosure, including, for example, a COL1A1 gene (see e.g., NCBI Gene ID: 1277; SEQ ID NO: 1), a COL1A2 gene (see e.g., NCBI Gene ID: 1278; SEQ ID NO: 3), a COL2A1 gene (see e.g., NCBI Gene ID: 1280), a COL3A1 gene (see e.g., NCBI Gene ID: 1281; SEQ ID NO: 5), a COL4A1 gene (see e.g., NCBI Gene ID: 1282; SEQ ID NO: 7), a COL4A2 gene (see e.g., NCBI Gene ID: 1284), a COL4A3 gene (see e.g., NCBI Gene ID: 1285), a COL4A4 gene (see e.g., NCBI Gene ID: 1286), a COL4A5 gene (see e.g., NCBI Gene ID: 1287), a COL4A6 gene (see e.g., NCBI Gene ID: 1288), a COL5A1 gene (see e.g., NCBI Gene ID: 1289), a COL5A2 gene (see e.g., NCBI Gene ID: 1290), a COL5A3 gene (see e.g., NCBI Gene ID: 50509), a COL6A1 gene (see e.g., NCBI Gene ID: 1291; SEQ ID NO: 9), a COL6A2 gene (see e.g., NCBI Gene ID: 1292), a COL6A3 gene (see e.g., NCBI Gene ID: 1293), a COL6A4 gene (see e.g., NCBI Gene ID: 344875), a COL6A5 gene (see e.g., NCBI Gene ID: 256076), a COL6A6 gene (see e.g., NCBI Gene ID: 131873), a COL7A1 gene (see e.g., NCBI Gene ID: 1294; SEQ ID NO: 10), a COL8A1 gene (see e.g., NCBI Gene ID: 1295), a COL9A1 gene (see e.g., NCBI Gene ID: 1297), a COL9A2 gene (see e.g., NCBI Gene ID: 1298), a COL9A3 gene (see e.g., NCBI Gene ID: 1299), a COL10A1 gene (see e.g., NCBI Gene ID: 1300), a COL11A1 gene (see e.g., NCBI Gene ID: 1301), a COL11A2 gene (see e.g., NCBI Gene ID: 1302), a COL12A1 gene (see e.g., NCBI Gene ID: 1303), a COL13A1 gene (see e.g., NCBI Gene ID: 1305), a COL14A1 gene (see e.g., NCBI Gene ID: 7373), a COL15A1 gene (see e.g., NCBI Gene ID: 1306), a COL16A1 gene (see e.g., NCBI Gene ID: 1307), a COL17A1 gene (see e.g., NCBI Gene ID: 1308; SEQ ID NO: 12), a COL18A1 gene (see e.g., NCBI Gene ID: 80781), a COL19A1 gene (see e.g., NCBI Gene ID: 1310), a COL20A1 gene (see e.g., NCBI Gene ID: 57642), a COL21A1 gene (see e.g., NCBI Gene ID: 81578), a COL22A1 gene (see e.g., NCBI Gene ID: 169044), a COL23A1 gene (see e.g., NCBI Gene ID: 91522), a COL24A1 gene (see e.g., NCBI Gene ID: 255631), a COL25A1 gene (see e.g., NCBI Gene ID: 84570), a COL26A1 gene (see e.g., NCBI Gene ID: 136227), a COL27A1 gene (see e.g., NCBI Gene ID: 85301), a COL28A1 gene (see e.g., NCBI Gene ID:340267), etc. In some embodiments, a polynucleotide (i.e., one or more first polynucleotides and/or one or more second polynucleotides) of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the human collagen genes (and/or coding sequences thereof) described herein or known in the art.

In some embodiments, a polynucleotide (i.e., one or more first polynucleotides and/or one or more second polynucleotides) of the present disclosure comprises a codon-optimized variant of any of the human collagen genes described herein. In some embodiments, use of a codon-optimized variant of a human collagen gene increases stability and/or yield of heterologous expression (RNA and/or protein) of the human collagen in a target cell (such as a human keratinocyte or fibroblast), as compared to the stability and/or yield of heterologous expression of a corresponding non-codon-optimized, wild-type sequence.

In some embodiments, a polynucleotide of the present disclosure comprises the coding sequence of the human COL1A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 1 or SEQ ID NO: 2. In some embodiments, a polynucleotide of the present disclosure comprises the sequence of SEQ ID NO: 1 or SEQ ID NO: 2.

In some embodiments, a polynucleotide of the present disclosure comprises a 5′ truncation, a 3′ truncation, or a fragment of the sequence of SEQ ID NO: 1 or SEQ ID NO: 2. In some embodiments, the 5′ truncation, 3′ truncation, or fragment of the sequence of SEQ ID NO: 1 or SEQ ID NO: 2 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, but fewer than 4395, consecutive nucleotides of SEQ ID NO: 1 or SEQ ID NO: 2. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-4392 of SEQ ID NO: 1 or SEQ ID NO: 2. In some embodiments, a polynucleotide of the present disclosure comprises the sequence of nucleic acids 1-4392 of SEQ ID NO: 1 or SEQ ID NO: 2.

In some embodiments, a polynucleotide of the present disclosure comprises the coding sequence of the human COL1A2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 3 or SEQ ID NO: 4. In some embodiments, a polynucleotide of the present disclosure comprises the sequence of SEQ ID NO: 3 or SEQ ID NO: 4.

In some embodiments, a polynucleotide of the present disclosure comprises a 5′ truncation, a 3′ truncation, or a fragment of the sequence of SEQ ID NO: 3 or SEQ ID NO: 4. In some embodiments, the 5′ truncation, 3′ truncation, or fragment of the sequence of SEQ ID NO: 3 or SEQ ID NO: 4 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, but fewer than 4101, consecutive nucleotides of SEQ ID NO: 3 or SEQ ID NO: 4. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-4098 of SEQ ID NO: 3 or SEQ ID NO: 4. In some embodiments, a polynucleotide of the present disclosure comprises the sequence of nucleic acids 1-4098 of SEQ ID NO: 3 or SEQ ID NO: 4.

In some embodiments, a polynucleotide of the present disclosure comprises the coding sequence of the human COL3A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 5 or SEQ ID NO: 6. In some embodiments, a polynucleotide of the present disclosure comprises the sequence of SEQ ID NO: 5 or SEQ ID NO: 6.

In some embodiments, a polynucleotide of the present disclosure comprises a 5′ truncation, a 3′ truncation, or a fragment of the sequence of SEQ ID NO: 5 or SEQ ID NO: 6. In some embodiments, the 5′ truncation, 3′ truncation, or fragment of the sequence of SEQ ID NO: 5 or SEQ ID NO: 6 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, but fewer than 4401, consecutive nucleotides of SEQ ID NO: 5 or SEQ ID NO: 6. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-4398 of SEQ ID NO: 5 or SEQ ID NO: 6. In some embodiments, a polynucleotide of the present disclosure comprises the sequence of nucleic acids 1-4398 of SEQ ID NO: 5 or SEQ ID NO: 6.

In some embodiments, a polynucleotide of the present disclosure comprises the coding sequence of the human COL4A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 7 or SEQ ID NO: 8. In some embodiments, a polynucleotide of the present disclosure comprises the sequence of SEQ ID NO: 7 or SEQ ID NO: 8.

In some embodiments, a polynucleotide of the present disclosure comprises a 5′ truncation, a 3′ truncation, or a fragment of the sequence of SEQ ID NO: 7 or SEQ ID NO: 8. In some embodiments, the 5′ truncation, 3′ truncation, or fragment of the sequence of SEQ ID NO: 7 or SEQ ID NO: 8 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, at least 4500, at least 5000, but fewer than 5010, consecutive nucleotides of SEQ ID NO: 7 or SEQ ID NO: 8. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-5007 of SEQ ID NO: 7 or SEQ ID NO: 8. In some embodiments, a polynucleotide of the present disclosure comprises the sequence of nucleic acids 1-5007 of SEQ ID NO: 7 or SEQ ID NO: 8.

In some embodiments, a polynucleotide of the present disclosure comprises the coding sequence of the human COL6A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 9 or SEQ ID NO: 10. In some embodiments, a polynucleotide of the present disclosure comprises the sequence of SEQ ID NO: 9 or SEQ ID NO: 10.

In some embodiments, a polynucleotide of the present disclosure comprises a 5′ truncation, a 3′ truncation, or a fragment of the sequence of SEQ ID NO: 9 or SEQ ID NO: 10. In some embodiments, the 5′ truncation, 3′ truncation, or fragment of the sequence of SEQ ID NO: 9 or SEQ ID NO: 10 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, but fewer than 3087, consecutive nucleotides of SEQ ID NO: 9 or SEQ ID NO: 10. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-3084 of SEQ ID NO: 9 or SEQ ID NO: 10. In some embodiments, a polynucleotide of the present disclosure comprises the sequence of nucleic acids 1-3084 of SEQ ID NO: 9 or SEQ ID NO: 10.

In some embodiments, a polynucleotide of the present disclosure comprises the coding sequence of the human COL7A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 11 or SEQ ID NO: 12. In some embodiments, a polynucleotide of the present disclosure comprises the sequence of SEQ ID NO: 11 or SEQ ID NO: 12.

In some embodiments, a polynucleotide of the present disclosure comprises a 5′ truncation, a 3′ truncation, or a fragment of the sequence of SEQ ID NO: 11 or SEQ ID NO: 12. In some embodiments, the 5′ truncation, 3′ truncation, or fragment of the sequence of SEQ ID NO: 11 or SEQ ID NO: 12 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, at least 4500, at least 5000, at least 5500, at least 6000, at least 6500, at least 7000, at least 7500, at least 8000, at least 8500, but fewer than 8835, consecutive nucleotides of SEQ ID NO: 11 or SEQ ID NO: 12. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-8832 of SEQ ID NO: 11 or SEQ ID NO: 12. In some embodiments, a polynucleotide of the present disclosure comprises the sequence of nucleic acids 1-8832 of SEQ ID NO: 11 or SEQ ID NO: 12.

In some embodiments, a polynucleotide of the present disclosure comprises the coding sequence of the human COL17A1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 13 or SEQ ID NO: 14. In some embodiments, a polynucleotide of the present disclosure comprises the sequence of SEQ ID NO: 13 or SEQ ID NO: 14.

In some embodiments, a polynucleotide of the present disclosure comprises a 5′ truncation, a 3′ truncation, or a fragment of the sequence of SEQ ID NO: 13 or SEQ ID NO: 14. In some embodiments, the 5′ truncation, 3′ truncation, or fragment of the sequence of SEQ ID NO: 13 or SEQ ID NO: 14 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, but fewer than 4494, consecutive nucleotides of SEQ ID NO: 13 or SEQ ID NO: 14. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-4491 of SEQ ID NO: 13 or SEQ ID NO: 14. In some embodiments, a polynucleotide of the present disclosure comprises the sequence of nucleic acids 1-4491 of SEQ ID NO: 13 or SEQ ID NO: 14.

In some embodiments, a polynucleotide of the present disclosure encoding one or more human collagen proteins (e.g., a first human collagen protein, a further human collagen protein, an additional human collagen protein, and/or a second human collagen protein) has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14. In some embodiments, a polynucleotide of the present disclosure encoding one or more human collagen proteins (e.g., a first human collagen protein, a further human collagen protein, an additional human collagen protein, and/or a second human collagen protein) comprises a sequence selected from SEQ ID NOS: 1-14.

### Polynucleotides Encoding Fibronectin Proteins

In some embodiments, the present disclosure relates to a recombinant nucleic acid comprising one or more polynucleotides comprising the coding sequence of a fibronectin gene. The coding sequence of any fibronectin gene (including any isoform thereof) from any suitable species known in the art may be encoded by a polynucleotide of the present disclosure, including, for example, a human fibronectin gene (see e.g., NCBI Gene ID: 2335), a mouse fibronectin gene (see, e.g., NCBI Gene ID: 14268), a chimpanzee fibronectin gene (see e.g., NCBI Gene ID: 459926), a rat fibronectin gene (see e.g., NCBI Gene ID: 25661), a rabbit fibronectin gene (see e.g., NCBI Gene ID: 100328589), etc. Methods of identifying fibronectin gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the fibronectin genes (and/or coding sequences thereof) described herein or known in the art. In some embodiments, a polynucleotide of the present disclosure comprises a codon-optimized variant of any of the fibronectin genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human fibronectin gene. In some embodiments, a polynucleotide of the present disclosure comprises the coding sequence of the human FN1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 35 or SEQ ID NO: 36. In some embodiments, a polynucleotide of the present disclosure comprises the sequence of SEQ ID NO: 35 or SEQ ID NO: 36.

In some embodiments, a polynucleotide of the present disclosure comprises a 5′ truncation, a 3′ truncation, or a fragment of the sequence of SEQ ID NO: 35 or SEQ ID NO: 36. In some embodiments, the 5′ truncation, 3′ truncation, or fragment of the sequence of SEQ ID NO: 35 or SEQ ID NO: 36 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, at least about 4500, at least about 5000, at least about 5500, at least about 6000, at least about 6500, at least about 7000, but fewer than 7434, consecutive nucleotides of SEQ ID NO: 35 or SEQ ID NO: 36. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-7431 of SEQ ID NO: 35 or SEQ ID NO: 36. In some embodiments, a polynucleotide of the present disclosure comprises the sequence of nucleic acids 1-7431 of SEQ ID NO: 35 or SEQ ID NO: 36.

### Polynucleotides Encoding Elastin Proteins

In some embodiments, the present disclosure relates to a recombinant nucleic acid comprising one or more polynucleotides comprising the coding sequence of an elastin gene. The coding sequence of any elastin gene (including any isoform thereof) from any suitable species known in the art may be encoded by a polynucleotide of the present disclosure, including, for example, a human elastin gene (see e.g., NCBI Gene ID: 2006), a mouse elastin gene (see, e.g., NCBI Gene ID: 13717), a chimpanzee elastin gene (see e.g., NCBI Gene ID: 463943), a rat elastin gene (see e.g., NCBI Gene ID: 25043), a rabbit elastin gene (see e.g., NCBI Gene ID: 100344271), etc. Methods of identifying elastin gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the elastin genes (and/or coding sequences thereof) described herein or known in the art. In some embodiments, a polynucleotide of the present disclosure comprises a codon-optimized variant of any of the elastin genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human elastin gene. In some embodiments, a polynucleotide of the present disclosure comprises the coding sequence of the human ELN gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 37 or SEQ ID NO: 38. In some embodiments, a polynucleotide of the present disclosure comprises the sequence of SEQ ID NO: 37 or SEQ ID NO: 38.

In some embodiments, a polynucleotide of the present disclosure comprises a 5′ truncation, a 3′ truncation, or a fragment of the sequence of SEQ ID NO: 37 or SEQ ID NO: 38. In some embodiments, the 5′ truncation, 3′ truncation, or fragment of the sequence of SEQ ID NO: 37 or SEQ ID NO: 38 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2250, but fewer than 2361, consecutive nucleotides of SEQ ID NO: 37 or SEQ ID NO: 38. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-2358 of SEQ ID NO: 37 or SEQ ID NO: 38. In some embodiments, a polynucleotide of the present disclosure comprises the sequence of nucleic acids 1-2358 of SEQ ID NO: 37 or SEQ ID NO: 38.

### Polynucleotides Encoding Lumican Proteins

In some embodiments, the present disclosure relates to a recombinant nucleic acid comprising one or more polynucleotides comprising the coding sequence of a lumican gene. The coding sequence of any lumican gene (including any isoform thereof) from any suitable species known in the art may be encoded by a polynucleotide of the present disclosure, including, for example, a human lumican gene (see e.g., NCBI Gene ID: 4060), a mouse lumican gene (see, e.g., NCBI Gene ID: 17022), a chimpanzee lumican gene (see e.g., NCBI Gene ID: 452119), a rat lumican gene (see e.g., NCBI Gene ID: 81682), a rabbit lumican gene (see e.g., NCBI Gene ID: 100008665), etc. Methods of identifying lumican gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the lumican genes (and/or coding sequences thereof) described herein or known in the art. In some embodiments, a polynucleotide of the present disclosure comprises a codon-optimized variant of any of the lumican genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human lumican gene. In some embodiments, a polynucleotide of the present disclosure comprises the coding sequence of the human LUM gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 39 or SEQ ID NO: 40. In some embodiments, a polynucleotide of the present disclosure comprises the sequence of SEQ ID NO: 39 or SEQ ID NO: 40.

In some embodiments, a polynucleotide of the present disclosure comprises a 5′ truncation, a 3′ truncation, or a fragment of the sequence of SEQ ID NO: 39 or SEQ ID NO: 40. In some embodiments, the 5′ truncation, 3′ truncation, or fragment of the sequence of SEQ ID NO: 39 or SEQ ID NO: 40 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1017, consecutive nucleotides of SEQ ID NO: 39 or SEQ ID NO: 40. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1014 of SEQ ID NO: 39 or SEQ ID NO: 40. In some embodiments, a polynucleotide of the present disclosure comprises the sequence of nucleic acids 1-1014 of SEQ ID NO: 39 or SEQ ID NO: 40.

### Polynucleotides Encoding Vitronectin and Vitronectin Receptor Proteins

In some embodiments, the present disclosure relates to a recombinant nucleic acid comprising one or more polynucleotides comprising the coding sequence of a vitronectin or vitronectin receptor gene. The coding sequence of any vitronectin or vitronectin receptor gene (including any isoform thereof) from any suitable species known in the art may be encoded by a polynucleotide of the present disclosure, including, for example, a human vitronectin or vitronectin receptor gene (see e.g., NCBI Gene IDs: 7448 and 3685), a mouse vitronectin or vitronectin receptor gene (see, e.g., NCBI Gene IDs: 22370 and 16410), a chimpanzee vitronectin or vitronectin receptor gene (see e.g., NCBI Gene IDs: 738261 and 459807), a rat vitronectin or vitronectin receptor gene (see e.g., NCBI Gene IDs: 29169 and 257645), a rabbit vitronectin or vitronectin receptor gene (see e.g., NCBI Gene IDs: 100009128 and 100008956), etc. Methods of identifying vitronectin or vitronectin receptor gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the vitronectin or vitronectin receptor genes (and/or coding sequences thereof) described herein or known in the art. In some embodiments, a polynucleotide of the present disclosure comprises a codon-optimized variant of any of the vitronectin or vitronectin receptor genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human vitronectin or vitronectin receptor gene. In some embodiments, a polynucleotide of the present disclosure comprises the coding sequence of the human VTN gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 41 or SEQ ID NO: 42. In some embodiments, a polynucleotide of the present disclosure comprises the sequence of SEQ ID NO: 41 or SEQ ID NO: 42.

In some embodiments, a polynucleotide of the present disclosure comprises a 5′ truncation, a 3′ truncation, or a fragment of the sequence of SEQ ID NO: 41 or SEQ ID NO: 42. In some embodiments, the 5′ truncation, 3′ truncation, or fragment of the sequence of SEQ ID NO: 41 or SEQ ID NO: 42 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least about 1250, but fewer than 1437, consecutive nucleotides of SEQ ID NO: 41 or SEQ ID NO: 42. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1034 of SEQ ID NO: 41 or SEQ ID NO: 42. In some embodiments, a polynucleotide of the present disclosure comprises the sequence of nucleic acids 1-1034 of SEQ ID NO: 41 or SEQ ID NO: 42.

### Polynucleotides Encoding Laminin Proteins

In some embodiments, the present disclosure relates to a recombinant nucleic acid comprising one or more polynucleotides comprising the coding sequence of a laminin gene. The coding sequence of any laminin gene (including any isoform thereof) from any suitable species known in the art may be encoded by a polynucleotide of the present disclosure, including, for example, human laminin genes (see e.g., NCBI Gene IDs: 284217, 3908, 3909, 3910, 3911, 3912, 3913, 3914, 3915, 3918, and 10319), mouse laminin genes (see e.g., NCBI Gene IDs: 16774, 16780, and 16782), chimpanzee laminin genes (see e.g., NCBI Gene IDs: 455339, 469668, and 457571), rat laminin genes (see e.g., NCBI Gene IDs: 307582, 305078, and 192362), rabbit laminin genes (see e.g., NCBI Gene IDs: 100346886 and 100342905), etc. Methods of identifying laminin gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the laminin genes (and/or coding sequences thereof) described herein or known in the art. In some embodiments, a polynucleotide of the present disclosure comprises a codon-optimized variant of any of the laminin genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human laminin gene, such as a human LAMA1 gene (see e.g., NCBI Gene ID: 284217), a human LAMA2 gene (see e.g., NCBI Gene ID: 3908), a human LAMA3 gene (see e.g., NCBI Gene ID: 3909), a human LAMA4 gene (see e.g., NCBI Gene ID: 3910), a human LAMA5 gene (see e.g., NCBI Gene ID: 3911), a human LAMB1 gene (see e.g., NCBI Gene ID: 3912), a human LAMB2 gene (see e.g., NCBI Gene ID: 3913), a human LAMB3 gene (see e.g., NCBI Gene ID: 3914), a human LAMC1 gene (see e.g., NCBI Gene ID: 3915), a human LAMC2 gene (see e.g., NCBI Gene ID: 3918), or a human LAMC3 gene (see e.g., NCBI Gene ID: 10319).

In some embodiments, a polynucleotide of the present disclosure comprises the coding sequence of the human LAMA3 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 43 or SEQ ID NO: 44. In some embodiments, a polynucleotide of the present disclosure comprises the sequence of SEQ ID NO: 43 or SEQ ID NO: 44.

In some embodiments, a polynucleotide of the present disclosure comprises a 5′ truncation, a 3′ truncation, or a fragment of the sequence of SEQ ID NO: 43 or SEQ ID NO: 44. In some embodiments, the 5′ truncation, 3′ truncation, or fragment of the sequence of SEQ ID NO: 43 or SEQ ID NO: 44 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, at least 4000, at least about 4500, at least about 5000, but fewer than 5175, consecutive nucleotides of SEQ ID NO: 43 or SEQ ID NO: 44. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-5172 of SEQ ID NO: 43 or SEQ ID NO: 44. In some embodiments, a polynucleotide of the present disclosure comprises the sequence of nucleic acids 1-5172 of SEQ ID NO: 43 or SEQ ID NO: 44.

In some embodiments, a polynucleotide of the present disclosure comprises the coding sequence of the human LAMB3 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 45 or SEQ ID NO: 46. In some embodiments, a polynucleotide of the present disclosure comprises the sequence of SEQ ID NO: 45 or SEQ ID NO: 46.

In some embodiments, a polynucleotide of the present disclosure comprises a 5′ truncation, a 3′ truncation, or a fragment of the sequence of SEQ ID NO: 45 or SEQ ID NO: 46. In some embodiments, the 5′ truncation, 3′ truncation, or fragment of the sequence of SEQ ID NO: 45 or SEQ ID NO: 46 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, but fewer than 3519, consecutive nucleotides of SEQ ID NO: 45 or SEQ ID NO: 46. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-3516 of SEQ ID NO: 45 or SEQ ID NO: 46. In some embodiments, a polynucleotide of the present disclosure comprises the sequence of nucleic acids 1-3516 of SEQ ID NO: 45 or SEQ ID NO: 46.

In some embodiments, a polynucleotide of the present disclosure comprises the coding sequence of the human LAMC2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 47 or SEQ ID NO: 48. In some embodiments, a polynucleotide of the present disclosure comprises the sequence of SEQ ID NO: 47 or SEQ ID NO: 48.

In some embodiments, a polynucleotide of the present disclosure comprises a 5′ truncation, a 3′ truncation, or a fragment of the sequence of SEQ ID NO: 47 or SEQ ID NO: 48. In some embodiments, the 5′ truncation, 3′ truncation, or fragment of the sequence of SEQ ID NO: 47 or SEQ ID NO: 48 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, at least 3000, at least 3500, but fewer than 3582, consecutive nucleotides of SEQ ID NO: 47 or SEQ ID NO: 48. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-3579 of SEQ ID NO: 47 or SEQ ID NO: 48. In some embodiments, a polynucleotide of the present disclosure comprises the sequence of nucleic acids 1-3579 of SEQ ID NO: 47 or SEQ ID NO: 48.

### Polynucleotides Encoding Neuromodulator Proteins

In some embodiments, the present disclosure relates to a recombinant nucleic acid comprising one or more polynucleotides comprising the coding sequence of a neuromodulator gene. The coding sequence of any neuromodulator gene (including any isoform thereof) from any suitable species known in the art may be encoded by a polynucleotide of the present disclosure, including, for example, a Clostridium botulinum neuromodulator gene (see e.g., NCBI Gene IDs: 5185061 and 39483740), etc. Methods of identifying neuromodulator gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the neuromodulator genes (and/or coding sequences thereof) described herein or known in the art. In some embodiments, a polynucleotide of the present disclosure comprises a codon-optimized variant of any of the neuromodulator genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a Clostridium botulinum neuromodulator gene.

In some embodiments, a polynucleotide of the present disclosure comprises the coding sequence of the Clostridium botulinum botA gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 49 or SEQ ID NO: 50. In some embodiments, a polynucleotide of the present disclosure comprises the sequence of SEQ ID NO: 49 or SEQ ID NO: 50.

In some embodiments, a polynucleotide of the present disclosure comprises a 5′ truncation, a 3′ truncation, or a fragment of the sequence of SEQ ID NO: 49 or SEQ ID NO: 50. In some embodiments, the 5′ truncation, 3′ truncation, or fragment of the sequence of SEQ ID NO: 49 or SEQ ID NO: 50 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least about 1250, at least 1500, at least 1750, at least 2000, at least 2500, at least 3000, at least 3500, but fewer than 3891, consecutive nucleotides of SEQ ID NO: 49 or SEQ ID NO: 50. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-3888 of SEQ ID NO: 49 or SEQ ID NO: 50. In some embodiments, a polynucleotide of the present disclosure comprises the sequence of nucleic acids 1-3888 of SEQ ID NO: 49 or SEQ ID NO: 50.

In some embodiments, a polynucleotide of the present disclosure comprises the coding sequence of the Clostridium botulinum botB gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 51 or SEQ ID NO: 52. In some embodiments, a polynucleotide of the present disclosure comprises the sequence of SEQ ID NO: 51 or SEQ ID NO: 52.

In some embodiments, a polynucleotide of the present disclosure comprises a 5′ truncation, a 3′ truncation, or a fragment of the sequence of SEQ ID NO: 51 or SEQ ID NO: 52. In some embodiments, the 5′ truncation, 3′ truncation, or fragment of the sequence of SEQ ID NO: 51 or SEQ ID NO: 52 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least about 1250, at least 1500, at least 1750, at least 2000, at least 2500, at least 3000, at least 3500, but fewer than 3876, consecutive nucleotides of SEQ ID NO: 51 or SEQ ID NO: 52. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-3873 of SEQ ID NO: 51 or SEQ ID NO: 52. In some embodiments, a polynucleotide of the present disclosure comprises the sequence of nucleic acids 1-3873 of SEQ ID NO: 51 or SEQ ID NO: 52.

### Polynucleotides Encoding Fibrillin Proteins

In some embodiments, the present disclosure relates to a recombinant nucleic acid comprising one or more polynucleotides comprising the coding sequence of a fibrillin gene. The coding sequence of any fibrillin gene (including any isoform thereof) from any suitable species known in the art may be encoded by a polynucleotide of the present disclosure, including, for example, human fibrillin genes (see e.g., NCBI Gene IDs: 2200, 2201, and 84467), mouse fibrillin genes (see e.g., NCBI Gene IDs: 14118 and 14119), chimpanzee fibrillin genes (see e.g., NCBI Gene IDs: 453411, 471621, and 455669), rat fibrillin genes (see e.g., NCBI Gene IDs: 83727 and 689008), rabbit fibrillin genes (see e.g., NCBI Gene IDs: 100350931, 100357126, and 100359336), etc. Methods of identifying fibrillin gene homologs/orthologs from additional species are known to one of ordinary skill in the art. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the fibrillin genes (and/or coding sequences thereof) described herein or known in the art. In some embodiments, a polynucleotide of the present disclosure comprises a codon-optimized variant of any of the fibrillin genes (and/or coding sequences thereof) described herein or known in the art.

In some embodiments, the present disclosure relates to one or more polynucleotides (i.e., one or more first polynucleotides and/or one or more second polynucleotides) comprising the coding sequence of a human fibrillin gene, such as a human FBN1 gene (see e.g., NCBI Gene ID: 2200), a human FBN2 gene (see e.g., NCBI Gene ID: 2201), or a human FBN3 gene (see e.g., NCBI Gene ID: 84467).

### Exemplary Polynucleotides

In some embodiments, a polynucleotide of the present disclosure encoding one or more cosmetic proteins (e.g., a first cosmetic protein, a further cosmetic protein, an additional cosmetic protein, and/or a second cosmetic protein) has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14 or 35-52. In some embodiments, a polynucleotide of the present disclosure encoding one or more cosmetic proteins (e.g., a first cosmetic protein, a further cosmetic protein, an additional cosmetic protein, and/or a second cosmetic protein) comprises a sequence selected from SEQ ID NOS: 1-14 or 35-52.

In some embodiments, a polynucleotide of the present disclosure encoding one or more cosmetic proteins (e.g., a first cosmetic protein, a further cosmetic protein, an additional cosmetic protein, and/or a second cosmetic protein) has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NOS: 1-14, 35-38, or 43-48. In some embodiments, a polynucleotide of the present disclosure encoding one or more cosmetic proteins (e.g., a first cosmetic protein, a further cosmetic protein, an additional cosmetic protein, and/or a second cosmetic protein) comprises a sequence selected from SEQ ID NOS: 1-14, 35-38, or 43-48.

A polynucleotide of the present disclosure encoding a cosmetic protein (e.g., a human collagen protein) may further encode additional coding and non-coding sequences. Examples of additional coding and non-coding sequences may include, but are not limited to, sequences encoding additional polypeptide tags (e.g., encoded in-frame with the cosmetic protein in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance cosmetic protein expression in specific cell types (such as human keratinocytes and/or fibroblasts).

In some embodiments, a polynucleotide of the present disclosure encoding a cosmetic protein (e.g., a human collagen protein) is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, HSV chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the 3-actin promoter), a ubiquitin promoter (e.g., a ubiquitin C (UbC) promoter), a phosphoglycerate kinase (PGK) promoter, an immunoglobulin promoter, from heat-shock promoters, and the like), promoters from homologous mammalian genes (e.g., native human collagen, fibronectin, elastin, lumican, vitronectin, laminin, and/or fibrillin promoters), synthetic promoters (such as the CAGG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure encoding cosmetic protein (e.g., a human collagen protein) is operably linked to one or more heterologous promoters. In some embodiments, the one or more heterologous promoters are one or more of constitutive promoters, tissue-specific promoters, temporal promoters, spatial promoters, inducible promoters and repressible promoters. In some embodiments, the one or more heterologous promoters are one or more of the human cytomegalovirus (HCMV) immediate early promoter, the human elongation factor-1 (EF1) promoter, the human β-actin promoter, the human UbC promoter, the human PGF promoter, the synthetic CAGG promoter, and any combinations thereof. In some embodiments, a polynucleotide of the present disclosure encoding a cosmetic protein (e.g., a human collagen protein) is operably linked to an HCMV promoter.

In some embodiments, a polynucleotide of the present disclosure does not comprise the coding sequence of (e.g., a transgene encoding) a Collagen alpha-1 (VII) chain polypeptide (COL7). In some embodiments, a polynucleotide of the present disclosure does not comprise the coding sequence of (e.g., a transgene encoding) a Lysyl hydroxylase 3 polypeptide (LH3). In some embodiments, a polynucleotide of the present disclosure does not comprise the coding sequence of (e.g., a transgene encoding) a Keratin type I cytoskeletal 17 polypeptide (KRT17). In some embodiments, a polynucleotide of the present disclosure does not comprise the coding sequence of (e.g., a transgene encoding) a transglutaminase (TGM) polypeptide (e.g., a human transglutaminase polypeptide such as a human TGM1 polypeptide). In some embodiments, a polynucleotide of the present disclosure does not comprise the coding sequence of (e.g., a transgene encoding) a laminin subunit beta-3 polypeptide (LAMB3). In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide (e.g., a human transglutaminase polypeptide such as a human TGM1 polypeptide), a laminin subunit beta-3 (LAMB3) polypeptide (e.g., a human LamB3 polypeptide) and/or any chimeric polypeptides thereof.

### Cosmetic Proteins

### Collagen Proteins

In some embodiments, the present disclosure relates to one or more polynucleotides encoding a full-length collagen protein or any isoforms or portions thereof. Any collagen protein from any suitable species known in the art may be encoded by a polynucleotide of the present disclosure, including, for example, human collagen proteins (see e.g., UniProt accession numbers P02452, P08123, P02461, P02462, P08572, P12109, Q02388, Q9UMD9. etc.), mouse collagen proteins (see, e.g., UniProt accession numbers P11087, Q01149, P08121, P02463, P08122, Q04857, Q63870, Q07563, etc.), chimpanzee collagen proteins (see e.g., UniProt accession numbers A0A2I3SM98, A0A2J8L483, H2QJ46, K7C8P4, K7C8W0, A0A2J8M8U9, H2QMJ5, H2Q2J4, etc.), rat collagen proteins (see e.g., UniProt accession numbers P02454, P02466, P13941, P02466, F1M6Q3, D3ZUL3, D3ZE04, D3ZE04, etc.), rabbit collagen proteins (see e.g., UniProt accession numbers G1T4A5, Q28668, G1T8J0, G1U9R7, G1T548, G1T380, G1T548, etc.) etc. Methods of identifying collagen protein homologs/orthologs from additional species are known to one of ordinary skill in the art, including, for example, using an amino acid sequence alignment program such as the BLAST® blastp suite or OrthoDB. In some embodiments, a collagen polypeptide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the collagen polypeptides described herein or known in the art.

In some embodiments, the present disclosure relates to one or more polynucleotides encoding a human collagen protein. Any suitable human collagen protein known in the art may be encoded by a polynucleotide of the present disclosure, including, for example, a Collagen alpha-1(I) chain polypeptide (COL1-1) (see e.g., UniProt accession number P02452; SEQ ID NO: 15), a Collagen alpha-2(I) chain polypeptide (COL1-2) (see e.g., UniProt accession number P08123; SEQ ID NO: 16), a Collagen alpha-1(II) chain polypeptide (COL2) (see e.g., UniProt accession number P02458), a Collagen alpha-1(III) chain polypeptide (COL3) (see e.g., UniProt accession number P2461; SEQ ID NO: 17), a Collagen alpha-1(IV) chain polypeptide (COL4-1) (see e.g., UniProt accession number P02462; SEQ ID NO: 18), a Collagen alpha-2(IV) chain polypeptide (COL4-2) (see e.g., UniProt accession number P08572), a Collagen alpha-3(IV) chain polypeptide (COL4-3) (see e.g., UniProt accession number Q01955), a Collagen alpha-4(IV) chain polypeptide (COL4-4) (see e.g., UniProt accession number P53420), a Collagen alpha-5(IV) chain polypeptide (COL4-5) (see e.g., UniProt accession number 29400), a Collagen alpha-6(IV) chain polypeptide (COL4-6) (see e.g., UniProt accession number Q14031), a Collagen alpha-1(V) chain polypeptide (COL5-1) (see e.g., UniProt accession number P20908), a Collagen alpha-2(V) chain polypeptide (COL5-2) (see e.g., UniProt accession number P05997), a Collagen alpha-3(V) chain polypeptide (COL5-3) (see e.g., UniProt accession number P25940), a Collagen alpha-1(VI) chain polypeptide (COL6-1) (see e.g., UniProt accession number P12109; SEQ ID NO: 19), a Collagen alpha-2(VI) chain polypeptide (COL6-2) (see e.g., UniProt accession number P12110), a Collagen alpha-3(VI) chain polypeptide (COL6-3) (see e.g., UniProt accession number P12111), a Collagen alpha-4(VI) chain polypeptide (COL6-4), a Collagen alpha-5(VI) chain polypeptide (COL6-5) (see e.g., UniProt accession number A8TX70), a Collagen alpha-6(VI) chain polypeptide (COL6-6) (see e.g., UniProt accession number A6NMZ7), a Collagen alpha-1(VII) chain polypeptide (COL7) (see e.g., UniProt accession number Q02388; SEQ ID NO: 20), a Collagen alpha-1(VIII) chain polypeptide (COL8) (see e.g., UniProt accession number P27658), a Collagen alpha-1(IX) chain polypeptide (COL9-1) (see e.g., UniProt accession number P20849), a Collagen alpha-2(IX) chain polypeptide (COL9-2) (see e.g., UniProt accession number Q14055), a Collagen alpha-3(IX) chain polypeptide (COL9-3) (see e.g., UniProt accession number Q14050), a Collagen alpha-1(X) chain polypeptide (COL10) (see e.g., UniProt accession number Q03692), a Collagen alpha-1(XI) chain polypeptide (COL11-1) (see e.g., UniProt accession number P12107), a Collagen alpha-2(XI) chain polypeptide (COL11-2) (see e.g., UniProt accession number P13942), a Collagen alpha-1(XII) chain polypeptide (COL12) (see e.g., UniProt accession number Q99715), a Collagen alpha-1(XIII) chain polypeptide (COL13) (see e.g., UniProt accession number Q5TAT6), a Collagen alpha-1(XIV) chain polypeptide (COL14) (see e.g., UniProt accession number Q05707), a Collagen alpha-1(XV) chain polypeptide (COL15) (see e.g., UniProt accession number P39059), a Collagen alpha-1(XVI) chain polypeptide (COL16) (see e.g., UniProt accession number Q07092), a Collagen alpha-1(XVII) chain polypeptide (COL17) (see e.g., UniProt accession number Q9UMD9; SEQ ID NO: 21), a Collagen alpha-1(XVIII) chain polypeptide (COL18) (see e.g., UniProt accession number P39060), a Collagen alpha-1(XIX) chain polypeptide (COL19) (see e.g., UniProt accession number Q14993), a Collagen alpha-1(XX) chain polypeptide (COL20) (see e.g., UniProt accession number Q9P218), a Collagen alpha-1(XXI) chain polypeptide (COL21) (see e.g., UniProt accession number Q96P44), a Collagen alpha-1(XXII) chain polypeptide (COL22) (see e.g., UniProt accession number Q8NFW1), a Collagen alpha-1(XXIII) chain polypeptide (COL23) (see e.g., UniProt accession number Q86Y22), a Collagen alpha-1(XXIV) chain polypeptide (COL24) (see e.g., UniProt accession number Q17RW2), a Collagen alpha-1(XXV) chain polypeptide (COL25) (see e.g., UniProt accession number Q9BXS0), a Collagen alpha-1(XXVI) chain polypeptide (COL26) (see e.g., UniProt accession number Q96A83), a Collagen alpha-1(XXVII) chain polypeptide (COL27) (see e.g., UniProt accession number Q8IZC6), a Collagen alpha-1(XXVIII) chain polypeptide (COL28) (see e.g., UniProt accession number Q2UY09), etc. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence encoding any of the human collagen polypeptides described herein or known in the art. Methods of identifying additional human collagen or collagen-like polypeptide homologs/orthologs are known to one of ordinary skill in the art, including, for example, using an amino acid sequence alignment program such as the BLAST® blastp suite or OrthoDB.

In some embodiments, a polynucleotide of the present disclosure encodes a human COL1-1 protein. In some embodiments, a polynucleotide encoding a COL1-1 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 15. In some embodiments, a polynucleotide encoding a human COL1-1 protein is a polynucleotide that encodes a polypeptide comprising the amino acid sequence of SEQ ID NO: 15.

In some embodiments, a polynucleotide encoding a COL1-1 protein is a polynucleotide that encodes an N-terminal truncation, a C-terminal truncation, or a fragment of the amino acid sequence of SEQ ID NO: 15. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, but fewer than 1464, consecutive amino acids of SEQ ID NO: 15.

In some embodiments, a polynucleotide of the present disclosure encodes a human COL1-2 protein. In some embodiments, a polynucleotide encoding a COL1-2 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 16. In some embodiments, a polynucleotide encoding a human COL1-2 protein is a polynucleotide that encodes a polypeptide comprising the amino acid sequence of SEQ ID NO: 16.

In some embodiments, a polynucleotide encoding a COL1-2 protein is a polynucleotide that encodes an N-terminal truncation, a C-terminal truncation, or a fragment of the amino acid sequence of SEQ ID NO: 16. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, but fewer than 1366, consecutive amino acids of SEQ ID NO: 16.

In some embodiments, a polynucleotide of the present disclosure encodes a human COL3 protein. In some embodiments, a polynucleotide encoding a COL3 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17. In some embodiments, a polynucleotide encoding a human COL3 protein is a polynucleotide that encodes a polypeptide comprising the amino acid sequence of SEQ ID NO: 17.

In some embodiments, a polynucleotide encoding a COL3 protein is a polynucleotide that encodes an N-terminal truncation, a C-terminal truncation, or a fragment of the amino acid sequence of SEQ ID NO: 17. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, but fewer than 1466, consecutive amino acids of SEQ ID NO: 17.

In some embodiments, a polynucleotide of the present disclosure encodes a human COL4-1 protein. In some embodiments, a polynucleotide encoding a COL4-1 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 18. In some embodiments, a polynucleotide encoding a human COL4-1 protein is a polynucleotide that encodes a polypeptide comprising the amino acid sequence of SEQ ID NO: 18.

In some embodiments, a polynucleotide encoding a COL4-1 protein is a polynucleotide that encodes an N-terminal truncation, a C-terminal truncation, or a fragment of the amino acid sequence of SEQ ID NO: 18. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, at least 1500, at least 1600, but fewer than 1669, consecutive amino acids of SEQ ID NO: 18.

In some embodiments, a polynucleotide of the present disclosure encodes a human COL6-1 protein. In some embodiments, a polynucleotide encoding a COL6-1 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 19. In some embodiments, a polynucleotide encoding a human COL6-1 protein is a polynucleotide that encodes a polypeptide comprising the amino acid sequence of SEQ ID NO: 19.

In some embodiments, a polynucleotide encoding a COL6-1 protein is a polynucleotide that encodes an N-terminal truncation, a C-terminal truncation, or a fragment of the amino acid sequence of SEQ ID NO: 19. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, but fewer than 1028, consecutive amino acids of SEQ ID NO: 19.

In some embodiments, a polynucleotide of the present disclosure encodes a human COL7 protein. In some embodiments, a polynucleotide encoding a COL7 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 20. In some embodiments, a polynucleotide encoding a human COL7 protein is a polynucleotide that encodes a polypeptide comprising the amino acid sequence of SEQ ID NO: 20.

In some embodiments, a polynucleotide encoding a COL7 protein is a polynucleotide that encodes an N-terminal truncation, a C-terminal truncation, or a fragment of the amino acid sequence of SEQ ID NO: 20. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2900, but fewer than 2944, consecutive amino acids of SEQ ID NO: 20.

In some embodiments, a polynucleotide of the present disclosure encodes a human COL17 protein. In some embodiments, a polynucleotide encoding a COL17 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 21. In some embodiments, a polynucleotide encoding a human COL17 protein is a polynucleotide that encodes a polypeptide comprising the amino acid sequence of SEQ ID NO: 21.

In some embodiments, a polynucleotide encoding a COL17 protein is a polynucleotide that encodes an N-terminal truncation, a C-terminal truncation, or a fragment of the amino acid sequence of SEQ ID NO: 21. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, but fewer than 1497, consecutive amino acids of SEQ ID NO: 21.

In some embodiments, one or more human collagen proteins of the present disclosure (e.g., a first human collagen protein, a further human collagen protein, an additional human collagen protein, and/or a second human collagen protein) comprise an amino acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21. In some embodiments, one or more human collagen proteins of the present disclosure (e.g., a first human collagen protein, a further human collagen protein, an additional human collagen protein, and/or a second human collagen protein) comprise a sequence selected from SEQ ID NOS: 15-21.

In some embodiments, one or more human collagen proteins of the present disclosure (e.g., a first human collagen protein, a further human collagen protein, an additional human collagen protein, and/or a second human collagen protein) comprise an amino acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-17. In some embodiments, one or more human collagen proteins of the present disclosure (e.g., a first human collagen protein, a further human collagen protein, an additional human collagen protein, and/or a second human collagen protein) comprise a sequence selected from SEQ ID NOS: 15-17.

### Fibronectin Proteins

In some embodiments, the present disclosure relates to one or more polynucleotides encoding a full-length fibronectin protein or any isoforms or portions thereof. Any fibronectin protein from any suitable species known in the art may be encoded by a polynucleotide of the present disclosure, including, for example, a human fibronectin protein (see e.g., UniProt accession number P02751), a mouse fibronectin protein (see, e.g., UniProt accession number P11276), a chimpanzee fibronectin protein (see e.g., UniProt accession number P11276), a rat fibronectin protein (see e.g., UniProt accession number P04937), a rabbit fibronectin protein (see e.g., UniProt accession number P04937), etc. Methods of identifying fibronectin protein homologs/orthologs from additional species are known to one of ordinary skill in the art. In some embodiments, a fibronectin protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the fibronectin proteins described herein or known in the art.

In some embodiments, a polynucleotide of the present disclosure encodes a human fibronectin protein. In some embodiments, a polynucleotide encoding a human fibronectin protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 53. In some embodiments, a polynucleotide encoding a human fibronectin protein is a polynucleotide that encodes a polypeptide comprising the amino acid sequence of SEQ ID NO: 53.

In some embodiments, a polynucleotide encoding a human fibronectin protein is a polynucleotide that encodes an N-terminal truncation, a C-terminal truncation, or a fragment of the amino acid sequence of SEQ ID NO: 53. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, but fewer than 2477, consecutive amino acids of SEQ ID NO: 53.

### Elastin and Associated Proteins

Elastic fibers in the extracellular matrix give elastic properties to the tissue. The elastic fibers generally contain two morphologically distinct components—the mature elastin fibers, and the micro-fibrils which mainly contain fibrillin and are associated with further proteins such as the micro-fibrils associated glycoproteins (MAGPs), fibulines, and the elastin-micro-fibrils-interface localized proteins (EMILIN). Elastin and its soluble precursor tropoelastin belong to the major structural proteins of the body.

In some embodiments, the present disclosure relates to one or more polynucleotides encoding an elastin or elastin-associated protein, including a tropoelastin, a fibrillin, a micro-fibrils associated glycoprotein, a fibuline, or an elastin-micro-fibrils-interface localized protein. In some embodiments, the present disclosure relates to one or more polynucleotides encoding a full-length elastin protein or any isoforms or portions thereof. Any elastin protein from any suitable species known in the art may be encoded by a polynucleotide of the present disclosure, including, for example, a human elastin protein (see e.g., UniProt accession number P15502), a mouse elastin protein (see, e.g., UniProt accession number P15502), a chimpanzee elastin protein (see e.g., UniProt accession number H2QUQ6), a rat elastin protein (see e.g., UniProt accession number Q99372), etc. Methods of identifying elastin protein homologs/orthologs from additional species are known to one of ordinary skill in the art. In some embodiments, an elastin protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the elastin proteins described herein or known in the art.

In some embodiments, a polynucleotide of the present disclosure encodes a human elastin protein. In some embodiments, a polynucleotide encoding a human elastin protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 54. In some embodiments, a polynucleotide encoding a human elastin protein is a polynucleotide that encodes a polypeptide comprising the amino acid sequence of SEQ ID NO: 54.

In some embodiments, a polynucleotide encoding a human elastin protein is a polynucleotide that encodes an N-terminal truncation, a C-terminal truncation, or a fragment of the amino acid sequence of SEQ ID NO: 54. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, but fewer than 786, consecutive amino acids of SEQ ID NO: 54.

### Lumican Proteins

In some embodiments, the present disclosure relates to one or more polynucleotides encoding a full-length lumican protein or any isoforms or portions thereof. Any lumican protein from any suitable species known in the art may be encoded by a polynucleotide of the present disclosure, including, for example, a human lumican protein (see e.g., UniProt accession number P51884), a mouse lumican protein (see, e.g., UniProt accession number P51885), a chimpanzee lumican protein (see e.g., UniProt accession number H2Q6L3), a rat lumican protein (see e.g., UniProt accession number H2Q6L3), a rabbit lumican protein (see e.g., UniProt accession number 046379), etc. Methods of identifying lumican protein homologs/orthologs from additional species are known to one of ordinary skill in the art. In some embodiments, a lumican protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the lumican proteins described herein or known in the art.

In some embodiments, a polynucleotide of the present disclosure encodes a human lumican protein. In some embodiments, a polynucleotide encoding a human lumican protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 55. In some embodiments, a polynucleotide encoding a human lumican protein is a polynucleotide that encodes a polypeptide comprising the amino acid sequence of SEQ ID NO: 55.

In some embodiments, a polynucleotide encoding a human lumican protein is a polynucleotide that encodes an N-terminal truncation, a C-terminal truncation, or a fragment of the amino acid sequence of SEQ ID NO: 55. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, but fewer than 338, consecutive amino acids of SEQ ID NO: 55.

### Vitronectin and Vitronectin Receptor Proteins

In some embodiments, the present disclosure relates to one or more polynucleotides encoding a full-length vitronectin or vitronectin receptor protein or any isoforms or portions thereof. Any vitronectin or vitronectin receptor protein from any suitable species known in the art may be encoded by a polynucleotide of the present disclosure, including, for example, a human vitronectin or vitronectin receptor protein (see e.g., UniProt accession numbers P04004 and P06756), a mouse vitronectin or vitronectin receptor protein (see, e.g., UniProt accession numbers P29788 and P43406), a chimpanzee vitronectin or vitronectin receptor protein (see e.g., UniProt accession numbers H2QCH3 and H2R6C3), a rat vitronectin or vitronectin receptor protein (see e.g., UniProt accession number Q7TQ11), a rabbit vitronectin or vitronectin receptor protein (see e.g., UniProt accession number P22458), etc. Methods of identifying vitronectin or vitronectin receptor protein homologs/orthologs from additional species are known to one of ordinary skill in the art. In some embodiments, a vitronectin or vitronectin receptor protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the vitronectin or vitronectin receptor proteins described herein or known in the art.

In some embodiments, a polynucleotide of the present disclosure encodes a human vitronectin protein. In some embodiments, a polynucleotide encoding a human vitronectin protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 56. In some embodiments, a polynucleotide encoding a human vitronectin protein is a polynucleotide that encodes a polypeptide comprising the amino acid sequence of SEQ ID NO: 56.

In some embodiments, a polynucleotide encoding a human vitronectin protein is a polynucleotide that encodes an N-terminal truncation, a C-terminal truncation, or a fragment of the amino acid sequence of SEQ ID NO: 56. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, but fewer than 478, consecutive amino acids of SEQ ID NO: 56.

### Laminin Proteins

In some embodiments, the present disclosure relates to one or more polynucleotides encoding a full-length laminin protein or any isoforms or portions thereof. Any laminin protein from any suitable species known in the art may be encoded by a polynucleotide of the present disclosure, including, for example, a human laminin protein (see e.g., UniProt accession numbers P25391, P24043, Q16787, Q16363, 015230, P07942, P55268, Q13751, P11047, Q13753, and Q9Y6N6), a mouse laminin protein (see e.g., UniProt accession numbers Q61789, Q61087, and Q61092), a chimpanzee laminin protein (see e.g., UniProt accession numbers H2QEC7, H2R041, and H2Q0R2), a rat laminin protein (see e.g., UniProt accession numbers D3ZN05, F1LPI5, and F1LRH4), a rabbit laminin protein (see e.g., UniProt accession numbers G1SY40 and A0A0B5JSH0), etc. Methods of identifying laminin protein homologs/orthologs from additional species are known to one of ordinary skill in the art. In some embodiments, a laminin protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the laminin proteins described herein or known in the art.

In some embodiments, a polynucleotide of the present disclosure encodes a human laminin protein, such as a human Laminin subunit alpha-1 (LamA1) polypeptide (see e.g., UniProt accession number P25391), a human Laminin subunit alpha-2 (LamA2) polypeptide (see e.g., UniProt accession number P24043), a human Laminin subunit alpha-3 (LamA3) polypeptide (see e.g., UniProt accession number Q16787), a human Laminin subunit alpha-4 (LamA4) polypeptide (see e.g., UniProt accession number Q16363), a human Laminin subunit alpha-5 (LamA5) polypeptide (see e.g., UniProt accession number 015230), a human Laminin subunit beta-1 (LamB1) polypeptide (see e.g., UniProt accession number P07942), a human Laminin subunit beta-2 (LamB2) polypeptide (see e.g., UniProt accession number P55268), a human Laminin subunit beta-3 (LamB3) polypeptide (see e.g., UniProt accession number Q13751), a human Laminin subunit gamma-1 (LamC1) polypeptide (see e.g., UniProt accession number P11047), a human Laminin subunit gamma-2 (LamC2) polypeptide (see e.g., UniProt accession number Q13753), a human Laminin subunit gamma-3 (LamC3) polypeptide (see e.g., UniProt accession number Q9Y6N6), etc.

In some embodiments, a polynucleotide of the present disclosure encodes a human LamA3 polypeptide. In some embodiments, a polynucleotide encoding a human LamA3 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 57. In some embodiments, a polynucleotide encoding a human LamA3 polypeptide is a polynucleotide that encodes a polypeptide comprising the amino acid sequence of SEQ ID NO: 57.

In some embodiments, a polynucleotide encoding a human LamA3 polypeptide is a polynucleotide that encodes an N-terminal truncation, a C-terminal truncation, or a fragment of the amino acid sequence of SEQ ID NO: 57. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 750, at least 1000, at least 1250, at least 1500, at least 1750, at least 2000, at least 2250, at least 2500, at least 2750, at least 3000, at least 3250, but fewer than 3333, consecutive amino acids of SEQ ID NO: 57.

In some embodiments, a polynucleotide of the present disclosure encodes a human LamB3 polypeptide. In some embodiments, a polynucleotide encoding a human LamB3 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 58. In some embodiments, a polynucleotide encoding a human LamB3 polypeptide is a polynucleotide that encodes a polypeptide comprising the amino acid sequence of SEQ ID NO: 58.

In some embodiments, a polynucleotide encoding a LamB3 polypeptide is a polynucleotide that encodes an N-terminal truncation, a C-terminal truncation, or a fragment of the amino acid sequence of SEQ ID NO: 58. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, but fewer than 1172, consecutive amino acids of SEQ ID NO: 58.

In some embodiments, a polynucleotide of the present disclosure encodes a human LamC2 polypeptide. In some embodiments, a polynucleotide encoding a human LamC2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 59. In some embodiments, a polynucleotide encoding a human LamC2 polypeptide is a polynucleotide that encodes a polypeptide comprising the amino acid sequence of SEQ ID NO: 59.

In some embodiments, a polynucleotide encoding a LamC2 polypeptide is a polynucleotide that encodes an N-terminal truncation, a C-terminal truncation, or a fragment of the amino acid sequence of SEQ ID NO: 59. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, but fewer than 1193, consecutive amino acids of SEQ ID NO: 59.

### Neuromodulator Proteins

In some embodiments, the present disclosure relates to one or more polynucleotides encoding a full-length neuromodulator protein or any isoforms or portions thereof. Any neuromodulator protein from any suitable species known in the art may be encoded by a polynucleotide of the present disclosure, including, for example, a Clostridium botulinum protein (see e.g., UniProt accession numbers P0DPI0, Q45894, P0DPI1, P10844, and B1INP5), etc. Methods of identifying neuromodulator protein homologs/orthologs from additional species are known to one of ordinary skill in the art. In some embodiments, a neuromodulator protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the neuromodulator proteins described herein or known in the art.

In some embodiments, a polynucleotide of the present disclosure encodes a Clostridium botulinum neuromodulator protein.

In some embodiments, a polynucleotide of the present disclosure encodes a Clostridium botulinum neurotoxin type A protein In some embodiments, a polynucleotide encoding a Clostridium botulinum neurotoxin type A protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 60. In some embodiments, a polynucleotide encoding a Clostridium botulinum neurotoxin type A protein is a polynucleotide that encodes a polypeptide comprising the amino acid sequence of SEQ ID NO: 60. In some embodiments, a Clostridium botulinum neurotoxin type A protein of the present disclosure comprises an alanine to valine mutation at a position corresponding to position 27 of SEQ ID NO: 60.

In some embodiments, a polynucleotide encoding a Clostridium botulinum neurotoxin type A protein is a polynucleotide that encodes an N-terminal truncation, a C-terminal truncation, or a fragment of the amino acid sequence of SEQ ID NO: 60. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, but fewer than 1296, consecutive amino acids of SEQ ID NO: 60.

In some embodiments, a polynucleotide of the present disclosure encodes a Clostridium botulinum neurotoxin type B protein In some embodiments, a polynucleotide encoding a Clostridium botulinum neurotoxin type B protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 61. In some embodiments, a polynucleotide encoding a Clostridium botulinum neurotoxin type B protein is a polynucleotide that encodes a polypeptide comprising the amino acid sequence of SEQ ID NO: 61.

In some embodiments, a polynucleotide encoding a Clostridium botulinum neurotoxin type B protein is a polynucleotide that encodes an N-terminal truncation, a C-terminal truncation, or a fragment of the amino acid sequence of SEQ ID NO: 61. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1100, at least 1200, but fewer than 1291, consecutive amino acids of SEQ ID NO: 61.

### Fibrillin Proteins

In some embodiments, the present disclosure relates to one or more polynucleotides encoding a full-length fibrillin protein or any isoforms or portions thereof. Any fibrillin protein from any suitable species known in the art may be encoded by a polynucleotide of the present disclosure, including, for example, a human fibrillin protein (see e.g., UniProt accession numbers P35555, P35556, and Q75N90), a mouse fibrillin protein (see, e.g., UniProt accession numbers Q61554 and Q61555), a chimpanzee fibrillin protein (see e.g., UniProt accession numbers A0A2I3RTE4 and K7CZX0), a rat fibrillin protein (see e.g., UniProt accession number G3V9M6 and F1M5Q4), a rabbit fibrillin protein (see e.g., UniProt accession number G1SKM2, G1SUS5, and G1T1H4), etc. Methods of identifying fibrillin protein homologs/orthologs from additional species are known to one of ordinary skill in the art. In some embodiments, a fibrillin protein of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the fibrillin proteins described herein or known in the art.

In some embodiments, a polynucleotide of the present disclosure encodes a human fibrillin protein.

In some embodiments, a polynucleotide of the present disclosure encodes a human fibrillin-1 protein. In some embodiments, a polynucleotide encoding a human fibrillin-1 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 62. In some embodiments, a polynucleotide encoding a human fibrillin-1 protein is a polynucleotide that encodes a polypeptide comprising the amino acid sequence of SEQ ID NO: 62.

In some embodiments, a polynucleotide encoding a human fibrillin-1 protein is a polynucleotide that encodes an N-terminal truncation, a C-terminal truncation, or a fragment of the amino acid sequence of SEQ ID NO: 62. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 750, at least 1000, at least 1250, at least 1500, at least 1750, at least 2000, at least 2250, at least 2500, at least 2750, but fewer than 2871, consecutive amino acids of SEQ ID NO: 62.

In some embodiments, a polynucleotide of the present disclosure encodes a human fibrillin-2 protein. In some embodiments, a polynucleotide encoding a human fibrillin-2 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 63. In some embodiments, a polynucleotide encoding a human fibrillin-2 protein is a polynucleotide that encodes a polypeptide comprising the amino acid sequence of SEQ ID NO: 63.

In some embodiments, a polynucleotide encoding a human fibrillin-2 protein is a polynucleotide that encodes an N-terminal truncation, a C-terminal truncation, or a fragment of the amino acid sequence of SEQ ID NO: 63. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 750, at least 1000, at least 1250, at least 1500, at least 1750, at least 2000, at least 2250, at least 2500, at least 2750, but fewer than 2912, consecutive amino acids of SEQ ID NO: 63.

In some embodiments, a polynucleotide of the present disclosure encodes a human fibrillin-3 protein. In some embodiments, a polynucleotide encoding a human fibrillin-3 protein is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 64. In some embodiments, a polynucleotide encoding a human fibrillin-3 protein is a polynucleotide that encodes a polypeptide comprising the amino acid sequence of SEQ ID NO: 64.

In some embodiments, a polynucleotide encoding a human fibrillin-3 protein is a polynucleotide that encodes an N-terminal truncation, a C-terminal truncation, or a fragment of the amino acid sequence of SEQ ID NO: 64. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 750, at least 1000, at least 1250, at least 1500, at least 1750, at least 2000, at least 2250, at least 2500, at least 2750, but fewer than 2809, consecutive amino acids of SEQ ID NO: 64.

### Exemplary Cosmetic Polypeptides

In some embodiments, one or more cosmetic proteins of the present disclosure (e.g., a first cosmetic protein, a further cosmetic protein, an additional cosmetic protein, and/or a second cosmetic protein) comprise an amino acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21 or 53-64. In some embodiments, one or more cosmetic proteins of the present disclosure (e.g., a first cosmetic protein, a further cosmetic protein, an additional cosmetic protein, and/or a second cosmetic protein) comprises a sequence selected from SEQ ID NOS: 15-21 or 53-64.

In some embodiments, one or more cosmetic proteins of the present disclosure (e.g., a first cosmetic protein, a further cosmetic protein, an additional cosmetic protein, and/or a second cosmetic protein) comprise an amino acid sequence comprising at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 15-21, 53-54, or 57-59. In some embodiments, one or more cosmetic proteins of the present disclosure (e.g., a first cosmetic protein, a further cosmetic protein, an additional cosmetic protein, and/or a second cosmetic protein) comprise a sequence selected from SEQ ID NOS: 15-21, 53-54, or 57-59.

### First Polynucleotides

In some embodiments, the present disclosure relates to a recombinant nucleic acid comprising a first polynucleotide encoding a first polypeptide comprising a first cosmetic protein. The first cosmetic protein may be any of the cosmetic proteins described herein or known in the art, including, for example a collagen protein, a fibronectin, an elastin, a lumican, a vitronectin/vitronectin receptor, a laminin, a neuromodulator, a fibrillin, etc. In some embodiments, the first cosmetic protein is a structural extracellular matrix protein (e.g., a collagen, elastin, fibronectin, laminin, fibrillin, etc.). In some embodiments, the first cosmetic protein is a collagen, elastin, fibronectin, or laminin protein (e.g., a human collagen, elastin, fibronectin, or laminin protein).

In some embodiments, a recombinant nucleic acid of the present disclosure comprises one copy of the first polynucleotide. In some embodiments, a recombinant nucleic acid of the present disclosure comprises two or more (e.g., two or more, three or more, four or more, five or more, ten or more, etc.) copies of the first polynucleotide. In some embodiments, a recombinant nucleic acid of the present disclosure comprises two copies of the first polynucleotide.

In some embodiments, the first cosmetic protein is a first human collagen protein. The first human collagen protein may be any of the human collagen proteins described herein or known in the art. In some embodiments, the first human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, or COL28. In some embodiments, the first human collagen protein is selected from COL1-1, COL1-2, COL3, COL4-1, COL4-2, COL6-1, COL7, or COL17. In some embodiments, the first human collagen protein is COL1-1. In some embodiments, the first human collagen protein is COL1-2. In some embodiments, the first human collagen protein is COL3. In some embodiments, the first human collagen protein is COL4-1. In some embodiments, the first human collagen protein is COL4-2. In some embodiments, the first human collagen protein is COL6-1. In some embodiments, the first human collagen protein is COL7. In some embodiments, the first human collagen protein is not COL7. In some embodiments, the first human collagen protein is COL17.

In some embodiments, the first polypeptide consists essentially of the first cosmetic protein. In some embodiments, the first polypeptide consists of the first cosmetic protein. In some embodiments, the first polypeptide is the first cosmetic protein.

### Chimeric Polypeptides

In some embodiments, the first polypeptide is a chimeric polypeptide comprising the first cosmetic protein. In some embodiments, the first polypeptide is a chimeric polypeptide comprising the first cosmetic protein and a further cosmetic protein. In some embodiments, the chimeric polypeptide comprises a linker polypeptide linking the first cosmetic protein and the further cosmetic protein. In some embodiments, the chimeric polypeptide comprises, from n-terminus to c-terminus, the first cosmetic protein—the linker polypeptide—the further cosmetic protein. The first and/or further cosmetic proteins may be any of the cosmetic proteins described herein or known in the art, including, for example a collagen protein, a fibronectin, an elastin, a lumican, a vitronectin/vitronectin receptor, a laminin, a neuromodulator, a fibrillin, etc. In some embodiments, the first and/or further cosmetic protein is a structural extracellular matrix protein (e.g., a collagen, elastin, fibronectin, laminin, fibrillin, etc.). In some embodiments, the first and/or further cosmetic protein is a collagen, elastin, fibronectin, or laminin protein (e.g., a human collagen, elastin, fibronectin, or laminin protein). In some embodiments, the first and further cosmetic proteins are the same. In some embodiments, the first and further cosmetic proteins are different.

In some embodiments, the linker polypeptide is a cleavable linker polypeptide. Any cleavable linker polypeptide known in the art may be used in the chimeric polypeptides of the present disclosure, including, for example, a T2A linker, a P2A linker, a E2A linker, and F2A linker, etc. In some embodiments, the linker polypeptide is a T2A linker polypeptide. An exemplary nucleic acid sequence encoding a T2A linker polypeptide is provided as SEQ ID NO: 24. An exemplary amino acid sequence of a T2A linker polypeptide is provided as SEQ ID NO: 28. In some embodiments, the linker polypeptide is a P2A linker polypeptide. An exemplary nucleic acid sequence encoding a P2A linker polypeptide is provided as SEQ ID NO: 25. An exemplary amino acid sequence of a P2A linker polypeptide is provided as SEQ ID NO: 29. In some embodiments, the linker polypeptide is an E2A linker polypeptide. An exemplary nucleic acid sequence encoding an E2A linker polypeptide is provided as SEQ ID NO: 26. An exemplary amino acid sequence of an E2A linker polypeptide is provided as SEQ ID NO: 30. In some embodiments, the linker polypeptide is an F2A linker polypeptide. An exemplary nucleic acid sequence encoding an F2A linker polypeptide is provided as SEQ ID NO: 27. An exemplary amino acid sequence of an F2A linker polypeptide is provided as SEQ ID NO: 31.

In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 28-31. In some embodiments, the linker polypeptide comprises a sequence selected from SEQ ID NOS: 28-31.

In some embodiments, the first cosmetic protein is a first collagen protein (e.g., a first human collagen protein), and the further cosmetic protein is a further collagen protein (e.g., a further human collagen protein). An exemplary nucleic acid sequence encoding a chimeric polypeptide comprising a first human collagen protein, a linker polypeptide, and a further human collagen protein is provided as SEQ ID NO: 32.

In some embodiments, the first cosmetic protein is a first human collagen protein, and the further cosmetic protein is a further human collagen protein. The further human collagen protein may be any of the human collagen proteins described herein or known in the art. In some embodiments, the further human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, or COL28. In some embodiments, the further human collagen protein is selected from COL1-1, COL1-2, COL3, COL4-1, COL4-2, COL6-1, COL7, or COL17. In some embodiments, the further human collagen protein is COL1-1. In some embodiments, the further human collagen protein is COL1-2. In some embodiments, the further human collagen protein is COL3. In some embodiments, the further human collagen protein is COL4-1. In some embodiments, the further human collagen protein is COL4-2. In some embodiments, the further human collagen protein is COL6-1. In some embodiments, the further human collagen protein is COL7. In some embodiments, the further human collagen protein is not COL7. In some embodiments, the further human collagen protein is COL17. In some embodiments, the first human collagen protein and the further human collagen protein are the same. In some embodiments, the first human collagen protein and the further human collagen protein are different.

In some embodiments, the first human collagen protein is COL1-1, and the further human collagen protein is selected from COL1-2, COL3, COL4-1, COL4-2, COL6-1, COL7, or COL17. In some embodiments, the first human collagen protein is COL1-1, and the further human collagen protein is COL1-2. In some embodiments, the first human collagen protein is COL1-1, and the further human collagen protein is COL3.

In some embodiments, the first human collagen protein is COL1-2, and the further human collagen protein is COL1-1, COL3, COL4-1, COL4-2, COL6-1, COL7, or COL17. In some embodiments, the first human collagen protein is COL1-2, and the further human collagen protein is COL1-1.

In some embodiments, the first human collagen protein is COL3, and the further human collagen protein is selected from COL1-1, COL1-2, COL4-1, COL4-2, COL6-1, COL7, or COL17.

In some embodiments, the first human collagen protein is COL4-1, and the further human collagen protein is COL1-1, COL1-2, COL3, COL4-2, COL6-1, COL7, or COL17. In some embodiments, the first human collagen protein is COL4-1, and the further human collagen protein is COL4-2.

In some embodiments, the first human collagen protein is COL6-1, and the further human collagen protein is selected from COL1-1, COL1-2, COL3, COL4-1, COL4-2, COL7, or COL17.

In some embodiments, the first human collagen protein is COL7, and the further human collagen protein is COL1-1, COL1-2, COL3, COL4-1, COL4-2, COL6-1, or COL17.

In some embodiments, the first human collagen protein is COL17, and the further human collagen protein is COL1-1, COL1-2, COL3, COL4-1, COL4-2, COL6-1, or COL7.

In some embodiments, the first cosmetic protein is a first laminin protein (e.g., a first human laminin protein), and the further cosmetic protein is a further laminin protein (e.g., a further human laminin protein). In some embodiments, the first cosmetic protein is a first human laminin protein, and the further cosmetic protein is a further human laminin protein. The further human laminin protein may be any of the human laminin proteins described herein or known in the art. In some embodiments, the first human laminin protein is a human LamA3 polypeptide and the further human laminin protein is a human LamB3 polypeptide. In some embodiments, the first human laminin protein is a human LamA3 polypeptide and the further human laminin protein is a human LamC2 polypeptide. In some embodiments, the first human laminin protein is a human LamB3 polypeptide and the further human laminin protein is a human LamC2 polypeptide.

In some embodiments, the first polynucleotide encodes a monocistronic mRNA. In some embodiments, the monocistronic mRNA comprises an open reading frame (ORF) encoding the first polypeptide.

In some embodiments, the first polynucleotide encodes a polycistronic mRNA. In some embodiments, the polycistronic mRNA comprises an open reading frame (ORF) encoding the first polypeptide.

Polycistronic mRNA

In some embodiments, the first polynucleotide encodes a polycistronic mRNA. In some embodiments, the polycistronic mRNA comprises an open reading frame (ORF) encoding the first polypeptide. In some embodiments, the first polynucleotide encodes a polycistronic mRNA comprising: 1) a first open reading frame (ORF) encoding the first polypeptide, and 2) a second open reading frame (ORF) encoding an additional cosmetic protein. In some embodiments, the polycistronic mRNA further comprises an internal ribosomal entry site (IRES) separating the first ORF and the second ORF. In some embodiments, the polycistronic mRNA comprises, from 5′ to 3′, the first ORF encoding the first polypeptide—the IRES—the second ORF encoding the additional cosmetic protein. The first polypeptide may be any of the first polypeptides described herein. The additional cosmetic protein may be any of the cosmetic proteins described herein or known in the art, including, for example a collagen protein, a fibronectin, an elastin, a lumican, a vitronectin/vitronectin receptor, a laminin, a neuromodulator, a fibrillin, etc. In some embodiments, the additional cosmetic protein is a structural extracellular matrix protein (e.g., a collagen, elastin, fibronectin, laminin, fibrillin, etc.). In some embodiments, the additional cosmetic protein is a collagen, elastin, fibronectin, or laminin protein (e.g., a human collagen, elastin, fibronectin, or laminin protein).

Any suitable IRES known in the art may be used in the polycistronic mRNAs of the present disclosure, including, for example, a virally-derived IRES (e.g. an IRES derived from a poliovirus, rhinovirus, encephalomyocarditis virus (EMCV), foot-and-mouth disease virus, hepatitis C virus, classic swine fever virus, rous sarcoma virus, human immunodeficiency virus, cricket paralysis virus, Kaposi's sarcoma-associated herpesvirus, etc.), a cellular mRNA-derived IRES (e.g. an IRES derived from growth factor mRNAs, such as fibroblast growth factor 2, platelet-derived growth factor B, and vascular endothelial growth factor; an IRES derived from transcription factor mRNAs, such as antennapedia, ultrabithorax, and NF-κB repressing factor; an IRES derived from oncogene mRNAs, such as c-myc, pim-1, and protein kinase p58^{PITSLRE}, etc.), a synthetic IRES (e.g., a CP148 IRES), and others (see e.g., Mokrejs et al. (2007) A Bioinformatical Approach to the Analysis of Viral and Cellular Internal Ribosome Entry Sites. Columbus F editors. New Messenger RNA Research Communications. Hauppauge, NY: Nova Science Publishers; pp. 133-166). In some embodiments, the IRES is a CP148 IRES. An exemplary nucleic acid sequence encoding a CP148 IRES is provided as SEQ ID NO: 22. In some embodiments, the IRES is an EMCV IRES. An exemplary nucleic acid sequence encoding an EMCV IRES is provided as SEQ ID NO: 23.

In some embodiments, the nucleic acid sequence encoding the IRES comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23. In some embodiments, the nucleic acid sequence encoding the IRES comprises the sequence of SEQ ID NO: 22 or SEQ ID NO: 23.

In some embodiments, the first polypeptide is a first collagen protein (e.g., a first human collagen protein), and the additional cosmetic protein is an additional collagen protein (e.g., an additional human collagen protein). An exemplary nucleic acid encoding a polycistronic mRNA comprising a first ORF, an IRES, and second ORF is provided as SEQ ID NO: 33 or SEQ ID NO: 34. The additional human collagen protein may be any of the human collagen proteins described herein. In some embodiments, the additional human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, or COL28. In some embodiments, the additional human collagen protein is selected from COL1-1, COL1-2, COL3, COL4-1, COL4-2, COL6-1, COL7, or COL17. In some embodiments, the additional human collagen protein is COL1-1. In some embodiments, the additional human collagen protein is COL1-2. In some embodiments, the additional human collagen protein is COL3. In some embodiments, the additional human collagen protein is COL4-1. In some embodiments, the additional human collagen protein is COL4-2. In some embodiments, the additional human collagen protein is COL6-1. In some embodiments, the additional human collagen protein is COL7. In some embodiments, the additional human collagen protein is not COL7. In some embodiments, the additional human collagen protein is COL17. In some embodiments, the first human collagen protein and the additional human collagen protein are the same. In some embodiments, the first human collagen protein and the additional human collagen protein are different.

In some embodiments, the first human collagen protein is COL1-1, and the additional human collagen protein is selected from COL1-2, COL3, COL4-1, COL4-2, COL6-1, COL7, or COL17. In some embodiments, the first human collagen protein is COL1-1, and the additional human collagen protein is COL1-2. In some embodiments, the first human collagen protein is COL1-1, and the additional human collagen protein is COL3.

In some embodiments, the first human collagen protein is COL1-2, and the additional human collagen protein is selected from COL1-1, COL3, COL4-1, COL4-2, COL6-1, COL7, or COL17. In some embodiments, the first human collagen protein is COL1-2, and the additional human collagen protein is COL1-1.

In some embodiments, the first human collagen protein is COL3, and the additional human collagen protein is selected from COL1-1, COL1-2, COL4-1, COL4-2, COL6-1, COL7, or COL17.

In some embodiments, the first human collagen protein is COL4-1, and the additional human collagen protein is selected from COL1-1, COL1-2, COL3, COL4-2, COL6-1, COL7, or COL17. In some embodiments, the first human collagen protein is COL4-1, and the additional human collagen protein is COL4-2.

In some embodiments, the first human collagen protein is COL6-1, and the additional human collagen protein is selected from COL1-1, COL1-2, COL3, COL4-1, COL4-2, COL7, or COL17.

In some embodiments, the first human collagen protein is COL7, and the additional human collagen protein is selected from COL1-1, COL1-2, COL3, COL4-1, COL4-2, COL6-1, or COL17.

In some embodiments, the first human collagen protein is COL17, and the additional human collagen protein is selected from COL1-1, COL1-2, COL3, COL4-1, COL4-2, COL6-1, or COL7.

In some embodiments, the first polypeptide is a first collagen protein (e.g., a first human collagen protein), and the additional cosmetic protein is an additional collagen protein (e.g., an additional human collagen protein).

In some embodiments, the first polypeptide is a first laminin protein (e.g., a first human laminin protein), and the additional cosmetic protein is an additional laminin protein (e.g., an additional human laminin protein). In some embodiments, the first polypeptide is a first human laminin protein, and the additional cosmetic protein is an additional human laminin protein. The additional human laminin protein may be any of the human laminin proteins described herein or known in the art. In some embodiments, the first human laminin protein is a human LamA3 polypeptide and the additional human laminin protein is a human LamB3 polypeptide. In some embodiments, the first human laminin protein is a human LamA3 polypeptide and the additional human laminin protein is a human LamC2 polypeptide. In some embodiments, the first human laminin protein is a human LamB3 polypeptide and the additional human laminin protein is a human LamC2 polypeptide.

### Second Polynucleotides

In some embodiments, the present disclosure relates to a recombinant nucleic acid further comprising a second polynucleotide encoding a second cosmetic protein. The second cosmetic protein may be any of the cosmetic proteins described herein or known in the art, including, for example a collagen protein, a fibronectin, an elastin, a lumican, a vitronectin/vitronectin receptor, a laminin, a neuromodulator, a fibrillin, etc. In some embodiments, the second cosmetic protein is a structural extracellular matrix protein (e.g., a collagen, elastin, fibronectin, laminin, fibrillin, etc.). In some embodiments, the second cosmetic protein is a collagen, elastin, fibronectin, or laminin protein (e.g., a human collagen, elastin, fibronectin, or laminin protein). In some embodiments, the first and second cosmetic proteins are the same. In some embodiments, the first and second cosmetic proteins are different. In some embodiments, the recombinant nucleic acid comprises one copy of the second polynucleotide. In some embodiments, the recombinant nucleic acid comprises two or more (e.g., two or more, three or more, four or more, five or more, ten or more, etc.) copies of the second polynucleotide. In some embodiments, the recombinant nucleic acid comprises two copies of the second polynucleotide.

In some embodiments, the second cosmetic protein is a collagen protein. In some embodiments, the second cosmetic protein is a second human collagen protein. The second human collagen protein may be any of the human collagen proteins described herein. In some embodiments, the second human collagen protein is selected from COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, or COL28. In some embodiments, the second human collagen protein is selected from COL1-1, COL1-2, COL3, COL4-1, COL6-1, COL7, or COL17. In some embodiments, the second human collagen protein is COL1-1. In some embodiments, the second human collagen protein is COL1-2. In some embodiments, the second human collagen protein is COL3. In some embodiments, the second human collagen protein is COL4-1. In some embodiments, the second human collagen protein is COL4-2. In some embodiments, the second human collagen protein is COL6-1. In some embodiments, the second human collagen protein is COL7. In some embodiments, the second human collagen protein is not COL7. In some embodiments, the second human collagen protein is COL17.

In some embodiments, the first polynucleotide encodes a first collagen protein and the second polynucleotide encodes a second collagen protein. In some embodiments, the first polynucleotide encodes a first human collagen protein and the second polynucleotide encodes a second human collagen protein. In some embodiments, the first human collagen protein (encoded by the first polynucleotide) and the second human collagen protein (encoded by the second polynucleotide) are the same. In some embodiments, the first human collagen protein (encoded by the first polynucleotide) and the second human collagen protein (encoded by the second polynucleotide) are different.

In some embodiments, the first human collagen protein is COL1-1, and the second human collagen protein is selected from COL1-2, COL3, COL4-1, COL4-2, COL6-1, COL7, or COL17. In some embodiments, the first human collagen protein is COL1-1, and the second human collagen protein is COL1-2. In some embodiments, the first human collagen protein is COL1-1, and the second human collagen protein is COL3.

In some embodiments, the first human collagen protein is COL1-2, and the second human collagen protein is selected from COL1-1, COL3, COL4-1, COL4-2, COL6-1, COL7, or COL17. In some embodiments, the first human collagen protein is COL1-2, and the second human collagen protein is COL1-1.

In some embodiments, the first human collagen protein is COL3, and the second human collagen protein is selected from COL1-1, COL1-2, COL4-1, COL4-2, COL6-1, COL7, or COL17.

In some embodiments, the first human collagen protein is COL4-1, and the second human collagen protein is selected from COL1-2, COL1-2, COL3, COL4-2, COL6-1, COL7, or COL17. In some embodiments, the first human collagen protein is COL4-1, and the second human collagen protein is COL4-2.

In some embodiments, the first human collagen protein is COL6-1, and the second human collagen protein is selected from COL1-1, COL1-2, COL3, COL4-1, COL4-2, COL7, or COL17.

In some embodiments, the first human collagen protein is COL7, and the second human collagen protein is selected from COL1-1, COL1-2, COL3, COL4-1, COL4-2, COL6-1, or COL17.

In some embodiments, the first human collagen protein is COL17, and the second human collagen protein is selected from COL1-1, COL1-2, COL3, COL4-1, COL4-2, COL6-1, or COL7.

In some embodiments, the first polynucleotide encodes a first laminin protein (e.g., a first human laminin protein), and the second polynucleotide encodes a second laminin protein (e.g., a second human laminin protein). In some embodiments, the first polynucleotide encodes a first human laminin polypeptide and the second polynucleotide encodes a second human laminin protein. The second human laminin protein may be any of the human laminin proteins described herein or known in the art. In some embodiments, the first human laminin protein is a human LamA3 polypeptide and the second human laminin protein is a human LamB3 polypeptide. In some embodiments, the first human laminin protein is a human LamA3 polypeptide and the second human laminin protein is a human LamC2 polypeptide. In some embodiments, the first human laminin protein is a human LamB3 polypeptide and the second human laminin protein is a human LamC2 polypeptide

### Recombinant Nucleic Acids

In some embodiments, the present disclosure relates to recombinant nucleic acids comprising any one or more of the polynucleotides described herein. In some embodiments, the recombinant nucleic acid comprises one copy of the first polynucleotide. In some embodiments, the recombinant nucleic acid comprises two copies of the first polynucleotide. In some embodiments, the recombinant nucleic acid comprises one copy of the first polynucleotide and one copy of the second polynucleotide. In some embodiments, the recombinant nucleic acid comprises one copy of the first polynucleotide and two copies of the second polynucleotide. In some embodiments, the recombinant nucleic acid comprises two copies of the first polynucleotide and one copy of the second polynucleotide. In some embodiments, the recombinant nucleic acid comprises two copies of the first polynucleotide and two copies of the second polynucleotide.

In some embodiments, the recombinant nucleic acid is a vector (e.g., an expression vector, a display vector, etc.). In some embodiments, the vector is a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, Sindbis-viral vectors, measles vectors, herpes viral vectors, lentiviral vectors, retroviral vectors, etc.). In some embodiments, the vector is a herpes viral vector. In some embodiments, the vector is capable of autonomous replication in a host cell. In some embodiments, the vector is incapable of autonomous replication in a host cell. In some embodiments, the vector can integrate into a host DNA. In some embodiments, the vector cannot integrate into a host DNA (e.g., is episomal). Methods of making vectors containing one or more polynucleotides of interest are well known to one of ordinary skill in the art, including, for example, by chemical synthesis, or by artificial manipulation of isolated segments of nucleic acids (e.g., by genetic engineering techniques).

In some embodiments, a recombinant nucleic acid of the present disclosure is a herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, a recombinant nucleic acid of the present disclosure is a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any combinations or any derivatives thereof. In some embodiments, the recombinant herpes virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes virus genes. In some embodiments, the recombinant herpes virus genome is attenuated (e.g., as compared to a corresponding, wild-type herpes virus genome). In some embodiments, the recombinant herpes virus genome is replication-competent. In some embodiments, the recombinant herpes virus genome is replication-defective

In some embodiments, the recombinant nucleic acid is a recombinant herpes simplex virus (HSV) genome. In some embodiments, the recombinant herpes simplex virus genome is a recombinant type 1 herpes simplex virus (HSV-1) genome, a recombinant type 2 herpes simplex virus (HSV-2) genome, or any derivatives thereof. In some embodiments, the recombinant herpes simplex virus genome is a recombinant HSV-1 genome. In some embodiments, the recombinant herpes simplex virus genome is replication-competent. In some embodiments, the recombinant herpes simplex virus genome is replication-defective. In some embodiments, the recombinant herpes simplex virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes simplex virus genes. As used herein, an “inactivating mutation” may refer to any mutation that results in a gene or regulon product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc.

In some embodiments, the recombinant herpes simplex virus genome comprises an inactivating mutation in at least one, at least two, at least three, at least four, at least five, at least six, at least seven, or all eight of the Infected Cell Protein (or Infected Cell Polypeptide) (ICP) 0, ICP4, ICP22, ICP27, ICP47, thymidine kinase (tk), Long Unique Region (UL) 41 and/or UL55 herpes simplex virus genes. In some embodiments, the recombinant herpes simplex virus genome does not comprise an inactivating mutation in the ICP34.5 and/or ICP47 herpes simplex virus genes (e.g., to avoid production of an immune-stimulating virus). In some embodiments, the recombinant herpes simplex virus genome does not comprise an inactivating mutation in the ICP34.5 herpes simplex virus gene (one or both copies). In some embodiments, the recombinant herpes simplex virus genome does not comprise an inactivating mutation in the ICP47 herpes simplex virus gene. In some embodiments, the recombinant herpes simplex virus genome does not comprise an inactivating mutation in the ICP34.5 (one or both copies) and ICP47 herpes simplex virus genes. In some embodiments, the recombinant herpes simplex virus genome is not oncolytic.

In some embodiments, the recombinant herpes simplex virus genome comprises an inactivating mutation in the ICP0 gene (one or both copies). In some embodiments, the recombinant herpes simplex virus genome comprises an inactivating mutation in the ICP0 gene (one or both copies), and further comprises an initiating mutation in the ICP4 (one or both copies) ICP22, ICP27, ICP47, UL41, and/or UL55 genes. In some embodiments, the recombinant herpes simplex virus genome comprises an inactivating mutation in the ICP0 gene (one or both copies), and an inactivating mutation in the ICP4 gene (one or both copies). In some embodiments, the recombinant herpes simplex virus genome comprises an inactivating mutation in the ICP0 gene (one or both copies), and an inactivating mutation in the ICP22 gene. In some embodiments, the recombinant herpes simplex virus genome comprises an inactivating mutation in the ICP0 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), and an inactivating mutation in the ICP22 gene. In some embodiments, the recombinant herpes simplex virus genome comprises an inactivating mutation in the ICP0 gene (one or both copies), an inactivating mutation in the ICP4 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP22 gene, and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP0 (one or both copies), ICP4 (one or both copies), ICP22, and/or UL41 genes. In some embodiments, the recombinant herpes simplex virus genome further comprises an inactivating mutation in the ICP27, ICP47, and/or UL55 genes.

In some embodiments, the recombinant herpes simplex virus genome comprises an inactivating mutation in the ICP4 gene (one or both copies). In some embodiments, the recombinant herpes complex virus genome comprises an inactivating mutation in the ICP4 (one or both copies, and further comprises an inactivating mutation in the ICP0 (one or both copies), ICP22, ICP27, ICP47, UL41, and/or UL55 genes. In some embodiments, the recombinant herpes simplex virus genome comprises an inactivating mutation in the ICP4 gene (one or both copies), and an inactivating mutation in the ICP22 gene. In some embodiments, the recombinant herpes simplex virus genome comprises an inactivating mutation in the ICP4 gene (one or both copies), and an inactivating mutation in the UL41 gene. In some embodiments, the recombinant herpes simplex virus genome comprises an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP4 (one or both copies), ICP22, and/or UL41 genes. In some embodiments, the recombinant herpes simplex virus genome further comprises an inactivating mutation in the ICP0, ICP27, ICP47, and/or UL55 genes.

In some embodiments, the recombinant herpes simplex virus genome comprises an inactivating mutation in the ICP22 gene. In some embodiments, the recombinant herpes simplex virus genome comprises an inactivating mutation in the ICP22 gene, and further comprises an inactivating mutation in the ICP0 (one or both copies), ICP4 (one or both copies), ICP27, ICP47, UL41, and/or UL55 genes. In some embodiments, the recombinant herpes simplex virus genome comprises an inactivating mutation in the ICP22 gene, and an inactivating mutation UL41 gene. In some embodiments, the inactivating mutation is a deletion of the coding sequence of the ICP22 and/or UL41 genes. In some embodiments, the recombinant herpes simplex virus genome further comprises an inactivating mutation in the ICP0 (one or both copies), ICP4 (one or both copies), ICP27, ICP47, and/or UL55 genes.

In some embodiments, the recombinant herpes simplex virus genome comprises an inactivating mutation in the ICP27 gene. In some embodiments, the recombinant herpes simplex virus genome comprises an inactivating mutation in the ICP27 gene, and further comprises an inactivating mutation in the ICP0 (one or both copies), ICP4 (one or both copies), ICP22, ICP47, UL41, and/or UL55 genes. In some embodiments, the inactivating mutation is a deletion of the coding sequence of the ICP27 gene.

In some embodiments, the recombinant herpes simplex virus genome comprises an inactivating mutation in the ICP47 gene. In some embodiments, the recombinant herpes simplex virus genome comprises an inactivating mutation in the ICP47 gene, and further comprises an inactivating mutation in the ICP0 (one or both copies), ICP4 (one or both copies), ICP22, ICP27, UL41, and/or UL55 genes. In some embodiments, the inactivating mutation is a deletion of the coding sequence of the ICP47 gene.

In some embodiments, the recombinant herpes simplex virus genome comprises an inactivating mutation in the UL41 gene. In some embodiments, the recombinant herpes simplex virus genome comprises an inactivating mutation in the UL41 gene, and further comprises an inactivating mutation in the ICP0 (one or both copies), ICP4 (one or both copies), ICP22, ICP27, ICP47, and/or UL55 genes. In some embodiments, the inactivating mutation is a deletion of the coding sequence of the UL41 gene.

In some embodiments, the recombinant herpes simplex virus genome comprises an inactivating mutation in the UL55 gene. In some embodiments, the recombinant herpes simplex virus genome comprises an inactivating mutation in the UL55 gene, and further comprises an inactivating mutation in the ICP0 (one or both copies), ICP4 (one or both copies), ICP22, ICP27, ICP47, and/or UL41 genes. In some embodiments, the inactivating mutation is a deletion of the coding sequence of the UL55 gene.

In some embodiments, the recombinant herpes simplex virus genome comprises an inactivating mutation in (e.g., a deletion of) the internal repeat (Joint) region comprising the internal repeat long (IRL) and internal repeat short (IRS) regions. In some embodiments, inactivation (e.g., deletion) of the Joint region eliminates one copy each of the ICP4 and ICP0 genes. In some embodiments, inactivation (e.g., deletion) of the Joint region further inactivates (e.g., deletes) the promoter for the ICP22 and ICP47 genes. If desired, expression of one or both of these genes can be restored by insertion of an immediate early promoter into the recombinant herpes simplex virus genome (see e.g., Hill et al. (1995). Nature 375(6530): 411-415; Goldsmith et al. (1998). J Exp Med 187(3): 341-348). Without wishing to be bound by theory, it is believed that inactivating (e.g., deleting) the Joint region may contribute to the stability of the recombinant herpes simplex virus genome and/or allow for the recombinant herpes simplex virus genome to accommodate more and/or larger transgenes.

In some embodiments, the recombinant herpes simplex virus genome comprises an inactivating mutation in the ICP4 (one or both copies), ICP22, and ICP27 genes. In some embodiments, the recombinant herpes simplex virus genome comprises an inactivating mutation in the ICP4 (one or both copies), ICP27, and UL55 genes. In some embodiments, the recombinant herpes simplex virus genome comprises an inactivating mutation in the ICP4 (one or both copies), ICP22, ICP27, ICP47, and UL55 genes. In some embodiments, the inactivating mutation in the ICP4 (one or both copies), ICP27, and/or UL55 genes is a deletion of the coding sequence of the ICP4 (one or both copies), ICP27, and/or UL55 genes. In some embodiments, the inactivating mutation in the ICP22 and ICP47 genes is a deletion in the promoter region of the ICP22 and ICP47 genes (e.g., the ICP22 and ICP47 coding sequences are intact but are not transcriptionally active). In some embodiments, the recombinant herpes simplex virus genome comprises a deletion in the coding sequence of the ICP4 (one or both copies), ICP27, and UL55 genes, and a deletion in the promoter region of the ICP22 and ICP47 genes. In some embodiments, the recombinant herpes simplex virus genome further comprises an inactivating mutation in the ICP0 and/or UL41 genes.

In some embodiments, the recombinant herpes simplex virus genome comprises an inactivating mutation in the ICP0 (one or both copies) gene. In some embodiments, the recombinant herpes simplex virus genome comprises an inactivating mutation in the ICP0 (one or both copies) and ICP4 (one or both copies) genes. In some embodiments, the recombinant herpes simplex virus genome comprises an inactivating mutation in the ICP0 (one or both copies), ICP4 (one or both copies), and ICP22 genes. In some embodiments, the recombinant herpes simplex virus genome comprises an inactivating mutation in the ICP0 (one or both copies), ICP4 (one or both copies), ICP22, and ICP27 genes. In some embodiments, the recombinant herpes simplex virus genome comprises an inactivating mutation in the ICP0 (one or both copies), ICP4 (one or both copies), ICP22, ICP27 and UL55 genes. In some embodiments, the inactivating mutation in the ICP0 (one or both copies), ICP4 (one or both copies), ICP22, ICP27 and/or UL55 genes comprises a deletion of the coding sequence of the ICP0, ICP4 (one or both copies), ICP22, ICP27 and/or UL55 genes. In some embodiments, the recombinant herpes simplex virus genome further comprises an inactivating mutation in the ICP47 and/or the UL41 genes.

In some embodiments, a recombinant herpes simplex virus genome comprises one or more polynucleotides of the present disclosure within one, two, three, four, five, six, seven or more viral gene loci. Examples of suitable viral loci may include, without limitation, the ICP0 (one or both copies), ICP4 (one or both copies), ICP22, ICP27, ICP47, tk, UL41 and UL55 herpes simplex viral gene loci. In some embodiments, a recombinant herpes simplex virus genome comprises one or more polynucleotides of the present disclosure within one or both of the viral ICP4 gene loci (e.g., a recombinant virus carrying a first polynucleotide encoding a first human collagen protein in one or both of the ICP4 loci; a recombinant virus carrying a second polynucleotide encoding a second human collagen protein in one or both of the ICP4 loci; etc.). In some embodiments, a recombinant herpes simplex virus genome comprises one or more polynucleotide of the present disclosure within the viral ICP22 gene locus (e.g., a recombinant virus carrying a first polynucleotide encoding a first human collagen protein in the ICP22 locus; a recombinant virus carrying a second polynucleotide encoding a second human collagen protein in the ICP22 locus; etc.). In some embodiments, a recombinant herpes simplex virus genome comprises one or more polynucleotide of the present disclosure within the viral UL41 gene locus (e.g., a recombinant virus carrying a first polynucleotide encoding a first human collagen protein in the UL41 locus; a recombinant virus carrying a second polynucleotide encoding a second human collagen protein in the UL41 locus; etc.). In some embodiments, a recombinant herpes simplex virus genome comprises one or more polynucleotides of the present disclosure within one or both of the viral ICP4 gene loci, and one or more polynucleotides of the present disclosure within the viral ICP22 locus (e.g., a recombinant virus carrying a first polynucleotide encoding a first human collagen protein in one or both of the ICP4 loci and a second polynucleotide encoding a second human collagen protein in the ICP22 locus; a recombinant virus carrying a second polynucleotide encoding a second human collagen protein in one or both of the ICP4 loci and a first polynucleotide encoding a first human collagen protein in the ICP22 locus; etc.). In some embodiments, a recombinant herpes simplex virus genome comprises one or more polynucleotides of the present disclosure within one or both of the viral ICP4 gene loci, and one or more polynucleotides of the present disclosure within the viral UL41 locus (e.g., a recombinant virus carrying a first polynucleotide encoding a first human collagen protein in one or both of the ICP4 loci and a second polynucleotide encoding a second human collagen protein in the UL41 locus; a recombinant virus carrying a second polynucleotide encoding a second human collagen protein in one or both of the ICP4 loci and a first polynucleotide encoding a first human collagen protein in the UL41 locus; etc.). In some embodiments, a recombinant herpes simplex virus genome comprises one or more polynucleotides of the present disclosure within one or both of the viral ICP4 gene loci, one or more polynucleotides of the present disclosure within the viral ICP22 locus, and one or more polynucleotides of the present disclosure within the viral UL41 locus (e.g., a recombinant virus carrying a first polynucleotide encoding a first human collagen protein in one or both of the ICP4 loci and a second polynucleotide encoding a second human collagen protein in the ICP22 and UL41 loci; a recombinant virus carrying a second polynucleotide encoding a second human collagen protein in one or both of the ICP4 loci and a first polynucleotide encoding a first human collagen protein in the ICP22 and UL41 loci; etc.).

In some embodiments, the recombinant herpes virus genome (e.g., a recombinant herpes simplex virus genome) has been engineered to decrease or eliminate expression of one or more toxic herpes simplex genes (such as one or both copies of the HSV ICP0 gene, one or both copied of the HSV ICP4 gene, the ICP22 gene, and/or the UL41 gene). In some embodiments, the recombinant herpes virus genome (e.g., a recombinant herpes simplex virus genome) has been engineered to reduce cytotoxicity of the recombinant genome (e.g., when introduced into a target cell) as compared to a corresponding wild-type herpes virus genome (e.g., a wild-type herpes simplex virus genome). In some embodiments, cytotoxicity (e.g., in human keratinocytes and/or fibroblast cells) of the recombinant virus genome (e.g., a recombinant herpes simplex virus genome) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). In some embodiments, cytotoxicity (e.g., in human keratinocytes and/or fibroblast cells) of the recombinant herpes virus genome (e.g., a recombinant herpes simplex virus genome) is reduced by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). Methods of measuring cytotoxicity are known to one of ordinary skill in the art, including, for example, through the use of vital dyes (formazan dyes), protease biomarkers, an MTT assay (or an assay using related tetrazolium salts such as XTT, MTS, water-soluble tetrazolium salts, etc.), measuring ATP content, etc.

In some embodiments, the recombinant herpes virus genome (e.g., a recombinant herpes simplex virus genome) has been engineered to reduce its impact on host cell proliferation after exposure of the target cell to the recombinant genome, as compared to a corresponding wild-type herpes virus genome (e.g., a wild-type herpes simplex virus genome). In some embodiments, the target cell is a human cell. In some embodiments, the target cell is a cell of the epidermis and/or dermis. In some embodiments, the target cell is a keratinocyte and/or fibroblast. In some embodiments, host cell proliferation (e.g., human keratinocytes and/or fibroblast cells) after exposure to the recombinant genome is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). In some embodiments, host cell proliferation (e.g., human keratinocytes and/or fibroblast cells) after exposure to the recombinant genome is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, or at least about 1000-fold faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). Methods of measuring cellular proliferation are known to one of ordinary skill in the art, including, for example, through the use of a Ki67 cell proliferation assay, a BrdU cell proliferation assay, etc.

A vector (e.g., herpes viral vector) may include one or more polynucleotides of the present disclosure in a form suitable for expression of the polynucleotide in a host cell. Vectors may include one or more regulatory sequences operatively linked to the polynucleotide to be expressed (e.g., as described above).

In some embodiments, a recombinant nucleic acid of the present disclosure (e.g., a recombinant herpes simplex virus genome) comprises one or more of the polynucleotides described herein inserted in any orientation in the recombinant nucleic acid. If the recombinant nucleic acid comprises two or more polynucleotides described herein (e.g., two or more, three or more, etc.), the polynucleotides may be inserted in the same orientation or opposite orientations to one another. Without wishing to be bound be theory, incorporating two polynucleotides (e.g., two transgenes) into a recombinant nucleic acid (e.g., a vector) in an antisense orientation may help to avoid read-through and ensure proper expression of each polynucleotide.

### IV. Viruses

Certain aspects of the present disclosure relate to viruses comprising any of the polynucleotides and/or recombinant nucleic acids described herein. In some embodiments, the virus is capable of infecting one or more target cells of a subject (e.g., a human). In some embodiments, the virus is suitable for delivering the polynucleotides and/or recombinant nucleic acids into one or more target cells of a subject (e.g., a human subject). In some embodiments, the one or more target cell are one or more human cells. In some embodiments, the one or more target cells are one or more cells of the skin (e.g., one or more cells of the epidermis, dermis, and/or subcutis). In some embodiments, the one or more cells are selected from keratinocytes, melanocytes, Langerhans cells, Merkel cells, mast cells, fibroblasts, and/or adipocytes. In some embodiments, the one or more cells are keratinocytes. In some embodiments, the one or more cells reside in the stratum corneum, stratum granulosum, stratum spinulosum, stratum basale, and/or basement membrane. In some embodiments, the one or more target cells are one or more epidermal cells.

Any suitable virus known in the art may be used, including, for example, adenovirus, adeno-associated virus, retrovirus, lentivirus, sendai virus, herpes virus (e.g., a herpes simplex virus), vaccinia virus, and/or any hybrid virus thereof. In some embodiments, the virus is attenuated. In some embodiments, the virus is replication-defective. In some embodiments, the virus is replication-competent. In some embodiments, the virus has been modified to alter its tissue tropism relative to the tissue tropism of an unmodified, wild-type virus. In some embodiments, the virus has reduced cytotoxicity as compared to a corresponding wild-type virus. Methods for producing a virus comprising recombinant nucleic acids are well known to one of ordinary skill in the art.

In some embodiments, the virus is a member of the Herpesviridae family of DNA viruses, including, for example, a herpes simplex virus, a varicella zoster virus, a human cytomegalovirus, a herpesvirus 6A, a herpesvirus 6B, a herpesvirus 7, and a Kaposi's sarcoma-associated herpesvirus, etc. In some embodiments, the herpes virus is attenuated. In some embodiments, the herpes virus is replication-defective. In some embodiments, the herpes virus is replication-competent. In some embodiments, the herpes virus has reduced cytotoxicity as compared to a corresponding wild-type herpes virus. In some embodiments, the herpes virus is not oncolytic.

In some embodiments, the virus is a herpes simplex virus. Herpes simplex viruses comprising recombinant nucleic acids may be produced by a process disclosed, for example, in WO2015/009952 and/or WO2017/176336. In some embodiments, the herpes simplex virus is attenuated. In some embodiments, the herpes simplex virus is replication-competent. In some embodiments, the herpes simplex virus is replication-defective. In some embodiments, the herpes simplex virus is a herpes simplex type 1 virus (HSV-1), a herpes simplex type 2 virus (HSV-2), or any derivatives thereof. In some embodiments, the herpes simplex virus is a herpes simplex type 1 virus (HSV-1). In some embodiments, the HSV-1 is attenuated. In some embodiments, the HSV-1 has reduced cytotoxicity as compared to a corresponding wild-type HSV-1. In some embodiments, the HSV-1 is not oncolytic.

In some embodiments, the herpes simplex virus has been modified to alter its tissue tropism relative to the tissue tropism of an unmodified, wild-type herpes simplex virus. In some embodiments, the herpes simplex virus comprises a modified envelope. In some embodiments, the modified envelope comprises one or more (e.g., one or more, two or more, three or more, four or more, etc.) mutant herpes simplex virus glycoproteins. Examples of herpes simplex virus glycoproteins may include, but are not limited to, the glycoproteins gB, gC, gD, gH, and gL. In some embodiments, the modified envelope alters the herpes simplex virus tissue tropism relative to a wild-type herpes simplex virus.

In some embodiments, the transduction efficiency (in vitro and/or in vivo) of a virus of the present disclosure (e.g., a herpes virus) for one or more target cells (e.g., one or more human keratinocytes and/or fibroblasts) is at least about 25%. For example, the transduction efficiency of the virus for one or more target cells may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 99.5%, or more. In some embodiments, the virus is a herpes simplex virus and the transduction efficiency of the virus for one or more target cells (e.g., one or more human keratinocytes and/or fibroblasts) is about 85% to about 100%. In some embodiments, the virus is a herpes simplex virus and the transduction efficiency of the virus for one or more target cells (e.g., one or more human keratinocytes and/or fibroblasts) is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100%. Methods of measuring viral transduction efficiency in vitro or in vivo are well known to one of ordinary skill in the art, including, for example, qPCR analysis, deep sequencing, western blotting, fluorometric analysis (such as fluorescent in situ hybridization (FISH), fluorescent reporter gene expression, immunofluorescence, FACS), etc.

### V. Compositions and Formulations

Certain aspects of the present disclosure relate to compositions and formulations (e.g., pharmaceutical compositions and formulations) comprising any of the recombinant nucleic acids (e.g., a recombinant herpes virus genome) and/or viruses (e.g., a herpes virus comprising a recombinant genome described herein (such as a herpes simplex virus comprising a recombinant herpes simplex virus genome), and an excipient or carrier (e.g., a pharmaceutically acceptable excipient or carrier). In some embodiments, the composition or formulation is a cosmetic composition or formulation (e.g., a skin care product).

In some embodiments, the composition or formulation comprises any one or more of the viruses (e.g., herpes viruses) described herein. In some embodiments, the composition or formulation comprises from about 10⁴ to about 10¹² plaque forming units (PFU)/mL of the virus. For example, the composition or formulation may comprise from about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹¹ to about 10¹¹, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10⁴ to about 10⁹, about 10⁵ to about 10⁹, about 10⁶ to about 10⁹, about 10⁷ to about 10⁹, about 10⁸ to about 10⁹, about 10⁴ to about 10⁸, about 10⁵ to about 10⁸, about 10⁶ to about 10⁸, about 10⁷ to about 10⁸, about 10⁴ to about 10⁷, about 10⁵ to about 10⁷, about 10⁶ to about 10⁷, about 10⁴ to about 10⁶, about 10⁵ to about 10⁶, or about 10⁴ to about 10⁵ PFU/mL of the virus. In some embodiments, the composition or formulation comprises about 10⁴, about 10⁵, about 10⁶, about 10⁷, about 10⁸, about 10⁹, about 10¹⁰, about 10¹¹, or about 10¹² PFU/mL of the virus.

Compositions and formulations (e.g., pharmaceutical compositions and formulations) as described herein can be prepared by mixing the active ingredient(s) (such as a recombinant nucleic acid or a virus) having the desired degree of purity with one or more acceptable carriers or excipients. Acceptable carriers or excipients (e.g., pharmaceutically acceptable carriers or excipients) are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 10% glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); liposomes (e.g., cationic lipids); nanoparticle carriers; and/or non-ionic surfactants (such as polyethylene glycol (PEG)). A thorough discussion of carriers is available in REMINGTON'S PHARMACEUTICAL SCIENCES (Mack Pub. Co., N.J. 1991).

In some embodiments, the composition or formulation comprises one or more lipid (e.g., cationic lipid) carriers. In some embodiments, the composition or formulation comprises one or more nanoparticle carriers. Nanoparticles are submicron (less than about 1000 nm) sized drug delivery vehicles that can carry encapsulated drugs (such as synthetic small molecules, proteins, peptides, cells, viruses, and nucleic acid-based biotherapeutics for rapid or controlled release. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule (such as a virus) that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly(lactic acid), poly(glycolic acid), PLGA, PLA, PGA, and any combinations thereof (see e.g., Vauthier et al. Adv Drug Del Rev. (2003) 55: 519-48; US2007/0148074; US2007/0092575; US2006/0246139; U.S. Pat. Nos. 5,753,234; 7,081,483; and WO2006/052285).

In some embodiments, the carrier or excipient (e.g., a pharmaceutically acceptable carrier or excipient) may be adapted for or suitable for any administration route known in the art, including, for example, intravenous, intramuscular, subcutaneous, cutaneous, intranasal, intratracheal, sublingual, buccal, topical, oral, transdermal, intradermal, intraperitoneal, intraorbital, intravitreal, subretinal, transmucosal, intraarticular, by superficial injection, by implantation, by inhalation, intrathecal, intraventricular, and/or intranasal administration. In some embodiments, the carrier or excipient (e.g., pharmaceutically acceptable carrier or excipient) is adapted for or suitable for topical, transdermal, subcutaneous, and/or intradermal administration. In some embodiments, the carrier or excipient is adapted for or suitable for topical, transdermal, and/or intradermal administration. In some embodiments, the carrier or excipient is adapted for or suitable for superficial injection.

Examples of carriers or excipients adapted for or suitable for use in a topical, transdermal, subcutaneous, superficial, and/or intradermal application/administration may include, but are not limited to, ointments, oils, pastes, creams, aerosols, suspensions, emulsions, fatty ointments, gels, powders, liquids, lotions, solutions, sprays, patches (e.g., transdermal patches or microneedle patches), adhesive strips, a microneedle or microneedle arrays, and inhalants. In some embodiments, the carrier or excipient (e.g., the pharmaceutically acceptable carrier or excipient) comprises one or more (e.g., one or more, two or more, three or more, four or more, five or more, etc.) of an ointment, oil, paste, cream, aerosol, suspension, emulsion, fatty ointment, gel, powder, liquid lotion, solution, spray, adhesive strip, and an inhalant. In some embodiments, the carrier comprises a patch (e.g. a patch that adheres to the skin), such as a transdermal patch or a microneedle patch. In some embodiments, the carrier comprises a microneedle or microneedle array. Methods for making and using microneedle arrays suitable for composition delivery are generally known in the art (Kim Y. et al. “Microneedles for drug and vaccine delivery”. Advanced Drug Delivery Reviews 2012, 64 (14): 1547-68).

In some embodiments, the composition or formulation (e.g., the pharmaceutical composition or formulation) is adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, transmucosal, intraarticular, by superficial injection, by implantation, by inhalation, intrathecal, intraventricular, and/or intranasal administration. In some embodiments, the composition or formulation is adapted for or suitable for cutaneous, topical, transdermal, subcutaneous, and/or intradermal administration. In some embodiments, the pharmaceutical composition or formulation is adapted for or suitable for topical, transdermal, and/or intradermal administration. In some embodiments, the composition or formulation is adapted for or suitable for intradermal administration. In some embodiments, the composition of formulation is adapted for or suitable for superficial injection.

In some embodiments, the composition or formulation (e.g., pharmaceutical composition or formulation) further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the composition or formulation comprises a hydroxypropyl methylcellulose gel. In some embodiments, the composition or formulation comprises a phosphate buffer. In some embodiments, the composition or formulation comprises glycerol (e.g., at about 1%, about 2%, about 3%, about 4%, about 5%, about 6%, about 7%, about 8%, about 9%, about 10%, about 15%, etc.).

Compositions and formulations (e.g., pharmaceutical compositions and formulations) to be used for in vivo administration are generally sterile. Sterility may be readily accomplished, e.g., by filtration through sterile filtration membranes.

In some embodiments, any of the recombinant nucleic acids, viruses, and/or compositions or formulations described herein may be used to deliver one or more polynucleotides encoding a collagen protein (e.g., a human collagen protein such as Collagen 3) into one or more cells of a subject (e.g., one or more collagen-deficient cells). In some embodiments, any of the recombinant nucleic acids, viruses, and/or compositions or formulations described herein may be used in a therapy. In some embodiments, any of the recombinant nucleic acids, viruses, and/or compositions or formulations described herein may be used in the treatment of a cosmetic or aesthetic condition that would benefit from the expression of a collagen polypeptide (e.g., a cosmetic or aesthetic condition associated with a collagen deficiency (such as aged and/or UV-damaged skin)). In some embodiments, any of the recombinant nucleic acids, viruses, and/or compositions or formulations described herein may be used in the treatment of dermatological aging (e.g., as described below).

In some embodiments, any of the recombinant nucleic acids, viruses, and/or compositions or formulations described herein may be used in the preparation or manufacture of a medicament. In some embodiments, any of the recombinant nucleic acids, viruses, and/or compositions or formulations described herein may be used in the preparation or manufacture of a medicament useful for delivering one or more polynucleotides encoding a collagen protein (e.g., a human collagen protein such as Collagen 3) into one or more cells of a subject (e.g., one or more collagen-deficient cells). In some embodiments, any of the recombinant nucleic acids, viruses, and/or compositions or formulations described herein may be used in the preparation or manufacture of a medicament useful for the treatment of a cosmetic or aesthetic condition that would benefit from the expression of a collagen polypeptide (e.g., a cosmetic or aesthetic condition associated with a collagen deficiency (such as aged and/or UV-damaged skin)). In some embodiments, any of the recombinant nucleic acids, viruses, and/or compositions or formulations described herein may be used in the preparation or manufacture of a medicament useful for the treatment of dermatological aging (e.g., as described below).

### VI. Methods

Certain aspects of the present disclosure relate to a method of enhancing, increasing, augmenting, and/or supplementing the levels of one or more dermal extracellular matrix proteins in a subject (e.g., in one or more cells of a subject) comprising administering to the subject any of the recombinant nucleic acids, viruses, medicaments, and/or compositions described herein. In some embodiments, the subject is a human.

Other aspects of the present disclosure relate to method of stabilizing or improving the structure and/or organization of the dermal extracellular matrix in a subject comprising administering to the subject any of the recombinant nucleic acids, viruses, medicaments, and/or compositions described herein. In some embodiments, the subject is a human.

Other aspects of the present disclosure relate to a method of enhancing, increasing, augmenting, and/or supplementing the levels of one or more human collagen proteins in a subject (e.g., in one or more cells of a subject) comprising administering to the subject any of the recombinant nucleic acids, viruses, medicaments, and/or compositions described herein. In some embodiments, the subject is a human.

In some embodiments, administration of the recombinant nucleic acid, virus, medicament, and/or composition to the subject increases collagen (e.g., COL1-1; COL1-2; COL3; COL1-1 and COL1-2; COL1-1 and COL3; etc.) levels (transcript or protein levels) in one or more cells of the subject by at least about 10%, as compared to the endogenous levels of the collagen(s) in one or more corresponding untreated cells (e.g., one or more cells prior to treatment, one or more uninfected cells during treatment, etc.) of the subject. For example, administration of the recombinant nucleic acid, virus, medicament, and/or composition may increase collagen levels (transcript or protein levels) in one or more cells of the subject by at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, or more, as compared to the endogenous levels of the collagen(s) in one or more corresponding untreated cells of the subject. In some embodiments, administration of the recombinant nucleic acid, virus, medicament, and/or composition to the subject increases collagen levels (transcript or protein levels) in one or more cells of the subject by at least about 2-fold, as compared to the endogenous levels of the collagen(s) in one or more corresponding untreated cells (e.g., one or more cells prior to treatment, one or more uninfected cells during treatment, etc.) of the subject. For example, administration of the recombinant nucleic acid, virus, medicament, and/or composition may increase collagen levels (transcript or protein levels) in one or more cells of the subject by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more, as compared to the endogenous levels of the collagen(s) in one or more corresponding untreated cells of the subject. Methods of measuring transcript or protein levels from a sample are well known to one of ordinary skill in the art, including, for example, by qPCR, RNAseq, ELISA, western blot, mass spectrometry, etc.

Other aspects of the present disclosure relate to a method of enhancing, increasing, augmenting, and/or supplementing the soft tissue of a subject comprising administering to the subject any of the recombinant nucleic acids, viruses, medicaments, and/or compositions described herein. In some embodiments, the subject is a human. In some embodiments, the recombinant nucleic acids, viruses, medicaments, and/or compositions are injected into a soft tissue of the subject. In some embodiments, the skin of the subject is aging skin. In some embodiments, the skin of the subject has been damaged due to exposure to ultraviolet light (e.g., from the sun, from a tanning bed, etc.). In some embodiments, the skin of the subject is wrinkled.

In some embodiments, the recombinant nucleic acids, viruses, medicaments, and/or compositions may be used in a method to repair and/or augment the soft tissue of a subject. In some embodiments, “tissue repair” refers to the restoration of tissue architecture and/or function and encompasses tissue regeneration and replacement. In some embodiments, repair or augmentation of the soft tissue refers to procedures that are used to restore the youthful appearance of skin (e.g., as compared to “aged” skin whose appearance is due to defects resulting from chronological aging or other physical, chemical, or UV damage). In some embodiments, the recombinant nucleic acids, viruses, medicaments, and/or compositions are useful in cosmetic soft tissue applications, such as to fill wrinkles, lines, folds, scars, and to enhance dermal tissue (e.g., plump thin lips, fill in sunken eyes and/or shallow cheeks, etc.).

Other aspects of the present disclosure relate to a method of improving skin quality, condition, and/or appearance in a subject in need thereof comprising administering to the subject any of the recombinant nucleic acids, viruses, medicaments, and/or compositions described herein. In some embodiments, the subject is a human. In some embodiments, the skin condition is one or more of sun damage, aging, UV exposure, rough texture, skin sagging, and/or wrinkles. Improvement of skin quality, condition, and/or appearance (e.g., as compared to before treatment) may be assessed using any appropriate method or scale known in the art, including, for example, FACE Q, GAIS, etc. In some embodiments, the skin of the subject is aging skin. In some embodiments, the skin of the subject has been damaged due to exposure to ultraviolet light (e.g., from the sun, from a tanning bed, etc.). In some embodiments, the skin of the subject is wrinkled.

Other aspects of the present disclosure relate to a method of reducing the appearance of one or more superficial depressions in the skin of a subject in need thereof comprising administering to the subject any of the recombinant nucleic acids, viruses, medicaments, and/or compositions described herein. In some embodiments, the subject is a human. In some embodiments, administration of the recombinant nucleic acid, virus, medicaments, and/or composition reduces the appearance of one or more superficial depressions in the skin of the subject for at least about three months, at least about six months, at least about nine months, or at least about 12 months. In some embodiments, the appearance of one or more superficial depressions in the skin of the subject is reduced after administration of the composition, as compared to the appearance of the one or more superficial depression in the skin of the subject prior to administration of the composition. In some embodiments, the one or more superficial depressions in the skin are one or more of fine lines and wrinkles (e.g., forehead wrinkles, “crow's feet”, wrinkles at the edges of the eye or mouth, etc.). In some embodiments, the treatment of one or more superficial skin depressions is measured by an improvement in skin texture or skin quality, such as smoothness, hydration, and elasticity, as compared to non-treated skin. In some embodiments, the treatment of one or more superficial skin depressions is measured by a reduction in the severity (e.g., depth) of the superficial depressions and/or a reduction in the number of fine lines or wrinkles in a given area of skin. In some embodiments, the skin of the subject is aging skin. In some embodiments, the skin of the subject has been damaged due to exposure to ultraviolet light (e.g., from the sun, from a tanning bed, etc.). In some embodiments, the skin of the subject is wrinkled.

Other aspects of the present disclosure relate to a method of increasing and/or improving at least one of texture, smoothness, elasticity, and/or tension of the skin of a subject comprising administering to the subject any of the recombinant nucleic acids, viruses, medicaments, and/or compositions described herein. In some embodiments, the subject is a human. In some embodiments, the skin of the subject maintains at least one of an increased and/or improved texture, smoothness, elasticity, or tension for at least about three months, at least about six months, at least about nine months, or at least about 12 months after administration of the composition. In some embodiments, at least one of texture, smoothness, elasticity, or tension of the skin of the subject is increased and/or improved after administration of the composition, as compared to the texture, smoothness, elasticity, or tension of the skin of the subject prior to administration of the composition. Methods of measuring texture, smoothness, elasticity, and/or tensions of the skin are known to one of skill in the art. In some embodiments, the skin of the subject is aging skin. In some embodiments, the skin of the subject has been damaged due to exposure to ultraviolet light (e.g., from the sun, from a tanning bed, etc.). In some embodiments, the skin of the subject is wrinkled.

Other aspects of the present disclosure relate to a method of diminishing one or more dermatological signs of aging in a subject in need thereof comprising administering to the subject an effective amount of any of the recombinant nucleic acids, viruses, medicaments, and/or compositions described herein. In some embodiments, the subject is a human. In some embodiments, diminishing one or more dermatological signs of aging include any one of more of the following: treatment, reduction, and/or prevention of fine lines and/or wrinkles; reduction of skin pore size; improvement in skin thickness, plumpness, and/or tautness; improvement in skin smoothness, suppleness, and/or softness; improvement in skin tone, radiance, and/or clarity; improvement in procollagen and/or collagen production; improvement in skin texture and or promotion of retexturization; improvement in appearance of skin contours; restoration of skin luster and/or brightness; improvement of skin appearance decreased by aging and/or menopause; improvement in skin moisturization; increase in skin elasticity and/or resiliency; treatment, reduction, and/or prevention or skin sagging; improvement in skin firmness; reduction of pigment spots, mottled skin, and/or scars (such as acne scars); and/or improvement of optical properties of skin by light diffraction or reflection. In some embodiments, the one or more dermatological signs of aging in the subject is diminished after administration of the composition, as compared to the one or more dermatological signs of aging in the subject prior to administration of the composition. Any suitable method for measuring one or more signs of dermatological aging known in the art may be used.

In some embodiments, one or more portions of the skin of the subject is abraded or made more permeable prior to treatment with an effective amount of any of the recombinant nucleic acids, viruses, medicaments, and/or compositions described herein. Any suitable method of abrading the skin or increasing skin permeability known in the art may be used, including, for example, use of a dermal roller, repeated use of adhesive strips to remove layers of skin cells (tape stripping), scraping with a scalpel or blade, use of sandpaper, use of chemical permeation enhancers or electrical energy, use of sonic or ultrasonic energy, use of light (e.g., laser) energy, use of micron-sized needles or blades with a length suitable to pierce but not completely pass through the epidermis, etc.

In some embodiments, the methods of the present disclosure are for cosmetic applications, such as to reduce or eliminate one or more superficial depressions in the skin, to reduce or eliminate one or more wrinkles, and/or to prevent the occurrence or reoccurrence of one or more wrinkles. In some embodiments, the one or more superficial depressions in the skin or wrinkles are selected from nasolabial folds, crows' feet, frown lines, worry lines, scars, glabellar lines, brow ptosis, tear troughs, nasojugal lines, bunny lines, cheek/mid-face ptosis, marionette lines, poppy dimpling, smile lines, laugh lines, chin creases, neck lines, platysma bands, and any combinations thereof.

In some embodiments, an “effective amount” is at least the minimum amount required to affect a measurable improvement in or prevention of one or more signs or symptoms of a particular condition (e.g., a cosmetic condition such as skin aging). An “effective amount” may vary according to factors such as the age, sex, and weight of the patient. An effective amount is also one in which any toxic or detrimental effects of the treatment are outweighed by the beneficial effects. An effective amount can be administered in one or more administrations. For purposes of the present disclosure, an effective amount of a recombinant nucleic acid, virus, medicament, and/or composition is an amount sufficient to accomplish a measurable improvement either directly or indirectly. As is understood in the clinical context, an effective amount of a recombinant nucleic acid, virus, medicament, and/or composition may or may not be achieved in conjunction with another drug, compound, or composition. Thus, an “effective amount” may be considered in the context of administering one or more agents, and a single agent may be considered to be given in an effective amount if, in conjunction with one or more other agents, a desirable result may be or is achieved.

In some embodiments, the recombinant nucleic acid, virus, medicament, and/or composition is administered once to the subject. In some embodiments, the recombinant nucleic acid, virus, medicament, and/or composition is administered at least twice (e.g., at least 2 times, at least 3 times, at least 4 times, at least 5 times, at least 10 times, etc.) to the subject. In some embodiments, at least about 15 days (e.g., at least about 15 days, at least about 20 days, at least about 30 days, at least about 40 days, at least about 50 days, at least about 60 days, at least about 70 days, at least about 80 days, at least about 90 days, at least about 100 days, at least about 120 days, etc.) pass between administrations (e.g., between the first and second administrations, between the second and third administrations, etc.).

The recombinant nucleic acids, viruses, medicaments, and/or compositions or formulations described herein may be administered by any suitable method or route known in the art, including, without limitation, by oral administration, sublingual administration, buccal administration, topical administration, rectal administration, via inhalation, transdermal administration, subcutaneous injection, intradermal injection, superficial injection, intravenous (IV) injection, intra-arterial injection, intramuscular injection, intracardiac injection, intraosseous injection, intraperitoneal injection, transmucosal administration, vaginal administration, intraurethral administration, intravitreal administration, intraorbital administration, subretinal administration, intra-articular administration, peri-articular administration, local administration, epicutaneous administration, or any combinations thereof. The present disclosure thus encompasses methods of delivering any of the recombinant nucleic acids, viruses, medicaments, and/or compositions or formulations described herein to an individual.

In some embodiments, the recombinant nucleic acids, viruses, medicaments, and/or compositions or formulations are administered cutaneously, topically, transdermally, subcutaneously, or intradermally to the subject. In some embodiments, the recombinant nucleic acids, viruses, medicaments, and/or compositions or formulations are administered intradermally and/or subcutaneously. In some embodiments, the recombinant nucleic acids, viruses, medicaments, and/or compositions or formulations are administered via superficial injection. In some embodiments, the recombinant nucleic acids, viruses, medicaments, and/or compositions or formulations are administered topically and/or transdermally. In some embodiments, the recombinant nucleic acids, viruses, medicaments, and/or compositions or formulations are administered one, two, three, four, five or more times per day. In some embodiments, the recombinant nucleic acids, viruses, medicaments, and/or compositions or formulations are administered to one or more affected (e.g., wrinkled) areas of an individual. In some embodiments, the composition is administered to one or more unaffected areas of the individual.

In some embodiments, the recombinant nucleic acids, viruses, medicaments, and/or compositions are administered at a superficial depth in the skin. In some embodiments, the recombinant nucleic acids, viruses, medicaments, and/or compositions are introduced into the skin at a depth of about 2000 microns or less. For example, the recombinant nucleic acids, viruses, medicaments, and/or compositions may be administered into the skin at a depth of about 2000 microns or less, at about 1750 microns or less, at about 1500 microns or less, at about 1250 microns or less, at about 1000 microns or less, at about 900 microns or less, at about 800 microns or less, at about 700 microns or less, at about 600 microns or less, or at about 500 microns or less. In some embodiments, the recombinant nucleic acids, viruses, medicaments, and/or compositions are introduced at an injection depth between about 0.5 mm and 5.0 mm. For example, the recombinant nucleic acids, viruses, medicaments, and/or compositions may be introduced at an injection depth of between about 0.5 mm and 5.0 mm, about 0.5 mm and 4.5 mm, about 0.5 mm and 4.0 mm, about 0.5 mm and 3.5 mm, about 0.5 mm and 3.0 mm, about 0.5 mm and 2.5 mm, about 0.5 mm and 2.0 mm, about 0.5 mm and 1.5 mm, about 0.5 mm and 1.0 mm, 1.0 mm and 5.0 mm, about 1.0 mm and 4.5 mm, about 1.0 mm and 4.0 mm, about 1.0 mm and 3.5 mm, about 1.0 mm and 3.0 mm, about 1.0 mm and 2.5 mm, about 1.0 mm and 2.0 mm, about 1.0 mm and 1.5 mm, 1.5 mm and 5.0 mm, about 1.5 mm and 4.5 mm, about 1.5 mm and 4.0 mm, about 1.5 mm and 3.5 mm, about 1.5 mm and 3.0 mm, about 1.5 mm and 2.5 mm, about 1.5 mm and 2.0 mm, etc.

In some embodiments, the recombinant nucleic acids, viruses, medicaments, and/or compositions are administered by injections spaced apart by a distance of between about 1 mm to about 30 mm. For example, the recombinant nucleic acids, viruses, medicaments, and/or compositions may be administered by injections spaced apart by a distance of between about 1 mm and 30 mm, 2 mm and 30 mm, 5 mm and 30 mm, 10 mm and 30 mm, 15 mm and 30 mm, 1 mm and 20 mm, 2 mm and 20 mm, 5 mm and 20 mm, 10 mm and 20 mm, 15 mm and 20 mm, 1 mm and 15 mm, 2 mm and 15 mm, 5 mm and 15 mm, 10 mm and 15 mm, 1 mm and 10 mm, 2 mm and 10 mm, 5 mm and 10 mm, 1 mm and 5 mm, 2 mm and 5 mm, etc. In some embodiments, the injections are spaced apart by a distance of at least about 1 mm, at least about 2 mm, at least about 5 mm, at least about 10 mm, at least about 15 mm, at least about 20 mm, at least about 30 mm or more.

Numerous areas of the body may be treated with the recombinant nucleic acids, viruses, medicaments, and/or compositions described herein, including, for example, the face, forehead, lips, scalp, neck, arms, hands, legs, knees, feet, chest, back, groin, buttocks, thighs, etc. In some embodiments, the recombinant nucleic acids, viruses, medicaments, and/or compositions are administered to the face. In some embodiments, the recombinant nucleic acids, viruses, medicaments, and/or compositions are administered to one or more nasolabial folds. In some embodiments, the recombinant nucleic acids, viruses, medicaments, and/or compositions are administered around one or both eyes. In some embodiments, the recombinant nucleic acids, viruses, medicaments, and/or compositions are administered to one or more crows' feet. In some embodiments, the recombinant nucleic acids, viruses, medicaments, and/or compositions are administered to one or more frown lines. In some embodiments, the recombinant nucleic acids, viruses, medicaments, and/or compositions are administered to one or more scars (e.g., acne scars). In some embodiments, the recombinant nucleic acids, viruses, medicaments, and/or compositions are administered to one or more glabellar lines. In some embodiments, the recombinant nucleic acids, viruses, medicaments, and/or compositions are administered to one or more brow ptosis. In some embodiments, the recombinant nucleic acids, viruses, medicaments, and/or compositions are administered to one or more deep tear troughs. In some embodiments, the recombinant nucleic acids, viruses, medicaments, and/or compositions are administered to one or more nasojugal lines. In some embodiments, the recombinant nucleic acids, viruses, medicaments, and/or compositions are administered to one or more bunny lines. In some embodiments, the recombinant nucleic acids, viruses, medicaments, and/or compositions are administered to one or more cheek/mid-face ptosis. In some embodiments, the recombinant nucleic acids, viruses, medicaments, and/or compositions are administered to one or more marionette lines. In some embodiments, the recombinant nucleic acids, viruses, medicaments, and/or compositions are administered to one or sites of poppy dimpling. In some embodiments, the recombinant nucleic acids, viruses, medicaments, and/or compositions are administered to one or more chin creases. In some embodiments, the recombinant nucleic acids, viruses, medicaments, and/or compositions are administered to one or more neck lines. In some embodiments, the recombinant nucleic acids, viruses, medicaments, and/or compositions are administered to one or more platysma bands.

In some embodiments, the recombinant nucleic acid expresses the cosmetic protein(s) (e.g., human collagens) when the recombinant nucleic acid is delivered into one or more target cells of a subject. In some embodiments, expression of the cosmetic protein(s) (e.g., human collagens) enhances, increases, augments, and/or supplements the levels of human collagen in one or more target cells. In some embodiments, expression of the cosmetic protein(s) (e.g., human collagens) enhances, increases, augments, and/or supplements the levels of human collagen secreted by one or more target cells. In some embodiments, expression of the cosmetic protein(s) (e.g., human collagens) enhances, increases, augments, and/or supplements the levels of human collagen in the extracellular matrix. In some embodiments, expression of the cosmetic protein(s) (e.g., human collagens) enhances, increases, augments, and/or supplements the stability of the extracellular matrix in the subject. In some embodiments, expression of the cosmetic protein(s) (e.g., human collagens) enhances, augments, and/supplements the soft tissue of the subject. In some embodiments, expression of the cosmetic protein(s) (e.g., human collagens) improves the skin quality, condition, and/or appearance of the individual. In some embodiments, expression of the cosmetic protein(s) (e.g., human collagens) reduces one or more superficial depressions (e.g., wrinkles) in the skin of the subject. In some embodiments, expression of the cosmetic protein(s) (e.g., human collagens) improves the texture, smoothness, elasticity, and/or tension of the skin of the subject. In some embodiments, expression of the cosmetic protein(s) (e.g., human collagens) reduces one or more dermatological signs of aging in the subject.

### VII. Host Cells

Certain aspects of the present disclosure relate to one or more host cells comprising any of the recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NS0 and Sp2/0. In some embodiments, the host cell is a human or non-human primate cell. In some embodiments, the host cell is a Vero cell. In some embodiments, the host cell is a complementing host cell. In some embodiments, the host cell (e.g., the Vero cell) expresses one or more herpes simplex virus genes (e.g., an ICP4 gene). In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VCaP, CHO cells, and the like.

In some embodiments, the recombinant nucleic acid is a herpes simplex viral vector. In some embodiments, the recombinant nucleic acid is a herpes simplex virus amplicon. In some embodiments, the recombinant nucleic acid is an HSV-1 amplicon or HSV-1 hybrid amplicon. In some embodiments, a host cell comprising a helper virus is contacted with an HSV-1 amplicon or HSV-1 hybrid amplicon described herein, resulting in the production of a virus comprising one or more recombinant nucleic acids described herein. In some embodiments, the virus is collected from the supernatant of the contacted host cell. Methods of generating virus by contacting host cells comprising a helper virus with an HSV-1 amplicon or HSV-1/hybrid amplicon are known in the art.

In some embodiments, the host cell is a complementing host cell. In some embodiments, the complementing host cell expresses one or more genes that are inactivated in any of the viral vectors described herein. In some embodiments, the complementing host cell is contacted with a recombinant herpes virus genome (e.g., a recombinant herpes simplex virus genome) described herein. In some embodiments, contacting a complementing host cell with a recombinant herpes virus genome results in the production of a herpes virus comprising one or more recombinant nucleic acids described herein. In some embodiments, the virus is collected from the supernatant of the contacted host cell. Methods of generating virus by contacting complementing host cells with a recombinant herpes simplex virus are generally described in WO2015/009952 and/or WO2017/176336.

### VIII. Articles of Manufacture or Kits

Certain aspects of the present disclosure relate to an article of manufacture or a kit comprising any of the recombinant nucleic acids, viruses, medicaments and/or compositions or formulations (e.g., pharmaceutical compositions or formulations) described herein. In some embodiments, the article of manufacture or kit comprises a package insert comprising instructions for administering the recombinant nucleic acid, virus, medicament, and/or composition or formulation (e.g., to treat a dermal extracellular matrix protein (e.g., collagen) deficiency and/or to correct one or more dermatological signs of aging).

Suitable containers for the recombinant nucleic acids, viruses, medicaments and/or compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, needles, syringes, package inserts, and the like.

### IX. Enumerated Embodiments

Embodiment 1: a composition comprising: (a) a herpes simplex virus (HSV) comprising a recombinant nucleic acid, wherein the recombinant nucleic acid comprises a first polynucleotide encoding a first polypeptide comprising a first human collagen protein, and (b) an excipient.

Embodiment 2: the composition of embodiments 1, wherein the recombinant nucleic acid comprises two or more copies of the first polynucleotide.

Embodiment 3: the composition of embodiment 1 or 2, wherein the HSV is replication-defective.

Embodiment 4: the composition of embodiment 1 or 2, wherein the HSV is replication-competent.

Embodiment 5: the composition of any one of embodiments 1-4, wherein the HSV is a herpes simplex type 1 virus, a herpes simplex type 2 virus, or any derivatives thereof.

Embodiment 6: the composition of any one of embodiments 1-5, wherein the recombinant nucleic acid is a herpes simplex virus amplicon.

Embodiment 7: the composition of embodiment 6, wherein the herpes simplex virus amplicon is an HSV-1 amplicon or an HSV-1 hybrid amplicon.

Embodiment 8: the composition of embodiment 7, wherein the HSV-1 hybrid amplicon is an HSV/AAV hybrid amplicon, an HSV/EBV hybrid amplicon, and HSV/EBV/RV hybrid amplicon, or an HSV/Sleeping Beauty hybrid amplicon.

Embodiment 9: the composition of any one of embodiments 1-5, wherein the recombinant nucleic acid is a recombinant herpes simplex virus genome.

Embodiment 10: the composition of embodiment 9, wherein the recombinant herpes simplex virus genome is a recombinant HSV-1 genome, a recombinant HSV-2 genome, or any derivatives thereof.

Embodiment 11: the composition of embodiment 9 or 10, wherein the recombinant herpes simplex virus genome comprises an inactivating mutation in a herpes simplex virus gene.

Embodiment 12: the composition of embodiment 11, wherein the herpes simplex virus gene is selected from the group consisting of Infected Cell Protein (ICP) 0, ICP4, ICP22, ICP27, ICP47, thymidine kinase (tk), Long Unique Region (UL) 41, and UL55.

Embodiment 13: the composition of embodiment 12, wherein the recombinant herpes simplex virus genome comprises an inactivation mutation in one or both copies of the ICP4 gene.

Embodiment 14: the composition of embodiment 12 or 13, wherein the recombinant herpes simplex virus genome comprises an inactivating mutation in the ICP22 gene.

Embodiment 15: the composition of any one of embodiments 12-14, wherein the recombinant herpes simplex virus genome comprises an inactivation mutation in the UL41 gene.

Embodiment 16: the composition of any one of embodiments 12-15, wherein the recombinant herpes simplex virus genome comprises an inactivation mutation in the ICP0 gene.

Embodiment 17: the composition of any one of embodiments 12-16, wherein the recombinant herpes simplex virus genome comprises an inactivation mutation in the ICP27 gene.

Embodiment 18: the composition of any one of embodiments 11-17, wherein the inactivating mutation is a deletion of the coding sequence of the gene(s).

Embodiment 19: the composition of any one of embodiments 9-18, wherein the recombinant herpes simplex virus genome comprises the first polynucleotide within a viral gene locus.

Embodiment 20: The composition of any one of embodiments 9-19, wherein the recombinant herpes simplex virus genome comprises the first polynucleotide within one or both copies of the ICP4 viral gene loci.

Embodiment 21: the composition of any one of embodiments 9-20, wherein the recombinant herpes simplex virus genome comprises the first polynucleotide within the ICP22 viral gene locus.

Embodiment 22: the composition of any one of embodiment 9-21, wherein the recombinant herpes simplex virus genome comprises the first polynucleotide within the UL41 viral gene locus.

Embodiment 23: the composition of any one of embodiments 1-22, wherein the HSV has reduced cytotoxicity as compared to a wild-type herpes simplex virus.

Embodiment 24: the composition of any one of embodiments 1-23, wherein the first human collagen protein is selected from the group consisting of Collagen alpha-1(I) chain polypeptide (COL1-1), Collagen alpha-2(I) chain polypeptide (COL1-2), a Collagen alpha-1(II) chain polypeptide (COL2), a Collagen alpha-1(III) chain polypeptide (COL3), a Collagen alpha-1(IV) chain polypeptide (COL4-1), a Collagen alpha-2(IV) chain polypeptide (COL4-2), a Collagen alpha-3(IV) chain polypeptide (COL4-3), a Collagen alpha-4(IV) chain polypeptide (COL4-4), a Collagen alpha-5(IV) chain polypeptide (COL4-5), a Collagen alpha-6(IV) chain polypeptide (COL4-6), a Collagen alpha-1(V) chain polypeptide (COL5-1), a Collagen alpha-2(V) chain polypeptide (COL5-2), a Collagen alpha-3(V) chain polypeptide (COL5-3), a Collagen alpha-1(VI) chain polypeptide (COL6-1), a Collagen alpha-2(VI) chain polypeptide (COL6-2), a Collagen alpha-3(VI) chain polypeptide (COL6-3), a Collagen alpha-4(VI) chain polypeptide (COL6-4), a Collagen alpha-5(VI) chain polypeptide (COL6-5), a Collagen alpha-6(VI) chain polypeptide (COL6-6), a Collagen alpha-1(VII) chain polypeptide (COL7), a Collagen alpha-1(VIII) chain polypeptide (COL8), a Collagen alpha-1(IX) chain polypeptide (COL9-1), a Collagen alpha-2(IX) chain polypeptide (COL9-2), a Collagen alpha-3(IX) chain polypeptide (COL9-3), a Collagen alpha-1(X) chain polypeptide (COL10), a Collagen alpha-1(XI) chain polypeptide (COL11-1), a Collagen alpha-2(XI) chain polypeptide (COL11-2), a Collagen alpha-1(XII) chain polypeptide (COL12), a Collagen alpha-1(XIII) chain polypeptide (COL13), a Collagen alpha-1(XIV) chain polypeptide (COL14), a Collagen alpha-1(XV) chain polypeptide (COL15), a Collagen alpha-1(XVI) chain polypeptide (COL16), a Collagen alpha-1(XVII) chain polypeptide (COL17), a Collagen alpha-1(XVIII) chain polypeptide (COL18), a Collagen alpha-1(XIX) chain polypeptide (COL19), a Collagen alpha-1(XX) chain polypeptide (COL20), a Collagen alpha-1(XXI) chain polypeptide (COL21), a Collagen alpha-1(XXII) chain polypeptide (COL22), a Collagen alpha-1(XXIII) chain polypeptide (COL23), a Collagen alpha-1(XXIV) chain polypeptide (COL24), a Collagen alpha-1(XXV) chain polypeptide (COL25), a Collagen alpha-1(XXVI) chain polypeptide (COL26), a Collagen alpha-1(XXVII) chain polypeptide (COL27), and a Collagen alpha-1(XXVIII) chain polypeptide (COL28).

Embodiment 25: the composition of any one of embodiments 1-24, wherein the first human collagen protein is selected from the group consisting of COL1-1, COL1-2, COL3, COL4-1, COL6-1, COL7, and COL17.

Embodiment 26: the composition of any one of embodiments 1-25, wherein the nucleic acid sequence encoding the first human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 27: the composition of any one of embodiments 1-26, wherein the first human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 28: the composition of any one of embodiments 1-27, wherein the first human collagen protein is not COL7.

Embodiment 29: the composition of any one of embodiments 1-28, wherein the first polypeptide comprises: (a) the first human collagen protein; (b) a further human collagen protein; and (c) a linker polypeptide linking (a) to (b).

Embodiment 30: the composition of embodiment 29, wherein the linker polypeptide is a cleavable linker polypeptide.

Embodiment 31: the composition of embodiment 29 or 30, wherein the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 28-31

Embodiment 32: the composition of any one of embodiments 29-31, wherein the further human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 33: the composition of any one of embodiments 29-32, wherein the further human collagen protein is selected from the group consisting of COL1-1, COL1-2, COL3, COL4-1, COL6-1, COL7, and COL17.

Embodiment 34: the composition of any one of embodiments 29-33, wherein the nucleic acid sequence encoding the further human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 35: the composition of any one of embodiments 29-34, wherein the further human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 36: the composition of any one of embodiments 29-35, wherein the first human collagen protein and the further human collagen protein are different.

Embodiment 37: the composition of any one of embodiments 1-36, wherein the first polynucleotide encodes a polycistronic mRNA comprising: (a) a first open reading frame (ORF) encoding the first polypeptide; (b) a second ORF encoding an additional human collagen protein; and (c) an internal ribosomal entry site (IRES) separating (a) and (b).

Embodiment 38: the composition of embodiment 37, wherein the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23.

Embodiment 39: the composition of embodiment 37 or 38, wherein the additional human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 40: the composition of any one of embodiments 37-39, wherein the additional human collagen protein is selected from the group consisting of COL1-1, COL1-2, COL3, COL4-1, COL6-1, COL7, and COL17.

Embodiment 41: the composition of any one of embodiments 37-40, wherein the nucleic acid sequence encoding the additional human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 42: the composition of any one of embodiments 37-41, wherein the additional human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 43: the composition of any one of embodiments 37-42, wherein the first human collagen protein and the additional human collagen protein are different.

Embodiment 44: the composition of any one of embodiments 1-43, wherein the recombinant nucleic acid further comprises a second polynucleotide encoding a second human collagen protein.

Embodiment 45: the composition of embodiment 44, wherein the recombinant nucleic acid comprises two or more copies of the second polynucleotide.

Embodiment 46, the composition of embodiment 44 or 45, wherein the second human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 47: the composition of any one of embodiments 44-46, wherein the second human collagen protein is selected from the group consisting of COL1-1, COL1-2, COL3, COL4-1, COL6-1, COL7, and COL17.

Embodiment 48: the composition of any one of embodiments 44-48, wherein the nucleic acid sequence encoding the second human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 49: the composition of any one of embodiments 44-48, wherein the second human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 50: the composition of any one of embodiments 44-49, wherein the first and second human collagen proteins are different.

Embodiment 51: the composition of any one of embodiments 44-50, wherein the recombinant nucleic acid is a recombinant herpes simplex virus genome, and wherein the recombinant herpes simplex virus genome comprises the second polynucleotide within a viral gene locus.

Embodiment 52: the composition of embodiment 51, wherein the recombinant herpes simplex virus genome comprises the second polynucleotide within one or both copies of the ICP4 viral gene loci.

Embodiment 53: the composition of embodiment 51 or 52, wherein the recombinant herpes simplex virus genome comprises the second polynucleotide within the ICP22 viral gene locus.

Embodiment 54: the composition of any one of embodiments 51-53, wherein the recombinant herpes simplex virus genome comprises the second polynucleotide within the UL41 viral gene locus.

Embodiment 55: the composition of any one of embodiments 51-54, wherein the recombinant herpes simplex virus genome comprises the first polynucleotide within one or both copies of the ICP4 viral gene loci and the second polynucleotide within the ICP22 viral gene locus.

Embodiment 56: the composition of any one of embodiments 51-54, wherein the recombinant herpes simplex virus genome comprises the first polynucleotide within one or both copies of the ICP4 viral gene loci and the second polynucleotide within the UL41 viral gene locus.

Embodiment 57: the composition of any one of embodiments 1-56, wherein the excipient is adapted for cutaneous (systemic or topical), transdermal, subcutaneous, and/or intradermal administration.

Embodiment 58: the composition of any one of embodiments 1-57, wherein the excipient comprises a hydroxypropyl methylcellulose gel.

Embodiment 59: the composition of any one of embodiments 1-58, wherein the excipient is adapted for intradermal administration.

Embodiment 60: the composition of any one of embodiments 1-59, wherein the excipient comprises a phosphate buffer.

Embodiment 61: the composition of any one of embodiments 1-60, wherein the excipient comprises glycerol.

Embodiment 62: the composition of any one of embodiments 1-61, wherein the excipient comprises a lipid carrier.

Embodiment 63: the composition of any one of embodiments 1-62, wherein the excipient comprises a nanoparticle carrier.

Embodiment 64: the composition of any one of embodiments 1-63, wherein the composition is a cosmetic composition.

Embodiment 65: the composition of embodiment 64, wherein the cosmetic composition is a skin care product.

Embodiment 66: a kit comprising: (a) the composition of any one of embodiment 1-65; and (b) instructions for administering the composition.

Embodiment 67: a method of enhancing, increasing, augmenting, and/or supplementing the levels of one or more human collagen proteins in a subject, the method comprising administering to the subject an effective amount of the composition of any one of embodiments 1-65.

Embodiment 68: a method of enhancing, increasing, augmenting, and/or supplementing soft tissue of a subject, the method comprising administering to the subject an effective amount of the composition of any one of embodiments 1-65.

Embodiment 69: the method of embodiment 68, wherein the composition is injected into a soft tissue of the subject.

Embodiment 70: a method of improving skin quality, condition and/or appearance in a subject in need thereof, the method comprising administering to the subject an effective amount of the composition of any one of embodiments 1-65.

Embodiment 71: the method of embodiment 70, wherein the condition is selected from the group consisting of sun damage, aging, UV exposure, rough texture, skin sagging, wrinkles, and any combinations thereof.

Embodiment 72: a method of reducing the appearance of one or more superficial depressions in the skin of a subject in need thereof, the method comprising administering to the subject an effective amount of the composition of any one of embodiments 1-65.

Embodiment 73: the method of embodiment 72, wherein administration of the composition reduces the appearance of the one or more superficial depressions in the skin of the subject for at least about three months, at least about six months, at least about nine months, or at least about 12 months.

Embodiment 74: the method of embodiment 72 or 73, wherein the appearance of the one or more superficial depressions in the skin of the subject is reduced after administration of the composition, as compared to the appearance of the one or more superficial depression in the skin of the subject prior to administration of the composition.

Embodiment 75: a method of increasing and/or improving at least one of texture, smoothness, elasticity, or tension of the skin of a subject in need thereof, the method comprising administering to the subject an effective amount of the composition of any one of embodiments 1-65.

Embodiment 76: the method of embodiment 75, wherein the skin of the subject maintains at least one of an increased and/or improved texture, smoothness, elasticity, or tension for at least about three months, at least about six months, at least about nine months, or at least about 12 months after administration of the composition.

Embodiment 77: the method of embodiment 75 or 76, wherein at least one of texture, smoothness, elasticity, or tension of the skin of the subject is increased and/or improved after administration of the composition, as compared to the texture, smoothness, elasticity, or tension of the skin of the subject prior to administration of the composition.

Embodiment 78: the method of any one of embodiments 70-77, wherein the skin of the subject is aging skin.

Embodiment 79: the method of any one of embodiments 70-78, wherein the skin of the subject has been damaged due to exposure to ultraviolet light.

Embodiment 80: the method of any one of embodiments 70-79, wherein the skin of the subject is wrinkled.

Embodiment 81: a method of diminishing one or more dermatological signs of aging in a subject in need thereof, the method comprising administering to the subject an effective amount of the composition of any one of embodiments 1-65.

Embodiment 82: the method of embodiment 81, wherein the diminishing of one or more dermatological signs of aging is selected from the group consisting of: (a) treatment, reduction, and/or prevention of fine lines and/or wrinkles; (b) reduction of skin pore size; (c) improvement in skin thickness, plumpness, and/or tautness; (d) improvement in skin smoothness, suppleness, and/or softness; (e) improvement in skin tone, radiance and/or clarity; (f) improvement in procollagen and/or collagen production; (g) improvement in skin texture and or promotion of retexturization; (h) improvement in appearance of skin contours; (i) restoration of skin luster and/or brightness; (j) improvement of skin appearance decreased by aging and/or menopause; (k) improvement in skin moisturization; (l) increase in skin elasticity and/or resiliency; (m) treatment, reduction, and/or prevention or skin sagging; (n) improvement in skin firmness; (o) reduction of pigment spots, mottled skin, and/or acne scars; (p) improvement of optical properties of skin by light diffraction or reflection; and (q) any combinations thereof.

Embodiment 83: the method of embodiment 81 or 82, wherein the one or more dermatological signs of aging in the subject is diminished after administration of the composition, as compared to the one or more dermatological signs of aging in the subject prior to administration of the composition.

Embodiment 84: the method of any one of embodiments 67-83, wherein the subject is a human.

Embodiment 85: the method of any one of embodiments 67-84, wherein the composition is administered cutaneously (systemically or topically), transdermally, subcutaneously, or intradermally to the subject.

Embodiment 86: the method of any one of embodiments 67-85, wherein the composition is administered by superficial injection.

Embodiment 87: the method of any one of embodiments 67-85, wherein the composition is administered intradermally to the subject.

Embodiment 88: the method of any one of embodiments 67-87, wherein the composition is administered once to the subject.

Embodiment 89: the method of any one of embodiments 67-87, wherein the composition is administered at least twice to the subject.

Embodiment 90: the method of embodiment 89, wherein at least about 15, at least about 30, at least about 60, at least about 90, or at least about 120 days passes between administrations.

Embodiment 91: the method of any one of embodiments 67-90, wherein the composition is administered to one or more affected and/or unaffected areas of the subject.

Embodiment 92: the method of any one of embodiments 67-91, wherein the skin of the subject is abraded prior to administration.

Embodiment 93: a recombinant nucleic acid comprising a first polynucleotide encoding a first polypeptide comprising a first human collagen protein, wherein the recombinant nucleic acid is a recombinant herpes simplex virus genome.

Embodiment 94: the recombinant nucleic acid of embodiment 93, wherein the recombinant nucleic acid comprises two or more copies of the first polynucleotide.

Embodiment 95: the recombinant nucleic acid of embodiment 93 or 94, wherein the recombinant herpes simplex virus genome is a recombinant HSV-1 genome, a recombinant HSV-2 genome, or any derivatives thereof.

Embodiment 96: the recombinant nucleic acid of any one of embodiments 93-95, wherein the recombinant herpes simplex virus genome comprises an inactivating mutation in a herpes simplex virus gene.

Embodiment 97: the recombinant nucleic acid of embodiment 96, wherein the herpes simplex virus gene is selected from the group consisting of ICP0, ICP4, ICP22, ICP27, ICP47, tk, UL41, and UL55.

Embodiment 98: the recombinant nucleic acid of embodiment 97, wherein the recombinant herpes simplex virus genome comprises an inactivation mutation in one or both copies of the ICP4 gene.

Embodiment 99: the recombinant nucleic acid of embodiment 97 or 98, wherein the recombinant herpes simplex virus genome comprises an inactivating mutation in the ICP22 gene.

Embodiment 100: the recombinant nucleic acid of any one of embodiments 97-99, wherein the recombinant herpes simplex virus genome comprises an inactivation mutation in the UL41 gene.

Embodiment 101: the recombinant nucleic acid of any one of embodiments 97-100, wherein the recombinant herpes simplex virus genome comprises an inactivation mutation in the ICP0 gene.

Embodiment 102: the recombinant nucleic acid of any one of embodiments 97-101, wherein the recombinant herpes simplex virus genome comprises an inactivation mutation in the ICP27 gene.

Embodiment 103: the recombinant nucleic acid of any one of embodiments 96-102, wherein the inactivating mutation is a deletion of the coding sequence of the gene(s).

Embodiment 104: the recombinant nucleic acid of any one of embodiments 93-103, wherein the recombinant herpes simplex virus genome comprises the first polynucleotide within a viral gene locus.

Embodiment 105: the recombinant nucleic acid of any one of embodiments 93-104, wherein the recombinant herpes simplex virus genome comprises the first polynucleotide within one or both copies of the ICP4 viral gene loci.

Embodiment 106: the recombinant nucleic acid of any one of embodiments 93-105, wherein the recombinant herpes simplex virus genome comprises the first polynucleotide within the ICP22 viral gene locus.

Embodiment 107: the recombinant nucleic acid of any one of 93-106, wherein the recombinant herpes simplex virus genome comprises the first polynucleotide within the UL41 viral gene locus.

Embodiment 108: the recombinant nucleic acid of any one of embodiments 93-107, wherein the first human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 109: the recombinant nucleic acid of any one of embodiments 93-108, wherein the first human collagen protein is selected from the group consisting of COL1-1, COL1-2, COL3, COL4-1, COL6-1, COL7, and COL17.

Embodiment 110: the recombinant nucleic acid of any one of embodiments 93-109, wherein the nucleic acid sequence encoding the first human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 111: the recombinant nucleic acid of any one of embodiments 93-110, wherein the first human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 112: the recombinant nucleic acid of any one of embodiments 93-111, wherein the first human collagen protein is not COL7.

Embodiment 113: the recombinant nucleic acid of any one of embodiments 93-112, wherein the first polypeptide comprises: (a) the first human collagen protein; (b) a further human collagen protein; and (c) a linker polypeptide linking (a) to (b).

Embodiment 114: The recombinant nucleic acid of embodiment 113, wherein the linker polypeptide is a cleavable linker polypeptide.

Embodiment 115: the recombinant nucleic acid of embodiment 113 or 114, wherein the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 28-31.

Embodiment 116: the recombinant nucleic acid of any one of embodiments 113-115, wherein the further human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 117: the recombinant nucleic acid of any one of embodiments 113-116, wherein the further human collagen protein is selected from the group consisting of COL1-1, COL1-2, COL3, COL4-1, COL6-1, COL7, and COL17.

Embodiment 118: the recombinant nucleic acid of any one of embodiments 113-117, wherein the nucleic acid sequence encoding the further human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 119: the recombinant nucleic acid of any one of embodiments 113-118, wherein the further human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 120: the recombinant nucleic acid of any one of embodiments 113-119, wherein the first human collagen protein and the further human collagen protein are different.

Embodiment 121: the recombinant nucleic acid of any one of embodiments 93-120, wherein the first polynucleotide encodes a polycistronic mRNA comprising: (a) a first open reading frame (ORF) encoding the first polypeptide; (b) a second ORF encoding an additional human collagen protein; and (c) an internal ribosomal entry site (IRES) separating (a) and (b).

Embodiment 122: the recombinant nucleic acid of embodiment 121, wherein the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 22 or SEQ ID NO: 23.

Embodiment 123: the recombinant nucleic acid of embodiment 121 or 122, wherein the additional human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 124: the recombinant nucleic acid of any one of embodiments 121-123, wherein the additional human collagen protein is selected from the group consisting of COL1-1, COL1-2, COL3, COL4-1, COL6-1, COL7, and COL17.

Embodiment 125: the recombinant nucleic acid of any one of embodiments 121-124, wherein the nucleic acid sequence encoding the additional human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 126: the recombinant nucleic acid of any one of embodiments 121-125, wherein the additional human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 127: the recombinant nucleic acid of any one of embodiments 121-126, wherein the first human collagen protein and the additional human collagen protein are different.

Embodiment 128: the recombinant nucleic acid of any one of embodiments 93-127, wherein the recombinant nucleic acid further comprises a second polynucleotide encoding a second human collagen protein.

Embodiment 129: the recombinant nucleic acid of embodiment 128, wherein the recombinant nucleic acid comprises two or more copies of the second polynucleotide.

Embodiment 130: the recombinant nucleic acid of embodiment 128 or 129, wherein the second human collagen protein is selected from the group consisting of COL1-1, COL1-2, COL2, COL3, COL4-1, COL4-2, COL4-3, COL4-4, COL4-5, COL4-6, COL5-1, COL5-2, COL5-3, COL6-1, COL6-2, COL6-3, COL6-4, COL6-5, COL6-6, COL7, COL8, COL9-1, COL9-2, COL9-3, COL10, COL11-1, COL11-2, COL12, COL13, COL14, COL15, COL16, COL17, COL18, COL19, COL20, COL21, COL22, COL23, COL24, COL25, COL26, COL27, and COL28.

Embodiment 131: the recombinant nucleic acid of any one of embodiments 128-130, wherein the second human collagen protein is selected from the group consisting of COL1-1, COL1-2, COL3, COL4-1, COL6-1, COL7, and COL17.

Embodiment 132: the recombinant nucleic acid of any one of embodiments 128-131, wherein the nucleic acid sequence encoding the second human collagen protein has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from the group consisting of SEQ ID NOS: 1-14.

Embodiment 133: the recombinant nucleic acid of any one of embodiments 128-132, wherein the second human collagen protein comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 15-21.

Embodiment 134: the recombinant nucleic acid of any one of embodiments 128-133, wherein the first and second human collagen proteins are different.

Embodiment 135: the recombinant nucleic acid of any one of embodiments 128-134, wherein the recombinant herpes simplex virus genome comprises the second polynucleotide within a viral gene locus.

Embodiment 136: the recombinant nucleic acid of embodiment 135, wherein the recombinant herpes simplex virus genome comprises the second polynucleotide within one or both copies of the ICP4 viral gene loci.

Embodiment 137: the recombinant nucleic acid of embodiment 135 or 136, wherein the recombinant herpes simplex virus genome comprises the second polynucleotide within the ICP22 viral gene locus.

Embodiment 138: the recombinant nucleic acid of any one of embodiments 135-137, wherein the recombinant herpes simplex virus genome comprises the second polynucleotide within the UL41 viral gene locus.

Embodiment 139: the recombinant nucleic acid of any one of embodiments 135-138, wherein the recombinant herpes simplex virus genome comprises the first polynucleotide within one or both copies of the ICP4 viral gene loci and the second polynucleotide within the ICP22 viral gene locus.

Embodiment 140: the recombinant nucleic acid of any one of embodiments 135-138, wherein the recombinant herpes simplex virus genome comprises the first polynucleotide within one or both copies of the ICP4 viral gene loci and the second polynucleotide within the UL41 viral gene locus.

Embodiment 141: a host cell comprising the recombinant nucleic acid of any one embodiments 93-140.

Embodiment 142: the host cell of embodiment 141, wherein the host cell is a eukaryotic cell.

Embodiment 143: the hose cell of embodiment 141 or 142, wherein the host cell is a mammalian cell.

Embodiment 144: the host cell of any one of embodiments 141-143, wherein the host cell is a human cell or a non-human primate cell.

Embodiment 145: the host cell of any one of embodiments 141-144, wherein the host cell is a Vero cell.

Embodiment 146: the host cell of any one of embodiments 141-145, wherein the cost cell is a complementing host cell.

Embodiment 147: a method of collecting a herpes simplex virus, the method comprising: (a) contacting a complementing host cell with the recombinant nucleic acid of any one of embodiments 93-140; and (b) collecting the herpes simplex virus generated by the complementing host cell.

Embodiment 148: a method of collecting a herpes simplex virus, the method comprising: (a) culturing the host cell of any one of embodiments 141-146; and (b) collecting the herpes simplex virus generated by the host cell.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### EXAMPLES

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art, and are to be included within the spirit and purview of this application and scope of the appended claims.

### Example 1: Modified Herpes Simplex Virus Vectors Encoding Human Collagen Protein(s)

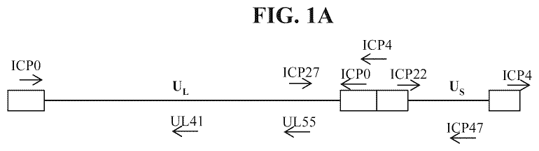

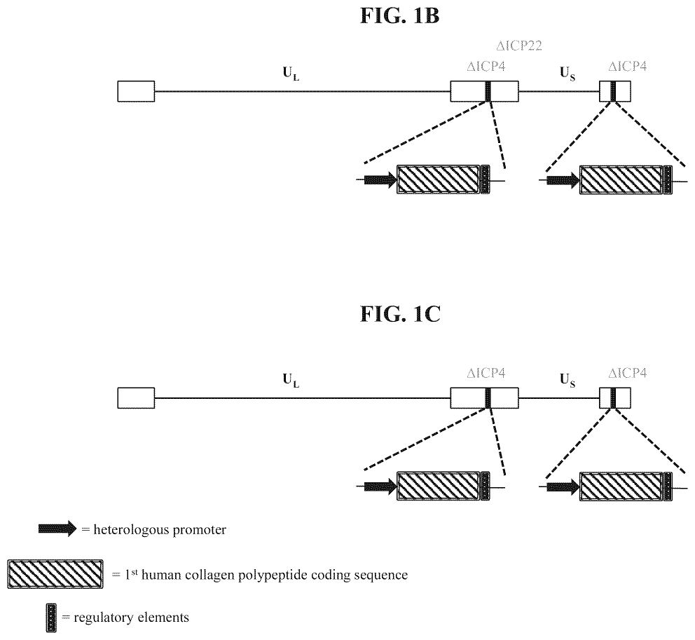

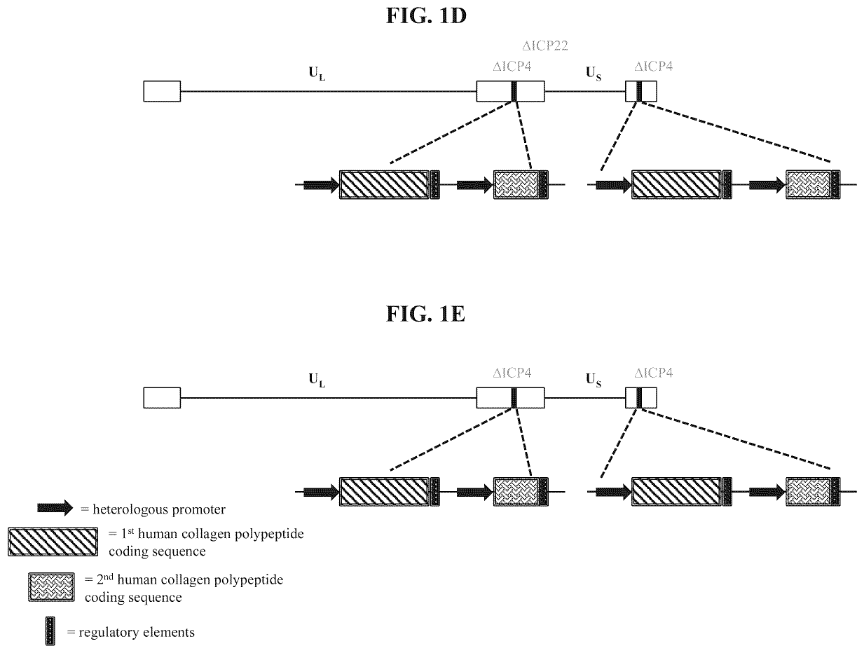

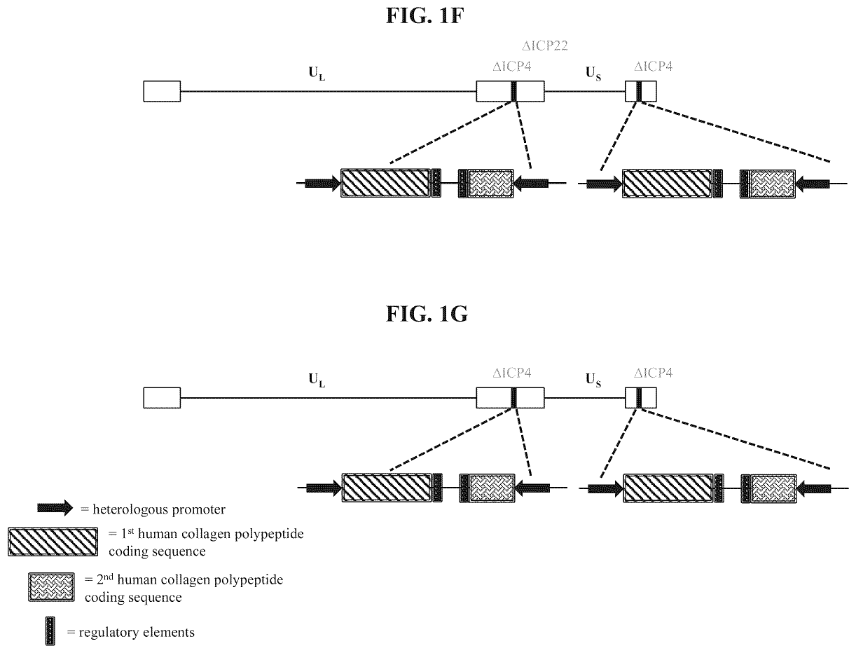

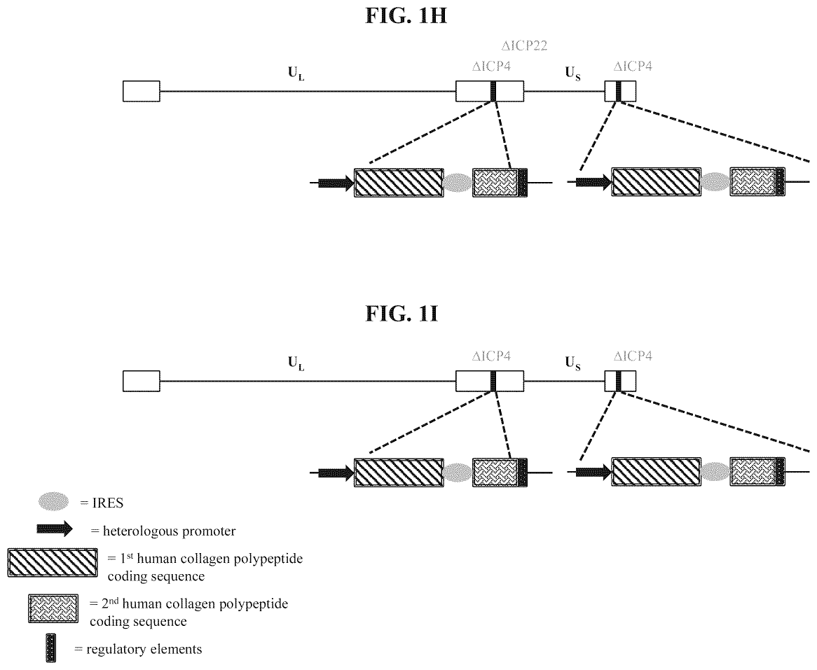

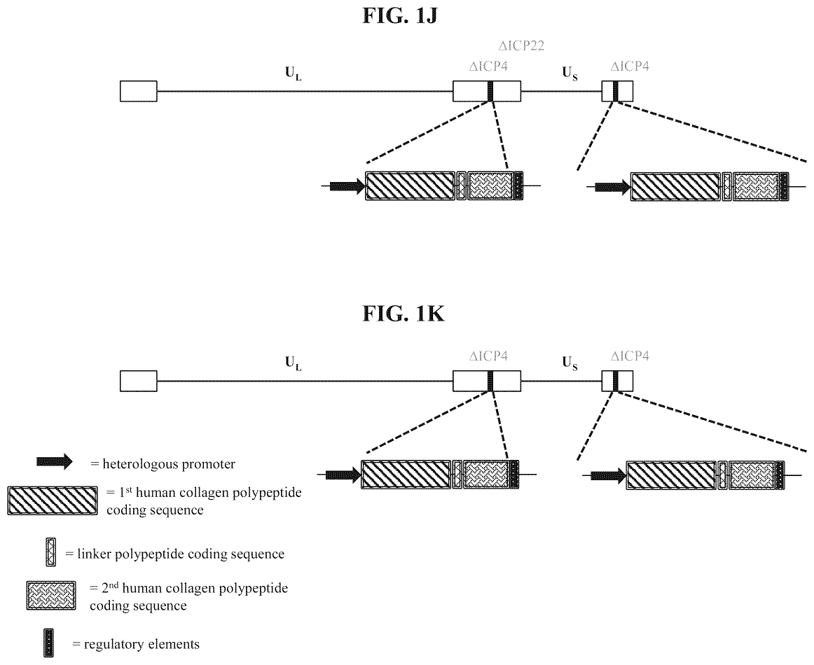

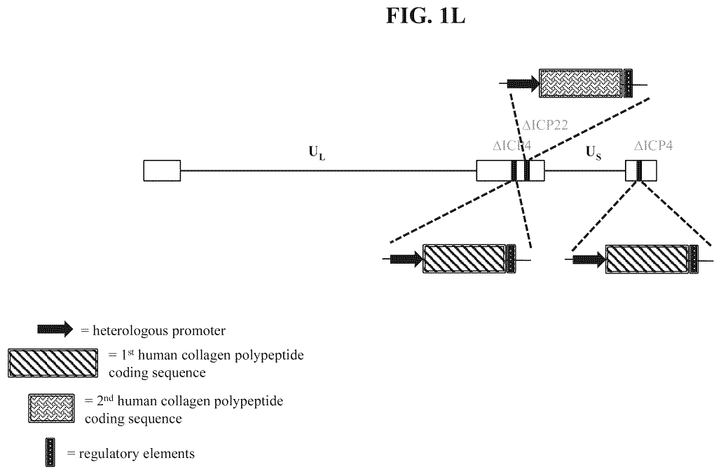

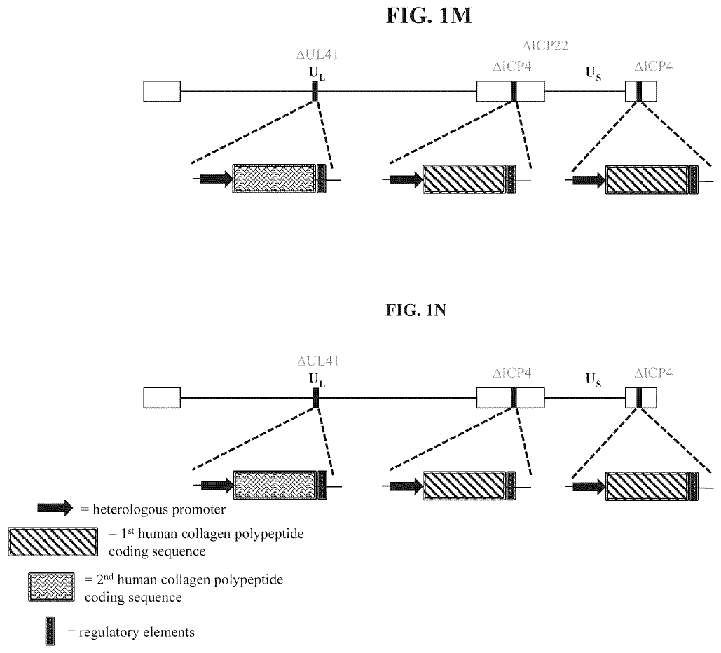

To make modified herpes simplex virus genome vectors capable of expressing human collagen protein(s) in a target mammalian cell (such as a human keratinocyte or fibroblast), a herpes simplex virus genome (FIG. 1A) is first modified to inactivate one or more herpes simplex virus genes. Such modifications may decrease the toxicity of the genome in mammalian cells. Next, variants of these modified/attenuated recombinant viral constructs are generated such that they carry one or more polynucleotides encoding human collagen protein(s). These variants include: 1) a recombinant ΔICP4/ΔICP22-modified HSV-1 genome comprising expression cassettes containing the coding sequence of a first human collagen protein under the control of a heterologous promoter integrated at each ICP4 locus (FIG. 1B); 2) a recombinant ΔICP4-modified HSV-1 genome comprising expression cassettes containing the coding sequence of a first human collagen protein under the control of a heterologous promoter integrated at each ICP4 locus (FIG. 1C); 3) a recombinant ΔICP4/ΔICP22-modified HSV-1 genome comprising expression cassettes containing the coding sequence of a first human collagen protein under the control of a first heterologous promoter and the coding sequence of a second human collagen protein under the control of a second heterologous promoter on the same strand of DNA integrated at each ICP4 locus (FIG. 1D); 4) a recombinant ΔICP4-modified HSV-1 genome comprising expression cassettes containing the coding sequence of a first human collagen protein under the control of a first heterologous promoter and the coding sequence of a second human collagen protein under the control of a second heterologous promoter on the same strand of DNA integrated at each ICP4 locus (FIG. 1E); 5) a recombinant ΔICP4/ΔICP22-modified HSV-1 genome comprising expression cassettes containing the coding sequence of a first human collagen protein under the control of a first heterologous promoter and the coding sequence of a second human collagen protein under the control of a second heterologous promoter on opposite strands of DNA integrated at each ICP4 locus (FIG. 1F); 6) a recombinant ΔICP4-modified HSV-1 genome comprising expression cassettes containing the coding sequence of a first human collagen protein under the control of a first heterologous promoter and the coding sequence of a second human collagen protein under the control of a second heterologous promoter on opposite strands of DNA integrated at each ICP4 locus (FIG. 1G); 7) a recombinant ΔICP4/ΔICP22-modified HSV-1 genome comprising expression cassettes encoding a polycistronic mRNA under the control of a heterologous promoter integrated at each of the ICP4 loci, where the polycistronic mRNA contains the coding sequence of a first human collagen protein and the coding sequence of a second human collagen protein separated by an internal ribosomal entry site (IRES) (FIG. 1H); 8) a recombinant ΔICP4-modified HSV-1 genome comprising expression cassettes encoding a polycistronic mRNA under the control of a heterologous promoter integrated at each of the ICP4 loci, where the polycistronic mRNA contains the coding sequence of a first human collagen protein and the coding sequence of a second human collagen protein separated by an internal ribosomal entry site (IRES) (FIG. 1I); 9) a recombinant ΔICP4/ΔICP22-modified HSV-1 genome comprising expression cassettes containing the coding sequence of a chimeric protein under the control of a heterologous promoter integrated at each of the ICP4 loci, where the chimeric protein contains the amino acid sequence of a first human collagen protein and the amino acid sequence of a second human collagen protein separated by the amino acid sequence of a linker polypeptide (FIG. 1J); 10) a recombinant ΔICP4-modified HSV-1 genome comprising expression cassettes containing the coding sequence of a chimeric protein under the control of a heterologous promoter integrated at each of the ICP4 loci, where the chimeric protein contains the amino acid sequence of a first human collagen protein and the amino acid sequence of a second human collagen protein separated by the amino acid sequence of a linker polypeptide (FIG. 1K); 11) a recombinant ΔICP4/ΔICP22-modified HSV-1 genome comprising expression cassettes containing the coding sequence of a first human collagen protein under the control of a heterologous promoter integrated at each of the ICP4 loci, and an expression cassette containing the coding sequence of a second human collagen protein under the control of a heterologous promoter integrated at the ICP22 locus (FIG. 1L); 12) a recombinant ΔICP4/ΔICP22/ΔUL41-modified HSV-1 genome comprising expression cassettes containing the coding sequence of a first human collagen protein under the control of a heterologous promoter integrated at each of the ICP4 loci, and an expression cassette containing the coding sequence of a second human collagen protein under the control of a heterologous promoter integrated at the UL41 locus (FIG. 1M); and 13) a recombinant ΔICP4/ΔUL41-modified HSV-1 genome comprising expression cassettes containing the coding sequence of a first human collagen protein under the control of a heterologous promoter integrated at each of the ICP4 loci, and an expression cassette containing the coding sequence of a second human collagen protein under the control of a heterologous promoter integrated at the UL41 locus (FIG. 1N).

These modified herpes simplex virus genome vectors are transfected into engineered Vero cells that are modified to express one or more herpes virus genes. These engineered Vero cells secrete replication-defective herpes simplex virus with the modified genomes packaged therein into the supernatant. The supernatant is then collected, concentrated, and sterile filtered through a 5 μm filter.

### Example 2: Construction and in Vitro Analysis of HSV Candidates Encoding Human COL7

Collagen alpha-1(VII) chain protein (COL7) functions to strengthen and stabilize the skin. Briefly, COL7A1 transcripts are translated, the resulting COL7 peptides are post-translationally modified by hydroxylation and glycosylation, and glycosylated COL7 tri-peptides form a triple helix known as pro-collagen, which is secreted from the cell. The pro-collagen associates into higher-order structures upon secretion, forming anchoring fibrils, which are then available to help organize, stabilize, and aid in the adherence of the epithelial basement membrane. The epithelial basement membrane is responsible for anchoring the epithelium to the underlying loose connective tissue and is essential for dermal-epidermal stability (dermo-epidermal junction integrity). Dystrophic epidermolysis bullosa is an inherited genetic condition caused by mutations in the COL7A1 gene; mutations in this gene impair the ability of COL7 to properly connect the epidermis to the dermis in dystrophic epidermolysis bullosa patients, leading to fragile skin. Recessive dystrophic epidermolysis bullosa (RDEB), the most severe form of epidermolysis bullosa, is most often characterized by extensive blistering and scarring of the skin and mucosal membranes.

The following example describes the construction of a recombinant herpes simplex type-1 viruses modified to express human Collagen alpha-1(VII) chain polypeptide (COL7), and further provides experiments showing that the recombinant HSV was capable of expressing functional human collagen in vitro in primary human keratinocytes and fibroblasts from healthy and RDEB patients.

### Materials and Methods

### Virus Construction

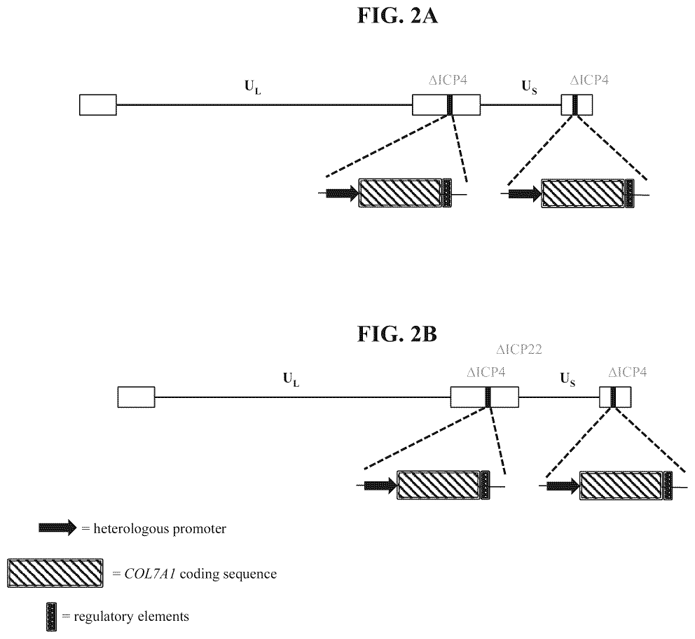

The “KCA211” viral vector (FIG. 2A) was generated as follows: a wild-type herpes simplex virus genome was first modified by deleting the coding sequence of both copies of the viral ICP4 gene (ΔICP4). The ΔICP4-modified viral genome was also engineered to contain an mCherry expression cassette in each of the ICP4 loci. The viral genome was then further modified to encode wild-type human COL7. Briefly, a plasmid containing the coding sequence for wild-type COL7 (under control of the hCMV promoter) flanked by the upstream (US) and downstream (DS) regions of ICP4 was transfected into Vero cells modified to express the herpes virus ICP4 gene. These transfected cells were then infected with the modified ΔICP4 mCherry-expressing virus described above. The US and DS ICP4 regions flanking COL7 allowed for a double crossover and replacement of each of the mCherry loci. Visual screening for the absence of mCherry fluorescence was then used to identify cells containing recombined virus.

The “SAR-COL7” viral vector (FIG. 2B) was generated as follows: a wild-type herpes simplex virus genome was first modified by deleting the coding sequence of both copies of the viral ICP4 gene as well as the single copy ICP22 gene (ΔICP4/ΔICP22). The ΔICP4/ΔICP22-modified viral genome was also engineered to contain an mCherry expression cassette in each of the ICP4 loci. The viral genome was then further modified to encode wild-type human COL7. Briefly, a plasmid containing the coding sequence for wild-type COL7 (under control of the hCMV promoter) flanked by the upstream (US) and downstream (DS) regions of ICP4 was transfected into Vero cells modified to express the herpes virus ICP4 gene. These transfected cells were then infected with the modified ΔICP4/ΔICP22 mCherry-expressing virus described above. The US and DS ICP4 regions flanking COL7 allowed for a double crossover and replacement of each of the mCherry loci. Visual screening for the absence of mCherry fluorescence was then used to identify cells containing recombined virus.

### Cell Culture

Cells were previously isolated from skin biopsies taken as part of routine surgical or diagnostic procedures. Informed written consent was obtained from each patient, or in the case of children, from the parent or legal guardian. This study was performed in accordance with the Helsinki declaration. All cells were cultured at 37° C. in 5% CO₂. Human fibroblasts were grown in Dulbecco's modified essential medium supplemented with 10% fetal bovine serum (PEAK® serum, cat. no. PS-FB1). RDEB and normal keratinocytes were cultured in DMEM/Ham's F12 medium (3:1) supplemented with 10% FBS, 10 ng/mL epidermal growth factor, 10-10 cholera toxin, 0.4 μg/mL hydrocortisone, 5 μg/mL transferrin, 5 μg/mL insulin, and 5 μg/mL liothyronine. All media contained ascorbic acid (150 μM). Keratinocytes were grown in the presence of a mitotically activated feeder layer of 3T3 cells and in the presence of 10 μM of the Rho-kinase inhibitor Y-27632.

### Virus Infections

Viral aliquots were stored at −80° C., and were left to defrost under the tissue culture laminar flow hood before use. Target cell number was determined prior to infection after detaching cells from the tissue culture plastic and counting on a hemocytometer. Multiplicity of infection (MOI) was calculated from the virus titer and target cell number, and the appropriate volume of virus stock was diluted in 10% serum containing DMEM and incubated with the target cells for 2 hours at 37° C. Virus was then removed, and fresh media was supplied to target cells after washing twice with pre-warmed media.

### Western Blots

Keratinocytes were plated in a 100 mm dish at 8×10⁵ to achieve 70-80% confluency the following day. 48 hours after infection, cells were lysed with radioimmunoprecipitation assay buffer. Lysate was placed in a centrifuge for 5 minutes at 4° C., and the supernatant was mixed with a 6× Laemmli loading buffer. Before loading onto SDS-PAGE, the samples were boiled for 5 minutes at 95° C. For COL7 detection, 5-30 μg of protein was loaded on a 6% acrylamide gel. The primary antibody used for COL7 detection was a rabbit antibody (Sigma, cat. no. HPA042420). Resolved proteins were transferred onto a nitrocellulose membrane, blocked in PBS-0.1% Tween with 5% milk or 5% BSA according to requirements of the primary antibody, and incubated overnight with the primary antibody. After incubation with IgG-HRP conjugated secondary antibody (Santa Cruz Biotechnology), the membrane was incubated with western blotting substrate (ThermoFisher Scientific, cat. no. 32106) and exposed to film (ThermoFisher Scientific, cat. no. 34090).

qRT-PCR

RNA was isolated using RNeasy® Mini Kit (Qiagen) according to the manufacturer's protocol. RNA extractions were quantified using a NanoDrop™ spectrophotometer (Fisher Scientific), and 1.5 μg RNA was used for cDNA synthesis using a SuperScript III First-Strand Synthesis system (Invitrogen). For qPCR, SYBR Select Master mix (Life Technologies) was used, and cDNA samples were diluted 1:25 to serve as template. Experiments were performed in triplicate.

### Adhesion Assay

96-well plates were left uncoated, or were coated with 10, 20, or 50 μg/mL rat tail collagen 1 (BD Biosciences) or human fibronectin (Millipore) in 100 μL reaction volume at 4° C. overnight, then washed with PBS, and blocked with PBS+0.1% BSA for 1 hour at 37° C. Mock (control) or SAR-COL7 infected RDEB keratinocytes (2.4×10⁴ cells in 100 μL of DMEM/HamF12+0.1% BSA) were added to the plates and incubated at 37° C. for 90 minutes. Wells were washed three times with PBS to remove any unbound cells, and adherent cells were fixed with PFE for 20 minutes. The fixed cells were then treated with 70% ethanol, stained with crystal violet, resolved in 100% ethanol, and quantified by measuring absorbance at 630 nM with a Flex Station 3 plate reader (Molecular Devices).

### Organotypic Skin Equivalents

Bovine fibrinogen (90% clottable, MP Biomedicals) was dissolved in 1.1% NaCl at 37° C. for 4 hours and then filtered with a 0.45 μm nylon membrane filter. Fibroblasts were collected with the use of trypsin and centrifugation and were resuspended in media to a final concentration of 2×10⁶ cells/mL. 150 μL of the cell suspension was mixed with 1 mL of thrombin (3 IU—Sigma Aldrich), and the cell/thrombin mix was added to fibrinogen at a ratio of 1:1. The mixture was quickly but gently distributed at 1 mL/well into a 12-well plate and incubated at 37° C. After 20 minutes, medium supplemented with ascorbic acid and aprotinin (Sigma) at a final concentration of 10 μg/mL was added. The matrices were left to mature for 5-7 days while medium was changed every other day. Keratinocytes were plated on top at 2×10⁶ cells/well, and on the next day the culture was raised to the air-liquid interface on a metal grid, and treatment with amlexanox was started. Medium was changed every other day with fresh drug, ascorbic acid and aprotinin. Cultures were collected at one or two weeks of treatment and frozen with OCT in liquid nitrogen-cooled isopentane. 8 μm sections were cut using a cryostat (Avantik QS11) and immunostained with polyclonal anti-COL7 antibody at a dilution of 1:800. Nuclei were counterstained with DAPI (Invitrogen).

### Results

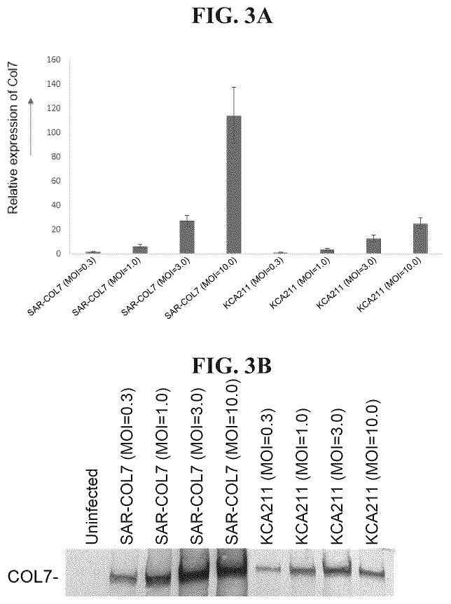

First, COL7 expression from modified HSV was assessed by qPCR and western blot analyses in the HaCaT human keratinocyte cell line to determine whether the modified HSVs were capable of expressing their cargo. HaCaT cells were transduced with either SAR-COL7 or KCA211 at MOIs ranging from 0.3-10. 48 hours after infection, cells were collected and processed for either qPCR (FIG. 3A) or western blot (FIG. 3B) analyses. The results demonstrated that full length COL7 was expressed in a dose-dependent manner from human keratinocytes infected with either modified HSV.

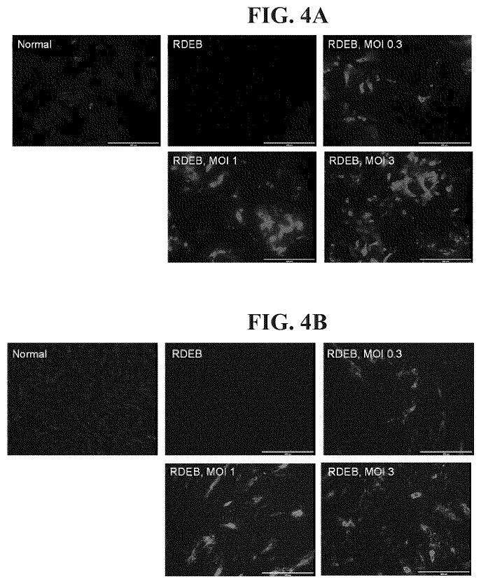

Next, immunofluorescence experiments were conducted to visualize COL7 expression in primary RDEB keratinocytes or RDEB fibroblasts infected for 24-48 hours with SAR-COL7 at various MOIs (ranging from 0.1 to 10). A strong COL7 signal was observed at all doses of SAR-COL7 tested for both keratinocytes (FIG. 4A) and fibroblasts (FIG. 4B), as compared to uninfected normal and RDEB keratinocytes and fibroblasts. The infection efficiencies of SAR-COL7 at MOIs of 0.1-1 in fibroblasts ranged from 16-36%. The infection efficiencies of SAR-COL7 at MOIs of 3.0 and above in fibroblasts were ≥90%.

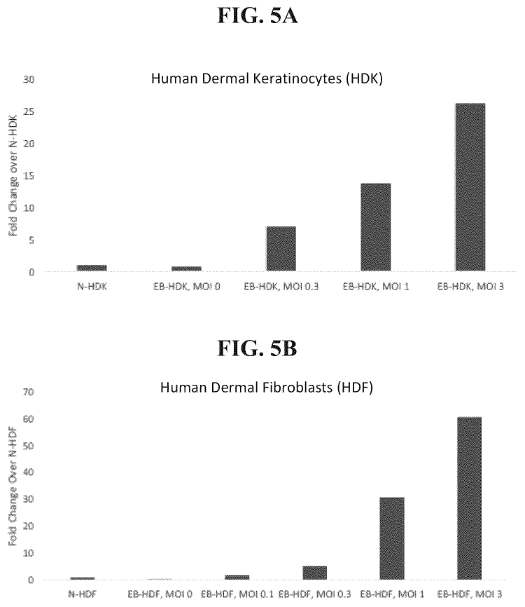

RDEB patient-derived human dermal keratinocytes (HDKs) and fibroblasts (HDFs) were infected with SAR-COL7 at varying MOIs in order to evaluate COL7A1 RNA expression. Normal HDKs and HDFs, as well as mock infected RDEB HDKs and HDFs, were used as negative controls. Dose-dependent increases in COL7A1 transcripts were observed in both HDKs (FIG. 5A) and HDFs (FIG. 5B) infected with SAR-COL7. The relative fold change in COL7A1 transcript expression after infection vs. uninfected healthy HDKs (Table 1A) or uninfected healthy HDFs (Table 1B) was also calculated. The use of COL7A1-encoding HSV was capable of increasing COL7A1 transcript expression by approximately 26-fold in RDEB HDKs and 60-fold in RDEB HDFs relative to wild-type COL7A1 transcript levels in healthy HDKs and HDFs at an MOI of 3.

TABLE 1A
COL7A1 expression in HDKs

| Cell type: | MOI: | Fold change over N-HDK: |
|---|---|---|
| Normal HDKs (N-HDK) | 0 | 1.00 |
| RDEB HDKs (EB-HDK) | 0 | 0.79 |
| RDEB HDKs (EB-HDK) | 0.3 | 7.00 |
| RDEB HDKs (EB-HDK) | 1.0 | 13.73 |
| RDEB HDKs (EB-HDK) | 3.0 | 26.25 |

TABLE 1B
COL7A1 expression in HDFs

| Cell type: | MOI: | Fold change over N-HDF: |
|---|---|---|
| Normal HDFs (N-HDF) | 0 | 1.000 |
| RDEB HDFs (EB-HDF) | 0 | 0.340 |
| RDEB HDFs (EB-HDF) | 0.1 | 1.805 |
| RDEB HDFs (EB-HDF) | 0.3 | 5.134 |
| RDEB HDFs (EB-HDF) | 1.0 | 30.788 |
| RDEB HDFs (EB-HDF) | 3.0 | 60.571 |

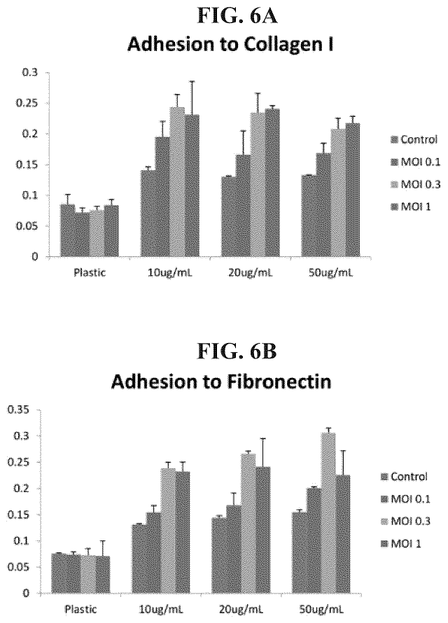

Next, the functionality of human COL7 expressed from SAR-COL7 was tested by a cell adhesion assay. The ability of uninfected RDEB keratinocytes, and RDEB keratinocytes infected with SAR-COL7 at varying MOIs, to adhere to wells treated with Collagen 1 or fibronectin was studied. Interestingly, RDEB keratinocytes infected with SAR-COL7 showed increased adhesion to Collagen 1 (FIG. 6A) and fibronectin (FIG. 6B) in dose-dependent manner using a plate-based adhesion assay.

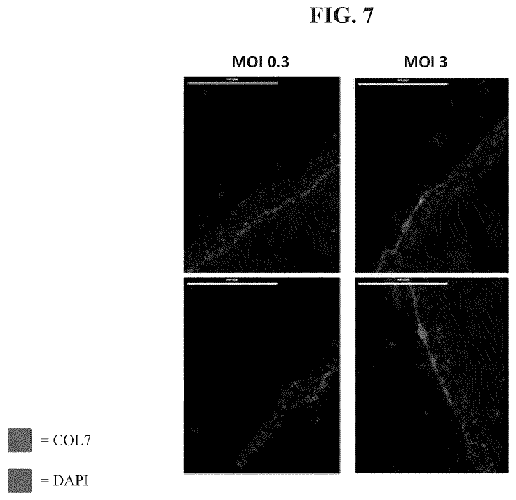

Finally, a skin equivalent (SE) organotypic culture composed of RDEB fibroblasts and keratinocytes was used to evaluate the expression of COL7 from SAR-COL7 at the Basement Membrane Zone (BMZ). Organ cultures were constructed with RDEB or normal fibroblasts and keratinocytes. RDEB cells were either infected with SAR-COL7 prior to organ culture construction (data not shown), or SAR-COL7 was added drop-wise onto the cultures prior to raising at the air-liquid interface (FIG. 7). The resulting skin equivalents (SEs) were isolated, sectioned, and stained for immunofluorescence to detect COL7 protein expression. COL7 was detected in these organotypic cultures from cells infected with SAR-COL7, and the initiation of COL7 protein deposition at the BMZ was observed. This data suggested that not only could SAR-COL7 deliver COL7A1 and express COL7 protein efficiently, but the COL7 protein began to organize in organotypic cultures similar to the pattern of organization expected for COL7 protein in vivo.

Taken together, the data provided herein indicated that replication-defective HSV may be employed as a vehicle for effectively delivering and expressing high levels of functional human collagen in wild-type primary human cells, as well as primary human cells isolated from patients suffering from a collagen deficiency, without any obvious toxicity in either 2D or 3D culture systems.

### Example 3: In Vivo Analysis of an HSV Candidate Encoding Human COL7 in Wild-Type Animals

The following example describes experiments showing that recombinant viruses constructed and validated in vitro in human cells (see Example 2 above) were capable of expressing the encoded human collagen in vivo in wild-type animals. The purpose of the study was, in part, to evaluate the skin biodistribution of HSV-mediated collagen expression in healthy immunocompetent animals.

### Materials and Methods

### Test Article

The active ingredient in the formulations administered to mice was the modified herpes simplex virus SAR-COL7 or KCA211 (see Example 2 above) at a titer of 4.8×10⁸ plaque forming units (PFU)/mL formulated in PBS+10% glycerol. The vehicle used for intradermal administration was Dulbecco's phosphate-buffered saline (DPBS)+10% glycerol. The vehicle used for topical administration was 3% hydroxypropyl methylcellulose (HPMC) gel formulated in sterile double distilled water.

### Animals

Healthy male BALB/c mice between 6 and 10 weeks of age were used. All procedures used in the protocol were in compliance with applicable animal welfare acts and were approved by the local Institutional Animal Care and Use Committee (IACUC).

### Intradermal Injections

Prior to and during test article administration, mice were anesthetized using a cocktail of Dexdomitor (30 μL or 0.05 mg/mL) and Telzol (50 μL of 10 mg/mL). The sedative was reversed with Antisedan (50 μL of 0.5 mg/mL). The back and flank areas were shaved using an electrical pet clipper, and the area was wiped with an alcohol wipe. Intradermal injections were performed using the Mantoux technique with a syringe and 27G needle (VWR, cat. no. BD305620), ensuring creation of a superficial “bleb” at each site. The virus was kept on dry ice, was thawed at room temperature, and was administered within 30 minutes of thawing. Two intradermal injections were administered to the back of each mouse, and the edges of the “bleb” were marked with a permanent marker.

### Topical Application

Topical administration was conducted either on open wounds or abraded or scarified skin. The back and flank regions were shaved using electrical clippers. Scarification was performed by gently abrading the skin with a mechanical Dremel followed by superficial perforation with a 22G needle. For creation of a wound, a 5-6 mm diameter biopsy of the skin was removed using sharp scissors. In order to contain the topical formulation to the abraded or wounded site, a well was created from cut and autoclaved tops of 1.5 mL micro-centrifuge tubes. The lids were retained, and the cut side was covered with transparent adhesive dressing. The “wells” were adhered to the abraded/wounded region using surgical glue with the lid side down. 100p L of SAR-COL7 was mixed with 20 μL of topical vehicle and was applied to the wound site by injection through the transparent adhesive in order to contain the topical gel on the wound and prevent leakage.

### Tissue Collection

At the indicated time points following SAR-COL7 administration, the mice were euthanized, and the injection site was removed using an 8 mm biopsy punch. One half of the biopsy was quick-frozen using liquid nitrogen, while the other half was embedded in OCT and cryopreserved for immunofluorescence staining.

### Real Time Quantitative PCR

Quick-frozen biopsy halves were stored at −80° C. until analysis. For processing and analysis, samples were resuspended in 350 μL RLT buffer prepared with fresh DTT following the manufacturer's protocol (Qiagen). The sample were sonicated 3 times at 25% amplitude with intermittent incubation for 1 minute on ice, and DNA and RNA extractions were performed according to the manufacturer's protocol (Qiagen AllPrep DNA and RNA extraction kit). Both RNA and DNA samples were resuspended in distilled, deionized RNAse free water and quantified spectrophotometrically on a Take3 microplate reader (BioRad).

Absolute quantification of COL7A1 DNA copies and RNA transcripts was performed by Tagman Real Time PCR analysis using a custom primer/probe assay that spanned the 3′ end of the human COL7A1 open reading frame and the 3′ UTR, specifically detecting the COL7A1 transgene. 100 ng of DNA and RNA was used for the qPCR and qRT-PCR assays respectively, and a plasmid standard containing the region to be amplified was prepared in 100 ng mouse genomic or RNA matrix. GAPDH was used as the control for both analyses.

### Immunofluorescent Staining

OCT frozen tissue was sectioned at 5-8 μm and left to air dry for up to 1 hour. The slides were dipped in 100% methanol for 10 minutes at −20° C. and left to air dry. The methanol-fixed sections were rehydrated through 3 washes in PBS (5 minutes each) at room temperature. The sections were incubated with a blocking solution composed of 10% serum (mixed species) for 1 hour at room temperature in a humid chamber. The excess blocking solution was removed and a drop of primary antibody (anti-human collagen 7, Sigma, cat. no. HPA042420; anti-integrin alpha 6 (clone goH3), BD Biosciences, cat. no. 555734) solution, prepared in 5% blocking solution, was applied on each section (30-50 μL/section). The sections were incubated with the primary antibody for 16 hours at 4° C., washed 3 time in PBS for 5 minutes each at room temperature, and secondary antibody (anti-rabbit AF 647, Invitrogen, cat. no. A21244; anti-rat AF-594, Invitrogen, cat. no. A11007) was applied at a 1:400 dilution in PBS for 1 hour at room temperature in a humid chamber. The 3 times PBS wash was repeated, then slides were immersed in Hoechst solution (1:1000) for 5 minutes at room temperature. The 3 times PBS wash was repeated, and the stained sections were mounted with mounting media (Fluorometer G, Southern Biotech, cat. no. 0100-01) and covered with a coverslip. The sections were imaged after dehydration (approximately 24 hours) using a Widefield Fluorescence Microscope.

### Results

A total of 30 male BALB/c mice divided into 6 groups were used for this study. SAR-COL7 was administered either by intradermal injection or topical application on day 1, and a subset of mice were harvested on day 3, and the remaining mice on day 6. Animals in group 2 received a low dose of SAR-COL7 (4.8×10⁶ pfu/site) in the same volume by intradermal injection, and group 1 served as a control for the intradermal cohorts. In groups 4, 5, and 6, the topical vehicle (group 4) or SAR-COL7 in topical gel (groups 5 and 6) was applied either to a wounded (groups 4 and 6) or abraded (group 5) area in a total volume of 120 μL. Tissues were harvested and processed for qPCR and immunofluorescence analysis as described above.

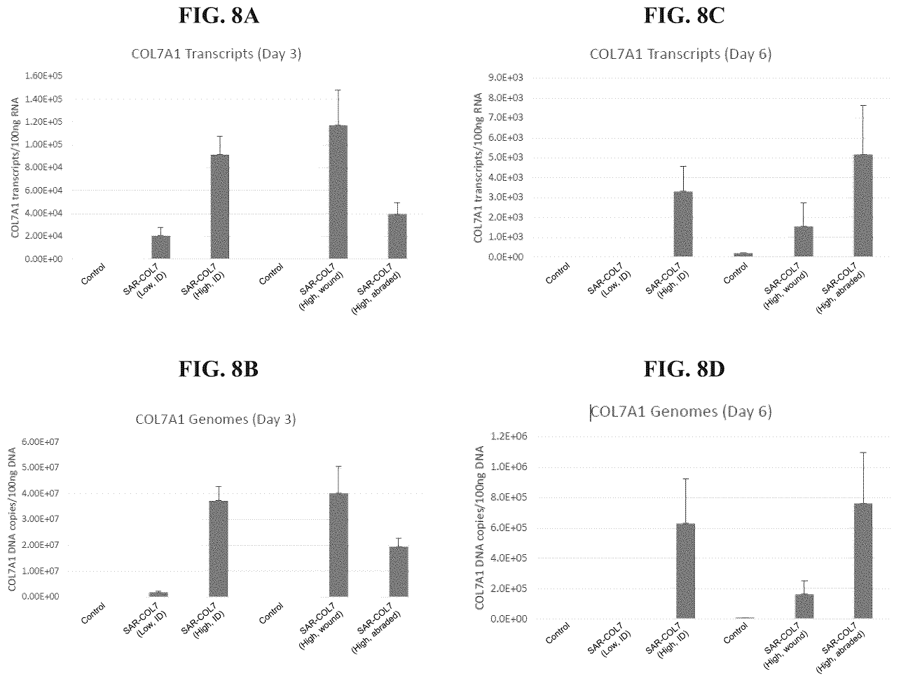

Post-sacrifice qPCR analysis was undertaken. COL7A1 transcripts and DNA levels were detected in all cohorts that received SAR-COL7 either by intradermal or topical application (FIGS. 8A-D), and a clear dose response was observed. Transcript and DNA levels were comparable between intradermal and topical cohorts (SAR-COL7 high ID, high wound, and high abraded), suggesting that topical application was as efficient at delivering COL7A1 as intradermal injection. Overall, the DNA and RNA levels were lower in the day 6 samples (FIGS. 8C-D) than the day 3 samples (FIGS. 8A-B), which was not unexpected since SAR-COL7 is a non-integrating vector that remains episomal (which would be expected to clear over time).

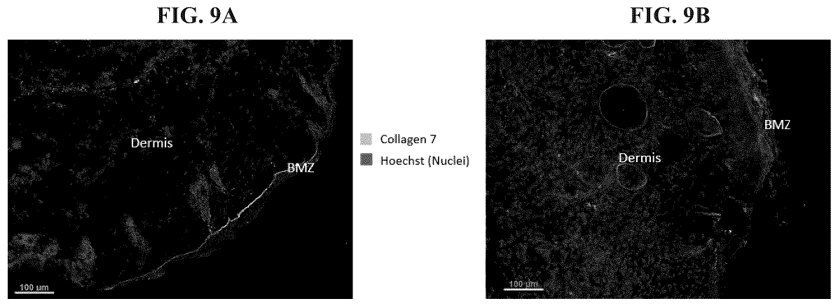

Next, immunofluorescence experiments were conducted to visualize COL7 expression after SAR-COL7 infection in vivo. As observed in the representative images provided in FIGS. 9A-B, COL7 was detected in most of the animal cohorts at both time points examined. Many samples showed correct localization of COL7 at the BMZ and around the hair follicles. In some instances, specifically with intradermal application, strong COL7 expression was observed in deeper layers of the skin, closer to the underlying fascia, possibly due to the injection being subcutaneous rather than intradermal. Similarly, in many of the abraded skin samples where the BMZ was likely removed during abrasion (as suggested by the lack of representative goH3 staining), the strong COL7 staining was limited to the skin surface. Overall, the presence of COL7 in the immunofluorescence samples strongly supported the robust efficacy of the modified HSV SAR-COL7.

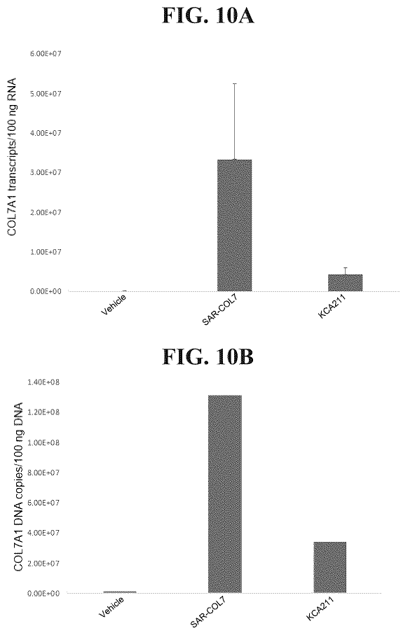

In addition, the ability of KCA211 to express human COL7 was tested in vivo and compared to SAR-COL7 administration. KCA211 was found to also express the COL7 transgene in vivo in immunocompetent mice (FIGS. 10A-B).

Taken together, the data indicated that modified HSV was capable of delivering and expressing a human collagen protein in vivo in healthy, immunocompetent animals after topical or intradermal administration, and further, that collagen expression from topically administered virus into compromised skin or open wounds was comparable to intradermal administration into intact skin.

### Example 4: In Vivo Analysis of Low and High Doses of an HSV Candidate Encoding Human COL7 in Hypomorphic Animals

The following example describes experiments showing that recombinant viruses constructed and validated in vitro in human cells (see Example 2 above) and in vivo in wild-type mice (see Example 3 above) were capable of expressing functional human collagen in vivo in COL7 hypomorphic mice. The purpose of the study was, in part, to evaluate the skin biodistribution of HSV-mediated collagen expression in COL7-deficient, immunocompetent animals.

### Materials and Methods

Unless indicated to the contrary, experiments were conducted as described in Example 3 above.

### Hypomorphic Mice

The COL7 hypomorphic mouse model (Fritsch et al. J Clin Invest. 2008 May; 118(5):1669-79) was used in this study. This hypomorphic mouse model is an immunocompetent animal model for dystrophic epidermolysis bullosa (DEB) in which the mice express about 10% of normal levels of COL7. Their phenotype closely resembles characteristics of severe human DEB, including mucocutaneous blistering, nail dystrophy, and mitten deformities of the extremities.

The mice were generated by flp/frt-mediated removal of exon 2 of mouse COL7A1. Animals lacking both functional copies of COL7A1 (Col7a1flNew/flNeo), referred to as COL7 “hypomorphic mice”, expressed about 10% of normal levels of COL7. From a total of 15 breeding pairs, 58 pups were obtained, with the litters ranging from 2-7 mice/litter. Out of these 58 pups, 6 were genotyped to be hypomorphs. Mice were genotyped with DNA extracted from an ear punch tissue sample. PCR analysis detected the presence of a loxP site upstream of exon 2 of COL7A1. Wild-type (WT) mice showed a band at 269 base pairs (bp), hypomorph mice showed a band at 435 bp, and heterozygous mice showed both bands. All procedures were in compliance with applicable animal welfare acts and were approved by the local Institutional Animal Care and Use Committee (IACUC).

### Intradermal Injections

Prior to and during test article administration, mice were maintained under inhalation anesthesia using 2% isoflurane. Eye ointment (Puralube® Vet) was applied on the eyes to prevent dryness. Intradermal injections were performed using the Mantoux technique with a 31G needle. Up to four intradermal injections were administered to the back of each mouse at the specified doses.

### Tissue Collection

Prior to tissue collection, animals were euthanized by CO₂ inhalation followed by cervical dislocation. The injection sites were biopsied using sharp scissors.

### Hematoxylin and Eosin (H&E) Staining

Cryopreserved tissues were sectioned at a thickness of 5-8 μm and left to air dry for up to one hour. The slides were dipped in 100% methanol for ten minutes at −20° C. and left to air dry. Methanol fixed sections were rehydrated in PBS for 5 minutes at room temperature. The sections were incubated in hematoxylin (Weigert's modified hematoxylin) for 5-10 minutes at room temperature, followed by a wash in PBS for 15 minutes at room temperature. The sections were then rinsed in Eosin (Eosin Y solution, cat. no. HT110116) 3 times followed by one rinse in water.

The sections were gradually dehydrated with ethanol by dipping in 70% ethyl alcohol 10 times, 95% ethyl alcohol ten time, and 100% ethyl alcohol ten times. The sections were set to dry, mounted with mounting media (Fischer Scientific, cat. no. SPF15-100) and covered with a coverslip. The sections were imaged after dehydration (approximately 24 hours) using a bright field microscope.

### Electron Microscopy

Skin was prepared for electron microscopy by immersion in 1.5% glutaraldehyde/1.5% paraformaldehyde in Dulbecco's serum free media (SFM) containing 0.05% tannic acid for a minimum of one hour, followed by an extensive rinse in SFM, and a post-fixation step in 1% OsO₄ for 60 minutes. The samples were washed in SFM then dehydrated in a graded series of ethanol to 100%, rinsed in propylene oxide, and infiltrated in Spurr's epoxy over a total time of two hours, accelerated via microwave energy. Samples were polymerized at 70° C. over 18 hours. Additional samples were prepared by extensively rinsing in SFM then immersing in mouse IgM LH24 antibody or mouse IgG NP185 antibody diluted 1:5 in SFM overnight at 4° C. The samples were then rinsed extensively in SFM, exposed to gold enhancement solution (Nanoprobes) for 15 minutes on ice, then rapidly warmed to 25° C. and incubated an additional 5 minutes. The samples were rinsed with ice cold SFM, fixed, and embedded.

### Results

Three hypomorphic mice were used for the high-dose SAR-COL7 study. All mice received a dose of 4.6×10⁷ PFU/50 μL/injection site by intradermal injection on day 1 (Table 2). Each animal was shaved and injected at 4 sites on the back, including 1 control injection and 3 SAR-COL7 injections. One animal (mouse 3) received a second injection at the same 4 sites on day 3. One mouse (mouse 1) was sacrificed on day 3, while mouse 2 and mouse 3 were sacrificed on day 7.

TABLE 2
study design for intradermal injection of high-dose SAR-COL7

| | | Treatment | Treatment | Day of |
|---|---|---|---|---|
| Sample: | Mouse: | (Day 1): | (Day 3): | Sacrifice: |
| 1 | 1 | HSV-GFP | PBS | Day 3 |
| 2 | | SAR-COL7 | — | |
| 3 | | SAR-COL7 | — | |
| 4 | | SAR-COL7 | — | |
| 5 | 2 | PBS | PBS | Day 7 |
| 6 | | SAR-COL7 | — | |
| 7 | | SAR-COL7 | — | |
| 8 | | SAR-COL7 | — | |
| 9 | 3 | PBS | PBS | Day 7 |
| 10 | | SAR-COL7 | SAR-COL7 | |
| 11 | | SAR-COL7 | SAR-COL7 | |
| 12 | | SAR-COL7 | SAR-COL7 | |

Post-sacrifice qPCR analysis was undertaken. COL7A1 transcripts (FIG. 11A) and DNA levels (FIG. 11B) were detected at each viral injection site in all three mice. Transcript levels in all of the control samples (PBS or HSV-GFP) were at or below the level of detection in the assay, so only 1 control (day 3, sample 1, HSV-GFP) was included for comparison in the graphs. Some decrease in DNA and transcript levels by day 7 after single administration of SAR-COL7 (mouse 2) was observed; however, DNA and transcript levels increased upon re-administration of SAR-COL7 (mouse 3).

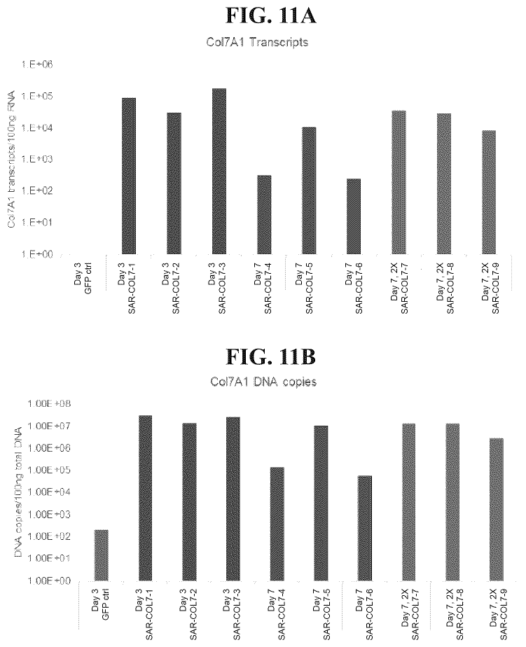

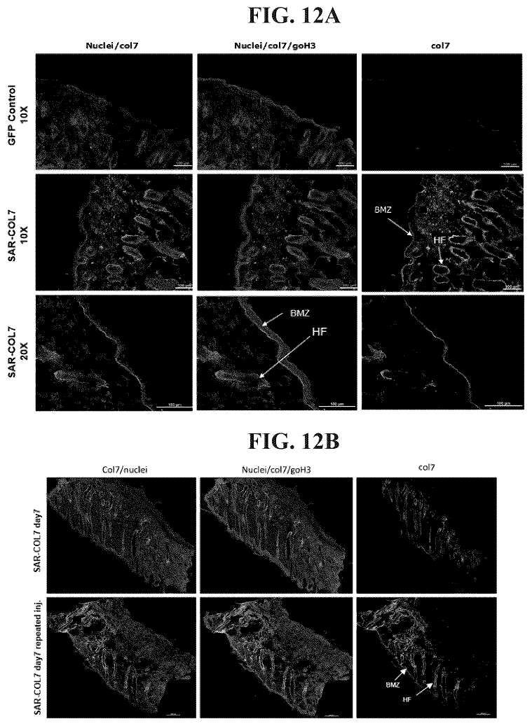

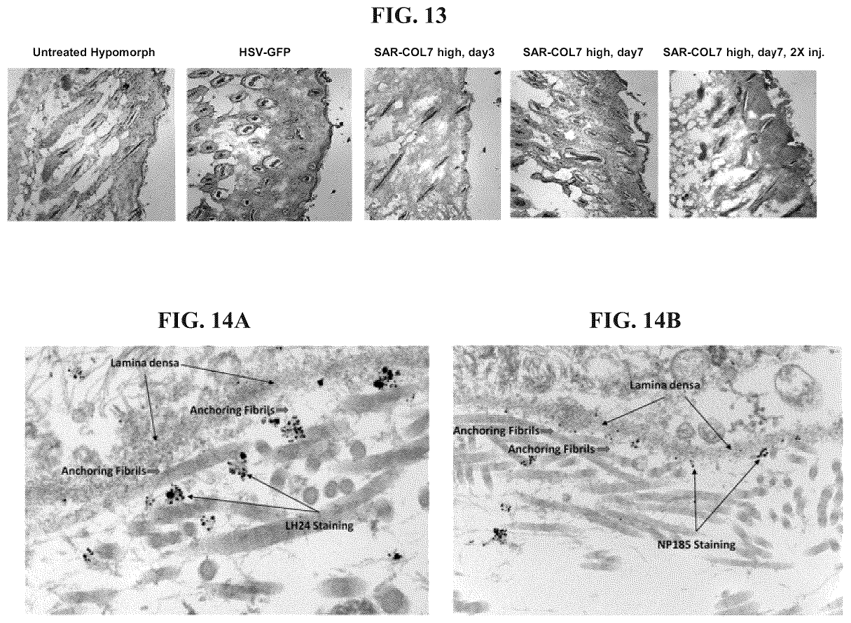

Next, immunofluorescence (IF) experiments were conducted to visualize COL7 expression in hypomorphic mice after SAR-COL7 infection in vivo (FIGS. 12A-B). The IF experiments demonstrated that robust and widespread COL7 protein expression was observed in the BMZ, as well as around the hair follicles (HF), at both the day 3 and day 7 timepoints. No negative impact on skin morphology (even after repeat administration) was observed, as the SAR-COL7 treated samples showed a normal skin morphology with no obvious signs of fibrosis or acute inflammation (FIG. 13). Overall, the presence of COL7 in the immunofluorescence samples strongly supported the robust efficacy of SAR-COL7 in delivering human collagen capable of being secreted and appropriately organized in the underlying skin substructures.

Day 3 biopsies were also evaluated for anchoring fibril formation by electron microscopy. In appropriately structured anchoring fibrils, the NC1 domain of COL7 (which is stained with the NP158 antibody) aligns towards the lamina densa, while the NC2 domain of COL7 (which is stained with the LH24 antibody) aligns away from the lamina densa. Biopsies from SAR-COL7-injected mice showed COL7 staining with both the LH24 (FIG. 14A) and NP185 (FIG. 14B) antibodies, and importantly, the electron microscopy (EM) images revealed the formation of anchoring fibrils. The lamina densa was observed as a dark band through the middle of the EM images. The NC2 domains of the exogenous human COL7 were positioned away from the lamina densa, while the NC1 domains of the exogenous human COL7 were positioned along the lamina densa, as would be expected in properly formed anchoring fibrils. This data indicated that SAR-COL7 could not only express an encoded human COL7 that was capable of being secreted and appropriately organized at the BMZ, but that the secreted COL7 was functional and properly positioned in the resulting anchoring fibrils, supporting the skin tissue of the hypomorphic mice.

Three additional hypomorphic mice were used for the low-dose SAR-COL7 study. All mice received a dose of 6.4×10⁶ PFU/50 μL/injection site of SAR-COL7 in 3 (mouse 1) or 2 (mouse 2 and 3) sites by intradermal injection on day 1 (Table 3).

TABLE 3
study design for intradermal injection of low-dose SAR-COL7

| Mouse: | Treatment Day: | No. of Injection Sites: | Termination Day: |
|---|---|---|---|
| 1 | 1 | 3 | 3 |
| 2 | 1 | 2 | 3 |
| 3 | 1 | 2 | 7 |

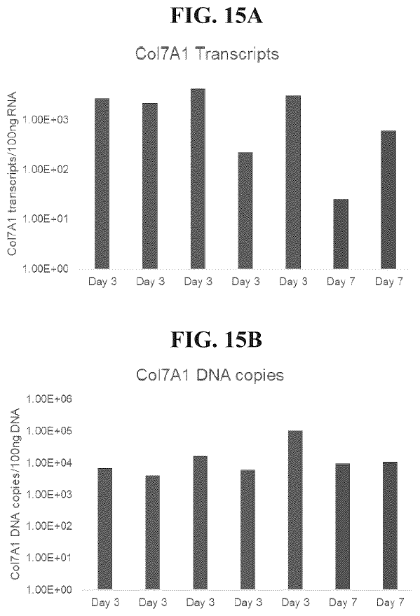

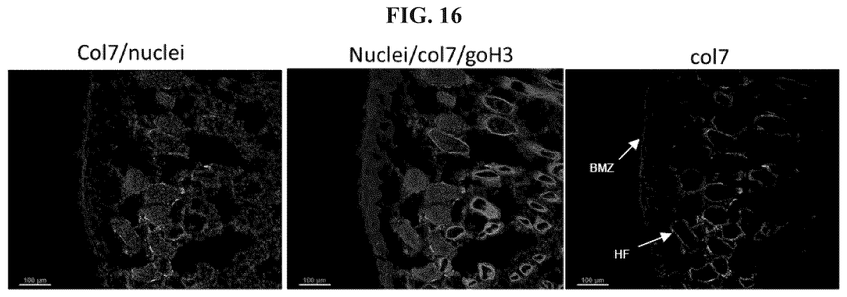

Post-sacrifice qPCR analysis was undertaken. COL7A1 transcripts (FIG. 15A) and DNA levels (FIG. 15B) were detected at each viral injection site in all three mice. A dose response was observed. While both DNA and transcript levels of COL7A1 were lower in these low-dose samples relative to those observed in high-dose study, COL7A1 expression was still detectable. Despite an approximate 2 log reduction in DNA or RNA levels, COL7 protein was still detectable in the skin of low-dose SAR-COL7 injected animals (FIG. 16). The COL7 protein was observed in both the BMZ and around the hair follicles. The data indicated that even at lower dosages, the modified HSV was still effective at delivering and expressing the encoded human collagen protein, which was then secreted and localized to the appropriate region of the skin.

Taken together, the data provided in the examples indicated that the modified HSVs described herein were able to: 1) transduce skin cells to produce functional human collagen in vitro and in organotypic culture (see Example 2); 2) transduce skin and deliver human collagen in vivo in healthy, immunocompetent animals after both topical and intradermal applications (see Example 3); and 3) transduce skin, deliver human collagen, and initiate the formation of anchoring fibrils in vivo in collagen-deficient animals (see Example 4). Without wishing to be bound by theory, it is believed that the recombinant vectors described herein provide a desirable strategy for delivering functional human collagen to the skin, allowing for novel approaches to support the dermis and/or epidermis in cosmetic settings.

### Example 5: Construction and Validation of Multiple Engineered HSVs Encoding Human Collagen 1

The following example describes two different approaches for engineering a recombinant HSV-1 to successfully express human Collagen 1. Because human Collagen 1 is a heterotrimeric macromolecule comprising two copies of a COL1A1 polypeptide and one copy of a COL1A2 polypeptide, two distinct strategies were undertaken to generate a single HSV genome encoding both human COL1A1 and COL1A2; these two approaches are generally depicted in FIGS. 1H-1I and 1L, and described in detail below.

As a first approach, a recombinant HSV-1 was initially engineered to incorporate a human COL1A1 expression cassette, containing a heterologous promoter and polyA sequence, into each of the ICP4 loci as described in Example 2 above. Multiple plaques of viruses putatively containing the human COL1A1 cassette were picked and screened by infection in Vero cells to test for COL1A1 expression (data not shown). At least three isolates (clones 1A1, 2A1, and 3A1) were positive for human COL1A1 transgene expression. Next, the human COL1A1-positive clones 1A1 and 2A1 were then engineered to incorporate a human COL1A2 expression cassette, containing a different heterologous promoter than the COL1A1 cassette and its own polyA sequence, inserted into the ICP22 locus. Seven isolates (clones 1A2-1A1, 1B2-1A1, 1C1-1A1, 1B3-2A1, 3B1-1A1, and 3B1-2A1) were then screened to identify attenuated HSV-1 viruses capable of expressing both human COL1A1 and COL1A2, and thus, full-length human Collagen 1.

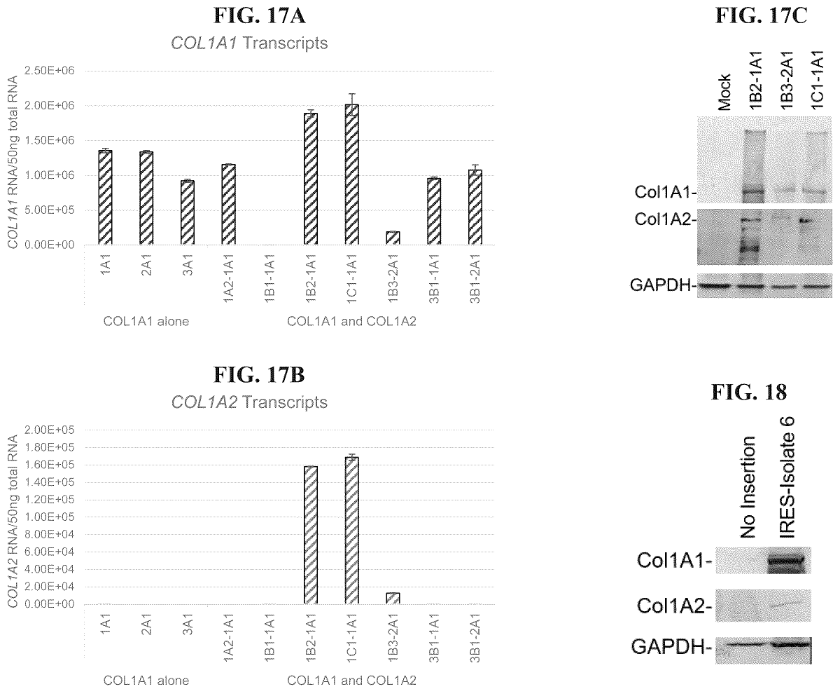

Vero cells were infected with one of the seven putative COL1A1-COL1A2-positive isolates or one of three of the COL1A1-alone isolates (as a negative control for COL1A2 expression) in order to identify recombinant HSV-1 clones capable of expressing both transgenes. Infections of the Vero cells were allowed to proceed for five days, cells were harvested by gentle scraping, and cell pellets were collected by centrifugation at 6000×g for five minutes. Half of each pellet was harvested for qRT-PCR analysis, while the other half was processed for western botting. Absolute quantification of COL1A1 or COL1A2 RNA copies was performed by Tagman Real-Time PCR analysis using custom primer/probe assays that span the 3′ end of the human COL1A1 or COL1A2 open reading frame (ORF) and the 3′ untranslated region (UTR), and are specific to the transgene. RNA extracted from cells infected with all three COL1A1-alone viruses, and cells infected with 6/7 of the COL1A1-COL1A2 viruses, were positive for human COL1A1 transgene expression (FIG. 17A). Interestingly, only 3/7 of the COL1A1-COL1A2 viruses (clones 1B2-1A1, 1C1-1A1, and 1B3-2A1) also expressed detectable levels of human COL1A2 (FIG. 17B). Western blotting of COL1A1 and COL1A2 protein expression in cell lysates infected with these three isolates was performed to ensure that the transgenes were expressed at both the nucleic acid and protein levels. Briefly, cell pellets were resuspended in RIPA buffer containing Halt protease inhibitor cocktail, incubated at 4° C. for ten minutes, and were then treated with benzonase for 10 minutes at room temperature. Samples were then diluted with 4×LDS sample buffer containing 5% 2-mercaptoethanol, and resolved on Tris-Glycine gels. Mock-infected Vero cell lysate was loaded on the gel as a negative control. Proteins were transferred to PVDF, blocked, and then stained overnight with primary antibody (anti-human COL1A1, ThermoFisher cat. no. PA5-29569; anti-human COL1A2, Abcam cat. no. ab96723; or anti-GAPDH, Abcam cat. no. ab9485). Stained membranes were then washed, incubated with secondary antibody, then developed. In agreement with the qRT-PCR analysis, all three clones were capable of expressing both human COL1A1 and COL1A2 proteins at detectable levels (FIG. 17C).

A second approach was pursued in parallel to generate COL1A1-COL1A2-positive recombinant, attenuated HSV vectors. Here, a polynucleotide construct was generated which encoded, from 5′ to 3′, a human COL1A1 ORF, a synthetic IRES, and a human COL1A2 ORF. This IRES-based construct (encoded in an expression cassette also containing a heterologous promoter and a polyA sequence) was then inserted into each of the ICP4 loci, as described in Example 2 above. Multiple plaques were picked and screened to identify vectors with correctly inserted IRES constructs. Briefly, Vero cells were infected with putative IRES viral isolates, infections were allowed to proceed for five days, and cells were harvested and processed for qPCR and qRT-PCR analysis. Absolute quantification of insert DNA and RNA copies was performed by Taqman Real-Time PCR analysis using custom primer/probe assays that recognize the synthetic IRES. 13 viral isolates were screened by qPCR and qRT-PCR analysis; only one of these isolates (isolate 6) tested positive for the IRES DNA and RNA transcripts comprising the IRES sequence (data not shown). To confirm that this isolate was capable of expressing both human COL1A1 and COL1A2 at the protein level, infected Vero cells were processed for western blotting as described above. A viral isolate (isolate 1) that showed no expression cassette incorporation by qPCR and transgene expression by qRT-PCR was used as a negative control. Paralleling the qPCR/qRT-PCR data, viral isolate 6 was capable of expressing both human COL1A1 and COL1A2 protein after infection (FIG. 18).

Taken together, the data presented in this sample indicate that: (1) multiple recombinant HSV-1 vectors were successfully constructed that were proficient in expressing both human COL1A1 and COL1A2 after infecting targeted cells; (2) vectors can be engineered to express heterotrimeric human collagen proteins; and (3) multiple different approaches can be taken to express multiple proteins from a single recombinant genome. Without wishing to be bound by theory, it is believed that successful expression of human Collagen 1 from a recombinant HSV-1 genome provides support for the use of engineered HSV to express any heterotrimeric collagen protein (e.g., human Collagen 4).

### Example 6: Construction, Validation, and in Vitro Characterization of an Engineered HSV Encoding Human Collagen 3

The following example describes the engineering of a recombinant HSV-1 that successfully expressed human Collagen 3 (termed C3vec01). In addition, the following example describes in vitro experiments establishing multiple relevant 2D cell culture model systems suitable for characterizing the efficacy of C3vec01, including the use of immortalized human keratinocytes and fibroblasts in dose-ranging studies, the use of primary human dermal fibroblasts biopsied from multiple aged human patients as a model of C3vec01-mediated Collagen 3 rescue in older patients, and the use of in vitro UV-irradiated immortalized human fibroblasts as a model for sun exposure/skin aging.

Human skin is largely composed of collagen-rich connective tissue which is produced, organized, and maintained by dermal fibroblasts. Dermal collagen represents >90% (dry weight) of human skin and is composed primarily of COL1 and COL3 fibrils at a typical ratio of about 85:15. These fibrils provide strength to the skin and are critical for the maintenance of skin tissue architecture.

Skin aging characteristics are largely due to aberrant collagen homeostasis, resulting in a net collagen deficiency; biosynthesis of collagen is reduced, collagen fibril fragmentation is increased, and there is a progressive loss of dermal collagen, all of which contribute to the aged phenotype. Skin aging is influenced by a combination of both internal and external factors: intrinsic factors—the passage of time, genetics, cellular metabolism, hormones, etc.; and extrinsic—chronic light exposure, pollution, ionizing radiation, etc. These factors together lead to cumulative structural and physiological alterations to the skin, ultimately leading to the appearance of, and worsening in, skin wrinkles.

Skin rejuvenation, the process of reversing or repairing irregularities in the skin (such as wrinkles), is achieved, in part, by the synthesis of new collagen (neocollagenesis). In the skin, neocollagenesis is affected by the deposition of, and complex interactions between, collagens 1 and 3. COL3 appears early during collagen fibrillogenesis, and the subsequent replacement of this COL3 by COL1 is a critical step for collagen fibril maturation and extracellular matrix reorganization (Wang, et al., 2018, Journal of the Chinese Medical Association, 81(2), pp. 94-101). In addition, COL3 both regulates the dimensions of COL1 fibers (Liu, et al., 1997, Proc Natl Acad Sci USA, 94(5), pp. 1853-6) and enhances COL1 elasticity (Asgari, et al., 2017, Sci Rep, 7(1), p. 1392). As such, the appearance of early COL3 expression, and ensuing replacement with COL1, has been used as a marker of efficacy for injectable facial fillers in humans (Yutskovskaya, et al., 2014, J Drugs Dermatol, 13(9), pp. 1047-52).

All experiments were conducted as described above unless noted otherwise.

To begin, a recombinant HSV-1 was engineered to incorporate a human COL3A1 expression cassette, containing a heterologous promoter and polyA sequence, into each of the ICP4 loci. Multiple plaques of viruses putatively containing the human COL3A1 cassette were picked and screened by infection in Vero cells to test for COL3A1 expression (data not shown). One of the high expressing clones, termed C3vec01, was subsequently selected for additional in vitro (described below) and in vivo (Example 7) analyses.

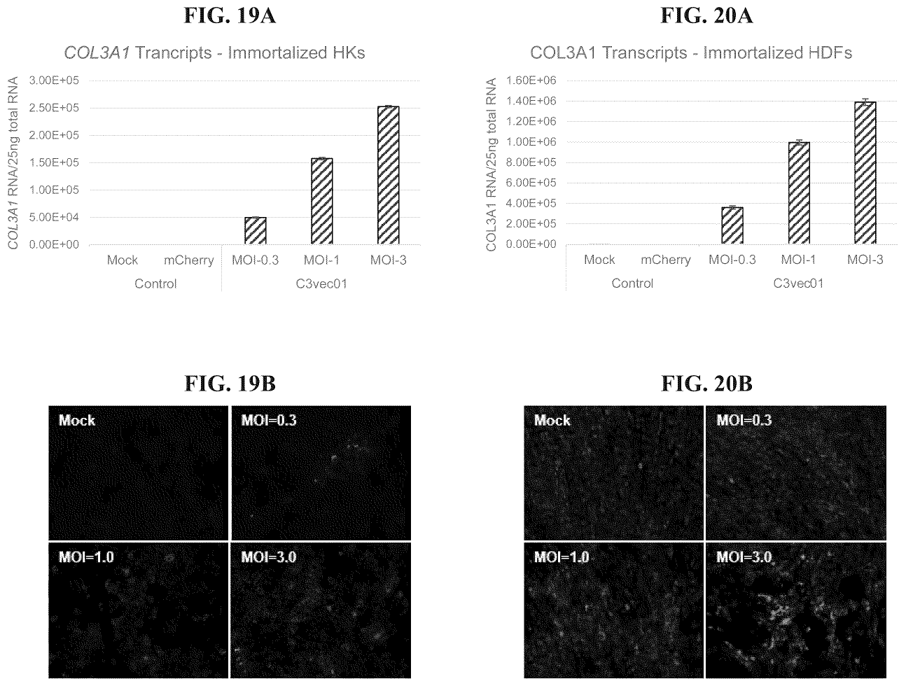

First, a dose-ranging study was conducted to determine the efficacy of C3vec01-mediated delivery of its encoded human cargo in both immortalized human keratinocytes (FIG. 19) and immortalized human dermal fibroblasts (FIG. 20). The immortalized cells were infected for 48 hours at various multiplicities of infection (MOI) ranging from 0.3 to 3, and human Collagen 3 expression was quantitatively and qualitatively measured via multiple assays. Dose-dependent increases were observed in both effector DNA by qPCR analysis (data not shown) and effector transcript levels by qRT-PCR analysis in the immortalized HKs and HDFs (FIGS. 19A and 20A, respectively). Mock infected cells, and cells infected with a virus containing the same HSV-1 backbone as C3vec01 but instead encoding an mCherry effector, were used as negative controls. Paralleling these results, a dose-dependent increase in COL3 protein expression after C3vec01 infection was observed by immunofluorescence in immortalized HKs (FIG. 19B) and HDFs (FIG. 20B). While the immortalized HDFs expressed endogenous human COL3 prior to infection (as expected), a significant increase in COL3 expression was observed after infection with C3vec01, even at a low dose (primary anti-COL3 antibody, Abcam cat. no. ab7778). Little-to-no detectable endogenous COL3 was observed in the uninfected immortalized keratinocytes. Importantly, no significant effect on cell morphology or viability was observed in immortalized keratinocytes or fibroblasts infected with C3vec01, even at high doses.

As the skin ages, resident dermal fibroblasts produce less Collagen 3, and the ratio of COL1:COL3 in the skin skews towards Collagen 1. In order to provide skin rejuvenation through the synthesis of new Collagen 3, it was important to understand whether C3vec01 would be capable of effectively infecting aged dermal fibroblasts and robustly express its encoded human Collagen 3. As such, the ability of C3vec01 to infect aged primary dermal fibroblasts and express exogenous Collagen 3 at multiple MOIs was tested in cells sourced from two different vendors. Table 4 below provides donor information for the four primary HDF samples used in this study.

TABLE 4
primary human dermal fibroblast donors

| Age | Sex | Race | Tissue | Cat. No. | Lot No. | Company |
|---|---|---|---|---|---|---|
| 73 | M | Caucasian | Skin/ | C-12302 | 435Z009.2 | PromoCell |
| | | | eyelid | | | |
| 65 | F | Caucasian | Skin/ | C-12302 | 417Z010.2 | PromoCell |
| | | | eyelid | | | |
| 73 | M | Caucasian | Left | CC-2511 | 0000633428 | Lonza |
| | | | lower | | | |
| | | | back | | | |
| 75 | F | Caucasian | Back | CC-2511 | 18TL057585 | Lonza |

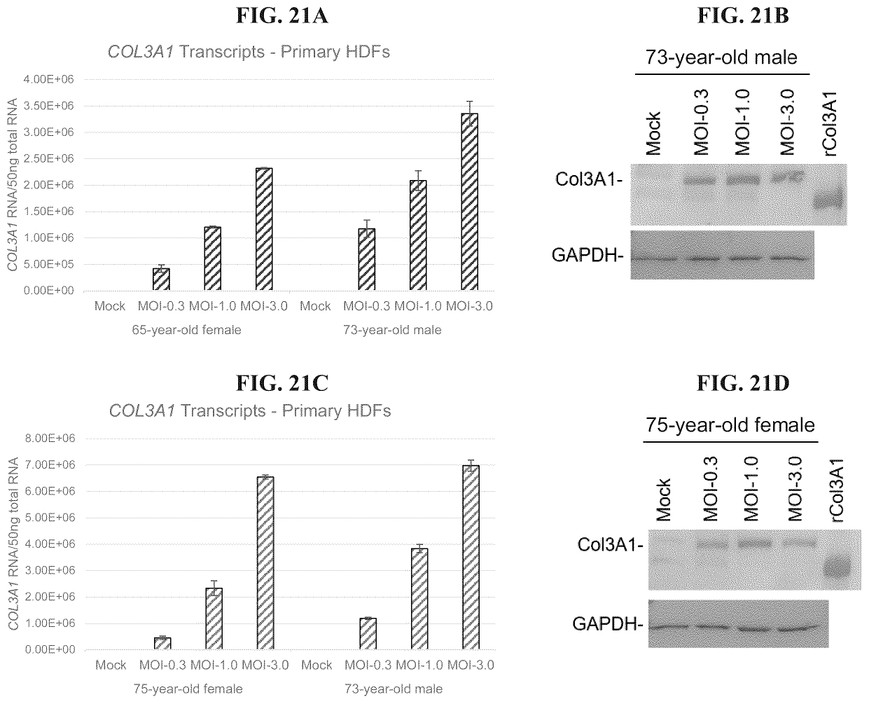

As compared to the immortalized HDFs, a similar dose-dependent increase with comparable or higher COL3 transcript levels was observed after C3vec01 infection of the primary HDFs from vendor 1 (FIG. 21A) and vendor 2 (FIG. 21C). A sample of representative primary cells from each vendor was also tested for COL3 expression by western blot analysis (primary anti-COL3 antibody, Abcam cat. no. ab7778). C3vec01 was capable of rescuing high levels of human Collagen 3 expression in primary HDFs biopsied from a 73-year-old male patient (FIR. 21B) and a 75-year-ol female patient (FIG. 21D), even at the lowest MOI tested.

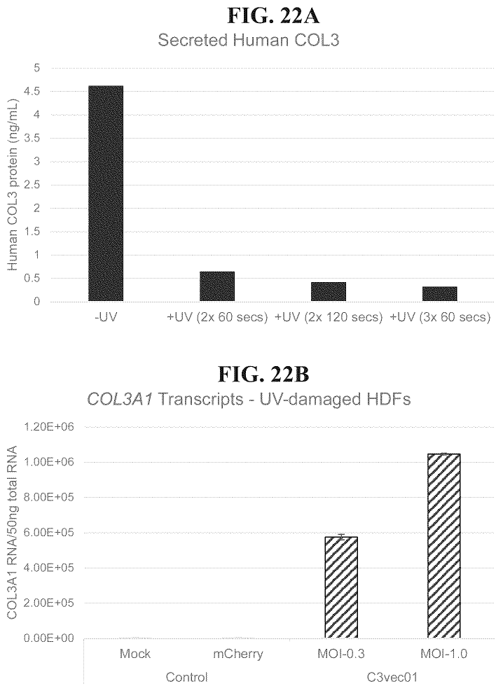

Finally, the ability of C3vec01 to induce robust Collagen 3 expression in UV-exposed immortalized human fibroblasts was tested. Sun exposure, and the corresponding UV damage, is known to be the single largest extrinsic contributor to the aged skin phenotype, and multiple groups have employed an in vitro skin fibroblast UV-exposure system to model certain aspects of photoaging (see e.g., Qin et al. 2018, Cell Physiol Biochem 46(5):1849-1860). To confirm that UV exposure caused human dermal fibroblasts to secrete less Collagen 3 (as would be expected given the phenotype of photo-aged skin), COL3 secretion into the supernatants of cultured immortalized human dermal fibroblasts was measured by ELISA before and after three different levels of UV exposure (FIG. 22A). Indeed, UV irradiation of cultured fibroblasts significantly reduced endogenous COL3 expression. COL1 expression were monitored in parallel in this experiment, and were not significantly affected by UV irradiation, indicating that UV exposure induced specific repression of COL3, as opposed to global suppression of protein synthesis. Next, the ability of C3vec01 to infect UV-irradiated immortalized HDFs and express exogenous COL3 was tested. Here, immortalized HDFs were exposed to UV-irradiation, and then allowed to recover for 24 hours prior to infection with C3vec01 at an MOI of 0.3 or 1. 48 hours after infection, exogenous human COL3 was assessed by qRT-PCR analysis. Strong COL3 expression was detected in C3vec01-infected, UV-irradiated HDFs at both tested MOIs (FIG. 22B), indicating that C3vec01 efficiently transduced photo-damaged cells and delivered its encoded cargo. Mock infected cells, and cells infected with a virus containing the same HSV-1 backbone as C3vec01 but instead encoding an mCherry effector, were used as negative controls to ensure specificity of transgene detection.

Taken together, the data presented in this example indicates that the recombinant HSV-1 vector C3vec01 efficiently transduces multiple human skin cell types, is capable of rescuing Collagen 3 expression in aged primary fibroblasts harvested from old patients, and is capable of salvaging Collagen 3 expression from UV-damaged HDFs. Without wishing to be bound by theory, it is believed that the data supports the use of a recombinant HSV encoding human Collagen 3 to correct the collagen defects of aged skin.

### Example 7: In Vivo Characterization of Intradermally Administered C3Vec01

The following example described in vivo experiments establishing methods of intradermally administering C3vec01 in young and old healthy immunocompetent animals.

All experiments were conducted as described above unless noted otherwise.

All procedures conducted in this example were in compliance with applicable animal welfare acts and were approved by the local Institutional Animal Care and Use Committee (IACUC).

The backs of mice were shaved before further manipulations. C3vec01 (or vehicle control) was then injected intradermally to four sites in the backs of the mice. After infection and the subsequent recovery period, the animals were euthanized, and the treatment sites were removed using an 8 mm punch biopsy. One half of each biopsy was quick-frozen in liquid nitrogen for qPCR/qRT-PCR analysis, while the other half was processed for immunofluorescence analysis.

Tissue samples were processed for nucleic acid and protein analysis as described above. For COL3 immunofluorescence staining, a rabbit anti-human Collagen 3 primary antibody (Abcam, cat. no. ab7778), and an Alexa Fluor® 488-conjugated secondary antibody were used. Tissue samples were mounted in mounting media containing DAPI to visualize nuclei.

An in vivo pharmacology study was conducted in young (6-8-week-old) and old (approximately 13-month-old) C57BL/6 mice to evaluate C3vec01-mediated expression of human COL3 in immunocompetent animals upon intradermal administration of the vector. A total of 10 animals were used for this study. The back of each mouse was first shaved and then intradermally injected with 2×10⁸ PFU/site of C3vec01 (or vehicle control) at 4 sites/animal. Injected sited were biopsied at either 48-hours or 1-week post-dosing, and were evaluated for human COL3 expression by qPCR and immunofluorescence. Table 5 below provides a synopsis of the experimental design.

TABLE 5
study design and test article administration

| | | | | | Vol- | Loca- | Ter- |
|---|---|---|---|---|---|---|---|
| | | | | Route of | ume | tion, | mina- |
| Group | | Test | | Adminis- | of Test | No. of | tion |
| No. | N | Article | Mouse | tration | Article | Sites | (day) |
| 1 | 1 | Vehicle | Young | Intra- | 100 μL | Back, 4 | 2 |
| | | | | dermal | | | |
| 2 | 2 | C3vec01 | Young | Intra- | 100 μL | Back, 4 | 2 |
| | | | | dermal | | | |
| 3 | 2 | C3vec01 | Young | Intra- | 100 μL | Back, 4 | 7 |
| | | | | dermal | | | |
| 4 | 1 | Vehicle | Old | Intra- | 100 μL | Back, 4 | 2 |
| | | | | dermal | | | |
| 5 | 2 | C3vec01 | Old | Intra- | 100 μL | Back, 4 | 2 |
| | | | | dermal | | | |
| 6 | 2 | C3vec01 | Old | Intra- | 100 μL | Back, 4 | 7 |
| | | | | dermal | | | |

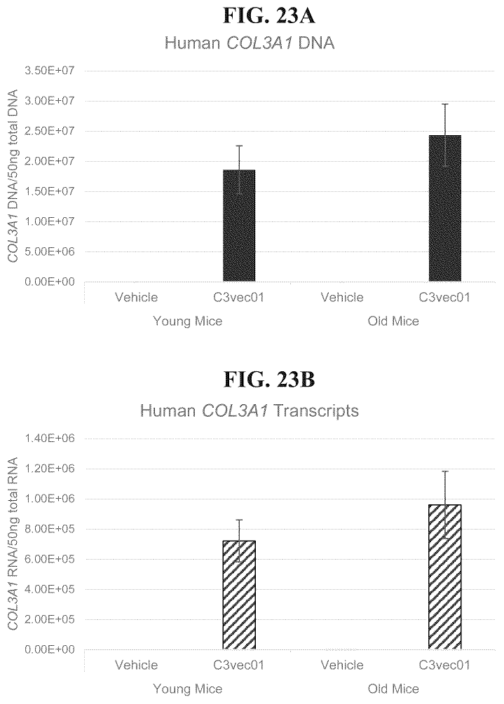

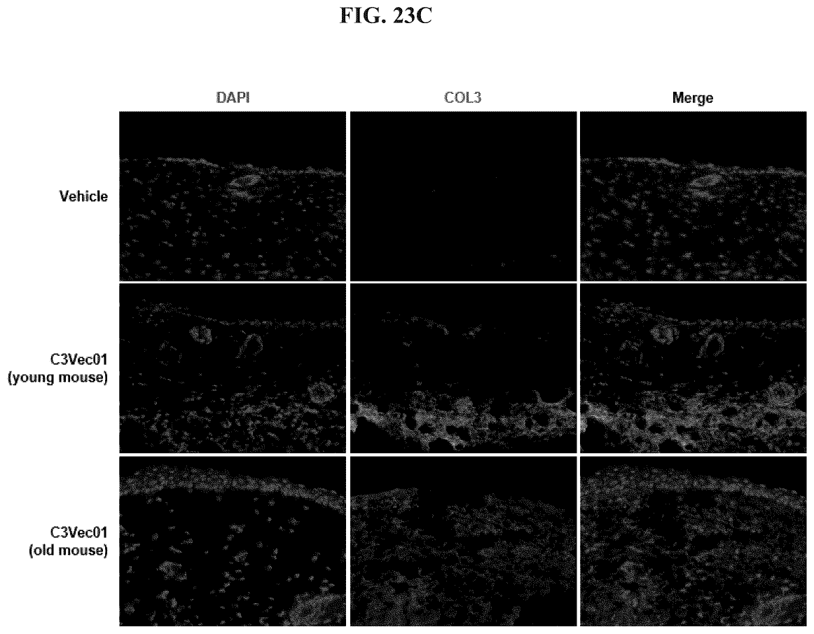

Intradermal delivery of C3vec01 led to high levels of transduced vector genomes detected in skin biopsies harvested 48-hours post-injection (FIG. 23A), as well as yielded high levels of human Collagen 3 transcripts in both young and old mice (FIG. 23B). Immunofluorescence-based detection also showed visibly increased levels of human COL3 throughout the dermis in C3vec01-treated skin relative to vehicle-treated skin (FIG. 23C), correlating with transcript levels.

Taken together, the data provided in this example indicates that can efficiently transduce skin and express human Collagen 3 in vivo after intradermal injection. Without wishing to be bound by theory, it is believed that the in vivo study presented here lends further support for the use of HSV-1 as a novel gene therapy to delivery human Collagen 3 in the aesthetic setting.

### Example 8: Generation and Validation of Modified Herpes Simplex Virus Vectors Encoding Human Laminins

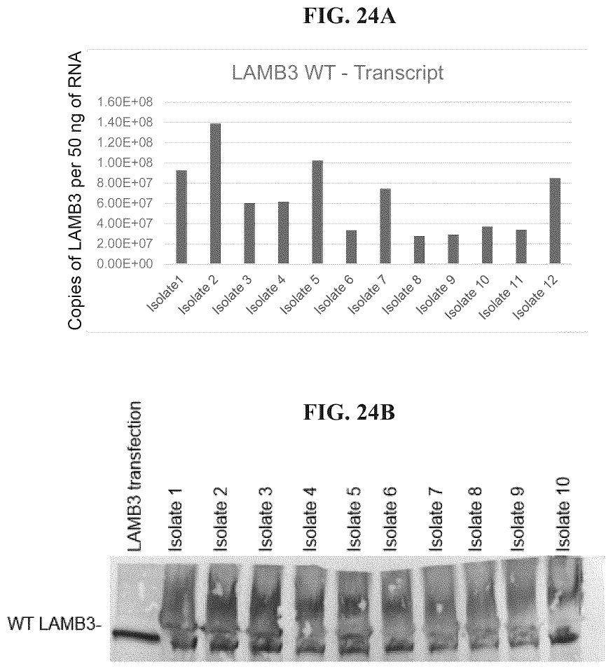

To begin, recombinant herpes virus vectors were engineered to incorporate either wild-type or codon-optimized variants of two human laminin proteins, LAMB3 or LAMC2, as described in Example 3 above. A number of isolates were picked for each type of virus. To test whether certain isolates were capable of expressing the encoded wild-type human LAMB3 protein, ICP4-complementing Vero cells were plated in 6-well plates and were infected with 12 untitered viral isolates of wild-type LamB3-encoding viruses until completion of infection. After infection, RNA was harvested, cDNA was generated, and expression of wild-type LamB3 from each isolate was determined by qPCR (FIG. 24A). All 12 isolates were capable of expressing wild-type human LamB3 in the transduced Vero cells at varying levels. The ability of 10 of these isolates to express human LamB3 was also tested by western blot. ICP4-complementing Vero cells were plated in 6-well plates and were infected with 10 untitered viral isolates of wild-type LamB3 expressing viruses until completion of infection. A well of Vero cells was transfected with a LamB3 expression plasmid as a positive control. After infection, the cells were collected by gentle scraping, centrifuged to collect cell pellets, culture medium was aspirated, and the cell pellets were washed once with PBS. Following washing, each cell pellet was resuspended in 200 μL RIPA buffer containing protease inhibitors, and the resuspensions were incubated at 4° C. for 20 minutes with gentile agitation every 5 minutes. After incubation, the samples were centrifuged at 17,000×g for 5 minutes, the supernatant was removed, and 4×LDS reducing sample buffer containing 5% 2-mercaptomethanol was added to each clarified supernatant. The samples were then boiled for 10 minutes before loading on a 4-20% Tris-Glycine polyacrylamide gel. After electrophoresis, the protein was transferred to a PVDF membrane, and the membrane was blocked for 30 minutes in 5% milk/TBS. Primary rabbit anti-LamB3 antibody (Abcam, cat. No. ab128864) was then added to the PVDF membrane at 1:1000 dilution in 5% milk/TBS and incubated overnight at RT° C. (˜16 hours). The blots were then washed 3× for 5 minutes each with TBS, and then stained with an AP-conjugated goat anti-rabbit IgG antibody (Sigma, cat. No. A3687) in 5% milk/TBS for 1 hour at RT° C. The membranes were then washed 3× for 5 minutes each with TBS, BCIP/NBT was added, and the blots were developed for −10 minutes at RT° C. In agreement with the qPCR data, all 10 viral isolates were capable of expressing the encoded wild-type human LamB3 at varying levels (FIG. 24B).

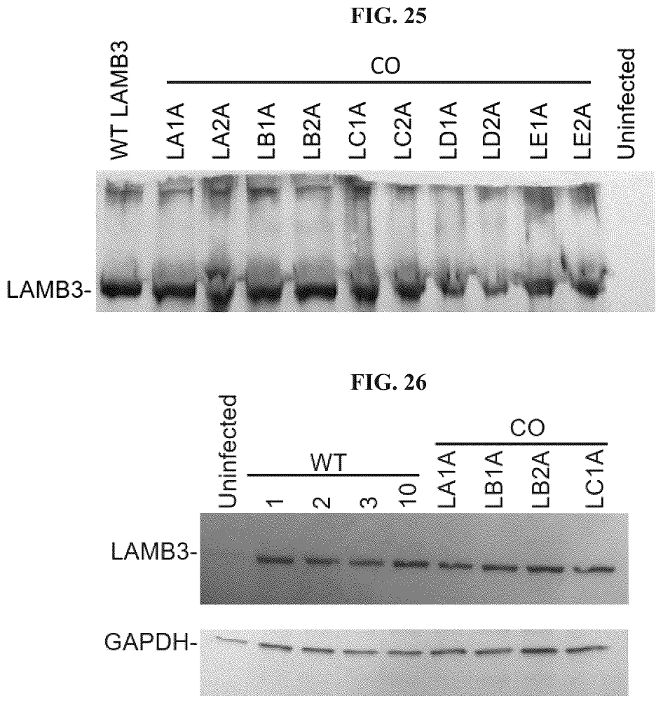

Viruses encoding codon-optimized variants of human LamB3 were also tested for their ability to express their cargo in Vero cells by western blot analysis. Briefly, 10 untitered viral isolates of codon-optimized (CO) LamB3-encoding viruses were used to infect Vero cells, cell pellets were collected, each pellet was resuspended in RIPA buffer containing protease inhibitors, and western blots were conducted using these cell lysates, as described above. All 10 viral isolates were capable of expressing the encoded codon-optimized human LamB3 in Vero cells (FIG. 25).

Next, four viral isolates encoding either wild-type or codon-optimized LAMB3 were tested for their capacity to transduce primary human cells and express their cargo. Immortalized primary normal keratinocytes were infected at a multiplicity of infection (MOI) of 1.0 for 48 hours. Uninfected cells were used as a negative control. Expression of LamB3 in the infected human keratinocytes was then examined by western blot. Western blots were carried out as described above using a primary rabbit anti-LamB3 antibody (Abcam, cat. No. ab 128864). In line with the data generated using Vero cells, the viral isolates expressing either wild-type and codon-optimized LamB3 were confirmed to effectively transduce primary human keratinocytes and express their encoded construct at suitable levels (FIG. 26).

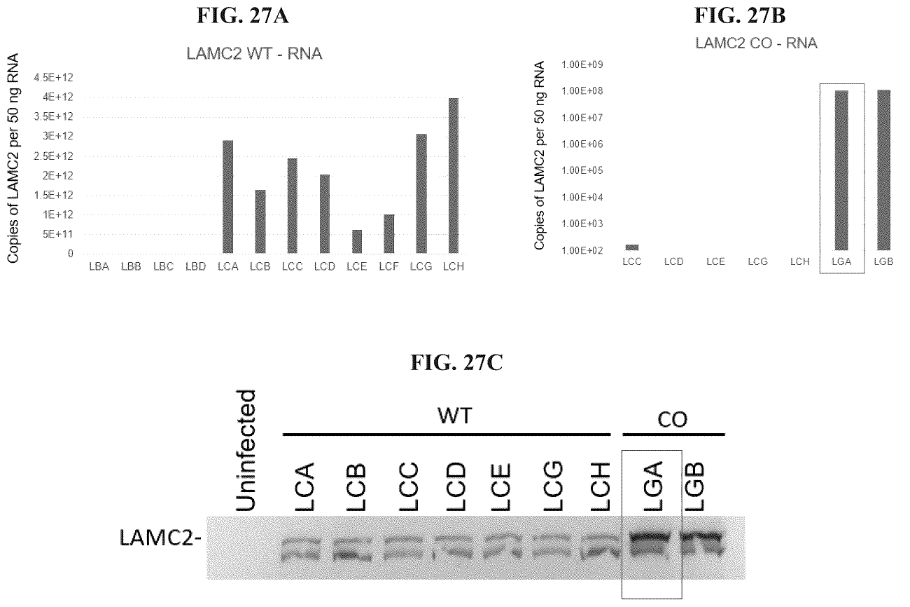

To test whether LamC2-containing isolates were capable of expressing the encoded wild-type or codon-optimized human LAMC2, ICP4-complementing Vero cells were plated in 6-well plates and were infected with a number of untitered wild-type or codon-optimized LamC2-expressing viral isolates until completion of infection. After infection, RNA was harvested, cDNA was generated, and expression of wild-type LamC2 (FIG. 27A) or codon-optimized LamC2 (FIG. 27B) from each isolate was determined by qPCR. 8/12 isolates were capable of expressing wild-type human LamC2 in the transduced Vero cells at varying levels, while 3/7 isolates were capable of expressing codon-optimized human LamC2. The ability of certain wild-type and codon-optimized isolates to express human LamC2 was next tested by western blot. ICP4-complementing Vero cells were plated in 6-well plates and were infected with untitered viral isolates until completion of infection. A well of Vero cells was left uninfected as a negative control. After infection, the cells were collected by gentle scraping, centrifuged to collect cell pellets, culture medium was aspirated, and the cell pellets were washed once with PBS. Following washing, each cell pellet was resuspended in 200 μL RIPA buffer containing protease inhibitors, and the resuspensions were incubated at 4° C. for 20 minutes with gentile agitation every 5 minutes. After incubation, the samples were centrifuged at 17,000×g for 5 minutes, the supernatant was removed, and 4×LDS reducing sample buffer containing 5% 2-mercaptomethanol was added to each clarified supernatant. The samples were then boiled for 10 minutes before loading on a 4-20% Tris-Glycine polyacrylamide gel. After electrophoresis, the protein was transferred to a PVDF membrane, and the membrane was blocked for 30 minutes in 5% milk/TBS. Primary rabbit anti-LamC2 antibody (Abcam, cat. No. ab96327) was then added to the PVDF membrane at 1:1000 dilution in 5% milk/TBS and incubated overnight at RT° C. (˜16 hours). The blots were then washed 3× for 5 minutes each with TBS, and then stained with an AP-conjugated goat anti-rabbit IgG antibody (Sigma, cat. No. A3687) in 5% milk/TBS for 1 hour at RT° C. The membranes were then washed 3× for 5 minutes each with TBS, BCIP/NBT was added, and the blots were developed for −10 minutes at RT° C. In agreement with the qPCR data, all 9 of the tested viral isolates were also able to express the encoded human LamC2 (FIG. 27C).

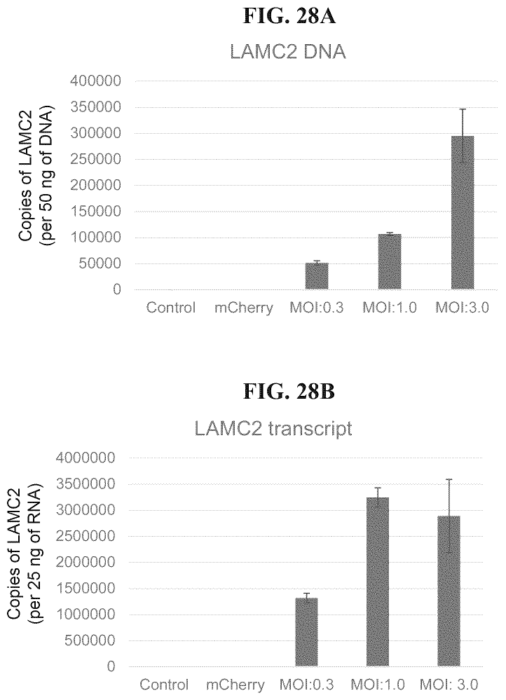

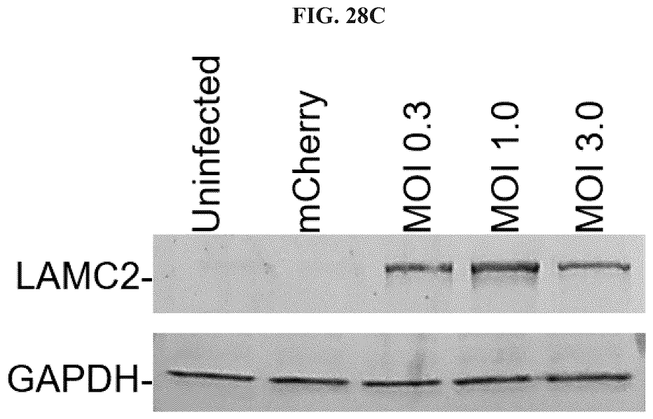

The codon-optimized LamC2-expressing viral isolate “LGA” was selected for further testing in human cells. Immortalized primary normal keratinocytes were infected with the LGA isolate at a multiplicity of infection (MOI) of 0.3, 1.0, or 3.0 for 48 hours. Uninfected (control) and mCherry-expressing virus infected cells were used as a negative control. DNA and RNA were extracted from the immortalized keratinocytes after 48 hours of infection, and qPCR/qRT-PCR was performed (FIGS. 28A-B). A good dose-response was observed for the LGA isolate in the immortalized keratinocytes, as assessed by viral genome copies detected per 50 ng of DNA (FIG. 28A). Interestingly, while a dose response was observed at the transcript level when increasing the MOI from 0.3 to 1.0, no additional increase in transcript levels were observed when increasing from an MOI of 1.0 to 3.0 (FIG. 28B). Expression of LamC2 in the infected human keratinocytes were also examined by western blot. Western blots were carried out as described above (primary rabbit anti-LamC2 antibody (Abcam, cat. No. ab96327) was used). In line with the transcript analysis, a dose response was observed at the protein level when increasing MOI from 0.3 to 1.0, but not from 1.0 to 3.0 (FIG. 28C).

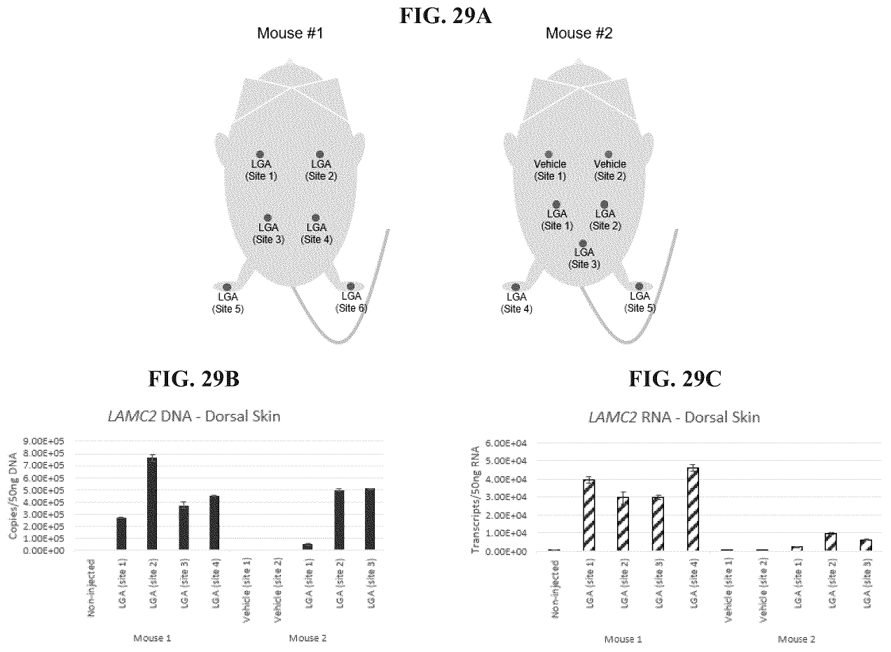

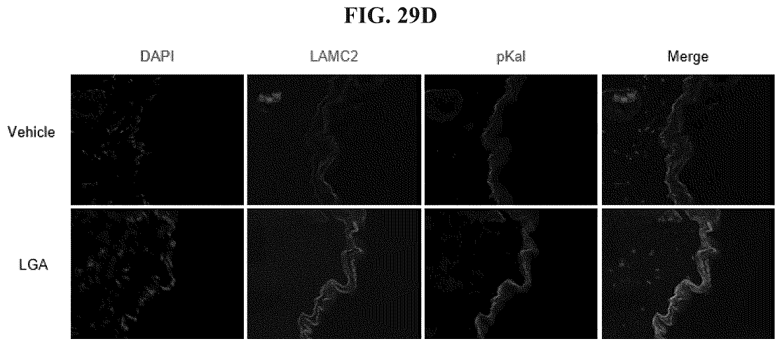

Finally, to test whether isolate “LGA” was capable of expressing its human laminin when delivered in vivo, human LAMC2 expression was assessed by qPCR, qRT-PCR, and immunofluorescence after intradermal injection in animals. 1×10⁸ PFUs of LGA formulated in PBS+10% glycerol (vehicle) was intradermally injected into the dorsal skin and footpads of two mice. An equivalent volume of vehicle alone was intradermally administered to the dorsal skin of one mouse to act as a negative control. A schematic of the injection sites for the two animals treated in this study is provided in FIG. 29A.

72 hours post-administration, a full thickness 8 mm biopsy was taken from each treatment site and split in half. One half of each section was flash frozen in liquid nitrogen and subsequently processed for qPCR and qRT-PCR analysis in order to quantify LAMC2 DNA copy numbers (FIG. 29B) and transcript levels (FIG. 29C) in the dorsal skin. The remaining half of each biopsy was embedded in OCT for immunofluorescence (IF). LAMC2 expression in cryosections was determined by immunofluorescent analysis using an anti-human LAMC2 antibody (Abcam, cat. no. ab96327). To confirm that the human LAMC2 expressed from LGA was correctly localized to the region of the skin where native laminin-332 is found, the dorsal skin samples were also counterstained for mouse laminin-332 (pKal). Intradermal administration of LGA led to successful transduction of mouse skin, with robust expression of the encoded human transgene in the correct layer of the epidermis. Histological evaluation showed no inflammatory infiltration at the treated site, demonstrating the safety of this therapy.

Taken together, the data presented in this example demonstrates that (1) HSV vectors could be successfully engineered to robustly express human laminin proteins (specifically, the R or γ subunit of human laminin-332), (2) an HSV-based vector (LGA) could successfully deliver a human laminin-332 subunit in vivo, and (3) the recombinant human laminin-332 subunit was expressed in, and localized to, the appropriate region of the epidermis of treated animals.

1. A composition comprising:
(a) a herpes virus comprising a recombinant herpes virus genome, wherein the recombinant herpes virus genome comprises a first polynucleotide encoding a first collagen protein; and
(b) an excipient,
wherein the recombinant herpes virus genome does not comprise a polynucleotide encoding a Collagen alpha-1(VII) chain (COL7) polypeptide.

2. The composition of claim 1, wherein the recombinant herpes virus genome is replication defective.

3. The composition of claim 1, wherein the recombinant herpes virus genome is a recombinant herpes simplex virus type 1 (HSV-1) genome.

4. The composition of claim 3, wherein the recombinant HSV-1 genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

5. (canceled)

6. (canceled)

7. The composition of claim 1, wherein the first collagen protein is selected from the group consisting of a human Collagen alpha-1(I) chain polypeptide (COL1-1), a human Collagen alpha-2(I) chain polypeptide (COL1-2), a human Collagen alpha-1(II) chain polypeptide (COL2), a human Collagen alpha-1(III) chain polypeptide (COL3), a human Collagen alpha-1(IV) chain polypeptide (COL4-1), a human Collagen alpha-2(IV) chain polypeptide (COL4-2), a human Collagen alpha-3(IV) chain polypeptide (COL4-3), a human Collagen alpha-4(IV) chain polypeptide (COL4-4), a human Collagen alpha-5(IV) chain polypeptide (COL4-5), a human Collagen alpha-6(IV) chain polypeptide (COL4-6), a human Collagen alpha-1(V) chain polypeptide (COL5-1), a human Collagen alpha-2(V) chain polypeptide (COL5-2), a human Collagen alpha-3(V) chain polypeptide (COL5-3), a human Collagen alpha-1(VI) chain polypeptide (COL6-1), a human Collagen alpha-2(VI) chain polypeptide (COL6-2), a human Collagen alpha-3(VI) chain polypeptide (COL6-3), a human Collagen alpha-4(VI) chain polypeptide (COL6-4), a human Collagen alpha-5(VI) chain polypeptide (COL6-5), a human Collagen alpha-6(VI) chain polypeptide (COL6-6), a human Collagen alpha-1(VIII) chain polypeptide (COL8), a human Collagen alpha-1(IX) chain polypeptide (COL9-1), a human Collagen alpha-2(IX) chain polypeptide (COL9-2), a human Collagen alpha-3(IX) chain polypeptide (COL9-3), a human Collagen alpha-1(X) chain polypeptide (COL10), a human Collagen alpha-1(XI) chain polypeptide (COL11-1), a human Collagen alpha-2(XI) chain polypeptide (COL11-2), a human Collagen alpha-1(XII) chain polypeptide (COL12), a human Collagen alpha-1(XIII) chain polypeptide (COL13), a human Collagen alpha-1(XIV) chain polypeptide (COL14), a human Collagen alpha-1(XV) chain polypeptide (COL15), a human Collagen alpha-1(XVI) chain polypeptide (COL16), a human Collagen alpha-1(XVII) chain polypeptide (COL17), a human Collagen alpha-1(XVIII) chain polypeptide (COL18), a human Collagen alpha-1(XIX) chain polypeptide (COL19), a human Collagen alpha-1(XX) chain polypeptide (COL20), a human Collagen alpha-1(XXI) chain polypeptide (COL21), a human Collagen alpha-1(XXII) chain polypeptide (COL22), a human Collagen alpha-1(XXIII) chain polypeptide (COL23), a human Collagen alpha-1(XXIV) chain polypeptide (COL24), a human Collagen alpha-1(XXV) chain polypeptide (COL25), a human Collagen alpha-1(XXVI) chain polypeptide (COL26), a human Collagen alpha-1(XXVII) chain polypeptide (COL27), and a human Collagen alpha-1(XXVIII) chain polypeptide (COL28).

8. (canceled)

9. (canceled)

10. The composition of claim 1, wherein the recombinant herpes virus genome further comprises a second polynucleotide encoding a second protein.

11. (canceled)

12. (canceled)

13. The composition of claim 1, wherein the composition is suitable for intradermal administration or superficial injection.

14. (canceled)

15. (canceled)

16. A method of diminishing one or more dermatological signs of aging in a subject, the method comprising administering to the subject an effective amount of a composition comprising:
(a) a herpes virus comprising a recombinant herpes virus genome, wherein the recombinant herpes virus genome comprises a first polynucleotide encoding a first collagen protein; and
(b) an excipient.

17. (canceled)

18. The method of claim 16, wherein the subject is a human.

19. The method of claim 16, the composition is administered topically, transdermally, subcutaneously, epicutaneously, intradermally, orally, sublingually, buccally, rectally, vaginally, intraurethrally, intravenously, intraarterially, intramuscularly, intraosseously, intracardially, intraperitoneally, transmucosally, intravitreally, subretinally, intraarticularly, peri-articularly, locally, or via inhalation to the subject.

20. The method of claim 16, wherein the composition is administered intradermally or via superficial injection to the subject.

21. (canceled)

22. The method of claim 16, wherein the recombinant herpes virus genome is a recombinant herpes simplex virus type 1 (HSV-1) genome.

23. The method of claim 22, wherein the recombinant HSV-1 genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

24. (canceled)

25. (canceled)

26. The method of claim 16, wherein the first collagen protein is selected from the group consisting of a human Collagen alpha-1(I) chain polypeptide (COL1-1), a human Collagen alpha-2(I) chain polypeptide (COL1-2), a human Collagen alpha-1(II) chain polypeptide (COL2), a human Collagen alpha-1(III) chain polypeptide (COL3), a human Collagen alpha-1(IV) chain polypeptide (COL4-1), a human Collagen alpha-2(IV) chain polypeptide (COL4-2), a human Collagen alpha-3(IV) chain polypeptide (COL4-3), a human Collagen alpha-4(IV) chain polypeptide (COL4-4), a human Collagen alpha-5(IV) chain polypeptide (COL4-5), a human Collagen alpha-6(IV) chain polypeptide (COL4-6), a human Collagen alpha-1(V) chain polypeptide (COL5-1), a human Collagen alpha-2(V) chain polypeptide (COL5-2), a human Collagen alpha-3(V) chain polypeptide (COL5-3), a human Collagen alpha-1(VI) chain polypeptide (COL6-1), a human Collagen alpha-2(VI) chain polypeptide (COL6-2), a human Collagen alpha-3(VI) chain polypeptide (COL6-3), a human Collagen alpha-4(VI) chain polypeptide (COL6-4), a human Collagen alpha-5(VI) chain polypeptide (COL6-5), a human Collagen alpha-6(VI) chain polypeptide (COL6-6), a human Collagen alpha-1(VIII) chain polypeptide (COL8), a human Collagen alpha-1(IX) chain polypeptide (COL9-1), a human Collagen alpha-2(IX) chain polypeptide (COL9-2), a human Collagen alpha-3(IX) chain polypeptide (COL9-3), a human Collagen alpha-1(X) chain polypeptide (COL10), a human Collagen alpha-1(XI) chain polypeptide (COL11-1), a human Collagen alpha-2(XI) chain polypeptide (COL11-2), a human Collagen alpha-1(XII) chain polypeptide (COL12), a human Collagen alpha-1(XIII) chain polypeptide (COL13), a human Collagen alpha-1(XIV) chain polypeptide (COL14), a human Collagen alpha-1(XV) chain polypeptide (COL15), a human Collagen alpha-1(XVI) chain polypeptide (COL16), a human Collagen alpha-1(XVII) chain polypeptide (COL17), a human Collagen alpha-1(XVIII) chain polypeptide (COL18), a human Collagen alpha-1(XIX) chain polypeptide (COL19), a human Collagen alpha-1(XX) chain polypeptide (COL20), a human Collagen alpha-1(XXI) chain polypeptide (COL21), a human Collagen alpha-1(XXII) chain polypeptide (COL22), a human Collagen alpha-1(XXIII) chain polypeptide (COL23), a human Collagen alpha-1(XXIV) chain polypeptide (COL24), a human Collagen alpha-1(XXV) chain polypeptide (COL25), a human Collagen alpha-1(XXVI) chain polypeptide (COL26), a human Collagen alpha-1(XXVII) chain polypeptide (COL27), and a human Collagen alpha-1(XXVIII) chain polypeptide (COL28).

27. (canceled)

28. (canceled)

29. The method of claim 16, wherein the recombinant herpes virus genome further comprises a second polynucleotide encoding a second protein.

30. (canceled)

31. The composition of claim 10, wherein the second protein is selected from the group consisting of a fibronectin protein, an elastin protein, a lumican protein, a vitronectin protein, a vitronectin receptor protein, a laminin protein, a neuromodulator protein, and a fibrillin protein.

32. The method of claim 29, wherein the second protein is selected from the group consisting of a fibronectin protein, an elastin protein, a lumican protein, a vitronectin protein, a vitronectin receptor protein, a laminin protein, a neuromodulator protein, and a fibrillin protein.

33. The method of claim 16, wherein the diminishing of one or more dermatological signs of aging is indicated by the: (a) treatment, reduction, and/or prevention of fine lines and/or wrinkles; (b) reduction of skin pore size; (c) improvement in skin thickness, plumpness, and/or tautness; (d) improvement in skin smoothness, suppleness, and/or softness; (e) improvement in skin tone, radiance, and/or clarity; (f) improvement in procollagen and/or collagen production; (g) improvement in skin texture and or promotion of retexturization; (h) improvement in appearance of skin contours; (i) restoration of skin luster and/or brightness; (j) improvement of skin appearance decreased by aging and/or menopause; (k) improvement in skin moisturization; (l) increase in skin elasticity and/or resiliency; (m) treatment, reduction, and/or prevention or skin sagging; (n) improvement in skin firmness; (o) reduction of pigment spots, mottled skin, and/or scars; (p) improvement of optical properties of skin by light diffraction or reflection; or (q) any combinations thereof.

34. The method of claim 16, wherein the recombinant herpes virus genome does not comprise a polynucleotide encoding a Collagen alpha-1(VII) chain polypeptide (COL7).


SEQUENCE LISTING (64 sequences)

SEQ ID NO: 1 - DNA, 4395 bp, Homo sapiens
  source 1..4395: mol_type = unassigned DNA
atgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaa

SEQ ID NO: 2 - DNA, 4395 bp, synthetic construct
  misc_feature 1..4395: note = Synthetic Construct
  source 1..4395: mol_type = other DNA
atgttcagcttcgtggacctgagactgctgctgctcctggctgctacagccctgctgacacacggacaagaggaaggccaggtcgaaggacaggacgaggacatccctcctatcacctgtgtgcagaacggcctgagataccacgaccgggatgtgtggaagcccgagccttgcagaatctgcgtgtgcgacaatggcaaggtgctgtgcgacgacgtgatctgcgacgagacaaagaattgccctggcgccgaagtgcctgagggcgaatgttgtcctgtgtgccctgatggcagcgagagccccacagatcaagagacaacaggcgtggaaggccccaagggcgatacaggacctagaggtcctagaggacctgccggacctcctggcagagatggaattcctggacagcctggactgcccggaccacctggacctccagggcctccaggtccaccaggactcggaggaaattttgccccacagctgagctacggctacgacgagaaaagcacaggcggcatctctgtgcctggacctatgggaccttctggcccaagaggacttcctggtcctcctggtgctccaggacctcagggatttcaaggaccaccaggcgaacctggcgaaccaggcgctagtggtccaatgggaccaagaggccctcctgggccaccaggcaaaaatggcgacgatggcgaagccggaaagcctggaaggcctggcgaaagaggcccgccaggaccgcaaggcgctagagggttgcctggaactgcaggactgcctggcatgaagggccacagaggcttttctggactggatggcgctaagggcgacgctggaccagcaggacctaaaggcgagcctggatctcctggcgagaatggtgcacctggacagatgggtcccagaggattgccaggcgagagaggtagacctggcgctccgggaccagccggtgctagaggaaatgatggcgcaacaggtgctgctgggcctcctggaccaaccggaccagctggcccacctggatttccaggcgctgttggagcaaagggcgaagcaggcccacaaggacctaggggatctgaaggtcctcagggcgttagaggcgagccagggccacctgggcctgccggtgcagctggacctgctggaaaccctggtgctgatggacagccaggtgccaaaggtgctaatggcgcccctggaattgccggcgctccaggttttcccggcgcaagagggccatctggacctcaaggcccaggcggacctccgggtcctaagggaaatagcggagagccaggcgctcctgggagtaaaggcgatactggcgcaaaaggcgaacccggacctgtgggagttcaaggacctcctggaccagctggcgaggaaggcaaaagaggcgctaggggagaaccaggaccaacagggctccctggtccacctggcgagcgcggaggacctggatctagaggattccctggcgcagatggcgtggccggaccaaaaggacctgcaggcgaaaggggatcaccaggtcctgcaggccctaagggttctccaggcgaggctggcagacccggcgaagctggactcccaggtgctaagggactgacaggctcaccaggatctcccggaccagacggaaaaacaggacctccaggaccggcaggacaggatggtagacccggtcctcctggaccgcctggtgcaagaggacaagctggcgtgatgggctttcctggaccaaaaggtgcagccggcgaacctggaaaagcaggcgagaggggagttcccggacctccaggtgctgttggacctgccggaaaagatggcgaagctggtgcacaaggtcctccagggccagccggaccagccggcgagagaggcgaacaaggaccagccggatctccaggatttcagggactgccagggcctgctggcccgcctggcgaggcagggaagccaggcgaacagggtgttcctggcgatcttggagcccctggtcctagcggagctagaggcgaaagaggatttcctggcgaaaggggcgttcagggtccaccgggaccagctggaccaaggggtgcaaatggtgccccaggcaatgacggtgctaaaggcgacgcaggcgccccaggtgctcctggatctcaaggcgcacctggacttcagggaatgcctggcgaacggggagctgctggacttcccggtccaaaaggcgataggggagatgctggtcctaagggcgctgatggctctcctggaaaggatggcgtcagaggcctgacaggcccaattggccctccgggacctgctggcgctccaggcgataagggcgaatctggacctagtggacccgctggtcctacaggtgctaggggagccccaggcgaccggggagagcctggtccaccaggacctgctggatttgctggacctcctggcgctgatggtcaacctggtgctaagggcgagccaggcgacgctggtgcaaaaggcgacgctggtccacctggaccggccggacctgctgggccgccaggacctattggaaatgttggtgcccctggcgccaaaggcgcaagaggatctgctggcccaccaggcgctacaggattcccaggtgccgctggaagagttggaccaccggggccaagtggaaatgctggaccaccgggaccgccaggaccagccggcaaagaaggtggaaaaggccctaggggcgaaactggccctgcaggcaggccaggcgaagtgggccctccaggacctccggggcctgccggcgaaaaaggatctccaggcgcagatggacccgcaggcgctcccggaacaccaggtccacagggaattgctggacaaaggggagttgtcggcctgccaggacagaggggagagagaggttttccaggactccctgggccaagcggagaacctggcaaacagggaccatctggtgccagcggagagagagggccaccaggaccaatgggtcctccaggattggcagggcctcctggcgaatctggtagagaaggtgctccaggcgccgagggatctcctggacgtgatggttctcctggcgccaagggcgatagaggcgaaacaggcccagctggacctccaggcgcacccggcgctccaggcgcaccaggacctgttggccctgctggaaaatctggcgacagaggcgaaactggacccgcaggaccagccggacctgttggacctgtgggtgctagaggacccgctggaccacaaggtcctagaggcgacaagggcgaaacaggcgagcaaggcgacagaggcatcaagggacacagaggattcagcggactgcagggaccaccagggccgcctggaagtcccggcgagcagggaccaagcggagctagtggtcccgccggacctagaggaccacctggttctgctggtgcacccggaaaggacggactgaatgggctccccggacctattgggccacctggacctagaggaagaacaggcgacgcaggaccagttggaccacctgggccacctggaccgcctggtcctcctggacctccttctgccggattcgacttcagcttcctgcctcagcctcctcaagagaaggcccatgacggcggcagatattacagagccgacgacgccaacgtcgtgcgggacagagatctggaagtggacaccacactgaagtccctgtctcagcagatcgagaacatcagaagccccgagggcagcagaaagaaccctgccagaacctgtcgggacctgaagatgtgccacagcgattggaagtctggcgagtactggatcgaccccaaccagggctgcaacctggatgccatcaaggtgttctgcaacatggaaaccggcgagacatgcgtgtaccccacacagccatctgtggctcagaagaactggtacatcagcaagaaccccaaggacaagcggcacgtttggttcggcgagagcatgaccgatggcttccagtttgagtatggcggccagggctctgaccctgccgatgttgctatccagctgaccttcctgcggctgatgtctacagaggccagccagaacatcacctaccactgcaagaacagcgtggcctacatggatcagcagaccggcaacctgaagaaggcactgctgcttcagggcagcaacgagatcgagatcagagccgagggcaacagccggttcacctacagcgtgacagtggatggctgcaccagccatacaggcgcttggggcaagaccgtgatcgagtacaagaccaccaagaccagcagactgcccatcatcgatgtggcccctctggatgttggggcacccgatcaagagttcggcttcgatgtgggcccagtgtgcttcctgtaa

SEQ ID NO: 3 - DNA, 4101 bp, Homo sapiens
  source 1..4101: mol_type = unassigned DNA
atgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataa

SEQ ID NO: 4 - DNA, 4101 bp, synthetic construct
  misc_feature 1..4101: note = Synthetic Construct
  source 1..4101: mol_type = other DNA
atgctgagcttcgtggacaccagaacactgctgctgctggccgtgacactgtgtctggccacttgtcagagcctgcaagaggaaacagtgcggaaaggacctgccggcgatagaggacctagaggcgaaagaggtcctcctggacctcctggtagagatggcgaggatggacctacaggaccacctggtccaccaggacctccagggcctcctggccttggaggaaattttgccgctcagtacgatggcaaaggcgtcggacttggccctggacctatgggacttatgggcccaagaggaccaccaggtgctgcaggcgctccaggaccacaaggatttcaaggaccagctggcgagcctggcgaacctggacaaacaggtcctgctggtgctagaggaccagccgggccacctggaaaagctggcgaagatgggcaccctggaaagcctggtagacccggcgaaaggggtgttgttggacctcaaggcgccagaggctttcctggaacacctggactgcctggcttcaagggcatcagaggccacaatggcctggacggactgaaaggacaacctggtgctcctggcgtgaaaggcgaaccaggcgcacctggcgaaaatggcacaccaggacaaaccggcgcaagaggacttcctggcgagagaggaagagttggagccccaggtccagcaggcgcacgaggatctgatggatctgtgggacctgttggccctgccggacctattggaagtgctggccctcctggatttcctggcgcacccggaccaaagggcgaaattggagctgtgggaaacgccggacctgcaggcccagctggaccaaggggagaagttggattgcctggactgagcggaccagttgggccaccagggaatcctggtgccaatggactgacaggcgctaaaggtgcagctggccttccaggcgttgccggtgcaccaggactgccaggaccaagaggtatccctggtcctgttggagctgctggcgctacgggtgccagaggacttgttggagaacctgggccagccggatctaagggcgagtctggaaacaagggcgagccaggatctgctggtccacaaggcccgcctggaccatcaggcgaagaaggcaaacgaggccctaatggcgaagccggtagtgccgggcctcctggaccaccaggccttagaggatctcctggctctagaggattgccaggcgctgatggtagagcaggcgttatgggtccacctggatcaagaggcgcttctggccctgctggcgttagaggtccaaatggcgacgctggcagaccaggcgagcccggtcttatggggcctagagggttgcctggaagccctggcaatatcggcccagccggaaaagaaggccctgttggactccctggcatcgacggtagacctggaccaatcggacccgcaggcgctaggggagagcctggaaatattggcttccctgggcctaaaggccccacaggcgatcctggaaagaacggcgataagggccatgctggactcgctggtgcaaggggagcacctggacctgacggaaacaatggtgctcaagggccgcctgggccacaaggtgttcaaggtggaaaaggcgagcagggcccacctgggcctccaggcttccaaggacttcccggaccatctgggccagcaggcgaagttggaaagcctggcgaaagaggactgcacggcgagtttggcttgccgggtcctgccggtccacggggagagagaggccctccaggcgaatctggcgccgcaggacctactggccctatcggaagcagaggacctagtggacctccaggacctgatggcaacaaaggcgaacctggtgttgtgggcgctgtgggaacagctggaccttctggtccttctggattgcccggcgagcgcggagcagctggtattcctggtggcaaaggcgaaaagggcgagcctggactcagaggcgagatcggcaatcccggacgagatggcgctagaggcgccccaggtgcagttggtgccccgggacctgctggcgcaacaggcgacagaggcgaggctggtgccgctggtcctgccgggccagccggtcctagaggaagtccaggcgagaggggcgaagtgggacccgctggacccaatggatttgctgggcccgctggcgctgctggtcaacctggcgccaaaggcgagcggggagctaaaggtcctaaaggcgagaatggcgtcgtgggccctactggaccagtgggagcagcaggccccgcaggtcctaacggaccacctggaccagctgggtctagaggcgacggcggaccgcctggaatgacaggttttccaggcgccgctggaagaacaggtcctccaggaccatctggcatctctggtccaccagggccacctggtcctgctggaaaagaaggactgagaggccctaggggcgatcagggtccagttggaagaaccggcgaagtcggagctgtcggcccaccaggttttgccggcgaaaaaggccctagcggagaagctggaactgcaggaccgccgggaactcccggtcctcaaggattgcttggcgcccctggaattctgggactgcccggtagtcgcggagaacgtggactcccaggtgttgctggcgccgtcggagaaccgggaccacttggaattgctggaccacctggtgcaagaggtccacctggtgcagttggaagtcctggcgttaacggtgctccaggcgaagccggcagagatggaaatcccggcaatgatggcccgcctgggagagatggacagcctggacataagggcgagcgaggctacccaggcaatattggacctgtcggcgcagccggtgctcccggacctcatggtccagtcggtccagccgggaagcacggaaataggggagaaacaggaccctccggtcctgttggcccagctggcgcagttggaccaagaggcccatccggacctcagggaatccgcggagataagggcgaacctggcgagaagggacctagaggactgcctgggctgaaaggccataacggactgcaaggcctgccaggcattgctggccatcatggcgatcaaggtgcacccggtagtgtgggtcccgccggaccgaggggtcccgctggtccatctggacccgccggaaaagatggcagaacaggacatcctggcacagtggggcctgccggaattagaggcccacagggacatcaaggccccgctgggccgccaggacctccgggaccgccagggccaccaggcgttagtggcggaggatacgatttcggctacgacggcgacttctacagagccgaccagcctagatctgcccctagcctgaggcctaaggactacgaagtggacgccacactgaagtccctgaacaaccagatcgagacactgctgacccctgagggcagcagaaagaaccctgccagaacctgcagggacctgagactgtctcaccccgaatggtcctccggctactactggatcgaccccaatcagggctgcaccatggacgccatcaaggtgtactgcgacttcagcaccggcgagacatgcatcagagcccagcctgagaacatccccgccaagaactggtacagaagcagcaaggacaagaaacacgtgtggctgggcgagacaatcaacgccggcagccagttcgagtacaacgtggaaggcgtgaccagcaaagagatggccacacagctggctttcatgagactgctggccaattacgccagccagaacatcacctaccactgcaagaacagcattgcctacatggacgaggaaaccggcaacctgaagaaagccgtgatcctgcagggctctaacgacgtggaactggtggccgagggcaacagcagattcacctacaccgtgctggtggacggctgcagcaaaaagaccaacgagtggggcaagaccatcatcgagtataagaccaacaagcccagcagactgcccttcctggatatcgccccactggatattggaggcgccgaccaagagttctttgtggacatcggccccgtgtgcttcaagtga

SEQ ID NO: 5 - DNA, 4401 bp, Homo sapiens
  source 1..4401: mol_type = unassigned DNA
atgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataa

SEQ ID NO: 6 - DNA, 4401 bp, synthetic construct
  misc_feature 1..4401: note = Synthetic Construct
  source 1..4401: mol_type = other DNA
atgatgagcttcgtgcagaaaggcagctggctgctgctggcactgctgcaccctacaatcattctggcccagcaagaggccgtggaaggcggatgttctcacctgggacagagctacgccgacagggatgtgtggaagcctgagccttgccagatctgcgtgtgtgatagcggcagcgtgctgtgcgacgacatcatctgcgacgaccaagagctggactgccccaatcctgagatccctttcggcgagtgctgtgccgtttgtccccaacctcctaccgctcctaccagacctcctaatggacagggacctcagggccctaaaggcgatcctggacctcctggaatccccggcagaaatggcgatccaggcattcctggacagcctggctctcctggaagtccaggtccacctggcatctgcgagagctgtcctacaggccctcagaactacagcccacagtacgacagctacgacgtgaagtctggcgtggccgttggaggactggctggttatccaggacctgctggaccaccagggcctccaggaccgcctggaacaagtggacatccaggatctcccggcagtcccggttatcagggaccacctggcgaacctggacaggcaggacctagtggtcctcctggtccaccaggtgccattggaccatctggacctgccggaaaggatggcgaatctggcagaccaggcagacctggcgagagaggattgcctggtcctccgggtattaagggcccagccggcattccaggattccccggaatgaagggccacagaggcttcgatggacggaatggcgagaagggcgaaacaggtgcccctggactgaaaggcgaaaatggactgccaggcgagaatggcgcacccggacctatgggacctagaggtgctccaggcgaaagaggaaggccaggacttcctggtgctgcaggcgctagaggcaatgatggcgccagaggatctgatggccaacctgggccgccaggacctccaggcacagctggctttcccggatctcctggtgcaaagggcgaagtgggaccagctggaagccctggatctaatggtgccccaggacagaggggagaaccaggaccacaaggacatgctggtgctcaaggccctccgggtcctccagggatcaatggatctccaggcggcaaaggcgagatgggccctgctggaattcctggcgctccaggtcttatgggagccagagggccacctggaccagcaggcgcaaatggcgctcctggacttagaggcggagcaggcgagcctggaaaaaatggcgcaaaaggcgagcccggaccaaggggagaaagaggcgaagctggtattccaggtgtccctggtgccaaaggcgaggacggcaaagatggtagtcctggcgagccaggcgccaatggactccctggcgcagctggcgaaaggggagcacctggttttagaggacccgccggacctaatggcattcccggcgaaaaaggtccagccggcgagcgtggtgctcccggacctgcaggcccaagaggtgctgctggcgaaccaggcagagatggtgtcccaggcggaccagggatgagaggcatgccaggctcacccggcggacctggttctgatggaaaacccgggcctcctggtagccagggcgaatccggtagacccggacctccgggaccatcaggaccaagaggacaacctggcgtgatgggcttccctggacctaagggaaatgacggcgctcccggaaagaacggcgaacgcggtggccctggcggtcccggtcctcaagggccaccaggcaaaaacggcgaaaccggtccacaaggaccacctggacctacaggacctggcggagataagggcgatacaggtccaccaggacctcagggactgcaaggactgcctggaactggcggacctcctggcgagaatggaaaaccaggcgaaccaggacctaagggcgacgctggtgcacctggcgcaccaggcggaaagggcgacgcaggcgctccaggcgagaggggacctccaggattggctggtgctccaggcttgagaggcggagctggtcctccaggacctgaaggtggaaaaggtgctgcaggacctcctgggccacctggcgctgctggaactccaggacttcaagggatgcctggcgaacgaggtggacttggaagcccaggaccaaaaggcgataagggcgaacctggcggaccgggtgcagatggtgttcccggaaaagatggaccacggggcccaacaggacctataggccctccagggccagcaggacagccgggcgacaaaggcgaaggtggcgcccctggcttgcctggaattgctggtcctagaggttcacctggcgagcggggagaaacaggccctcctggaccggccggatttcccggtgctcctggccaaaatggcgagcctggcggaaaaggcgaaagaggtgcaccgggcgaaaaaggcgaaggcggacctcctggtgttgctggacctcctggcggatctggaccagctgggcctcctggtcctcaaggtgttaagggcgaaagaggctctccaggcggacccggtgctgctggatttcccggcgcaagaggattgcccggaccaccaggctctaatggcaatccaggtcctcctggacctagcggctctcctggcaaagatggcccaccaggaccagccggaaatactggtgctcctggatcacctggcgtgtccggaccgaaaggcgacgccggacaaccaggcgaaaaaggatctcctggcgctcaagggcctcctggcgcacctggtccattgggaattgccggaattacaggtgccagaggcctggctggcccacctggaatgcctgggccaagaggtagcccgggacctcaaggcgtgaaaggcgaatctggaaagcctggcgccaacggactgagcggagaaagaggacctccaggtccacaaggcctgcctggattggctggaacagctggcgaacctggaagagatggcaatcctggctctgatggcttgccggggagagatggctccccaggtggcaagggcgatcgcggagaaaatggtagcccaggcgctcccggcgctccaggacacccaggaccacctggtccagtcggacctgctggaaagtctggcgatagaggcgagtctggacccgcaggtcccgctggcgccccagggcctgccggatctaggggagcccctggaccgcaaggacctaggggagacaaaggcgagactggcgaacgcggagccgctggaatcaaaggccatagaggattcccaggcaaccctggtgcacccggatcaccaggaccggcaggacaacaaggcgctattggcagtccggggcctgctgggccgagaggaccagttggacctagtggaccaccgggcaaagatggaacaagcggacaccctggacctatcggaccaccaggacctagaggcaatagaggcgagagaggcagcgagggatctcccggacatcctggacaacccggtccaccggggccaccaggcgcaccagggccatgttgtggcggagttggagctgctgccattgctggaatcggcggagagaaagccggcggatttgccccttattacggcgacgagcccatggatttcaagatcaacaccgacgagatcatgaccagcctgaagtccgtgaacggccagatcgagagcctgatcagccctgacggcagcagaaagaaccccgccagaaactgccgcgacctgaagttctgtcaccccgagctgaaaagcggcgagtactgggttgaccccaaccagggctgtaaactggacgccattaaggtgttctgcaacatggaaaccggcgagacatgcatcagcgccaatcctctgaacgtgcccagaaagcactggtggacagatagcagcgccgagaagaagcacgtttggttcggcgagagcatggacggcggcttccagttctcttacggcaatcccgagctgcccgaggacgtgctggatgtgcaactggcctttctgagactgctgagcagccgcgccagccagaatatcacctaccactgcaagaacagcattgcctacatggatcaggccagcggcaacgtgaagaaagccctgaagctgatgggcagcaacgagggcgagtttaaggccgagggcaacagcaagttcacctacaccgtgctggaagatggctgcaccaagcacacaggcgagtggtccaagaccgtgttcgagtaccggacaagaaaggccgtgcggctgcctatcgtggatatcgccccttacgatatcggaggccccgatcaagagttcggcgttgacgtgggccctgtgtgtttcctgtaa

SEQ ID NO: 7 - DNA, 5010 bp, Homo sapiens
  source 1..5010: mol_type = unassigned DNA
atggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgctggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccctgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccagggctgcgaggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggccaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataa

SEQ ID NO: 8 - DNA, 5010 bp, synthetic construct
  misc_feature 1..5010: note = Synthetic Construct
  source 1..5010: mol_type = other DNA
atgggccctagactgtctgtgtggctgctgcttcttcctgccgctctgctgctgcacgaggaacattctagagccgccgctaaaggcggatgtgccggatctggatgcggcaagtgtgattgtcacggcgtgaagggccagaagggcgaaagaggacttcctggactgcagggcgtgatcggatttcctggaatgcaaggacctgagggaccacagggacctccaggacaaaaaggcgatacaggcgaacctggcctgcctggcacaaagggaacaagaggaccacctggcgcctctggctatcctggaaatccaggactgcctggaatcccaggacaggatggacctcctggtcctcctggcattcctggctgtaatggcaccaaaggcgagagaggtccactcggaccaccaggactcccaggatttgctggaaaccctgggccacctggattgcctggcatgaagggcgatcctggcgaaattctgggacacgttcccggcatgctgctgaaaggcgaaaggggctttcccggaattcctggcacacctggtccaccaggcttgccaggacttcaaggaccagttggcccacctggctttacaggtcctccaggtccaccggggcctccagggccgccaggcgaaaaaggacaaatgggactgagctttcagggccccaagggcgacaaaggcgatcaaggtgttagtggccctccaggtgttcctggacaggcccaggttcaagagaaaggcgatttcgccaccaagggcgagaagggacagaaaggcgaaccaggctttcagggaatgcctggcgtgggagaaaagggcgaacccggaaaacctggacctagaggcaaacccggcaaggatggcgataagggcgagaaaggatctcctggattccctggcgaacctggatatcctggactgatcggtagacagggccctcaaggcgagaagggcgaagctggaccgcctggaccaccgggaatagtgattggaacaggccctctcggagaaaaaggcgagcgaggataccctggaactcccggacctaggggagaaccaggacctaaaggttttcccggactgccaggacaaccagggcctcctgggcttcctgtgcctggacaagctggtgctccaggatttcccggcgaacgtggcgaaaagggcgatagaggttttcctggcacaagcctgcctgggccttctggaagagatggacttcccgggccaccaggttctcccggaccacctggacagcctggctataccaatggcatcgtcgagtgtcaacccggtccacctggcgatcaaggacctcctggaataccaggccagcctggctttatcggcgagattggagagaaagggcaaaaaggggagagctgcctgatctgcgacatcgatggatacagaggccctcctggacctcaaggcccaccaggcgagataggttttccagggcaacctggcgcaaaaggcgaccgtggactccctggtagagatggtgttgctggcgttccaggaccacaaggcacccctggacttattggacagccaggtgcaaagggcgagccaggcgagttctacttcgacctgagacttaaaggcgacaagggcgaccctggctttcctggacaacctggaatgcccggtagagctggatcaccaggccgtgatggacatccagggttgcccggaccaaaaggctctccaggatctgtgggcctcaaaggcgaaagaggccctccaggcggagttggatttcctggctctagaggcgatactggcccaccaggtccacctggatatggacctgctggccctattggagataagggccaagcaggattcccaggcggacccggttctccaggccttccgggtcctaaaggcgagcctggaaaaatcgttccactgcctggacctccaggcgctgaaggattgcctggatctcccggttttcctggaccgcaaggcgatagaggattccccggaacacccggtagaccaggccttcctggcgagaaaggtgctgtgggtcaacctggaatcggctttcctgggcctcctggtccaaaaggtgttgatggactgcccggcgatatgggcccaccgggaacaccaggcagacccggctttaatggattgcccggaaatcccggcgtccaaggccagaaaggcgagcccggtgttggccttcctggacttaaaggacttccaggcctgccaggcatacctgggacacctggcgaaaagggatctatcggagttcctggcgtgccaggcgaacatggtgcaattggtccacctgggctgcaaggcattagaggcgaacccgggcctccaggactccctggctctgttggaagtccaggcgtccccggaattggaccaccaggtgctaggggacctcctggcggacaaggtccaccaggattgtctggaccacctgggatcaaaggcgagaaaggcttccccggctttcccggccttgatatgcctggacctaaaggcgataagggtgcccagggcctgcctggaattactggacaaagcggcttgcccgggcttcccggacaacagggtgctccgggtattcctgggtttcccggatctaagggcgaaatgggcgtgatgggtacacctgggcaaccaggatcaccgggacctgttggagcaccggggttgcccggcgaaaaaggcgaccacggattcccaggatcaagcggaccaagaggcgatccgggattgaaaggcgataagggcgacgtgggacttcctggcaaaccaggctctatggacaaggtggacatgggctccatgaagggacaaaagggcgatcagggcgaaaagggccagatcggacctatcggcgaaaagggtagtagaggcgatcctggaacacccggcgttcccggaaaagatggacaagcaggccaaccggggcagccagggccaaaaggcgatcctggtatttctggaacaccaggtgcaccaggactgcccggacctaaaggatctgttggaggaatgggattgccagggacacccggcgagaaaggtgttcctggaatacctggacctcagggctctcctggactgccaggcgacaaaggtgctaaaggcgaaaagggacaagccggacctcctggcattggcatacctggacttaggggagagaagggcgaccagggaattgctggttttcctgggagcccaggcgagaaaggcgaaaaaggctctatcggcatccccggcatgcccggatctccaggtcttaaaggttcacctggcagcgtgggctatccgggatcacctggccttccaggcgaaaagggcgacaaaggactgcctggccttgatggcatacctggcgtgaaaggcgaagcaggacttcccggtacacctggacctacaggaccagctggccaaaaaggcgaaccgggatctgatggaattcccggctctgctggcgaaaaaggcgagccaggccttcctggaagaggcttcccaggatttcctggcgcaaagggcgataagggctctaagggcgaagtcggctttccaggacttgccggttctcctggcatcccaggttccaagggcgaacaaggattcatgggtcctccgggtcctcagggtcaaccagggttgcctggaagccctggacatgccacagaaggaccaaaaggcgacagaggacctcagggacaacctgggcttcccggccttccaggaccaatgggtcctcctggactccccggtattgatggcgtcaagggcgacaagggaaatccaggatggccaggtgctccaggcgttcccggtccaaagggcgatcccgggtttcaagggatgcctggtatcggaggaagccccggcattactggaagcaaaggcgacatgggaccaccaggcgtgcccggttttcagggacctaaagggttgccaggcctgcagggaatcaaaggcgaccagggcgatcaaggcgttccaggtgccaagggattgcctgggccaccaggaccgccaggaccttacgatatcattaagggcgagcccggactgcctggtcctgagggtcctccaggattgaaaggacttcaggggctccctggaccaaaaggacagcagggtgttacaggcctcgtcggtattcctggacctccggggatacctggatttgatggtgctcctgggcagaaaggcgaaatgggtccagcaggaccaacaggcccaagaggtttccccggacctccagggcctgatggcctgccaggatctatgggtccaccagggacaccatccgtggatcacggctttctggtcaccagacacagccagaccatcgacgatcctcagtgtcctagcggcaccaagatcctgtatcacggctacagcctgctgtacgtgcagggcaatgagagagcacacggacaggatctgggcacagccggcagctgtctgcggaagtttagcaccatgccttttctgttctgcaacatcaacaacgtgtgcaacttcgccagccggaacgactacagctactggctgtctacccctgagcctatgcctatgagcatggcccctatcaccggggagaacatcagacccttcatcagcagatgtgccgtgtgcgaagcccctgccatggttatggctgtgcactcccagaccattcagatccctccatgtccaagcggctggtctagcctgtggatcggctactcctttgtgatgcacacatctgccggcgcagaaggatcaggacaagcccttgctagccccggctcctgtctggaagaattcagaagcgcccctttcatcgagtgccacggcagaggcacctgtaactactacgccaacgcctacagcttttggctggccaccatcgagcggagcgagatgttcaagaagcccacaccttctacactgaaggccggcgagctgagaacacacgtgtccagatgtcaagtgtgcatgcggcggacctga

SEQ ID NO: 9 - DNA, 3087 bp, Homo sapiens
  source 1..3087: mol_type = unassigned DNA
atgagggcggcccgtgctctgctgcccctgctgctgcaggcctgctggacagccgcgcaggatgagccggagaccccgagggccgtggccttccaggactgccccgtggacctgttctttgtgctggacacctctgagagcgtggccctgaggctgaagccctacggggccctcgtggacaaagtcaagtccttcaccaagcgcttcatcgacaacctgagggacaggtactaccgctgtgaccgaaacctggtgtggaacgcaggcgcgctgcactacagtgacgaggtggagatcatccaaggcctcacgcgcatgcctggcggccgcgacgcactcaaaagcagcgtggacgcggtcaagtactttgggaagggcacctacaccgactgcgctatcaagaaggggctggagcagctcctcgtggggggctcccacctgaaggagaataagtacctgattgtggtgaccgacgggcaccccctggagggctacaaggaaccctgtggggggctggaggatgctgtgaacgaggccaagcacctgggcgtcaaagtcttctcggtggccatcacacccgaccacctggagccgcgtctgagcatcatcgccacggaccacacgtaccggcgcaacttcacggcggctgactggggccagagccgcgacgcagaggaggccatcagccagaccatcgacaccatcgtggacatgatcaaaaataacgtggagcaagtgtgctgctccttcgaatgccagcctgcaagaggacctccggggctccggggcgaccccggctttgagggagaacgaggcaagccggggctcccaggagagaagggagaagccggagatcctggaagacccggggacctcggacctgttgggtaccagggaatgaagggagaaaaagggagccgtggggagaagggctccaggggacccaagggctacaagggagagaagggcaagcgtggcatcgacggggtggacggcgtgaagggggagatggggtacccaggcctgccaggctgcaagggctcgcccgggtttgacggcattcaaggaccccctggccccaagggagaccccggtgcctttggactgaaaggagaaaagggcgagcctggagctgacggggaggcggggagaccagggagctcgggaccatctggagacgagggccagccgggagagcctgggccccccggagagaaaggagaggcgggcgacgaggggaacccaggacctgacggtgcccccggggagcggggtggccctggagagagaggaccacgggggaccccaggcacgcggggaccaagaggagaccctggtgaagctggcccgcagggtgatcagggaagagaaggccccgttggtgtccctggagacccgggcgaggctggccctatcggacctaaaggctaccgaggcgatgagggtcccccagggtccgagggtgccagaggagccccaggacctgccggaccccctggagacccggggctgatgggtgaaaggggagaagacggccccgctggaaatggcaccgagggcttccccggcttccccgggtatccgggcaacaggggcgctcccgggataaacggcacgaagggctaccccggcctcaagggggacgagggagaagccggggaccccggagacgataacaacgacattgcaccccgaggagtcaaaggagcaaaggggtaccggggtcccgagggcccccagggacccccaggacaccaaggaccgcctgggccggacgaatgcgagattttggacatcatcatgaaaatgtgctcttgctgtgaatgcaagtgcggccccatcgacctcctgttcgtgctggacagctcagagagcattggcctgcagaacttcgagattgccaaggacttcgtcgtcaaggtcatcgaccggctgagccgggacgagctggtcaagttcgagccagggcagtcgtacgcgggtgtggtgcagtacagccacagccagatgcaggagcacgtgagcctgcgcagccccagcatccggaacgtgcaggagctcaaggaagccatcaagagcctgcagtggatggcgggcggcaccttcacgggggaggccctgcagtacacgcgggaccagctgctgccgcccagcccgaacaaccgcatcgccctggtcatcactgacgggcgctcagacactcagagggacaccacaccgctcaacgtgctctgcagccccggcatccaggtggtctccgtgggcatcaaagacgtgtttgacttcatcccaggctcagaccagctcaatgtcatttcttgccaaggcctggcaccatcccagggccggcccggcctctcgctggtcaaggagaactatgcagagctgctggaggatgccttcctgaagaatgtcaccgcccagatctgcatagacaagaagtgtccagattacacctgccccatcacgttctcctccccggctgacatcaccatcctgctggacggctccgccagcgtgggcagccacaactttgacaccaccaagcgcttcgccaagcgcctggccgagcgcttcctcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggaagcccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggtggcgctgggctag

SEQ ID NO: 10 - DNA, 3087 bp, synthetic construct
  misc_feature 1..3087: note = Synthetic Construct
  source 1..3087: mol_type = other DNA
atgagagccgctagagcccttctgcctctgctgctgcaagcctgttggacagccgctcaggatgagcccgaaacacctagagccgtggcattccaggactgccccgtggatctgttcttcgtgctggataccagcgagagcgtggccctgagactgaaaccttatggcgccctggtggacaaagtgaagtccttcaccaagcggttcatcgacaacctgcgcgaccggtactacagatgcgacagaaacctcgtgtggaacgcaggcgccctgcactactctgacgaggtggaaatcatccagggcctgaccagaatgcctggcggaagagatgccctgaagtctagcgtggacgccgtgaagtactttggcaagggcacctacaccgactgcgccatcaagaaaggcctggaacagctgctcgtcggcggcagccatctgaaagagaacaagtacctgatcgtggttaccgacggacaccctctggaaggctacaaagaaccttgtggcggactggaagatgccgtgaacgaggccaaacacctgggcgtgaaggtgttcagcgtggccatcacacccgaccacctggaacctcggctgagcatcattgccaccgaccacacctaccggcggaatttcacagccgctgattggggccagagcagggatgccgaagaggccatcagccagaccatcgacaccatcgtggacatgatcaagaacaacgtggaacaagtgtgctgcagcttcgagtgccagcctgctagaggacctcctggacttagaggcgaccctggctttgagggcgagagaggaaaacctggactgcccggcgaaaaaggcgaagctggcgatcctggtagacctggcgatctgggacctgtgggctatcagggaatgaagggcgagaaaggctcccggggagagaagggatctagaggccctaagggctacaagggcgaaaaaggcaagaggggcatcgatggcgtggacggcgtcaaaggcgagatgggatatcctggactccctggctgtaaaggcagccctggcttcgatggaatccagggacctccaggacctaagggcgatccaggcgcctttggactgaaaggcgaaaagggcgaaccaggtgccgatggcgaagcaggcagacctggatcttctggccctagcggagatgaaggacagcctggcgaacctggaccacctggcgagaagggcgaagccggcgacgagggaaatccaggaccagatggtgctcctggcgaaagaggtggaccaggcgaaaggggacctagaggaacaccaggcacaagaggcccaagaggcgatcccggcgaggctggacctcaaggcgatcagggaagagaaggaccagtgggagttcctggcgacccaggcgaagcaggacctatcggccctaagggatatagaggcgacgaaggccctcctggatctgaaggtgctagaggcgcaccaggtccagcaggccctccaggcgaccccggacttatgggagaacgcggagaagatggccctgccggcaatggcacagagggctttccaggctttcctggctaccccggaaatagaggcgctcctggaatcaacggcaccaagggatatccagggctcaaaggcgacgaaggcgaggcaggcgatccaggggatgacaacaacgatatcgcccctagaggcgtgaaaggcgccaaaggctatagaggaccagagggaccacaaggcccacctggtcatcaagggccaccaggacctgacgagtgcgagatcctggacatcattatgaagatgtgcagctgctgcgagtgcaagtgcggccctatcgatctgctgtttgtgctggacagctccgagagcatcggcctgcagaatttcgagatcgccaaggacttcgtggtcaaagtgatcgacagactgagccgggacgagctggtcaagtttgagcctggccagtcttatgccggcgtggtgcagtacagccacagccagatgcaagagcacgtgtccctgagaagccccagcatcagaaacgtgcaagagctgaaagaagccatcaagtccctgcagtggatggctggcggaacctttactggcgaggccctgcagtacaccagagatcaactgctgcctccttctcctaacaaccggattgccctcgtgatcaccgacggcagaagcgacacccagagagacaccacacctctgaacgtgctgtgcagccccggcattcaggtggtgtctgtgggcatcaaggacgtgttcgacttcatccccggcagcgaccagctgaacgtgatctcttgtcaaggactggcccctagccaaggcagaccaggactgtctctggtcaaagagaactacgccgagctgctcgaggacgccttcctgaagaatgtgacagcccagatctgcatcgacaagaagtgccccgactacacatgccccatcacctttagcagccctgccgacatcaccatcctgctggatggctctgctagcgtgggcagccacaacttcgacaccaccaagagattcgccaagcggctggccgagagatttctgacagccggcagaaccgatcctgctcacgatgtgcgagtggccgtggtccagtattctggcacaggccagcaaagacccgagagagcctctctgcagttcctgcagaactacactgccctggcctctgccgtggatgccatggactttatcaacgacgccaccgacgtgaacgacgccctgggctatgtgacccggttttacagagaagcctctagcggagccgccaagaagagactgctgctgttcagcgacggcaactcccaaggtgctacaccagccgccattgagaaggccgtgcaagaagctcagagagccggcatcgagatcttcgtggtggtcgtgggcagacaagtgaacgagcctcacatcagagtgctggtcaccggcaagaccgccgagtacgatgtggcttatggcgagagccacctgttcagagtgcccagctatcaggctctgctgagaggcgtgttccaccagaccgtgtctagaaaggtggccctgggatga

SEQ ID NO: 11 - DNA, 8835 bp, Homo sapiens
  source 1..8835: mol_type = unassigned DNA
atgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactga

SEQ ID NO: 12 - DNA, 8835 bp, synthetic construct
  misc_feature 1..8835: note = Synthetic Construct
  source 1..8835: mol_type = other DNA
atgaccctgagactgctggtggctgccctgtgtgctggaattctggctgaggctcctagagtgcgggcccagcatagagaaagagtgacctgtacacggctgtacgccgccgatatcgtgtttctgctggatggcagcagcagcatcggccggtccaatttcagagaagtgcggagctttctggaaggcctggtgctgcctttttctggcgctgcttctgcccagggcgtcagatttgccaccgtgcagtacagcgacgaccctagaacagagtttggcctggatgctctcggcagcggcggagatgtgatcagagccatcagagagctgagctacaaaggcggcaataccagaacaggcgccgctattctgcacgtggccgaccatgtttttctgccccaactggctagacccggcgtgccaaaagtgtgcatcctgatcacagacggcaagagccaggacctggtggatacagccgctcagagactgaaaggacagggcgtgaaactgttcgccgtgggcatcaagaacgccgatcctgaggaactgaagagagtggccagccagcctaccagcgatttcttcttcttcgtgaacgacttcagcatcctgcggaccctgctgcctctggtgtctagaagagtgtgtacaacagccggcggagtgcctgtgaccagacctcctgatgatagcacaagcgcccctagagatctggtgctgtctgagccaagcagccagagtctgagagtgcagtggacagctgctagcggccctgtgacaggctacaaggtgcagtatacacctctgacaggcctgggccagcctctgccttctgagagacaagaagtgaacgtcccagccggcgaaacatctgtcagactgagaggactgaggcccctgaccgagtaccaagtgacagtgatcgccctgtacgccaattctatcggcgaggccgtgtctggcacagccagaacaacagctctggaaggaccagagctgaccatccagaacacaacagcccattctctgctggttgcttggagatctgtgcctggcgccaccggctatagagtgacttggagagttctgagcggaggccccacacagcagcaagaacttggacctggacagggctccgtgctgctgagagatctcgagcctggcaccgattacgaagtgaccgtgtctaccctgttcggcagatctgtgggacctgccacaagcctgatggccagaacagatgccagcgtggaacagaccctgaggcctgtgattctgggccctacatctatcctgctgagctggaatctggtgcccgaggccagaggctatagactggaatggcggagagaaaccggcctggaacctcctcagaaagtggtcctgcctagcgacgtgacaagataccagctggatggactgcagcccggcaccgagtacagactgacactgtatacactgctcgagggccacgaagtggccacaccagctacagttgtgccaacaggacctgagctgcctgtgtctcctgtgactgatctgcaggccacagaactgcctggccagagagtcagagttagctggtcacctgttccaggcgccacacagtacagaatcatcgtgcggtctacacagggcgtcgagagaacactggttctgcctggaagccagaccgccttcgatctggatgatgtgcaggccggactgtcctacacagtcagagtgtctgccagagtgggccctagagaaggatctgcctctgtgctgaccgtgcggagagagcctgaaacaccactggcagtgcctggactgagagtggtggtgtcagatgccaccagagttagagtggcttggggaccagttcctggcgcctctggctttagaatcagctggtctacaggctctggccccgaaagctctcagacactgcctccagatagcaccgccaccgatattacaggcctgcagcctggaactacctaccaggtggccgttagcgtgctgagaggaagagaagaaggccctgccgccgtcatcgtggctagaactgatcctctgggacctgtgcggacagtgcacgtgacacaagcctctagcagcagcgtgaccatcacctggacaagagtgccaggcgctacaggatacagagtgtcctggcattctgctcacggccctgagaagtctcagctggtgtctggcgaagccacagtggctgaactcgacggactggaacctgacacagagtatactgtgcacgtgcgggctcatgtggctggtgttgatggacctcctgcttctgtggtcgtcagaacagcccctgaacctgtgggaagagtgtccagactgcagatcctgaatgccagcagcgacgtgctgaggatcacatgggttggagtgaccggcgccacagcttatagactcgcctggggtagaagcgaaggcggccctatgagacatcagatcctgcctggcaataccgactccgccgagattagaggccttgaaggcggagtgtcttacagcgttagagtgacagccctcgtgggcgatagagaaggcacacctgtgtccatcgtggtcaccacacctccagaagctcctccagctctgggaacactgcatgtggtgcagagaggcgagcactctctgagactgagatgggaacctgtgccacgcgctcagggatttctgctgcattggcaaccagaaggcggacaagagcagagcagagtgctgggaccagaactgagcagctaccacctcgatggacttgagcctgccactcagtatagagtcagactgtcagtgctggggcctgccggcgaaggaccttctgctgaagtgacagctagaaccgagtctcccagagtgcccagcatcgagctgagagtcgtggatacctccatcgatagcgtgacactggcctggacacccgtgtctagagccagctcttacatcctgtcttggcggcctctcagaggaccaggccaagaagttcctggatctccacagacactcccaggcatcagtagctcccagagagtgactggacttgaaccaggcgtgtcctacatcttcagcctgacacctgtgctggacggcgttagaggacctgaagcctctgtgacccagactccagtgtgtcctagaggactggccgatgtggtgttcctgcctcacgctacacaggacaatgcccatagagccgaggccacaagacgcgtgctggaaagactggttcttgcccttggaccactgggacctcaggctgttcaagtgggcctgctgtcctactctcacagaccctctccactgttccctctgaacggctctcacgacctgggcatcatcctgcagagaatccgggacatgccctacatggacccctctggcaacaatctgggcacagccgttgtgactgcccacagatatatgctggccccagatgctcctggcagacgacaacatgtccctggcgttatggtgctgctggtcgatgaacccctgcggggcgatatctttagccctattagagaagcccaggccagcggcctgaatgtggttatgcttggaatggctggcgccgatccagagcagcttagaaggcttgcccctggcatggatagcgtgcagaccttctttgccgtggacgatggaccttctctggatcaggctgtgtctggactggctacagcactgtgccaggcaagcttcaccacacagcctagacctgagccttgtccagtgtactgccctaagggacagaagggcgaacctggcgaaatgggacttagaggccaagtgggaccacctggcgatcctggacttcctggaagaactggtgctcctggacctcaaggtcctcctggaagtgccacagccaagggcgaaagaggattccctggcgctgatggcagaccaggatctcctggtagagccggcaatcctggaacacctggcgcaccaggactcaaaggatctccaggactgcctggacctagaggcgatccaggcgagagaggaccaagaggtccaaaaggcgaacccggtgctccaggacaagtgattggcggagaaggacccggattgcccggaagaaaaggcgaccctggaccaagtggacctccaggacctaggggacctttgggagatcccggtccaagaggccctcctggattgcctggtacagccatgaagggcgacaaaggcgataggggagaaagaggaccaccaggaccaggcgaaggtggaattgctcctggcgaaccagggttgcctggactccctggctcacctggaccacaaggacctgttggcccacctggaaagaaaggcgaaaagggcgattctgaggatggcgccccagggcttcctggacaaccaggctctccaggcgaacaaggacccagagggcctccaggtgctattggccctaaaggcgacagagggtttcccggaccacttggagaagctggcgaaaaaggcgaacgaggacctcctggacctgccggatctagaggacttccaggtgttgctggcagacctggcgctaaaggtcctgaaggcccaccagggcctacaggcagacaaggcgagaaaggcgagccaggcagacccggcgatcctgctgttgttggaccagctgttgcaggcccaaagggcgagaagggcgacgttggacctgctggaccaaggggagctacaggcgttcaaggcgaaaggggtccacctggacttgttttgccgggcgatcccggacctaagggcgaccccggcgacaggggaccaattggattgacaggcagagctgggccaccaggcgatagtgggcctccaggcgaaaaaggcgatcctggtagacctggacctcctgggccagttggaccaagaggaagagatggcgaagtcggagagaaaggcgacgaaggtcctccaggcgacccaggactccctggaaaagcaggcgaaagaggtcttagaggcgctcctggtgttagaggccctgttggagaaaagggcgaccaaggcgaccctggcgaggatggaagaaatggctcccctggatctagcggcccaaaaggcgatcgcggcgagcccggtccaccgggtccaccaggcaggcttgttgatactggacccggcgctcgtgaaaaaggcgagcccggcgatcgtggacaagaaggcccaagaggacccaagggcgatccgggactgccaggtgcaccaggcgagcgaggtattgaaggattcagaggccctccaggaccacaaggcgatcccggtgtcagaggacctgctggcgagaagggcgatagaggtcctccaggactggatggcagatccggacttgatggaaaacccggcgcagctggcccttctggacctaatggtgcagccggaaaagctggcgacccaggcagagatggattgccaggattgaggggagaacagggactccccggaccttctgggccgcctgggttgcccggaaagcctggcgaagatggcaaacctggcctgaacggcaaaaacggcgaaccgggcgatccaggcgaagatggcagaaagggcgaaaaaggcgacagcggagcctctggccgagagggtagagatggacctaaaggcgagaggggcgctcctggaattttgggaccccaaggaccgcctgggctgccaggtccagttgggcctcctggtcaaggttttcctggtgtcccaggcggaactggaccaaaaggcgatagaggcgaaacaggctctaagggcgagcaaggacttcctggcgaacgtggactgagaggcgaacctggaagcgtgcccaatgtggacagactcctggaaaccgccggaatcaaagccagcgctctgcgggaaatcgtggaaacctgggatgagagcagcggctcttttctgcctgtgcctgaaagaagaaggggaccgaaaggcgattctggcgaacaaggtccacctggcaaagagggccctatcggatttcccggcgagcggggtcttaaaggcgaccggggagatccgggacctcaagggccaccaggacttgctcttggagaaagaggtcctccggggccatctggacttgctggcgagcctggaaaacctggaataccaggacttcccggcagagcaggcggagttggagaggccggacgacctggcgaacgcggagaaaggggagagaagggcgagcgaggcgaacagggacgagatggaccaccggggctccctggaactcctggaccgccaggaccacctggtcctaaagtgtcagtggatgagcctggaccaggtcttagcggagaacaaggacctccaggcctgaaaggcgctaagggcgaaccaggttcaaatggcgatcagggacccaaaggcgatcggggcgttccaggcattaagggcgaccgtggcgaaccaggacctagaggacaagatggcaatcccggactgcctggcgaaagaggaatggccggacctgagggaaagccagggctgcaagggcctcgcgggccacctgggcctgtcggaggacatggcgaccccggaccaccaggtgctcccggacttgctggaccagcaggacctcaaggaccatctggacttaaaggcgaaccaggcgaaacagggccaccaggcagaggacttacaggacctacaggtgctgttgggttgcctggtccaccagggccaagtggacttgttggtcctcaaggttcacctggactgcccggacaagtgggagaaactggcaaaccaggcgcacctgggcgagatggcgcatctggaaaagacggcgatcgaggtagccctggtgttcccggttctcctgggctcccaggacctgtgggccccaagggcgaacccggaccaactggcgcacctggtcaagcagttgttggattgcctggcgcaaagggcgagaaaggtgctccaggcggattggctggcgatcttgttggagaaccaggtgccaaaggcgacaggggcttgccaggtcctaggggagagaaaggcgaagctggtagagcaggcgagcctggcgatcccggcgaggacggacaaaaaggtgcccctgggcctaaaggctttaagggcgatcctggtgtcggcgtcccaggttctcctggaccacctgggccgccaggcgttaagggcgatttgggacttcctgggctgcctggcgctcctggcgttgtgggatttccaggacaaactggccctcggggagaaatgggtcaacctgggcctagcggagaaagaggccttgccggacctcctggaagagagggaatacctggaccacttggaccgcctggacctccaggttctgttggacctcctggcgcttcaggattgaagggcgataagggcgaccctggtgttggacttcccggtcctcgtggcgaaaggggagaaccaggcattagaggcgaagatggacggcctggacaagagggacctagaggtcttacaggcccaccaggctctagaggcgaaaggggcgagaaaggcgacgtgggttctgcaggacttaagggcgacaagggcgattccgctgtgattttgggaccaccgggaccaagaggcgctaaaggcgatatgggcgagagaggccctagaggcctggatggcgataaggggccaagaggcgacaacggcgacccaggcgataagggatctaaaggcgaacccggcgataagggctctgctggactgccaggacttcgaggacttctgggtccacaaggccaacctggcgccgcaggcattcccggcgatccgggttctcctggcaaagatggtgttcctggcatcaggggcgaaaagggcgacgtcggttttatgggccctcgcggattgaaaggcgagagaggtgttaagggcgcctgtggacttgacggcgaaaaaggcgataagggcgaagctggacctcctggcagacctggattggctggacacaagggcgagatgggagagcccggtgttcctggacaaagtggcgcccctggaaaagaaggactgatcggtccaaagggcgaccgcggatttgatggccaaccgggtcctaaaggcgatcagggcgagaaaggcgaaagaggcactcctggcatcggaggctttccaggaccaagcggcaatgatggatctgctggcccgccagggcctcctggatctgttggtccaagaggaccagaaggcctgcagggacaaaaaggcgagcgcggaccaccaggcgaaagagttgttggagcacccggcgttcccggtgctcccggcgaaagaggcgaacaaggcagacctggaccagctggccctagaggcgaaaaaggggaagccgctctgaccgaggacgatatcagaggctttgtgcggcaagagatgagccagcattgtgcctgtcaggggcagtttatcgccagcggttctagacctctgcctagctatgccgctgataccgccggatctcagctgcatgctgtgcctgtgcttagagtgtctcacgccgaggaagaggaaagagtccctccagaggacgacgagtactccgagtatagcgagtactctgtggaagagtatcaggaccccgaggctccttgggatagcgacgatccatgttctctgccactggatgagggcagctgtaccgcctacacactgaggtggtatcacagagccgtgaccggaagcaccgaggcctgccatccttttgtttatggcggctgcggcggcaacgccaatagatttggaacaagagaggcctgcgagcggagatgccctccaagagtggttcagtctcaaggcaccggcactgcccaggactaa

SEQ ID NO: 13 - DNA, 4494 bp, Homo sapiens
  source 1..4494: mol_type = unassigned DNA
atggatgtaaccaagaaaaacaaacgagatggaactgaagtcactgagagaattgtcactgaaacagtaaccacaagacttacatccttaccaccaaaaggcgggaccagcaatggctatgctaaaacagcctctcttggtggagggagccggctggagaaacaaagcctgactcatggcagcagcggctacataaactcaactggaagcacacgaggccatgcctccacctctagttacaggagggctcactcacctgcctccactctgcccaactccccaggctcaacctttgaaaggaaaactcacgttacccgccatgcgtatgaagggagctccagtggcaactcttctccggagtaccctcggaaggaatttgcatcttcttcaaccagaggacggagtcaaacacgagagagtgaaattcgagttcgactgcagagtgcgtccccatccacccgatggacagaattggatgatgttaagcgtttgctcaaggggagtcgatcggcaagtgtgagccccacccggaattcctccaacacactccccatccccaagaaaggcactgtggagaccaaaattgtgacagcgagctcccagtcggtgtcaggcacctacgatgcaacgatcctggatgccaaccttccctcccatgtgtggtcctccaccctgcccgcggggtcctccatggggacctatcacaacaacatgacaacccagagctcatccctcctcaacaccaatgcctactctgcgggatcagtcttcggagttccaaacaacatggcgtcctgctcacccactttgcaccctggactcagcacatcctcctcagtgtttggcatgcagaacaatctggcccccagcttgaccaccctgtcccatggcaccaccaccacttccacagcatatggggtgaagaaaaacatgccccagagtcctgcggctgtgaacactggcgtttccacctccgccgcctgcaccacaagtgtgcagagcgatgaccttttgcacaaggactgcaagttcctgatcctagagaaagacaacacacctgccaagaaggagatggagctgctcatcatgaccaaggacagcgggaaggtctttacagcctcccctgccagcatcgctgcaacttctttttcagaagacaccctaaaaaaagaaaagcaagctgcctacaatgctgactcaggcctaaaagccgaagctaatggagacctgaagactgtgtccacaaagggcaagaccaccactgcagatatccacagctacggcagcagtggtggtggtggcagtggaggaggtggcggtgttggtggcgctggcggcggcccttggggaccagcgccagcctggtgcccctgcggctcctgctgcagctggtggaagtggctgctgggcctgctgctcacctggctgctactcctggggctgctcttcggcctcattgctctggcggaggaggtgaggaagctgaaggcgcgtgtggatgagctggagaggatcaggaggagcatactgccctatggggacagcatggatagaatagaaaaggaccgcctccagggcatggcacccgcggcgggagcagacctggacaaaattgggctgcacagtgacagccaggaggagctctggatgttcgtgaggaagaagctaatgatggaacaggaaaatggaaatctccgaggaagccctggccctaaaggtgacatgggaagtccaggccctaaaggagatcgagggttccctgggactccaggtatccctgggcccttgggccacccaggtccacaaggaccaaagggtcaaaaaggcagcgtgggagatcctggcatggaaggccccatgggccagagagggcgagaaggccccatgggacctcgtggtgaggcagggcctcctggatctggagagaaaggggaaagaggggctgctggtgaaccaggtcctcatggcccacctggtgtcccaggttctgtgggtcccaaaggttccagcggctctcctggcccacagggccctccaggtcctgtaggtctccaagggctccgaggtgaagtaggacttcctggtgtcaaaggtgacaaaggaccaatgggaccaccaggacccaaaggtgaccagggtgagaaaggacctcgaggcctcacaggcgagcctggcatgagaggtttgcctggtgctgttggtgagcccggggctaaaggagcaatgggtcctgctggcccagacggacaccaaggcccaagaggtgaacaaggtcttactgggatgcctggaatccgtggcccaccaggaccttctggagacccaggaaagccaggtctcacaggaccccagggacctcagggacttcccggtacccctggccgaccaggaataaaaggtgaaccaggagctccaggcaagatcgtgacttcggaggggtcatcgatgctcactgtcccaggccccccaggacctcctggagccatgggacccccaggacctccaggtgccccaggccctgccggcccagctggtctcccaggacatcaagaagttcttaatttacaaggtcccccaggcccacccggcccacgcgggccaccagggccttccattccaggcccaccaggaccccgaggcccaccaggggagggtttgccaggcccaccaggcccaccaggatcgttcctgtccaactcagaaaccttcctctccggccccccaggcccacctggccccccaggtcccaagggagaccaaggtcccccaggccccagaggacaccaaggcgagcaaggcctcccaggtttctcaacctcagggtccagttctttcggactcaaccttcagggaccaccaggcccacctggcccccagggacccaaaggtgacaaaggtgatccaggtgttccaggggctcttggcattcctagtggtccttctgaagggggatcatcaagtaccatgtacgtgtcaggcccgccagggccccctgggccccctgggcctccgggctctatcagcagctctggccaggagattcagcagtacatctctgagtacatgcagagtgacagtattagatcttacctatccggagttcagggtcccccaggcccacctggtcccccaggacctgtcaccaccatcacaggcgagactttcgactactcagagctggcaagccacgttgtgagctacttacggacttcggggtacggtgtcagcttgttctcgtcctccatctcttctgaagacattctggctgtgctgcagcgggatgacgtgcgtcagtacctacgtcagtacttgatgggccctcggggtccgccagggccaccaggagccagtggagatgggtccctcctgtctttggactatgcagagctgagtagtcgcattctcagctacatgtcgagttctgggatcagcattgggcttcctggtcccccggggccccctggcttgccgggaacctcctatgaggagctcctctccttgctgcgagggtctgaattcagaggcatcgttggacccccaggtcccccgggtccaccagggatcccaggcaatgtgtggtccagcatcagcgtggaggacctctcgtcttacttacatactgccggcttgtcattcatcccaggccctccaggacctcctggtcccccagggcctcgagggcccccgggtgtctcaggagccctggcaacctatgcagctgaaaacagcgacagcttccggagcgagctgatcagctacctcacaagtcctgatgtgcgcagcttcattgttggccccccaggccctcctgggccgcagggaccccctggggacagccgcctcctgtccacggatgcctcccacagtcggggtagcagctcctcctcacacagctcatctgtcaggcggggcagctcctacagctcttccatgagcacaggaggaggtggtgcaggctccctgggtgcaggcggtgcctttggtgaagctgcaggagacaggggtccctatggcactgacatcggcccaggcggaggctatggggcagcagcagaaggcggcatgtatgctggcaatggcggactattgggagctgactttgctggagatctggattacaatgagctggctgtgagggtgtcagagagcatgcagcgtcagggcctactgcaagggatggcctacactgtccagggcccaccaggccagcctgggccacaggggccacccggcatcagcaaggtcttctctgcctacagcaacgtgactgcggacctcatggacttcttccaaacttatggagccattcaaggaccccctgggcaaaaaggagagatgggcactccaggacccaaaggtgacaggggccctgctgggccaccaggtcatcctgggccacctggccctcgaggacacaagggagaaaaaggagacaaaggtgaccaagtctatgctgggcggagaaggagaagaagtattgctgtcaagccgtga

SEQ ID NO: 14 - DNA, 4494 bp, synthetic construct
  misc_feature 1..4494: note = Synthetic Construct
  source 1..4494: mol_type = other DNA
atggacgtgaccaagaagaacaagcgcgacggcaccgaagtgaccgagagaatcgtgaccgaaaccgtgaccaccagactgaccagcctgcctcctaaaggcggcacctctaatggctacgccaagacagcttctctcggcggaggcagcagactggaaaagcagtctctgacacacggcagcagcggctacatcaatagcaccggctctacaagaggccacgccagcacaagcagctacagaagggctcacagccctgccagcacactgcctaatagcccaggcagcaccttcgagagaaagacccacgtgaccagacacgcctacgagggctctagcagcggcaatagcagccctgagtaccccagaaaagagttcgccagcagcagcaccagaggcagaagccagacacgggaaagcgagatcagagtgcggctgcagtctgctagccctagcaccagatggaccgagctggacgacgtgaagaggctgctgaagggaagcagatccgccagcgtgtcccctaccagaaacagcagcaacaccctgcctattcctaagaaaggcaccgtcgagactaagatcgtgacagccagcagccagagcgtgtccggcacatacgatgccacaatcctggacgccaacctgcctagccatgtgtggtcctctacactgcctgccggaagcagcatgggcacctaccacaacaacatgaccacacagagcagcagcctgctgaacaccaatgcctactctgccggctccgtgttcggcgtgccaaacaatatggccagctgcagccctacactgcaccctggcctgagcacaagctcctccgtgtttggaatgcagaacaatctggcccctagcctgacaaccctgagccacggaacaaccaccaccagcacagcctacggcgtgaagaaaaacatgcctcagtctccagccgccgtgaacacaggcgttagcacatctgccgcctgtaccacaagcgtgcagagcgacgatctgctgcacaaggactgcaagtttctgatcctggaaaaggacaacacgcccgccaagaaagagatggaactgctgatcatgaccaaggacagcggcaaggtgttcaccgcctctccagcctctatcgccgccacaagctttagcgaggacaccctgaagaaagaaaagcaggccgcctacaacgccgactctggactgaaagccgaggccaacggcgacctgaaaaccgtgtctaccaagggcaagaccaccaccgccgacatccacagctatggatctagcggaggcggaggatctggtggtggcggcggagttggaggtgctggcggaggaccttggggacctgctcctgcttggtgtccttgtggctcctgttgcagctggtggaagtggctgcttggcctgctgctgacttggctgcttcttctgggcctgctgtttggcctgattgccctggctgaggaagtgcggaagctgaaggccagagtggatgagctggaacggatcaggcggagcatcctgccttacggcgacagcatggaccggatcgagaaggacagactgcaaggcatggctccagcagctggcgccgatctggataagatcggactgcacagcgacagccaagaggaactctggatgttcgtgcggaagaaactgatgatggaacaagagaacggcaacctgagaggcagccctggacctaagggcgatatgggaagcccaggaccaaaaggcgacagaggctttcctggcacacctggcattccaggacctctgggacatcctggtcctcagggccctaaaggccagaaaggctctgtgggagatcccggcatggaaggccctatgggacagagaggtagagaaggaccaatgggccctagaggcgaagctggacctcctggatctggcgagaaaggcgaaaggggagctgcaggcgaaccaggaccacatggacctccaggtgttcccggatctgtgggccctaagggatcttctggttcccctggaccacaaggcccacctggacctgttggactccaaggactgaggggcgaagttggactgccaggcgtcaagggcgacaagggtccaatgggaccacctggtccaaagggcgatcagggcgaaaagggacctagaggactgacaggcgagcccggaatgagaggacttcctggtgctgttggagagcctggcgctaaaggtgctatgggacctgcaggccccgacggacatcaaggacctaggggagaacagggcctgaccggcatgcctggaattagaggtcctcctggaccttccggcgatcctggcaaaccaggattgactggacctcagggaccacagggactgcctggaacaccaggcagacctggaatcaaaggcgaacctggcgctcccggcaagattgtgacatctgagggcagctccatgctgaccgtgcctggtccacctgggcctccaggcgccatgggtcctccgggtccaccaggtgctccaggaccagccggaccagcaggacttccaggccatcaagaagtgctgaacctgcaggggcctcctgggccgcctggaccaagagggccaccagggccatctattccaggaccaccaggtcctagaggccctccaggcgaaggattgccaggtcctccagggccacctggcagctttctgagcaacagcgagacattcctgagcggccctccaggacctcctggaccaccaggacctaaaggcgatcaaggacctccaggaccgagaggacatcagggcgaacaaggactgcctggctttagcacaagcggcagctctagcttcggcctcaatctgcagggtccaccagggccaccaggacctcaaggtcctaaaggcgataagggcgatccaggcgttccaggcgctctgggtattccttctggaccatctgaaggcggctcctccagcactatgtacgtgtcaggtccaccgggtccacctggaccgccaggaccacctggatctatctctagctccggccaagagatccagcagtacatcagcgagtacatgcagtctgacagcatccggtcctacctgtctggcgttcaaggtccaccgggacctccggggcctcctggacctgttacaacaatcaccggcgagactttcgactacagcgagctggcctctcacgtggtgtcctatctgagaaccagcggctatggcgtgtccctgttcagctccagcatcagctccgaggacattctggcagtgctgcagagggatgacgtgcggcagtacctgagacagtatctgatggggcccagagggccacctggtccaccaggcgctagcggagatggatctctgctgagcctggattacgccgagctgagcagcagaatcctgagctacatgagcagctccggcatctccattgggttgcctggacctccaggtccaccaggattgcctggcacaagctacgaggaactgctgtccctgctgaggggcagcgagtttagaggaatcgttggaccacctgggccaccaggtccacctggtatccctggaaatgtgtggtctagcatcagcgtggaagatctgagcagctacctgcacacagccggcctgagctttattccaggacctccagggccgccagggcctcctggtcctcggggaccgcctggcgttagcggagcacttgcaacatatgccgccgagaacagcgactccttcagaagcgagctgatctcctacctgactagccccgatgtgcggagctttatcgttggcccgcctggtcctccaggacctcaaggacctcctggcgattctagactgctgagcacagatgccagccacagcagaggcagctcctctagctctcactccagttctgtgcggagaggctccagctacagcagctctatgtcaacaggtggcggaggcgctggaagtcttggagctggtggcgcttttggagaagccgctggcgatcgtggcccatacggaacagatattggacccggcggtggatatggcgctgccgctgaaggcgggatgtatgccggaaatggtggactgctgggcgccgattttgctggcgacctggactataatgagctggccgtcagagtgtccgagagcatgcaaagacaggggctgcttcaaggcatggcctacacagttcaaggcccaccaggacagcctggtccacagggaccaccgggaatcagcaaagtgttctctgcctacagcaacgtgaccgccgacctgatggacttcttccagacctacggcgccattcagggacctccaggccaaaagggcgaaatgggtacacctgggccgaaaggcgaccgaggacctgctgggccacctggacatcccgggcctccagggcctagaggacacaaaggcgaaaaaggcgacaaaggggaccaagtctacgccggcagacggcggagaagatccattgccgtgaagccctaa

SEQ ID NO: 15 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 16 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 17 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 18 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 19 - protein, 1028 aa, Homo sapiens
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGSSGPSGDEGQPGEPGPPGEKGEAGDEGNPGPDGAPGERGGPGERGPRGTPGTRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVAYGESHLFRVPSYQALLRGVFHQTVSRKVALG

SEQ ID NO: 20 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 21 - protein, 1497 aa, Homo sapiens
MDVTKKNKRDGTEVTERIVTETVTTRLTSLPPKGGTSNGYAKTASLGGGSRLEKQSLTHGSSGYINSTGSTRGHASTSSYRRAHSPASTLPNSPGSTFERKTHVTRHAYEGSSSGNSSPEYPRKEFASSSTRGRSQTRESEIRVRLQSASPSTRWTELDDVKRLLKGSRSASVSPTRNSSNTLPIPKKGTVETKIVTASSQSVSGTYDATILDANLPSHVWSSTLPAGSSMGTYHNNMTTQSSSLLNTNAYSAGSVFGVPNNMASCSPTLHPGLSTSSSVFGMQNNLAPSLTTLSHGTTTTSTAYGVKKNMPQSPAAVNTGVSTSAACTTSVQSDDLLHKDCKFLILEKDNTPAKKEMELLIMTKDSGKVFTASPASIAATSFSEDTLKKEKQAAYNADSGLKAEANGDLKTVSTKGKTTTADIHSYGSSGGGGSGGGGGVGGAGGGPWGPAPAWCPCGSCCSWWKWLLGLLLTWLLLLGLLFGLIALAEEVRKLKARVDELERIRRSILPYGDSMDRIEKDRLQGMAPAAGADLDKIGLHSDSQEELWMFVRKKLMMEQENGNLRGSPGPKGDMGSPGPKGDRGFPGTPGIPGPLGHPGPQGPKGQKGSVGDPGMEGPMGQRGREGPMGPRGEAGPPGSGEKGERGAAGEPGPHGPPGVPGSVGPKGSSGSPGPQGPPGPVGLQGLRGEVGLPGVKGDKGPMGPPGPKGDQGEKGPRGLTGEPGMRGLPGAVGEPGAKGAMGPAGPDGHQGPRGEQGLTGMPGIRGPPGPSGDPGKPGLTGPQGPQGLPGTPGRPGIKGEPGAPGKIVTSEGSSMLTVPGPPGPPGAMGPPGPPGAPGPAGPAGLPGHQEVLNLQGPPGPPGPRGPPGPSIPGPPGPRGPPGEGLPGPPGPPGSFLSNSETFLSGPPGPPGPPGPKGDQGPPGPRGHQGEQGLPGFSTSGSSSFGLNLQGPPGPPGPQGPKGDKGDPGVPGALGIPSGPSEGGSSSTMYVSGPPGPPGPPGPPGSISSSGQEIQQYISEYMQSDSIRSYLSGVQGPPGPPGPPGPVTTITGETFDYSELASHVVSYLRTSGYGVSLFSSSISSEDILAVLQRDDVRQYLRQYLMGPRGPPGPPGASGDGSLLSLDYAELSSRILSYMSSSGISIGLPGPPGPPGLPGTSYEELLSLLRGSEFRGIVGPPGPPGPPGIPGNVWSSISVEDLSSYLHTAGLSFIPGPPGPPGPPGPRGPPGVSGALATYAAENSDSFRSELISYLTSPDVRSFIVGPPGPPGPQGPPGDSRLLSTDASHSRGSSSSSHSSSVRRGSSYSSSMSTGGGGAGSLGAGGAFGEAAGDRGPYGTDIGPGGGYGAAAEGGMYAGNGGLLGADFAGDLDYNELAVRVSESMQRQGLLQGMAYTVQGPPGQPGPQGPPGISKVFSAYSNVTADLMDFFQTYGAIQGPPGQKGEMGTPGPKGDRGPAGPPGHPGPPGPRGHKGEKGDKGDQVYAGRRRRRSIAVKP

SEQ ID NO: 22 - DNA, 148 bp, synthetic construct
  misc_feature 1..148: note = Synthetic Construct
  source 1..148: mol_type = other DNA
gaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaa

SEQ ID NO: 23 - DNA, 491 bp, synthetic construct
  misc_feature 1..491: note = Synthetic Construct
  source 1..491: mol_type = other DNA
gttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaagcgtctaggccccccgaaccacggggacgtggttttcctttgaaaaacacg

SEQ ID NO: 24 - DNA, 75 bp, synthetic construct
  misc_feature 1..75: note = Synthetic Construct
  source 1..75: mol_type = other DNA
agggccaagaggggcagcggcgagggcaggggcagcctgctgacctgcggcgacgtggaggagaaccccggcccc

SEQ ID NO: 25 - DNA, 66 bp, synthetic construct
  misc_feature 1..66: note = Synthetic Construct
  source 1..66: mol_type = other DNA
ggaagcggagctactaacttcagcctgctgaagcaggctggagacgtggaggagaaccctggacct

SEQ ID NO: 26 - DNA, 69 bp, synthetic construct
  misc_feature 1..69: note = Synthetic Construct
  source 1..69: mol_type = other DNA
ggaagcggacagtgtactaattatgctctcttgaaattggctggagatgttgagagcaaccctggacct

SEQ ID NO: 27 - DNA, 75 bp, synthetic construct
  misc_feature 1..75: note = Synthetic Construct
  source 1..75: mol_type = other DNA
ggaagcggagtgaaacagactttgaattttgaccttctcaagttggcgggagacgtggagtccaaccctggacct

SEQ ID NO: 28 - protein, 25 aa, synthetic construct
  REGION 1..25: note = Synthetic Construct
RAKRGSGEGRGSLLTCGDVEENPGP

SEQ ID NO: 29 - protein, 22 aa, synthetic construct
  REGION 1..22: note = Synthetic Construct
GSGATNFSLLKQAGDVEENPGP

SEQ ID NO: 30 - protein, 23 aa, synthetic construct
  REGION 1..23: note = Synthetic Construct
GSGQCTNYALLKLAGDVESNPGP

SEQ ID NO: 31 - protein, 25 aa, synthetic construct
  REGION 1..25: note = Synthetic Construct
GSGVKQTLNFDLLKLAGDVESNPGP

SEQ ID NO: 32 - DNA, 8565 bp, synthetic construct
  misc_feature 1..8565: note = Synthetic Construct
  source 1..8565: mol_type = other DNA
atgttcagcttcgtggacctgagactgctgctgctcctggctgctacagccctgctgacacacggacaagaggaaggccaggtcgaaggacaggacgaggacatccctcctatcacctgtgtgcagaacggcctgagataccacgaccgggatgtgtggaagcccgagccttgcagaatctgcgtgtgcgacaatggcaaggtgctgtgcgacgacgtgatctgcgacgagacaaagaattgccctggcgccgaagtgcctgagggcgaatgttgtcctgtgtgccctgatggcagcgagagccccacagatcaagagacaacaggcgtggaaggccccaagggcgatacaggacctagaggtcctagaggacctgccggacctcctggcagagatggaattcctggacagcctggactgcccggaccacctggacctccagggcctccaggtccaccaggactcggaggaaattttgccccacagctgagctacggctacgacgagaaaagcacaggcggcatctctgtgcctggacctatgggaccttctggcccaagaggacttcctggtcctcctggtgctccaggacctcagggatttcaaggaccaccaggcgaacctggcgaaccaggcgctagtggtccaatgggaccaagaggccctcctgggccaccaggcaaaaatggcgacgatggcgaagccggaaagcctggaaggcctggcgaaagaggcccgccaggaccgcaaggcgctagagggttgcctggaactgcaggactgcctggcatgaagggccacagaggcttttctggactggatggcgctaagggcgacgctggaccagcaggacctaaaggcgagcctggatctcctggcgagaatggtgcacctggacagatgggtcccagaggattgccaggcgagagaggtagacctggcgctccgggaccagccggtgctagaggaaatgatggcgcaacaggtgctgctgggcctcctggaccaaccggaccagctggcccacctggatttccaggcgctgttggagcaaagggcgaagcaggcccacaaggacctaggggatctgaaggtcctcagggcgttagaggcgagccagggccacctgggcctgccggtgcagctggacctgctggaaaccctggtgctgatggacagccaggtgccaaaggtgctaatggcgcccctggaattgccggcgctccaggttttcccggcgcaagagggccatctggacctcaaggcccaggcggacctccgggtcctaagggaaatagcggagagccaggcgctcctgggagtaaaggcgatactggcgcaaaaggcgaacccggacctgtgggagttcaaggacctcctggaccagctggcgaggaaggcaaaagaggcgctaggggagaaccaggaccaacagggctccctggtccacctggcgagcgcggaggacctggatctagaggattccctggcgcagatggcgtggccggaccaaaaggacctgcaggcgaaaggggatcaccaggtcctgcaggccctaagggttctccaggcgaggctggcagacccggcgaagctggactcccaggtgctaagggactgacaggctcaccaggatctcccggaccagacggaaaaacaggacctccaggaccggcaggacaggatggtagacccggtcctcctggaccgcctggtgcaagaggacaagctggcgtgatgggctttcctggaccaaaaggtgcagccggcgaacctggaaaagcaggcgagaggggagttcccggacctccaggtgctgttggacctgccggaaaagatggcgaagctggtgcacaaggtcctccagggccagccggaccagccggcgagagaggcgaacaaggaccagccggatctccaggatttcagggactgccagggcctgctggcccgcctggcgaggcagggaagccaggcgaacagggtgttcctggcgatcttggagcccctggtcctagcggagctagaggcgaaagaggatttcctggcgaaaggggcgttcagggtccaccgggaccagctggaccaaggggtgcaaatggtgccccaggcaatgacggtgctaaaggcgacgcaggcgccccaggtgctcctggatctcaaggcgcacctggacttcagggaatgcctggcgaacggggagctgctggacttcccggtccaaaaggcgataggggagatgctggtcctaagggcgctgatggctctcctggaaaggatggcgtcagaggcctgacaggcccaattggccctccgggacctgctggcgctccaggcgataagggcgaatctggacctagtggacccgctggtcctacaggtgctaggggagccccaggcgaccggggagagcctggtccaccaggacctgctggatttgctggacctcctggcgctgatggtcaacctggtgctaagggcgagccaggcgacgctggtgcaaaaggcgacgctggtccacctggaccggccggacctgctgggccgccaggacctattggaaatgttggtgcccctggcgccaaaggcgcaagaggatctgctggcccaccaggcgctacaggattcccaggtgccgctggaagagttggaccaccggggccaagtggaaatgctggaccaccgggaccgccaggaccagccggcaaagaaggtggaaaaggccctaggggcgaaactggccctgcaggcaggccaggcgaagtgggccctccaggacctccggggcctgccggcgaaaaaggatctccaggcgcagatggacccgcaggcgctcccggaacaccaggtccacagggaattgctggacaaaggggagttgtcggcctgccaggacagaggggagagagaggttttccaggactccctgggccaagcggagaacctggcaaacagggaccatctggtgccagcggagagagagggccaccaggaccaatgggtcctccaggattggcagggcctcctggcgaatctggtagagaaggtgctccaggcgccgagggatctcctggacgtgatggttctcctggcgccaagggcgatagaggcgaaacaggcccagctggacctccaggcgcacccggcgctccaggcgcaccaggacctgttggccctgctggaaaatctggcgacagaggcgaaactggacccgcaggaccagccggacctgttggacctgtgggtgctagaggacccgctggaccacaaggtcctagaggcgacaagggcgaaacaggcgagcaaggcgacagaggcatcaagggacacagaggattcagcggactgcagggaccaccagggccgcctggaagtcccggcgagcagggaccaagcggagctagtggtcccgccggacctagaggaccacctggttctgctggtgcacccggaaaggacggactgaatgggctccccggacctattgggccacctggacctagaggaagaacaggcgacgcaggaccagttggaccacctgggccacctggaccgcctggtcctcctggacctccttctgccggattcgacttcagcttcctgcctcagcctcctcaagagaaggcccatgacggcggcagatattacagagccgacgacgccaacgtcgtgcgggacagagatctggaagtggacaccacactgaagtccctgtctcagcagatcgagaacatcagaagccccgagggcagcagaaagaaccctgccagaacctgtcgggacctgaagatgtgccacagcgattggaagtctggcgagtactggatcgaccccaaccagggctgcaacctggatgccatcaaggtgttctgcaacatggaaaccggcgagacatgcgtgtaccccacacagccatctgtggctcagaagaactggtacatcagcaagaaccccaaggacaagcggcacgtttggttcggcgagagcatgaccgatggcttccagtttgagtatggcggccagggctctgaccctgccgatgttgctatccagctgaccttcctgcggctgatgtctacagaggccagccagaacatcacctaccactgcaagaacagcgtggcctacatggatcagcagaccggcaacctgaagaaggcactgctgcttcagggcagcaacgagatcgagatcagagccgagggcaacagccggttcacctacagcgtgacagtggatggctgcaccagccatacaggcgcttggggcaagaccgtgatcgagtacaagaccaccaagaccagcagactgcccatcatcgatgtggcccctctggatgttggggcacccgatcaagagttcggcttcgatgtgggcccagtgtgcttcctgagggccaagaggggcagcggcgagggcaggggcagcctgctgacctgcggcgacgtggaggagaaccccggccccctgagcttcgtggacaccagaacactgctgctgctggccgtgacactgtgtctggccacttgtcagagcctgcaagaggaaacagtgcggaaaggacctgccggcgatagaggacctagaggcgaaagaggtcctcctggacctcctggtagagatggcgaggatggacctacaggaccacctggtccaccaggacctccagggcctcctggccttggaggaaattttgccgctcagtacgatggcaaaggcgtcggacttggccctggacctatgggacttatgggcccaagaggaccaccaggtgctgcaggcgctccaggaccacaaggatttcaaggaccagctggcgagcctggcgaacctggacaaacaggtcctgctggtgctagaggaccagccgggccacctggaaaagctggcgaagatgggcaccctggaaagcctggtagacccggcgaaaggggtgttgttggacctcaaggcgccagaggctttcctggaacacctggactgcctggcttcaagggcatcagaggccacaatggcctggacggactgaaaggacaacctggtgctcctggcgtgaaaggcgaaccaggcgcacctggcgaaaatggcacaccaggacaaaccggcgcaagaggacttcctggcgagagaggaagagttggagccccaggtccagcaggcgcacgaggatctgatggatctgtgggacctgttggccctgccggacctattggaagtgctggccctcctggatttcctggcgcacccggaccaaagggcgaaattggagctgtgggaaacgccggacctgcaggcccagctggaccaaggggagaagttggattgcctggactgagcggaccagttgggccaccagggaatcctggtgccaatggactgacaggcgctaaaggtgcagctggccttccaggcgttgccggtgcaccaggactgccaggaccaagaggtatccctggtcctgttggagctgctggcgctacgggtgccagaggacttgttggagaacctgggccagccggatctaagggcgagtctggaaacaagggcgagccaggatctgctggtccacaaggcccgcctggaccatcaggcgaagaaggcaaacgaggccctaatggcgaagccggtagtgccgggcctcctggaccaccaggccttagaggatctcctggctctagaggattgccaggcgctgatggtagagcaggcgttatgggtccacctggatcaagaggcgcttctggccctgctggcgttagaggtccaaatggcgacgctggcagaccaggcgagcccggtcttatggggcctagagggttgcctggaagccctggcaatatcggcccagccggaaaagaaggccctgttggactccctggcatcgacggtagacctggaccaatcggacccgcaggcgctaggggagagcctggaaatattggcttccctgggcctaaaggccccacaggcgatcctggaaagaacggcgataagggccatgctggactcgctggtgcaaggggagcacctggacctgacggaaacaatggtgctcaagggccgcctgggccacaaggtgttcaaggtggaaaaggcgagcagggcccacctgggcctccaggcttccaaggacttcccggaccatctgggccagcaggcgaagttggaaagcctggcgaaagaggactgcacggcgagtttggcttgccgggtcctgccggtccacggggagagagaggccctccaggcgaatctggcgccgcaggacctactggccctatcggaagcagaggacctagtggacctccaggacctgatggcaacaaaggcgaacctggtgttgtgggcgctgtgggaacagctggaccttctggtccttctggattgcccggcgagcgcggagcagctggtattcctggtggcaaaggcgaaaagggcgagcctggactcagaggcgagatcggcaatcccggacgagatggcgctagaggcgccccaggtgcagttggtgccccgggacctgctggcgcaacaggcgacagaggcgaggctggtgccgctggtcctgccgggccagccggtcctagaggaagtccaggcgagaggggcgaagtgggacccgctggacccaatggatttgctgggcccgctggcgctgctggtcaacctggcgccaaaggcgagcggggagctaaaggtcctaaaggcgagaatggcgtcgtgggccctactggaccagtgggagcagcaggccccgcaggtcctaacggaccacctggaccagctgggtctagaggcgacggcggaccgcctggaatgacaggttttccaggcgccgctggaagaacaggtcctccaggaccatctggcatctctggtccaccagggccacctggtcctgctggaaaagaaggactgagaggccctaggggcgatcagggtccagttggaagaaccggcgaagtcggagctgtcggcccaccaggttttgccggcgaaaaaggccctagcggagaagctggaactgcaggaccgccgggaactcccggtcctcaaggattgcttggcgcccctggaattctgggactgcccggtagtcgcggagaacgtggactcccaggtgttgctggcgccgtcggagaaccgggaccacttggaattgctggaccacctggtgcaagaggtccacctggtgcagttggaagtcctggcgttaacggtgctccaggcgaagccggcagagatggaaatcccggcaatgatggcccgcctgggagagatggacagcctggacataagggcgagcgaggctacccaggcaatattggacctgtcggcgcagccggtgctcccggacctcatggtccagtcggtccagccgggaagcacggaaataggggagaaacaggaccctccggtcctgttggcccagctggcgcagttggaccaagaggcccatccggacctcagggaatccgcggagataagggcgaacctggcgagaagggacctagaggactgcctgggctgaaaggccataacggactgcaaggcctgccaggcattgctggccatcatggcgatcaaggtgcacccggtagtgtgggtcccgccggaccgaggggtcccgctggtccatctggacccgccggaaaagatggcagaacaggacatcctggcacagtggggcctgccggaattagaggcccacagggacatcaaggccccgctgggccgccaggacctccgggaccgccagggccaccaggcgttagtggcggaggatacgatttcggctacgacggcgacttctacagagccgaccagcctagatctgcccctagcctgaggcctaaggactacgaagtggacgccacactgaagtccctgaacaaccagatcgagacactgctgacccctgagggcagcagaaagaaccctgccagaacctgcagggacctgagactgtctcaccccgaatggtcctccggctactactggatcgaccccaatcagggctgcaccatggacgccatcaaggtgtactgcgacttcagcaccggcgagacatgcatcagagcccagcctgagaacatccccgccaagaactggtacagaagcagcaaggacaagaaacacgtgtggctgggcgagacaatcaacgccggcagccagttcgagtacaacgtggaaggcgtgaccagcaaagagatggccacacagctggctttcatgagactgctggccaattacgccagccagaacatcacctaccactgcaagaacagcattgcctacatggacgaggaaaccggcaacctgaagaaagccgtgatcctgcagggctctaacgacgtggaactggtggccgagggcaacagcagattcacctacaccgtgctggtggacggctgcagcaaaaagaccaacgagtggggcaagaccatcatcgagtataagaccaacaagcccagcagactgcccttcctggatatcgccccactggatattggaggcgccgaccaagagttctttgtggacatcggccccgtgtgcttcaagtga

SEQ ID NO: 33 - DNA, 8644 bp, synthetic construct
  misc_feature 1..8644: note = Synthetic Construct
  source 1..8644: mol_type = other DNA
atgttcagcttcgtggacctgagactgctgctgctcctggctgctacagccctgctgacacacggacaagaggaaggccaggtcgaaggacaggacgaggacatccctcctatcacctgtgtgcagaacggcctgagataccacgaccgggatgtgtggaagcccgagccttgcagaatctgcgtgtgcgacaatggcaaggtgctgtgcgacgacgtgatctgcgacgagacaaagaattgccctggcgccgaagtgcctgagggcgaatgttgtcctgtgtgccctgatggcagcgagagccccacagatcaagagacaacaggcgtggaaggccccaagggcgatacaggacctagaggtcctagaggacctgccggacctcctggcagagatggaattcctggacagcctggactgcccggaccacctggacctccagggcctccaggtccaccaggactcggaggaaattttgccccacagctgagctacggctacgacgagaaaagcacaggcggcatctctgtgcctggacctatgggaccttctggcccaagaggacttcctggtcctcctggtgctccaggacctcagggatttcaaggaccaccaggcgaacctggcgaaccaggcgctagtggtccaatgggaccaagaggccctcctgggccaccaggcaaaaatggcgacgatggcgaagccggaaagcctggaaggcctggcgaaagaggcccgccaggaccgcaaggcgctagagggttgcctggaactgcaggactgcctggcatgaagggccacagaggcttttctggactggatggcgctaagggcgacgctggaccagcaggacctaaaggcgagcctggatctcctggcgagaatggtgcacctggacagatgggtcccagaggattgccaggcgagagaggtagacctggcgctccgggaccagccggtgctagaggaaatgatggcgcaacaggtgctgctgggcctcctggaccaaccggaccagctggcccacctggatttccaggcgctgttggagcaaagggcgaagcaggcccacaaggacctaggggatctgaaggtcctcagggcgttagaggcgagccagggccacctgggcctgccggtgcagctggacctgctggaaaccctggtgctgatggacagccaggtgccaaaggtgctaatggcgcccctggaattgccggcgctccaggttttcccggcgcaagagggccatctggacctcaaggcccaggcggacctccgggtcctaagggaaatagcggagagccaggcgctcctgggagtaaaggcgatactggcgcaaaaggcgaacccggacctgtgggagttcaaggacctcctggaccagctggcgaggaaggcaaaagaggcgctaggggagaaccaggaccaacagggctccctggtccacctggcgagcgcggaggacctggatctagaggattccctggcgcagatggcgtggccggaccaaaaggacctgcaggcgaaaggggatcaccaggtcctgcaggccctaagggttctccaggcgaggctggcagacccggcgaagctggactcccaggtgctaagggactgacaggctcaccaggatctcccggaccagacggaaaaacaggacctccaggaccggcaggacaggatggtagacccggtcctcctggaccgcctggtgcaagaggacaagctggcgtgatgggctttcctggaccaaaaggtgcagccggcgaacctggaaaagcaggcgagaggggagttcccggacctccaggtgctgttggacctgccggaaaagatggcgaagctggtgcacaaggtcctccagggccagccggaccagccggcgagagaggcgaacaaggaccagccggatctccaggatttcagggactgccagggcctgctggcccgcctggcgaggcagggaagccaggcgaacagggtgttcctggcgatcttggagcccctggtcctagcggagctagaggcgaaagaggatttcctggcgaaaggggcgttcagggtccaccgggaccagctggaccaaggggtgcaaatggtgccccaggcaatgacggtgctaaaggcgacgcaggcgccccaggtgctcctggatctcaaggcgcacctggacttcagggaatgcctggcgaacggggagctgctggacttcccggtccaaaaggcgataggggagatgctggtcctaagggcgctgatggctctcctggaaaggatggcgtcagaggcctgacaggcccaattggccctccgggacctgctggcgctccaggcgataagggcgaatctggacctagtggacccgctggtcctacaggtgctaggggagccccaggcgaccggggagagcctggtccaccaggacctgctggatttgctggacctcctggcgctgatggtcaacctggtgctaagggcgagccaggcgacgctggtgcaaaaggcgacgctggtccacctggaccggccggacctgctgggccgccaggacctattggaaatgttggtgcccctggcgccaaaggcgcaagaggatctgctggcccaccaggcgctacaggattcccaggtgccgctggaagagttggaccaccggggccaagtggaaatgctggaccaccgggaccgccaggaccagccggcaaagaaggtggaaaaggccctaggggcgaaactggccctgcaggcaggccaggcgaagtgggccctccaggacctccggggcctgccggcgaaaaaggatctccaggcgcagatggacccgcaggcgctcccggaacaccaggtccacagggaattgctggacaaaggggagttgtcggcctgccaggacagaggggagagagaggttttccaggactccctgggccaagcggagaacctggcaaacagggaccatctggtgccagcggagagagagggccaccaggaccaatgggtcctccaggattggcagggcctcctggcgaatctggtagagaaggtgctccaggcgccgagggatctcctggacgtgatggttctcctggcgccaagggcgatagaggcgaaacaggcccagctggacctccaggcgcacccggcgctccaggcgcaccaggacctgttggccctgctggaaaatctggcgacagaggcgaaactggacccgcaggaccagccggacctgttggacctgtgggtgctagaggacccgctggaccacaaggtcctagaggcgacaagggcgaaacaggcgagcaaggcgacagaggcatcaagggacacagaggattcagcggactgcagggaccaccagggccgcctggaagtcccggcgagcagggaccaagcggagctagtggtcccgccggacctagaggaccacctggttctgctggtgcacccggaaaggacggactgaatgggctccccggacctattgggccacctggacctagaggaagaacaggcgacgcaggaccagttggaccacctgggccacctggaccgcctggtcctcctggacctccttctgccggattcgacttcagcttcctgcctcagcctcctcaagagaaggcccatgacggcggcagatattacagagccgacgacgccaacgtcgtgcgggacagagatctggaagtggacaccacactgaagtccctgtctcagcagatcgagaacatcagaagccccgagggcagcagaaagaaccctgccagaacctgtcgggacctgaagatgtgccacagcgattggaagtctggcgagtactggatcgaccccaaccagggctgcaacctggatgccatcaaggtgttctgcaacatggaaaccggcgagacatgcgtgtaccccacacagccatctgtggctcagaagaactggtacatcagcaagaaccccaaggacaagcggcacgtttggttcggcgagagcatgaccgatggcttccagtttgagtatggcggccagggctctgaccctgccgatgttgctatccagctgaccttcctgcggctgatgtctacagaggccagccagaacatcacctaccactgcaagaacagcgtggcctacatggatcagcagaccggcaacctgaagaaggcactgctgcttcagggcagcaacgagatcgagatcagagccgagggcaacagccggttcacctacagcgtgacagtggatggctgcaccagccatacaggcgcttggggcaagaccgtgatcgagtacaagaccaccaagaccagcagactgcccatcatcgatgtggcccctctggatgttggggcacccgatcaagagttcggcttcgatgtgggcccagtgtgcttcctgtaagaattcgtcgattcggttgcagcatttaaagcggttgacaactttaaaagaaggaaaaagaaggttgaagaaaagggtgtagtaagtaagtataagtacagaccggagaagtacgccggtcctgattcgtttaatttgaaagaagaaaatgctgagcttcgtggacaccagaacactgctgctgctggccgtgacactgtgtctggccacttgtcagagcctgcaagaggaaacagtgcggaaaggacctgccggcgatagaggacctagaggcgaaagaggtcctcctggacctcctggtagagatggcgaggatggacctacaggaccacctggtccaccaggacctccagggcctcctggccttggaggaaattttgccgctcagtacgatggcaaaggcgtcggacttggccctggacctatgggacttatgggcccaagaggaccaccaggtgctgcaggcgctccaggaccacaaggatttcaaggaccagctggcgagcctggcgaacctggacaaacaggtcctgctggtgctagaggaccagccgggccacctggaaaagctggcgaagatgggcaccctggaaagcctggtagacccggcgaaaggggtgttgttggacctcaaggcgccagaggctttcctggaacacctggactgcctggcttcaagggcatcagaggccacaatggcctggacggactgaaaggacaacctggtgctcctggcgtgaaaggcgaaccaggcgcacctggcgaaaatggcacaccaggacaaaccggcgcaagaggacttcctggcgagagaggaagagttggagccccaggtccagcaggcgcacgaggatctgatggatctgtgggacctgttggccctgccggacctattggaagtgctggccctcctggatttcctggcgcacccggaccaaagggcgaaattggagctgtgggaaacgccggacctgcaggcccagctggaccaaggggagaagttggattgcctggactgagcggaccagttgggccaccagggaatcctggtgccaatggactgacaggcgctaaaggtgcagctggccttccaggcgttgccggtgcaccaggactgccaggaccaagaggtatccctggtcctgttggagctgctggcgctacgggtgccagaggacttgttggagaacctgggccagccggatctaagggcgagtctggaaacaagggcgagccaggatctgctggtccacaaggcccgcctggaccatcaggcgaagaaggcaaacgaggccctaatggcgaagccggtagtgccgggcctcctggaccaccaggccttagaggatctcctggctctagaggattgccaggcgctgatggtagagcaggcgttatgggtccacctggatcaagaggcgcttctggccctgctggcgttagaggtccaaatggcgacgctggcagaccaggcgagcccggtcttatggggcctagagggttgcctggaagccctggcaatatcggcccagccggaaaagaaggccctgttggactccctggcatcgacggtagacctggaccaatcggacccgcaggcgctaggggagagcctggaaatattggcttccctgggcctaaaggccccacaggcgatcctggaaagaacggcgataagggccatgctggactcgctggtgcaaggggagcacctggacctgacggaaacaatggtgctcaagggccgcctgggccacaaggtgttcaaggtggaaaaggcgagcagggcccacctgggcctccaggcttccaaggacttcccggaccatctgggccagcaggcgaagttggaaagcctggcgaaagaggactgcacggcgagtttggcttgccgggtcctgccggtccacggggagagagaggccctccaggcgaatctggcgccgcaggacctactggccctatcggaagcagaggacctagtggacctccaggacctgatggcaacaaaggcgaacctggtgttgtgggcgctgtgggaacagctggaccttctggtccttctggattgcccggcgagcgcggagcagctggtattcctggtggcaaaggcgaaaagggcgagcctggactcagaggcgagatcggcaatcccggacgagatggcgctagaggcgccccaggtgcagttggtgccccgggacctgctggcgcaacaggcgacagaggcgaggctggtgccgctggtcctgccgggccagccggtcctagaggaagtccaggcgagaggggcgaagtgggacccgctggacccaatggatttgctgggcccgctggcgctgctggtcaacctggcgccaaaggcgagcggggagctaaaggtcctaaaggcgagaatggcgtcgtgggccctactggaccagtgggagcagcaggccccgcaggtcctaacggaccacctggaccagctgggtctagaggcgacggcggaccgcctggaatgacaggttttccaggcgccgctggaagaacaggtcctccaggaccatctggcatctctggtccaccagggccacctggtcctgctggaaaagaaggactgagaggccctaggggcgatcagggtccagttggaagaaccggcgaagtcggagctgtcggcccaccaggttttgccggcgaaaaaggccctagcggagaagctggaactgcaggaccgccgggaactcccggtcctcaaggattgcttggcgcccctggaattctgggactgcccggtagtcgcggagaacgtggactcccaggtgttgctggcgccgtcggagaaccgggaccacttggaattgctggaccacctggtgcaagaggtccacctggtgcagttggaagtcctggcgttaacggtgctccaggcgaagccggcagagatggaaatcccggcaatgatggcccgcctgggagagatggacagcctggacataagggcgagcgaggctacccaggcaatattggacctgtcggcgcagccggtgctcccggacctcatggtccagtcggtccagccgggaagcacggaaataggggagaaacaggaccctccggtcctgttggcccagctggcgcagttggaccaagaggcccatccggacctcagggaatccgcggagataagggcgaacctggcgagaagggacctagaggactgcctgggctgaaaggccataacggactgcaaggcctgccaggcattgctggccatcatggcgatcaaggtgcacccggtagtgtgggtcccgccggaccgaggggtcccgctggtccatctggacccgccggaaaagatggcagaacaggacatcctggcacagtggggcctgccggaattagaggcccacagggacatcaaggccccgctgggccgccaggacctccgggaccgccagggccaccaggcgttagtggcggaggatacgatttcggctacgacggcgacttctacagagccgaccagcctagatctgcccctagcctgaggcctaaggactacgaagtggacgccacactgaagtccctgaacaaccagatcgagacactgctgacccctgagggcagcagaaagaaccctgccagaacctgcagggacctgagactgtctcaccccgaatggtcctccggctactactggatcgaccccaatcagggctgcaccatggacgccatcaaggtgtactgcgacttcagcaccggcgagacatgcatcagagcccagcctgagaacatccccgccaagaactggtacagaagcagcaaggacaagaaacacgtgtggctgggcgagacaatcaacgccggcagccagttcgagtacaacgtggaaggcgtgaccagcaaagagatggccacacagctggctttcatgagactgctggccaattacgccagccagaacatcacctaccactgcaagaacagcattgcctacatggacgaggaaaccggcaacctgaagaaagccgtgatcctgcagggctctaacgacgtggaactggtggccgagggcaacagcagattcacctacaccgtgctggtggacggctgcagcaaaaagaccaacgagtggggcaagaccatcatcgagtataagaccaacaagcccagcagactgcccttcctggatatcgccccactggatattggaggcgccgaccaagagttctttgtggacatcggccccgtgtgcttcaagtga

SEQ ID NO: 34 - DNA, 8987 bp, synthetic construct
  misc_feature 1..8987: note = Synthetic Construct
  source 1..8987: mol_type = other DNA
atgttcagcttcgtggacctgagactgctgctgctcctggctgctacagccctgctgacacacggacaagaggaaggccaggtcgaaggacaggacgaggacatccctcctatcacctgtgtgcagaacggcctgagataccacgaccgggatgtgtggaagcccgagccttgcagaatctgcgtgtgcgacaatggcaaggtgctgtgcgacgacgtgatctgcgacgagacaaagaattgccctggcgccgaagtgcctgagggcgaatgttgtcctgtgtgccctgatggcagcgagagccccacagatcaagagacaacaggcgtggaaggccccaagggcgatacaggacctagaggtcctagaggacctgccggacctcctggcagagatggaattcctggacagcctggactgcccggaccacctggacctccagggcctccaggtccaccaggactcggaggaaattttgccccacagctgagctacggctacgacgagaaaagcacaggcggcatctctgtgcctggacctatgggaccttctggcccaagaggacttcctggtcctcctggtgctccaggacctcagggatttcaaggaccaccaggcgaacctggcgaaccaggcgctagtggtccaatgggaccaagaggccctcctgggccaccaggcaaaaatggcgacgatggcgaagccggaaagcctggaaggcctggcgaaagaggcccgccaggaccgcaaggcgctagagggttgcctggaactgcaggactgcctggcatgaagggccacagaggcttttctggactggatggcgctaagggcgacgctggaccagcaggacctaaaggcgagcctggatctcctggcgagaatggtgcacctggacagatgggtcccagaggattgccaggcgagagaggtagacctggcgctccgggaccagccggtgctagaggaaatgatggcgcaacaggtgctgctgggcctcctggaccaaccggaccagctggcccacctggatttccaggcgctgttggagcaaagggcgaagcaggcccacaaggacctaggggatctgaaggtcctcagggcgttagaggcgagccagggccacctgggcctgccggtgcagctggacctgctggaaaccctggtgctgatggacagccaggtgccaaaggtgctaatggcgcccctggaattgccggcgctccaggttttcccggcgcaagagggccatctggacctcaaggcccaggcggacctccgggtcctaagggaaatagcggagagccaggcgctcctgggagtaaaggcgatactggcgcaaaaggcgaacccggacctgtgggagttcaaggacctcctggaccagctggcgaggaaggcaaaagaggcgctaggggagaaccaggaccaacagggctccctggtccacctggcgagcgcggaggacctggatctagaggattccctggcgcagatggcgtggccggaccaaaaggacctgcaggcgaaaggggatcaccaggtcctgcaggccctaagggttctccaggcgaggctggcagacccggcgaagctggactcccaggtgctaagggactgacaggctcaccaggatctcccggaccagacggaaaaacaggacctccaggaccggcaggacaggatggtagacccggtcctcctggaccgcctggtgcaagaggacaagctggcgtgatgggctttcctggaccaaaaggtgcagccggcgaacctggaaaagcaggcgagaggggagttcccggacctccaggtgctgttggacctgccggaaaagatggcgaagctggtgcacaaggtcctccagggccagccggaccagccggcgagagaggcgaacaaggaccagccggatctccaggatttcagggactgccagggcctgctggcccgcctggcgaggcagggaagccaggcgaacagggtgttcctggcgatcttggagcccctggtcctagcggagctagaggcgaaagaggatttcctggcgaaaggggcgttcagggtccaccgggaccagctggaccaaggggtgcaaatggtgccccaggcaatgacggtgctaaaggcgacgcaggcgccccaggtgctcctggatctcaaggcgcacctggacttcagggaatgcctggcgaacggggagctgctggacttcccggtccaaaaggcgataggggagatgctggtcctaagggcgctgatggctctcctggaaaggatggcgtcagaggcctgacaggcccaattggccctccgggacctgctggcgctccaggcgataagggcgaatctggacctagtggacccgctggtcctacaggtgctaggggagccccaggcgaccggggagagcctggtccaccaggacctgctggatttgctggacctcctggcgctgatggtcaacctggtgctaagggcgagccaggcgacgctggtgcaaaaggcgacgctggtccacctggaccggccggacctgctgggccgccaggacctattggaaatgttggtgcccctggcgccaaaggcgcaagaggatctgctggcccaccaggcgctacaggattcccaggtgccgctggaagagttggaccaccggggccaagtggaaatgctggaccaccgggaccgccaggaccagccggcaaagaaggtggaaaaggccctaggggcgaaactggccctgcaggcaggccaggcgaagtgggccctccaggacctccggggcctgccggcgaaaaaggatctccaggcgcagatggacccgcaggcgctcccggaacaccaggtccacagggaattgctggacaaaggggagttgtcggcctgccaggacagaggggagagagaggttttccaggactccctgggccaagcggagaacctggcaaacagggaccatctggtgccagcggagagagagggccaccaggaccaatgggtcctccaggattggcagggcctcctggcgaatctggtagagaaggtgctccaggcgccgagggatctcctggacgtgatggttctcctggcgccaagggcgatagaggcgaaacaggcccagctggacctccaggcgcacccggcgctccaggcgcaccaggacctgttggccctgctggaaaatctggcgacagaggcgaaactggacccgcaggaccagccggacctgttggacctgtgggtgctagaggacccgctggaccacaaggtcctagaggcgacaagggcgaaacaggcgagcaaggcgacagaggcatcaagggacacagaggattcagcggactgcagggaccaccagggccgcctggaagtcccggcgagcagggaccaagcggagctagtggtcccgccggacctagaggaccacctggttctgctggtgcacccggaaaggacggactgaatgggctccccggacctattgggccacctggacctagaggaagaacaggcgacgcaggaccagttggaccacctgggccacctggaccgcctggtcctcctggacctccttctgccggattcgacttcagcttcctgcctcagcctcctcaagagaaggcccatgacggcggcagatattacagagccgacgacgccaacgtcgtgcgggacagagatctggaagtggacaccacactgaagtccctgtctcagcagatcgagaacatcagaagccccgagggcagcagaaagaaccctgccagaacctgtcgggacctgaagatgtgccacagcgattggaagtctggcgagtactggatcgaccccaaccagggctgcaacctggatgccatcaaggtgttctgcaacatggaaaccggcgagacatgcgtgtaccccacacagccatctgtggctcagaagaactggtacatcagcaagaaccccaaggacaagcggcacgtttggttcggcgagagcatgaccgatggcttccagtttgagtatggcggccagggctctgaccctgccgatgttgctatccagctgaccttcctgcggctgatgtctacagaggccagccagaacatcacctaccactgcaagaacagcgtggcctacatggatcagcagaccggcaacctgaagaaggcactgctgcttcagggcagcaacgagatcgagatcagagccgagggcaacagccggttcacctacagcgtgacagtggatggctgcaccagccatacaggcgcttggggcaagaccgtgatcgagtacaagaccaccaagaccagcagactgcccatcatcgatgtggcccctctggatgttggggcacccgatcaagagttcggcttcgatgtgggcccagtgtgcttcctgtaagttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaagcgtctaggccccccgaaccacggggacgtggttttcctttgaaaaacacgatgctgagcttcgtggacaccagaacactgctgctgctggccgtgacactgtgtctggccacttgtcagagcctgcaagaggaaacagtgcggaaaggacctgccggcgatagaggacctagaggcgaaagaggtcctcctggacctcctggtagagatggcgaggatggacctacaggaccacctggtccaccaggacctccagggcctcctggccttggaggaaattttgccgctcagtacgatggcaaaggcgtcggacttggccctggacctatgggacttatgggcccaagaggaccaccaggtgctgcaggcgctccaggaccacaaggatttcaaggaccagctggcgagcctggcgaacctggacaaacaggtcctgctggtgctagaggaccagccgggccacctggaaaagctggcgaagatgggcaccctggaaagcctggtagacccggcgaaaggggtgttgttggacctcaaggcgccagaggctttcctggaacacctggactgcctggcttcaagggcatcagaggccacaatggcctggacggactgaaaggacaacctggtgctcctggcgtgaaaggcgaaccaggcgcacctggcgaaaatggcacaccaggacaaaccggcgcaagaggacttcctggcgagagaggaagagttggagccccaggtccagcaggcgcacgaggatctgatggatctgtgggacctgttggccctgccggacctattggaagtgctggccctcctggatttcctggcgcacccggaccaaagggcgaaattggagctgtgggaaacgccggacctgcaggcccagctggaccaaggggagaagttggattgcctggactgagcggaccagttgggccaccagggaatcctggtgccaatggactgacaggcgctaaaggtgcagctggccttccaggcgttgccggtgcaccaggactgccaggaccaagaggtatccctggtcctgttggagctgctggcgctacgggtgccagaggacttgttggagaacctgggccagccggatctaagggcgagtctggaaacaagggcgagccaggatctgctggtccacaaggcccgcctggaccatcaggcgaagaaggcaaacgaggccctaatggcgaagccggtagtgccgggcctcctggaccaccaggccttagaggatctcctggctctagaggattgccaggcgctgatggtagagcaggcgttatgggtccacctggatcaagaggcgcttctggccctgctggcgttagaggtccaaatggcgacgctggcagaccaggcgagcccggtcttatggggcctagagggttgcctggaagccctggcaatatcggcccagccggaaaagaaggccctgttggactccctggcatcgacggtagacctggaccaatcggacccgcaggcgctaggggagagcctggaaatattggcttccctgggcctaaaggccccacaggcgatcctggaaagaacggcgataagggccatgctggactcgctggtgcaaggggagcacctggacctgacggaaacaatggtgctcaagggccgcctgggccacaaggtgttcaaggtggaaaaggcgagcagggcccacctgggcctccaggcttccaaggacttcccggaccatctgggccagcaggcgaagttggaaagcctggcgaaagaggactgcacggcgagtttggcttgccgggtcctgccggtccacggggagagagaggccctccaggcgaatctggcgccgcaggacctactggccctatcggaagcagaggacctagtggacctccaggacctgatggcaacaaaggcgaacctggtgttgtgggcgctgtgggaacagctggaccttctggtccttctggattgcccggcgagcgcggagcagctggtattcctggtggcaaaggcgaaaagggcgagcctggactcagaggcgagatcggcaatcccggacgagatggcgctagaggcgccccaggtgcagttggtgccccgggacctgctggcgcaacaggcgacagaggcgaggctggtgccgctggtcctgccgggccagccggtcctagaggaagtccaggcgagaggggcgaagtgggacccgctggacccaatggatttgctgggcccgctggcgctgctggtcaacctggcgccaaaggcgagcggggagctaaaggtcctaaaggcgagaatggcgtcgtgggccctactggaccagtgggagcagcaggccccgcaggtcctaacggaccacctggaccagctgggtctagaggcgacggcggaccgcctggaatgacaggttttccaggcgccgctggaagaacaggtcctccaggaccatctggcatctctggtccaccagggccacctggtcctgctggaaaagaaggactgagaggccctaggggcgatcagggtccagttggaagaaccggcgaagtcggagctgtcggcccaccaggttttgccggcgaaaaaggccctagcggagaagctggaactgcaggaccgccgggaactcccggtcctcaaggattgcttggcgcccctggaattctgggactgcccggtagtcgcggagaacgtggactcccaggtgttgctggcgccgtcggagaaccgggaccacttggaattgctggaccacctggtgcaagaggtccacctggtgcagttggaagtcctggcgttaacggtgctccaggcgaagccggcagagatggaaatcccggcaatgatggcccgcctgggagagatggacagcctggacataagggcgagcgaggctacccaggcaatattggacctgtcggcgcagccggtgctcccggacctcatggtccagtcggtccagccgggaagcacggaaataggggagaaacaggaccctccggtcctgttggcccagctggcgcagttggaccaagaggcccatccggacctcagggaatccgcggagataagggcgaacctggcgagaagggacctagaggactgcctgggctgaaaggccataacggactgcaaggcctgccaggcattgctggccatcatggcgatcaaggtgcacccggtagtgtgggtcccgccggaccgaggggtcccgctggtccatctggacccgccggaaaagatggcagaacaggacatcctggcacagtggggcctgccggaattagaggcccacagggacatcaaggccccgctgggccgccaggacctccgggaccgccagggccaccaggcgttagtggcggaggatacgatttcggctacgacggcgacttctacagagccgaccagcctagatctgcccctagcctgaggcctaaggactacgaagtggacgccacactgaagtccctgaacaaccagatcgagacactgctgacccctgagggcagcagaaagaaccctgccagaacctgcagggacctgagactgtctcaccccgaatggtcctccggctactactggatcgaccccaatcagggctgcaccatggacgccatcaaggtgtactgcgacttcagcaccggcgagacatgcatcagagcccagcctgagaacatccccgccaagaactggtacagaagcagcaaggacaagaaacacgtgtggctgggcgagacaatcaacgccggcagccagttcgagtacaacgtggaaggcgtgaccagcaaagagatggccacacagctggctttcatgagactgctggccaattacgccagccagaacatcacctaccactgcaagaacagcattgcctacatggacgaggaaaccggcaacctgaagaaagccgtgatcctgcagggctctaacgacgtggaactggtggccgagggcaacagcagattcacctacaccgtgctggtggacggctgcagcaaaaagaccaacgagtggggcaagaccatcatcgagtataagaccaacaagcccagcagactgcccttcctggatatcgccccactggatattggaggcgccgaccaagagttctttgtggacatcggccccgtgtgcttcaagtga

SEQ ID NO: 35 - DNA, 7434 bp, Homo sapiens
  source 1..7434: mol_type = unassigned DNA
atgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaa

SEQ ID NO: 36 - DNA, 7434 bp, synthetic construct
  misc_feature 1..7434: note = Synthetic Construct
  source 1..7434: mol_type = other DNA
atgctgagaggacctggaccaggactgctgctgctggctgttcagtgtctgggaacagccgtgcctagcacaggcgccagcaagtctaaaagacaggcccagcagatggtgcagcctcagtctcctgtggccgtgtctcagtctaagcccggctgctacgacaacggcaagcactaccagatcaaccagcagtgggagagaacctacctgggcaacgccctcgtgtgtacatgttacggcggcagcagaggcttcaactgcgagtctaaacccgaggccgaggaaacctgcttcgacaagtacaccggcaacacctacagagtgggcgacacctacgagaggcccaaggacagcatgatctgggactgcacatgtatcggagccggcagaggcagaatcagctgcacaatcgccaaccggtgtcacgaaggcggccagagctataagatcggcgacacttggagaaggccccacgagacaggcggctacatgctggaatgcgtgtgtctcggcaacggcaaaggcgagtggacctgcaagcctatcgccgagaagtgcttcgatcatgccgccggaacaagctacgtcgtgggcgagacatgggagaagccttaccaaggctggatgatggtggactgtacctgcctcggcgaaggcagcggaagaatcacctgtaccagccggaaccggtgcaacgaccaggataccagaaccagctaccggatcggcgatacctggtccaagaaggacaaccggggcaacctgctgcagtgcatctgtaccggaaatggaagaggcgaatggaagtgcgagcggcacacaagcgtgcagacaacatcttctggcagcggcccctttaccgatgtgcgggctgctgtttatcagccccagcctcatcctcagcctccaccttatggccactgcgtgacagatagcggcgtggtgtatagcgtgggcatgcagtggctgaaaacccagggcaacaagcagatgctgtgcacatgcctcggcaatggcgtgtcctgccaagagacagccgtgacacagacctatggcggcaatagcaatggcgagccttgcgtgctgcccttcacctacaatggccggaccttctacagctgcaccaccgagggtagacaggacggacacctgtggtgtagcaccaccagcaactacgagcaggaccagaagtacagcttctgcaccgaccacaccgtgctggtgcaaaccagaggcggcaattctaacggcgccctgtgtcacttcccctttctgtacaacaaccacaactacaccgactgcacctccgaaggcagacgggacaacatgaagtggtgcggcacaacccagaactacgacgccgatcagaagttcggcttctgtcccatggccgctcacgaggaaatctgtaccaccaacgaaggcgtgatgtacagaatcggcgaccagtgggacaagcagcacgacatgggccacatgatgcggtgtacctgcgttggcaatggtcgcggagagtggacatgcattgcctacagccagctgcgggaccagtgcatcgtggacgacatcacatacaacgtgaacgacaccttccacaagcggcacgaagagggacacatgctgaactgcacctgtttcggccaaggcagaggccggtggaagtgtgatcctgtggatcagtgccaggacagcgaaaccggcaccttttaccagatcggagactcctgggagaaatacgtgcacggcgtgcgctaccagtgctactgttacggcagaggaattggcgagtggcactgtcagcccctgcagacataccctagctctagcggacctgtcgaggtgttcatcaccgagacacccagccagcctaactctcaccccatccagtggaatgcccctcagcctagccacatcagcaagtacatcctgagatggcggcccaagaactccgtcggcagatggaaagaggccaccattcctggccacctgaacagctacaccatcaagggactgaagcccggcgtggtctatgagggccagctgatctctatccagcagtacggccaccaagaagtgaccagattcgacttcaccaccacctccaccagcacacccgtgaccagcaataccgtgaccggcgagacaacccctttctctccactggtggccacaagcgagagcgtgaccgagattaccgccagcagctttgtggtgtcttgggtgtccgccagcgatacagtgtccggcttcagagtggaatacgagctgagcgaggaaggcgacgagccccagtatctggatctgccaagcaccgccaccagcgtgaacatccctgatctgctgcccggcagaaagtacatcgtgaacgtgtaccagatctccgaggacggcgagcagagcctgatcctgagcacaagccagacaacagcccctgacgctcctcctgatcctaccgttgatcaggtggacgataccagcatcgtcgtgcggtggtcaagaccccaggctcctatcaccggctacaggatcgtgtacagccctagcgtggaaggcagcagcaccgagctgaatctgcccgagacagccaatagcgtgaccctgtctgatctgcagcctggcgtgcagtacaatatcaccatctacgccgtggaagagaatcaagagtctacccctgtggtcatccagcaagagacaaccggcactcccagatccgacaccgttccatctccacgggatctgcagttcgtggaagtgaccgacgtgaaagtgacaatcatgtggaccccacctgagagcgccgtgacaggctatagagtggacgtgatccccgtgaacctgccaggcgaacatggacagagactgcccatcagcagaaacacctttgccgaagtgacaggactgtcccctggcgtgacctactacttcaaggtgttcgctgtgtcccacggcagagagagcaaacctctgacagctcagcagaccaccaagctggacgcccctaccaacctgcagtttgtgaacgagacagacagcacagtgcttgtgcggtggacccctccaagagcacagatcacaggataccggctgaccgtgggcctgaccagaagaggacagcccagacagtacaacgtgggccccagcgtgtccaagtatcccctgagaaatctccagcctgccagcgagtacaccgtgtctctggtggctatcaagggcaatcaagagagccctaaggccaccggcgtgttcactacactgcagcccggaagcagcatccctccatacaacacagaagtgactgaaaccaccatcgtgatcacctggacacccgctcctcggatcggctttaagctgggcgtcagaccttctcaaggcggcgaagctcccagagaagtgacaagcgatagcggcagcatcgtggtgtctggactgacaccaggcgtggaatatgtgtacaccatccaggtgctgcgcgacggccaagaaagggatgcccctatcgtgaacaaggtggtcacccctctgagcccaccaacaaacctgcacctggaagccaatcctgataccggcgtgctgactgtgtcctgggaaagaagcaccacacctgacattaccggctatcggatcaccacaacacccaccaatggccagcagggcaactccctggaagaggtggtgcatgccgatcagtccagctgtaccttcgacaatctgagccctggcctcgagtacaatgtgtccgtgtacacagtgaaggacgacaaagaaagcgtgcccatctccgacacaatcatccctgaggtgccccagctgaccgacctgagcttcgtggatatcaccgacagcagcatcggcctgaggtggacacctctgaactcctctaccatcatcggatacagaatcaccgtggtggccgctggcgagggcatcccaatcttcgaggactttgtggacagcagcgtgggctactacaccgtgactggactggaacccggcatcgactacgacatcagcgtcatcaccctgatcaatggcggcgagagcgcccctacaacactgacacaacagactgccgtgcctcctcctaccgatctgcggttcacaaatatcggccccgacaccatgagagtgacttgggctcctccaccaagtatcgacctgaccaacttcctcgtgcggtacagccccgtgaagaacgaggaagatgtggccgagctgtccatctctcccagcgataatgccgtggtgctgaccaatctgctccccggaacagagtacgtggtgtccgttagcagcgtgtacgaacagcacgagagcacacccctgcggggcagacaaaaaacaggcctggatagccccaccggaatcgacttcagcgatatcacagccaacagcttcaccgtgcattggatcgcccctagagccaccatcaccgggtatagaatccggcatcaccccgagcacttcagcggcagacctagagaagatagagtgccccactccagaaacagcatcaccctcaccaatctgacacccggcaccgaatatgtggtgtccatcgtggccctgaacggcagggaagagtctcctctgctgatcggccagcagagcacagtgtctgacgtgcccagagatctggaagtggtggctgccacacctaccagcctgctgattagctgggatgctcctgctgtgacagtgcggtattaccggatcacctacggcgagactggcggcaactctcccgtgcaagagtttacagtgcctggcagcaagagcaccgctaccatctctggactcaagccaggcgtcgactacaccattaccgtgtacgctgtgaccggcaggggcgattctcctgcctcttctaagcctatcagcatcaactaccggaccgagatcgacaagccaagccagatgcaagtgacagatgtgcaggacaacagcatctccgtgaagtggctgcctagcagctctccagtgaccgggtacagagtgaccaccacaccaaagaacggccctggacctaccaagaccaagaccgctggacctgatcagaccgagatgaccattgagggcctgcagcctaccgtcgagtatgtcgtgtctgtgtacgcccagaatcctagcggcgagtctcagcctcttgtgcagaccgccgtgaccaacatcgacagacctaaaggcctggccttcaccgacgtggacgtggactctatcaagatcgcctgggagtcccctcagggccaagtgtccagatatagagtgacctacagctcccctgaggacggcatccacgagctgtttccagcaccagacggcgaagaggataccgccgaactgcaaggactgaggcctggctccgagtatacagtctcagtggtggccctgcacgacgacatggaaagccagcctctgatcggaacccagtccaccgctattcccgctcctacagacctgaagttcacccaagtgaccccaaccagcctgagcgcacaatggactcctccaaacgtccagctgactggttatagagtgcgcgtgacacccaaagaaaagactggccccatgaaggaaatcaatctggcccctgactccagctccgtggttgtgtctggtcttatggtggctaccaaatacgaggtttccgtgtatgccctgaaggacaccctgacctccagacctgcacagggcgttgtgaccacactggaaaacgtgtccccacctcggagagccagagtgacagacgccaccgaaaccacaatcaccatttcttggcggaccaagacagagacaatcaccggattccaggtcgacgccgtgcctgccaatggacagacacctatccagcggaccatcaagcctgacgtgcggagctacacaatcacaggcctgcaacctggcaccgactacaagatctacctgtacaccctgaacgacaacgcccgctctagccccgtggttatcgatgcctctaccgccatcgacgccccaagcaatctgagatttctggccacaactcccaacagtctgctcgtgtcttggcagcctcctcgggccagaatcactggctacatcattaagtacgagaagccaggcagccctcctagagaggtggtccctagacctagacctggcgtcacagaggccacaattaccggactcgagcccggcactgagtacacaatctacgtgatcgccctgaagaacaaccagaagtccgagccactgattggccggaagaaaaccgacgagctgcctcagctggtcaccctgcctcatccaaatctgcacggccccgagatcctggatgtgccatctaccgtgcagaaaaccccatttgtgacacaccccggctacgacaccggaaacggaattcagctgcctggaacctctgggcagcagccttctgtgggacagcagatgatctttgaggaacacggcttccggcggaccacacctcctacaacagccacaccaatcaggcacagaccccggccttatcctcctaacgtgggcgaagagattcagatcggacacatccccagagaggatgtcgactaccacctgtatcctcacggcccaggactgaaccctaatgccagcacaggacaagaggccctgagccagaccactatcagctgggctccattccaggacacctccgagtacatcatctcttgtcaccccgtgggcaccgacgaggaaccactgcaattcagagtgcccggcacctctaccagcgccacacttacaggactgactagaggcgccacctataacatcatcgtggaagccctgaaagaccagcagcggcacaaagtgcgcgaagaggttgtgactgtgggcaattccgtgaacgagggcctgaatcagcccaccgacgacagctgctttgacccctacacagtgtcccactatgccgtgggagatgagtgggaacgcatgtccgagagcggcttcaagctgctctgtcagtgcctcggctttggctccggccacttcagatgcgatagctccaggtggtgccacgataacggcgtgaactacaaaatcggagagaagtgggacagacagggcgagaacggccagatgatgagctgcacttgtctcggaaatggaaagggcgagttcaagtgcgaccctcacgaggccacctgttacgacgatggcaagacctaccacgtgggagagcagtggcagaaagagtacctgggcgccatctgtagctgcacatgctttggcgggcagcgcggctggcgctgtgataattgccgtagaccaggcggcgagccatctcctgagggaacaacaggccagtcctacaaccagtacagccagagataccaccagcgcaccaacaccaatgtgaactgccccatcgagtgcttcatgcccctggacgtgcaggccgacagggaagattctagagagtga

SEQ ID NO: 37 - DNA, 2361 bp, Homo sapiens
  source 1..2361: mol_type = unassigned DNA
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctggtgtcggaggtgttcccggagtcggaggtgtcccgggagttggcatttcccccgaagctcaggcagcagctgccgccaaggctgccaagtacggtgctgcaggagcaggagtgctgggtgggctagtgccaggtgccccaggcgcagtcccaggtgtgccgggcacgggaggagtgccaggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcaggtgcagatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 38 - DNA, 2361 bp, synthetic construct
  misc_feature 1..2361: note = Synthetic Construct
  source 1..2361: mol_type = other DNA
atggctggactgacagccgctgctcctagacctggtgttctgctgctgctcctgagcattctgcacccttctagaccaggcggagtgcctggtgctattcctggcggagttcccggcggagtgttttatcctggtgctggactgggagcccttggcggaggtgctcttggacctggtggaaagcctctgaaacctgtgcctggcggacttgctggtgcaggtcttggagccggacttggagcttttcccgccgtgacatttcctggcgctcttgtgccaggtggcgtggcagatgctgccgctgcttataaggctgccaaagccggcgctggactcggaggtgttcctggtgttggaggcctgggagtttctgctggtgctgtggttcctcaacctggcgctggtgttaagcctggcaaagttccaggcgttggactgcctggtgtttaccctggtggtgttctccctggcgctagattccctggcgttggagttcttcctggcgtgccaacaggtgccggcgtgaaacctaaagctcctggtgtcggcggagccttcgctggaatacctggcgtgggaccttttggcggacctcaaccaggtgtccctctgggctatcctatcaaggcccctaaactgccaggcggctacggcctgccttacacaactggcaagctgccctatggctatggacctggcggtgttgctggcgccgctggaaaagctggataccctactggaacaggcgtgggccctcaagcagcagctgccgcagcagctaaagccgccgctaaatttggagctggcgctgcaggcgttttgcccggcgttggcggagcaggcgtcccaggtgttccaggggcaatacctggaatcggaggaattgccggcgtcggaactccagctgcagcagccgcagccgccgctgccgcaaaagctgctaaatatggcgcagctgcaggcttggtcccaggcggacctggatttggaccaggtgttgttggagtgccaggcgctggcgtccccggcgtgggagttcctggtgccggaattcctgttgttcctggcgctggtattcctggcgctgctgttccaggtgttgtgtctcctgaagccgctgccaaggccgctgctaaggcagctaaatacggtgcccgaccaggcgtcggagttggcggaattccaacatatggtgtcggagccggcggattcccaggatttggagttggagtcggaggcatcccgggtgttgcaggcgttccaggcgtcggcggagttcctggcgttggtggtgttccaggcgtgggaatttctcctgaagctcaggccgctgccgctgccaaagcagccaaatatggcgctgccggtgctggcgttctcggaggattggttccgggtgctccaggtgctgttccgggcgttcccggaactggcggtgtccctggtgtcggaacaccagccgctgcagcagcaaaggctgctgctaaagccgctcagtttggactggttcctggcgtcggagtggcaccaggtgttggagttgcacctggcgttggcgtggcccctggcgtgggtcttgctcctggtgttggtgttgccccaggtgtcggagtcgctcccggtgtcggtgtcgcacctggtattggtcctggtggcgttgccgcagctgccaaatctgctgcaaaggtggccgccaaagcacagctgagagctgctgccggtcttggcgctggaatcccaggactcggtgttggagtgggagtgccaggtcttggtgttggagcaggcgtgcccggactcggagtcggagctggtgtcccaggttttggagctggtgcagatgaaggcgtgcggagatctctgagccctgagctgagagagggcgatcctagctctagccagcatctccctagcacacctagcagccctagagtccctggggctcttgctgcagccaaagccgctaagtatggggctgctgtccctggtgttcttggaggacttggagcactcggcggagtgggaattccaggcggtgtcgttggtgcaggacctgctgctgccgccgcagctgcaaaagcagcagctaaggcagcccagtttggccttgttggagccgctggacttggtggcctcggagttggtggtcttggtgtccccggtgttggcggacttggaggaattccaccagccgcagcagccaaggctgctaaatacggcgctgcaggacttggcggtgttcttggcggagctggacagtttccacttggaggcgttgcagccagacctggctttggcctgtctcctatttttcctggcggcgcttgtctgggcaaagcctgcggcagaaagcggaagtaa

SEQ ID NO: 39 - DNA, 1017 bp, Homo sapiens
  source 1..1017: mol_type = unassigned DNA
atgagtctaagtgcatttactctcttcctggcattgattggtggtaccagtggccagtactatgattatgattttcccctatcaatttatgggcaatcatcaccaaactgtgcaccagaatgtaactgccctgaaagctacccaagtgccatgtactgtgatgagctgaaattgaaaagtgtaccaatggtgcctcctggaatcaagtatctttaccttaggaataaccagattgaccatattgatgaaaaggcctttgagaatgtaactgatctgcagtggctcattctagatcacaaccttctagaaaactccaagataaaagggagagttttctctaaattgaaacaactgaagaagctgcatataaaccacaacaacctgacagagtctgtgggcccacttcccaaatctctggaggatctgcagcttactcataacaagatcacaaagctgggctcttttgaaggattggtaaacctgaccttcatccatctccagcacaatcggctgaaagaggatgctgtttcagctgcttttaaaggtcttaaatcactcgaataccttgacttgagcttcaatcagatagccagactgccttctggtctccctgtctctcttctaactctctacttagacaacaataagatcagcaacatccctgatgagtatttcaagcgttttaatgcattgcagtatctgcgtttatctcacaacgaactggctgatagtggaatacctggaaattctttcaatgtgtcatccctggttgagctggatctgtcctataacaagcttaaaaacataccaactgtcaatgaaaaccttgaaaactattacctggaggtcaatcaacttgagaagtttgacataaagagcttctgcaagatcctggggccattatcctactccaagatcaagcatttgcgtttggatggcaatcgcatctcagaaaccagtcttccaccggatatgtatgaatgtctacgtgttgctaacgaagtcactcttaattaa

SEQ ID NO: 40 - DNA, 1017 bp, synthetic construct
  misc_feature 1..1017: note = Synthetic Construct
  source 1..1017: mol_type = other DNA
atgagcctgagcgccttcacactgtttctggccctgatcggcggcacaagcggccagtactacgactacgatttcccactgagcatctacggccagagcagccctaattgcgcccctgagtgcaattgccccgagagctatcctagcgccatgtactgcgacgagctgaagctgaagtccgtgcctatggtgcctcctggcatcaagtacctgtacctgcggaacaaccagatcgaccacatcgacgagaaggcctttgagaacgtgaccgacctgcagtggctgatcctggaccacaacctgctggaaaacagcaagatcaagggccgcgtgttcagcaagctgaagcagctgaagaaactgcacatcaaccacaacaacctgaccgagagcgtgggccctctgcctaagtctctggaagatctgcagctgacccacaacaagatcaccaagctgggcagcttcgagggcctcgtgaacctgaccttcatccatctgcagcacaaccggctgaaagaggatgccgttagcgccgccttcaagggcctgaagagtctggaatacctggacctgagcttcaatcagatcgccagactgcctagcggcctgcctgtttctctgctgacactgtacctggacaacaacaaaatcagcaacatccccgacgagtacttcaagcggttcaacgccctgcagtacctgagactgagccacaacgagctggccgattctggcatccccggcaacagcttcaatgtgtccagcctggtggaactggacctgtcctacaacaagctgaaaaacatccccaccgtgaacgagaacctcgagaactactacctggaagtgaaccagctcgagaagttcgatatcaagagcttctgcaagatcctggggcctctgagctactccaagattaagcacctgaggctggacggcaaccggatcagcgaaacaagcctgcctcctgatatgtacgagtgcctgagagtggccaacgaagtgaccctgaactga

SEQ ID NO: 41 - DNA, 1437 bp, Homo sapiens
  source 1..1437: mol_type = unassigned DNA
atggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtag

SEQ ID NO: 42 - DNA, 1437 bp, synthetic construct
  misc_feature 1..1437: note = Synthetic Construct
  source 1..1437: mol_type = other DNA
atggcccctcttagacccctgctgattctggctctgctggcttgggttgccctggccgatcaagagtcttgcaagggcagatgcaccgagggcttcaacgtggacaagaaatgccagtgcgacgagctgtgcagctactaccagagctgctgcaccgattacaccgccgagtgcaagccccaagtgacaagaggcgacgtgttcaccatgcctgaggacgagtacaccgtgtacgacgacggcgaggaaaagaacaacgccaccgtgcacgagcaagttggcggaccttctctgaccagcgatctgcaggctcagagcaagggcaatcctgagcagacccctgtgctgaagcctgaggaagaagcccctgctcctgaagtgggagcctctaagcctgaaggcatcgacagcagacccgagacactgcatccaggcagacctcagcctcctgccgaagaggaactgtgtagcggcaagcctttcgacgccttcaccgacctgaagaacggcagcctgttcgcctttagaggccagtactgctacgagctggacgagaaggccgtgcggcctggatatcctaagctgatcagagatgtgtggggcatcgagggccccatcgacgccgctttcaccagaatcaactgtcagggcaagacctacctgttcaagggcagccagtattggagattcgaggacggcgtgctggaccctgactaccccagaaatatcagcgacggcttcgacggcatccccgacaatgttgatgctgctctggccctgcctgctcacagctactctggcagagaacgggtgtacttctttaagggcaaacagtactgggagtaccagttccagcaccagcctagccaagaggaatgcgagggcagctctctgagcgccgtgtttgagcacttcgccatgatgcagagagacagctgggaagatattttcgagctgctgttctggggcagaaccagcgccggaacaagacagcctcagttcatcagcagagactggcatggcgtgccaggacaagtggatgctgccatggccggcagaatctacatcagcggaatggcccctagacctagcctggccaagaagcagcggttccggcacagaaaccggaagggctacagaagccagagaggccactccagaggccggaaccagaatagcagaaggccctctagagccacctggctgagcctgtttagcagcgaggaaagcaacctgggcgccaacaactacgacgactaccggatggattggctggtgcctgccacatgcgagcctatccagagcgtgttcttcttcagcggcgacaaatattaccgcgtgaacctgcggactcggagagtggatacagtggaccctccttatcctcggtctatcgctcagtattggctgggctgtccagctcctggacacctgtaa

SEQ ID NO: 43 - DNA, 5175 bp, Homo sapiens
  source 1..5175: mol_type = unassigned DNA
atgcctccagcagtgaggcggtcagcctgcagcatgggatggctgtggatctttggggcagccctggggcagtgtctgggctacagttcacagcagcaaagggtgccatttcttcagcctcccggtcaaagtcaactgcaagcgagttatgtggagtttagacccagccagggttgtagccctggatactatcgggatcataaaggcttgtataccggacggtgtgttccctgcaattgcaacggacattcaaatcaatgccaggatggctcaggcatatgtgttaactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggagactgcataaaccaagaacccaaagatagcagccctgcagaagaatgtgatgattgcgacagctgtgtgatgaccctcctgaacgacctggccaccatgggcgagcagctccgcctggtcaagtctcagctgcagggcctgagtgccagcgcagggcttctggagcagatgaggcacatggagacccaggccaaggacctgaggaatcagttgctcaactaccgttctgccatttcaaatcatggatcaaaaatagaaggcctggaaagagaactgactgatttgaatcaagaatttgagactttgcaagaaaaggctcaagtaaattccagaaaagcacaaacattaaacaacaatgttaatcgggcaacacaaagcgcaaaagaactggatgtgaagattaaaaatgtcatccggaatgtgcacattcttttaaagcagatctctgggacagatggagagggaaacaacgtgccttcaggtgacttttccagagagtgggctgaagcccagcgcatgatgagggaactgcggaacaggaactttggaaagcacctcagagaagcagaagctgataaaagggagtcgcagctcttgctgaaccggataaggacctggcagaaaacccaccagggggagaacaatgggcttgctaacagtatccgggattctttaaatgaatacgaagccaaactcagtgaccttcgtgctcggctgcaggaggcagctgcccaagccaagcaggcaaatggcttgaaccaagaaaacgagagagctttgggagccattcagagacaagtgaaagaaataaattccctgcagagtgatttcaccaagtatctaaccactgcagactcatctttgttgcaaaccaacattgcgctgcagctgatggagaaaagccagaaggaatatgaaaaattagctgccagtttaaatgaagcaagacaagaactaagtgacaaagtaagagaactttccagatctgctggcaaaacatcccttgtggaggaggcagaaaagcacgcgcggtccttacaagagctggcaaagcagctggaagagatcaagagaaacgccagcggggatgagctggtgcgctgtgctgtggatgccgccaccgcctacgagaacatcctcaatgccatcaaagcggccgaggacgcagccaacagggctgccagtgcatctgaatctgccctccagacagtgataaaggaagatctgccaagaaaagctaaaaccctgagttccaacagtgataaactgttaaatgaagccaagatgacacaaaagaagctaaagcaagaagtcagtccagctctcaacaacctacagcaaaccctgaatattgtgacagttcagaaagaagtgatagacaccaatctcacaactctccgagatggtcttcatgggatacagagaggtgatattgatgctatgatcagtagtgcaaagagcatggtcagaaaggccaacgacatcacagatgaggttctggatgggctcaaccccatccagacagatgtggaaagaattaaggacacctatgggaggacacagaacgaagacttcaaaaaggctctgactgatgcagataactcggtgaataagttaaccaacaaactacctgatctttggcgcaagattgaaagtatcaaccaacagctgttgcccttgggaaacatctctgacaacatggacagaatacgagaactaattcagcaggccagagatgctgccagtaaggttgctgtccccatgaggttcaatggtaaatctggagtcgaagtccgactgccaaatgacctggaagatttgaaaggatatacatctctgtccttgtttctccaaaggcccaactcaagagaaaatgggggtactgagaatatgtttgtgatgtaccttggaaataaagatgcctcccgggactacatcggcatggcagttgtggatggccagctcacctgtgtctacaacctgggggaccgtgaggctgaactccaagtggaccagatcttgaccaagagtgagactaaggaggcagttatggatcgggtgaaatttcagagaatttatcagtttgcaaggcttaattacaccaaaggagccacatccagtaaaccagaaacacccggagtctatgacatggatggtagaaatagcaatacactccttaatttggatcctgaaaatgttgtattttatgttggaggttacccacctgattttaaacttcccagtcgactaagtttccctccatacaaaggttgtattgaattagatgacctcaatgaaaatgttctgagcttgtacaacttcaaaaaaacattcaatctcaacacaactgaagtggagccttgtagaaggaggaaggaagagtcagacaaaaattattttgaaggtacgggctatgctcgagttccaactcaaccacatgctcccatcccaacctttggacagacaattcagaccaccgtggatagaggcttgctgttctttgcagaaaacggggatcgcttcatatctctaaatatagaagatggcaagctcatggtgagatacaaactgaattcagagctaccaaaagagagaggagttggagacgccataaacaacggcagagaccattcgattcagatcaaaattggaaaactccaaaagcgtatgtggataaatgtggacgttcaaaacactataattgatggtgaagtatttgatttcagcacatattatctgggaggaattccaattgcaatcagggaaagatttaacatttctacgcctgctttccgaggctgcatgaaaaatttgaagaaaaccagtggtgtcgttagattgaatgatactgtgggagtaaccaaaaagtgctcggaagactggaagcttgtgcgatctgcctcattctccagaggaggacaattgagtttcactgatttgggcttaccacctactgaccacctccaggcctcatttggatttcagacctttcaacccagtggcatattattagatcatcagacatggacaaggaacctgcaggtcactctggaagatggttacattgaattgagcaccagcgatagcggcggcccaatttttaaatctccacagacgtatatggatggtttactgcattatgtatctgtaataagcgacaactctggactacggcttctcatcgatgaccagcttctgagaaatagcaaaaggctaaaacacatttcaagttcccggcagtctctgcgtctgggcgggagcaattttgagggttgtattagcaatgtttttgtccagaggttatcactgagtcctgaagtcctagatttgaccagtaactctctcaagagagatgtgtccctgggaggctgcagtttaaacaaaccaccttttctaatgttgcttaaaggttctaccaggtttaacaagaccaagacttttcgtatcaaccagctgttgcaggacacaccagtggcctccccaaggagcgtgaaggtgtggcaagatgcttgctcaccacttcccaagacccaggccaatcatggagccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgcttgggtggtcctttggagaaaggcatttatttctctgaagaaggaggtcatgtcgtcttggctcactctgtattgttggggccagaatttaagcttgttttcagcatccgcccaagaagtctcactgggatcctaatacacatcggaagtcagcccgggaagcacttatgtgtttacctggaggcaggaaaggtcacggcctctatggacagtggggcaggtgggacctcaacgtcggtcacaccaaagcagtctctgtgtgatggacagtggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaa

SEQ ID NO: 44 - DNA, 5175 bp, synthetic construct
  misc_feature 1..5175: note = Synthetic Construct
  source 1..5175: mol_type = other DNA
atgcctcctgctgtgcggagaagcgcctgttctatgggatggctgtggatctttggcgccgctctgggacagtgtctgggctactcttctcagcagcagcgggtgccatttctgcagccacctggacagtctcagctgcaggccagctacgtggaattcagacctagccagggctgtagccccggctactacagagatcacaagggcctgtacaccggcagatgcgtgccctgcaactgtaacggccacagcaaccagtgtcaggacggctctggcatctgcgtgaactgccagcataatactgccggcgagcactgcgagagatgccaagagggctactacggcaatgccgtgcatggcagctgtcgggcttgtccttgtcctcacaccaacagctttgccaccggctgcgttgtgaacggcggagatgttcggtgttcttgcaaggccggctacacaggcacacagtgcgaaagatgtgcccctggctactttggcaaccctcagaagtttggcggctcctgccagccttgctcctgcaattctaatggccagctgggctcttgtcaccctctgaccggcgactgcatcaatcaagagcctaaggacagcagccctgccgaggaatgcgacgattgcgatagctgcgtgatgaccctgctgaacgacctggccacaatgggagaacagctgcggctggttaagagccagctccagggactgtctgcctctgctggactgctggaacagatgcggcacatggaaacccaggccaaggacctgagaaaccagctgctgaactacagaagcgccatctccaaccacggcagcaagatcgaaggcctggaaagagagctgaccgacctgaatcaagagttcgagacactgcaagagaaggcccaagtgaacagccggaaggcccagactctgaacaacaacgtgaaccgggccacacagtccgccaaagaactggacgtgaagatcaagaacgtgatccggaacgtgcacatcctgctgaagcagatcagcggcacagatggcgagggcaacaatgtgcctagcggcgactttagcagagagtgggccgaagctcagcggatgatgagagagctgcggaaccggaacttcggcaagcacctgagagaagccgaggccgacaagagagagagccaactgctgctcaaccggatcagaacctggcagaaaacccaccagggcgagaacaacggcctggccaacagcatcagagacagcctgaatgagtacgaggccaagctgagcgatctgcgggccagacttcaagaagctgccgctcaggccaagcaggccaacggccttaatcaagagaacgagagagccctgggcgccatccagagacaagtgaaagagatcaacagcctgcagagcgacttcaccaagtacctgaccaccgccgatagcagcctgctgcagacaaatatcgccctgcagctcatggaaaagagccagaaagagtacgaaaagctggccgccagcctgaacgaggccaggcaagaactgtctgacaaagtgcgcgagctgagcagatccgccggcaagacatctctggtggaagaggccgagaagcacgccagatctctgcaagagctggccaaacagctggaagagattaagcggaacgccagcggcgacgaactcgtcagatgtgcagtggatgccgccaccgcctacgagaacatcctgaatgccatcaaggccgccgaggacgccgctaatagagccgcttctgcttctgagtctgccctgcagaccgtgatcaaagaggacctgcctagaaaggccaagacactgagcagcaacagcgacaaactgctgaatgaggccaagatgacccagaagaaactgaagcaagaggtgtcccctgcactgaacaacctgcagcagaccctgaacatcgtgaccgtgcagaaagaagtgatcgacaccaacctgacaaccctgagagatggcctgcacggaatccagagaggcgacatcgacgccatgatcagcagcgccaagagcatggttcgaaaagccaacgacatcaccgacgaggtgctggacggcctgaatcctatccagaccgacgtggaacggatcaaggacacctacggcagaacccagaacgaggatttcaagaaggccctgaccgacgccgacaactccgtgaacaagctgaccaacaagctgcccgatctgtggcggaagatcgagagcatcaaccagcaactgctccctctgggcaacatcagcgacaacatggacagaatccgggaactgatccagcaggccagagatgccgcctccaaagtggctgtgcccatgagattcaacggcaagagcggagtggaagtgcggctgcccaacgatctggaagatctgaagggctataccagcctgagcctgttcctgcagaggcccaacagcagagagaatggcggcaccgagaatatgttcgtgatgtacctgggaaacaaggacgccagccgggactatatcggaatggccgttgtggacggccagctgacctgcgtgtacaacctgggagacagagaagctgaactgcaggtcgaccagatcctgaccaagagcgagacaaaagaggccgtgatggacagagtgaagttccagcggatctaccagttcgcccggctgaattacaccaagggcgccacaagcagcaagcccgaaacacctggcgtgtacgacatggacggccggaactctaacactctgctgaatctggaccccgagaacgtggtgttttacgtcggcggctaccctcctgacttcaagctgcctagcagactgagcttcccaccttacaagggctgcatcgagctggatgacctgaacgaaaacgtgctgtccctgtacaacttcaaaaagaccttcaacctgaacaccaccgaggtggaaccctgcaggcgcagaaaagaggaatccgacaagaactacttcgaaggcaccggctacgccagagtgcctacacaacctcacgctcccattcctaccttcggccagaccatccagacaaccgtggatagaggcctgctgttcttcgccgagaacggcgacagattcatctccctgaatatcgaggatggcaagctgatggtccgatacaagctgaatagcgagctgcccaaagaaagaggcgtgggcgacgccatcaacaacggcagggatcacagcatccagatcaagatcggcaaactgcagaaacggatgtggatcaacgtggacgtgcagaacaccatcatcgacggcgaggtgttcgacttcagcacctactatctcggcggaatccctatcgccatcagagagcggttcaatatcagcacccctgccttccggggctgcatgaagaacctgaaaaagaccagcggcgtcgtgcggctgaatgatacagtgggcgtgaccaagaagtgcagcgaggactggaagcttgtgcggagcgccagtttttctagaggcggacagctgagctttaccgacctgggactgcctcctaccgatcatctgcaggcaagcttcggattccagaccttccagccaagcggaatcctgctggaccaccagacctggaccagaaacctgcaagtgaccctggaagatggctacatcgaactgagcaccagcgactctggcggccctatctttaagagccctcagacctacatggatgggctgctgcactacgtgtccgtgatcagcgataacagcggcctgagactgctgatcgacgaccagctcctgcggaacagcaagcggctgaagcacatctccagcagcagacagagtctgagactcggcggcagcaatttcgagggctgtatcagcaacgtgttcgtgcagcgcctgagtctgtctccagaagtgctggacctgaccagcaatagcctgaagagggatgtgtctctcggcggctgctccctgaacaaacctcctttcctgatgctgctgaagggcagcacccggttcaacaagaccaagacctttcggatcaatcagctgctccaggacacccctgtggctagccctagaagcgtgaaagtgtggcaggacgcctgcagtcccctgcctaaaacacaggccaatcacggggctctgcagttcggcgatatccccacaagccatctgctgtttaagctgccccaagagctgctcaagcctcggagccagttcgctgtggatatgcagaccacctcctccagaggactggtgtttcacaccggcaccaagaacagcttcatggccctgtacctgagcaaaggcaggctggtgtttgccctgggcaccgacggaaagaaactgcggatcaagagcaaagagaagtgcaacgacggcaagtggcacaccgtggtgttcggacacgatggcgagaaaggcagactcgtggtggatggcctgagagccagagagggatctctgcctggcaactccaccatctccatcagagcccctgtgtatctgggcagccctcctagcggaaagcctaagagcctgcctaccaactccttcgtgggctgtctgaagaactttcagctggacagcaagcctctgtacacccctagcagcagctttggcgtgtcctcctgtctcggaggccctctggaaaagggcatctacttctctgaggaaggcggccacgttgtcctggctcattctgttctgctgggccccgagttcaagctggtgttctctatccggcctagaagcctgaccggcatcctgattcacatcggcagccagcctgggaagcacctgtgtgtgtatctcgaggccggcaaagtgaccgccagcatggattctggtgctggcggcacaagcacctccgtgacacctaagcagagcctgtgtgatggccagtggcacagtgtggccgtgacaatcaagcagcacattctgcacctggaactggacaccgacagcagctataccgccggacagatcccatttcctccagccagcacacaagagcctctgcaccttggaggcgcccctgccaatctgaccacactgagaatccccgtgtggaagtccttcttcggctgcctgcggaatatccatgtgaaccacattccagtgcctgtgacagaggccctggaagtgcagggacccgtgtctctgaatggatgccccgatcagtga

SEQ ID NO: 45 - DNA, 3519 bp, Homo sapiens
  source 1..3519: mol_type = unassigned DNA
atgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcacagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgtaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtga

SEQ ID NO: 46 - DNA, 3519 bp, synthetic construct
  misc_feature 1..3519: note = Synthetic Construct
  source 1..3519: mol_type = other DNA
atgaggcccttcttcctgctgtgctttgccctgcctggactgctgcatgctcagcaggcttgtagcagaggcgcctgctatcctcctgtgggcgatctgcttgtgggcagaaccagattcctgcgggccagctctacatgcggcctgacaaagcctgagacatactgcacccagtacggcgagtggcagatgaagtgctgcaagtgcgacagcagacagccccacaactactacagccacagagtggaaaacgtggccagcagcagcggccctatgagatggtggcagagccagaacgacgtgaaccccgttagcctgcagctggacctggacagacggtttcagctgcaagaagtgatgatggaatttcagggccccatgcctgccggcatgctgatcgagagaagcagcgatttcggcaagacctggcgggtgtaccagtatctggccgccgattgcaccagcacattccccagagttagacagggcagaccccagagctggcaggatgttcgttgtcagtctctgccccagcggcctaacgctagactgaatggcggaaaggtgcagctcaacctgatggacctggtgtctggcatccctgccacacagtcccagaaaatccaagaagtgggcgagatcaccaacctgagagtgaacttcacccggctggctcccgttcctcagagaggatatcatcctcctagcgcctactacgccgtgtctcagcttagactgcagggcagctgcttctgtcacggccacgctgatagatgcgcccctaaacctggtgcctctgccggaccttctacagccgtgcaagtgcacgatgtgtgcgtgtgccagcacaataccgccggacctaactgcgagagatgtgcccctttctacaacaaccggccttggaggcctgccgaaggacaggatgctcacgagtgccagagatgcgactgcaacggccacagcgagacatgccactttgaccctgccgtgtttgccgcttctcagggcgcttatggcggcgtgtgtgacaactgcagagatcacaccgagggcaagaactgcgagcgctgtcagctgcactacttccggaatagaaggccaggcgccagcatccaagagacatgcatcagctgcgagtgcgatcccgatggtgctgttcctggcgctccttgtgatcctgtgacaggccagtgcgtgtgtaaagaacacgtgcagggcgaaagatgcgacctgtgcaagcctggctttaccggcctgacctacgccaatcctcagggctgccacagatgtgattgcaacatcctgggcagcagacgggacatgccctgtgatgaagagtctggcagatgcctgtgcctgcctaatgtcgtgggccccaagtgcgatcagtgtgccccatatcactggaagctggcctctggccagggatgcgaaccttgtgcctgcgatccccacaacagcctgtctccacagtgcaaccagttcaccggccagtgtccttgcagagaaggctttggcggcctgatgtgttctgccgccgctatcagacagtgccccgatagaacatatggcgacgtggccacaggctgcagagcctgcgattgtgacttccggggaacagaaggacccggctgcgataaggccagcggaagatgtctgtgtcggcctggactcacaggccccagatgtgaccagtgtcagcggggctactgcaacagataccctgtgtgtgtggcctgccatccttgcttccagacctacgacgccgacctgagagaacaggccctgagattcggcagactgagaaatgccaccgccagcctttggagcggacctggccttgaagatagaggcctggcctccagaatcctggacgccaagtctaagatcgagcagatcagagccgtgctgtctagcccagccgtgaccgaacaagaggtggcccaagtggctagcgccatcctgagcctgagaagaactctgcagggactgcagctcgatctgcccctggaagaggaaacactgagcctgcctagagatctggaaagcctggatcggagcttcaacggcctgctgacaatgtaccagagaaagagagagcagttcgagaagatcagcagcgccgatcctagcggcgccttcagaatgctgagcacagcctatgagcagagcgcccaggctgctcagcaagtgtccgatagcagcagactgctggaccagctgcgggactctagaagagaagccgaaagacttgtgcggcaggcaggcggcggaggtggaacaggatctcctaaactggtggccctgcggctggaaatgtcctctctgcctgatctgacccctaccttcaacaagctgtgcggcaacagccggcagatggcctgcacacctattagctgtcctggcgagctgtgccctcaggataatggaaccgcctgcggctccagatgtagaggcgttttgccaagagccggcggagcctttctgatggctggacaagttgccgagcagctgagaggcttcaacgctcagctgcagcggaccagacagatgattagagccgccgaggaaagcgccagccagattcaatctagcgcccagagactggaaacccaggtgtccgccagcagatcccagatggaagaagatgtgcggcggacaagactgctgatccagcaagtgcgggacttcctgaccgatcctgataccgatgccgccacaatccaagaggtgtccgaagctgttctggcactgtggctgcctaccgatagcgctacagtgctgcagaagatgaacgagatccaggcaatcgccgccagactgcccaatgtggatctggtgctgagccagaccaagcaggatatcgccagagctagaaggctgcaggccgaggccgaagaggcaagatctagagcccatgccgtggaaggccaagtcgaggacgttgtgggcaatctgagacagggaaccgtggctctgcaagaggcccaggatacaatgcagggcaccagcagaagcctgcgcctgatccaggatagagtggccgaagtgcagcaggtcctgaggccagccgaaaagctggtcaccagcatgaccaaacagctgggcgatttctggacgcgcatggaagaactgaggcatcaggcaagacagcagggcgctgaagcagtgcaggctcaacaacttgccgagggcgcttctgaacaggctctgtctgcccaagagggcttcgagcggatcaagcagaagtacgccgagctgaaggacagactgggccagagttctatgctgggcgaacagggcgccagaattcagagcgtgaaaacagaggccgaggaactgttcggcgagacaatggaaatgatggaccggatgaaggacatggaactggaactgctgaggggcagccaggccatcatgctgagaagtgccgatctgacaggcctggaaaagagagtggaacagatccgggaccacatcaacggccgggtgctgtactacgccacatgcaagtaa

SEQ ID NO: 47 - DNA, 3582 bp, Homo sapiens
  source 1..3582: mol_type = unassigned DNA
atgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatggaccagcctctcagtgtagatgaagaggggctggtcttactggagcagaagctttcccgagccaagacccagatcaacagccaactgcggcccatgatgtcagagctggaagagagggcacgtcagcagaggggccacctccatttgctggagacaagcatagatgggattctggctgatgtgaagaacttggagaacattagggacaacctgcccccaggctgctacaatacccaggctcttgagcaacagtga

SEQ ID NO: 48 - DNA, 3582 bp, synthetic construct
  misc_feature 1..3582: note = Synthetic Construct
  source 1..3582: mol_type = other DNA
atgcctgctctgtggctgggctgctgcctgtgttttagtctgctgctgccagccgccagagccacatctagaagagaagtgtgcgactgcaacggcaagagccggcagtgcatcttcgacagagagctgcacagacagaccggcaacggcttcagatgcctgaactgcaacgacaacaccgacggcatccactgcgagaagtgcaagaacggcttctaccggcaccgcgagagggatagatgcctgccttgcaactgcaactccaagggcagcctgagcgccagatgcgacaatagcggcagatgtagctgcaagcctggcgtgacaggcgctagatgcgatagatgtctccccggcttccacatgctgaccgatgccggatgtacccaggaccagagactgctggacagcaagtgcgattgcgaccctgccggaattgccggaccttgtgatgccggaagatgcgtgtgtaaacctgccgtgaccggcgagagatgtgacagatgtagaagcggctactacaacctggacggcggcaatcctgaaggctgcacccagtgcttttgctacggccacagcgccagctgtagaagcagcgccgaatactccgtgcacaagatcaccagcaccttccaccaggatgtggacggatggaaggccgtgcagagaaatggctctcctgccaagctgcagtggtcccagagacaccaggacgtgttcagcagcgctcagagactggaccccgtgtactttgtggcccctgccaagttcctgggcaaccagcaagtgtcttacggccagagcctgagcttcgactacagagtggatagaggcggcagacaccccagcgctcacgatgtgattcttgaaggcgccggactgcggatcacagcccctcttatgcctctgggcaagaccctgccttgtggcctgaccaagacctacaccttccggctgaatgagcaccccagcaacaactggtccccacagctgagctacttcgagtacagacggctgctgcggaacctgacagccctgagaatcagagccacctacggcgagtacagcaccggctacatcgacaacgtgaccctgatcagcgccagacctgtttctggtgctcctgctccttgggtcgagcagtgtatctgtcccgtgggctacaagggccagttctgccaggattgtgccagcggctacaagagagactctgccagactgggccccttcggcacatgcatcccttgtaattgtcaaggcggcggagcctgcgatcccgatacaggcgattgctacagcggcgacgagaaccccgatatcgagtgcgccgattgtcccatcggcttttacaacgaccctcacgaccccagatcctgcaagccatgtccttgccacaatggcttcagctgcagcgtgatgcccgaaaccgaagaggtcgtgtgcaacaattgcccaccaggcgttacaggggccagatgtgaactgtgtgccgacggctacttcggcgatccttttggagaacacggacccgtgcgaccttgccagccttgtcagtgcaacaacaacgtggacccaagcgccagcggcaactgcgatagactgacaggcagatgtctgaagtgcatccacaataccgccgggatctactgtgaccagtgcaaggccggctattttggcgaccctctggctcccaatcctgccgataagtgcagagcctgcaactgtaaccctatgggctctgagcctgtgggctgcagatctgatggaacctgcgtgtgcaagccaggctttggcggacctaattgtgaacacggcgcctttagctgccccgcctgctacaatcaagtgaagatccagatggaccagttcatgcagcagctgcagaggatggaagccctgatctctaaagcccaaggcggagatggcgtggtgcctgatacagagctggaaggcagaatgcagcaggccgaacaggccctgcaggacattctgagagatgcccagattagcgagggcgcctctagaagtctgggactgcagctggctaaagtgcggagccaagagaacagctaccagagcagactggacgacctgaagatgaccgtggaaagagtcagagccctgggcagccagtaccagaacagagtgcgggatacccaccggctgatcacccagatgcaactgtctctggccgagagcgaagccagcctgggcaataccaatattcccgccagcgaccactacgtgggccccaacggttttaagagcctggctcaagaggccaccagactggccgaaagccatgtggaaagcgcctccaacatggaacagctgacccgggaaaccgaggactactctaagcaggccctgagcctcgtcagaaaagccctgcatgaaggcgtcggcagcggctctggatctcctgatggtgctgtggtgcagggactcgtggaaaagctggaaaagaccaaatctctggcccagcagctgaccagagaagccacacaggccgagatcgaggccgacagaagctaccagcactcactgaggctgctggactccgtgtctagactgcagggcgtgtccgaccagagcttccaggtggaagaggccaagcggatcaagcagaaggccgatagcctgagcagcctggtcaccagacacatggacgagttcaagcggacccagaagaacctcggcaactggaaagaggaagcccagcaactgctgcagaacggcaagtctggaagagagaagtctgaccagctgctgagcagagccaacctggccaagtctagagcccaagaggccctgtctatgggcaacgccaccttctacgaggtggaatccatcctgaagaacctgcgcgagttcgacctgcaagtggacaacagaaaggccgaggccgaggaagccatgaagagactgagctacatcagccagaaagtgtccgacgcctccgacaagacacagcaggcagaaagagcactgggatctgccgcagccgatgctcagagagctaaaaacggcgctggcgaggccctggaaatcagctctgagatcgagcaagagatcggctccctgaatctggaagccaatgtgacagccgatggcgccctggccatggaaaaaggactggcctctctgaagtccgagatgagagaggtggaaggcgagctggaacggaaagaactggaattcgacaccaatatggacgctgtgcagatggtcatcacagaggcccagaaggtggacaccagagccaaaaatgccggcgtgaccatccaggacaccctgaatactctggacggactgctgcacctgatggatcagcctctgagcgtggacgaggaaggactggttctgctggaacagaagctgagccgggccaagactcagatcaacagccagctgaggcccatgatgagcgaactggaagaacgggccagacagcagaggggccatctgcatctgctcgaaaccagcatcgatggcatcctggccgacgtgaagaatctcgagaacatccgggacaacctgccacctggctgctacaacacacaggcactggaacagcagtga

SEQ ID NO: 49 - DNA, 3891 bp, Clostridium botulinum
  source 1..3891: mol_type = unassigned DNA
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 50 - DNA, 3891 bp, synthetic construct
  misc_feature 1..3891: note = Synthetic Construct
  source 1..3891: mol_type = other DNA
atgcccttcgtgaacaagcagttcaactacaaggaccccgtgaacggcgtggacattgcctacatcaagatccccaacgccggccagatgcagcccgtgaaggcctttaagatccacaacaagatctgggtcatccccgagcgggacaccttcacaaatcccgaggaaggcgacctgaatcctccacctgaagccaaacaggtgcccgtgtcctactacgacagcacctacctgagcaccgacaacgagaaggacaactacctgaagggcgtgaccaagctgttcgagcggatctacagcaccgatctgggcagaatgctgctgacctctatcgtgcggggcatcccattttggggcggcagcacaatcgacaccgagctgaaagtgatcgacaccaactgcatcaacgtgatccagcctgacggcagctacagaagcgaggaactgaacctggtcatcatcggccccagcgccgacatcatccagttcgagtgcaagagcttcggccacgaggtgctgaacctgaccagaaatggctacggcagcacccagtacatccggttcagccccgatttcaccttcggcttcgaggaatccctggaagtggacaccaatcctctgctcggagccggcaagtttgccaccgatcctgctgtgacactggcccacgaactgattcacgccggacacagactgtacggaatcgccatcaatcccaaccgggtgttcaaagtgaacaccaacgcctactatgagatgagcggactggaagtgtcctttgaggaactgcggaccttcggcggacacgacgccaagtttatcgacagcctgcaagagaacgagttccggctgtactactacaacaagttcaaggatatcgccagcacgctgaacaaggccaagagcatcgtgggcacaacagccagcctgcagtacatgaagaacgttttcaaagagaagtacctgctgagcgaggacaccagcggcaagttctccgtggacaagctgaagttcgacaagctgtacaagatgctgaccgagatctacaccgaggacaacttcgtgaagttcttcaaggtgctcaaccgcaagacctacctcaacttcgacaaggccgtgttcaagatcaacatcgtgcccaaagtcaactacaccatctacgacggcttcaacctgcgcaacaccaacctggccgccaacttcaacggccagaacaccgagatcaacaacatgaacttcaccaagctgaaaaacttcaccggcctgttcgagttctataagctgctgtgcgtgcgcggcatcatcaccagcaagaccaagagcctggacaagggctataacaaggccctgaacgacctgtgcatcaaagttaacaactgggacctgttcttcagccccagcgaggataatttcaccaacgacctgaacaaaggcgaggaaatcaccagcgacaccaatatcgaggccgccgaggaaaacatcagcctggacctgatccagcagtactacctgaccttcaatttcgataacgagcccgagaacatcagcatcgagaacctgagcagcgatatcatcggacagctggaactgatgcccaacatcgagagattccccaacggcaagaagtacgagctggacaagtacaccatgttccactacctgcgggcccaagagttcgagcacggcaagtctagaatcgccctgaccaacagcgtgaacgaggccctgctgaaccccagcagagtgtacaccttcttcagcagcgactacgtgaagaaagtcaacaaggctacagaggccgccatgttcctcggctgggttgagcagctggtgtacgacttcaccgacgagacaagcgaggtgtccaccaccgacaagatcgccgatatcaccatcatcatcccttacatcggccctgctctgaacatcggcaacatgctgtataaggacgatttcgtgggcgccctgatcttctctggcgccgtgattctgctcgagttcatccctgagatcgctatccccgtgctgggcacatttgctctggtgtcttatatcgccaacaaggtgctgacagtgcagaccatcgacaacgccctgagcaagcggaacgagaagtgggacgaagtgtacaagtacatcgtgaccaactggctggccaaagtgaatacccagatcgacctgattcggaagaagatgaaggaagccctcgagaaccaggccgaggccacaaaggccatcatcaactaccagtacaatcagtacacagaggaagagaagaacaacatcaatttcaacatcgacgacctgtcctccaagctcaacgagagcatcaacaaagccatgatcaatatcaacaagtttctgaaccagtgcagcgtcagctacctgatgaacagcatgatcccctacggcgtgaagcggctggaagatttcgatgccagcctgaaggacgctctgctgaagtacatctacgataaccggggcaccctgatcggccaggtggacagactgaaggacaaagtgaacaataccctgtccaccgacattccgtttcagctgagcaaatacgtggacaaccagagactgctgagcacattcaccgagtatatcaagaatatcatcaacacctccatcctgaacctccgctacgagagcaaccacctgatcgatctgagcagatacgccagcaagatcaatattggctctaaagtgaacttcgacccgatcgacaagaaccagatccagctgttcaatctcgagtctagcaagatcgaagtgatcctgaagaacgccatcgtgtacaactctatgtacgagaacttctccaccagcttttggatcagaatccccaagtacttcaacagcatctccctgaacaacgagtacacgatcatcaattgcatggaaaacaactccggctggaaagtgtccctgaactacggcgagatcatctggacactgcaggacacccaagagatcaagcagagagtggtgttcaagtactctcagatgatcaacattagcgactacatcaaccggtggatcttcgtgaccatcaccaacaaccggctgaacaactccaagatctacatcaatggccggctcatcgaccagaagcctatcagcaacctgggaaacatccacgcctccaacaatatcatgttcaagctggacggctgccgggacacccaccggtatatctggatcaagtactttaacctgttcgacaaagagctgaacgagaaagagattaaggacctgtacgacaaccagtccaacagcggcatcctgaaggatttctggggcgactacctgcagtatgacaagccctactacatgctgaatctgtacgaccccaacaaatatgtggacgtgaacaacgtggggatcagaggctacatgtacctgaaaggccccagaggcagcgtgatgaccaccaacatctacctgaactccagcctgtacagaggcaccaagttcatcatcaagaagtatgcctccggcaacaaggacaacattgtgcggaacaacgaccgggtgtacattaacgtggtggtcaagaacaaagagtaccggctggccaccaatgcctctcaggcaggcgtggaaaagatcctgagcgccctggaaatccccgacgtgggcaatctgtctcaggtggtcgtgatgaagtccaagaacgaccagggcatcacaaacaagtgcaagatgaacctccaggacaacaacggcaacgacatcggctttatcggcttccaccagtttaacaacattgccaagctggtcgccagcaactggtacaaccggcagatcgagagaagcagcagaaccctgggctgcagctgggagtttatccctgtggatgatggctggggcgaaagacccctgtaa

SEQ ID NO: 51 - DNA, 3876 bp, Clostridium botulinum
  source 1..3876: mol_type = unassigned DNA
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 52 - DNA, 3876 bp, synthetic construct
  misc_feature 1..3876: note = Synthetic Construct
  source 1..3876: mol_type = other DNA
atgcccgtgaccatcaacaacttcaactacaacgaccccatcgacaacaacaacatcattatgatggaaccgcctttcgccagaggcacaggccggtactacaaggccttcaagatcaccgaccggatctggatcatccccgagagatacaccttcggctacaagcccgaggacttcaacaagagcagcggcatcttcaaccgggacgtgtgcgagtactacgaccccgactacctgaacaccaacgacaagaagaacatcttcctgcagaccatgatcaagctgttcaaccggatcaagagcaagcccctgggcgagaagctgctggaaatgatcatcaacggcatcccttacctgggcgacagaagagtgcccctggaagagttcaacaccaatatcgccagcgtgaccgtgaacaagctgatctctaaccccggcgaggtggaacggaagaagggcatcttcgccaacctgatcatcttcggccctggacctgtgctgaacgagaacgagacaatcgacatcggcatccagaaccacttcgccagcagagaaggcttcggcggcatcatgcagatgaagttctgccccgagtacgtgtccgtgttcaacaacgtgcaagagaacaagggcgccagcatctttaacagacggggctacttcagcgaccccgctctgattctgatgcacgagctgatccacgtgctgcacggcctgtatggcatcaaggtggacgacctgcctatcgtgcccaacgagaagaaattcttcatgcagtccaccgacgccatccaggccgaggaactgtacacatttggcggacaggaccccagcatcatcacccctagcaccgacaagagcatctacgacaaggtgctgcagaatttccggggcatcgtggaccggctgaacaaagtgctcgtgtgcatcagcgatcccaacatcaatatcaacatctacaagaacaagttcaaggataagtacaagttcgtcgaggacagcgagggcaagtacagcatcgacgtggaaagcttcgacaagctgtacaagagcctgatgttcggcttcaccgagacaaatatcgccgagaactacaagatcaagacccgggccagctacttctccgactctctgcctcctgtgaagattaagaacctgctggacaacgagatctacaccatcgaggaaggcttcaacatcagcgacaaggacatggaaaaagagtaccggggccagaacaaggccattaacaagcaggcctacgaggaaatcagcaaagaacacctggccgtgtacaagattcagatgtgcaagagcgtgaaggcccctggcatctgcattgacgtggacaatgaggacctgttctttatcgccgacaagaacagctttagcgacgacctgagcaagaacgagcggatcgagtacaacacccagagcaactacatcgagaacgacttccccatcaacgaactgatcctggacaccgacctgatcagcaagatcgagctgcccagcgagaacaccgagagcctgaccgacttcaatgtggacgtgcccgtgtacgagaagcagcccgccatcaagaagatctttaccgacgagaataccatcttccagtacctgtacagccagacctttcctctggacatccgggacatcagcctgacctccagcttcgatgatgccctgctgttcagcaacaaggtctacagcttcttcagcatggactacatcaagaccgccaacaaggtggtggaagccggcctgtttgccggctgggttaagcagatcgtgaacgatttcgtgatcgaggccaacaagtccaacaccatggacaagatcgccgatatctccctgatcgtgccctacatcggactggccctgaacgtgggaaacgagacagccaagggcaacttcgagaatgccttcgagattgccggcgctagcatcctgctcgagttcatccctgagctgctgatccctgtcgtgggcgcttttctgctggaatcctacatcgataacaaaaacaagatcatcaagacgatcgacaacgccctgaccaagcggaacgagaagtggagcgatatgtacggactgatcgtggcccagtggctgagcaccgtgaatacccagttttacaccatcaaagaagggatgtacaaggccctgaattaccaggctcaggctctggaagagatcattaagtaccgctacaatatctacagcgagaaagagaagtctaacatcaacatcgacttcaacgacatcaacagcaagctcaacgagggcatcaaccaggccattgacaacattaacaactttatcaacggctgcagcgtgtcctacctgatgaagaagatgattcctctggccgtggaaaagctgctcgacttcgacaataccctgaagaagaacctcctgaactacattgacgagaacaagctctacctgatcggcagcgccgagtacgagaaaagcaaagtgaacaagtacctcaagaccatcatgcccttcgacctgtccatctacacaaacgacaccatcctgatcgagatgtttaacaagtacaacagcgagatcctgaacaatatcatcctgaacctgcggtacaaggacaacaatctgatcgatctgagcggctacggcgccaaggtggaagtgtatgatggcgtggaactgaacgataagaatcagttcaagctgaccagcagcgccaactccaagatcagagtgacccagaaccagaacattatcttcaacagcgtgttcctggacttctccgtgtccttctggatcagaatccccaagtacaagaacgacggcattcagaactacatccacaacgagtacacaatcatcaactgtatgaagaacaacagcggctggaagatcagcatccggggcaacagaatcatctggaccctgatcgacatcaatggcaagacaaagagcgtgttcttcgagtataacatccgcgaggacatctccgagtacatcaaccggtggttcttcgtgacaatcaccaacaacctgaacaacgccaagatctacatcaacgggaagctcgagagcaacaccgacatcaaggatatccgggaagtgatcgccaacggcgagatcatctttaagctggacggcgacatcgaccggacacagttcatctggatgaagtacttttccatcttcaataccgagctgagccagagcaatatcgaagagaggtacaagatccagtcttacagcgagtacctgaaggacttctggggcaaccctctgatgtacaacaaagaatattacatgttcaacgccggcaacaagaactcttacatcaagctgaagaaagacagccccgtgggagaaatcctgaccaggtctaagtacaaccagaactccaagtatatcaactaccgggacctgtacatcggcgagaagttcatcatccggcgcaagagcaacagccagtccatcaatgacgacatcgtgcggaaagaggactatatctacctcgacttcttcaacctcaatcaagagtggcgcgtgtacacctacaagtactttaagaaagaggaagagaagctgttcctggctcctatcagcgactccgacgagttctacaataccatccagatcaaagagtacgacgagcagcccacctacagctgccagctgctgtttaagaaggacgaggaatccaccgatgagatcggcctgattggcatccaccggttctacgagagcggcatcgtgttcgaagagtacaaggattacttctgcatcagcaagtggtatctgaaagaagtgaagcggaagccctacaatctgaagctgggctgcaactggcagtttatccccaaggacgaaggctggaccgagtga

SEQ ID NO: 53 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 54 - protein, 786 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGAPGAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 55 - protein, 338 aa, Homo sapiens
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

SEQ ID NO: 56 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 57 - protein, 1724 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 58 - protein, 1172 aa, Homo sapiens [sequence omitted]

SEQ ID NO: 59 - protein, 1193 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMDQPLSVDEEGLVLLEQKLSRAKTQINSQLRPMMSELEERARQQRGHLHLLETSIDGILADVKNLENIRDNLPPGCYNTQALEQQ

SEQ ID NO: 60 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 61 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 62 - protein, 2871 aa, Homo sapiens
MRRGRLLEIALGFTVLLASYTSHGADANLEAGNVKETRASRAKRRGGGGHDALKGPNVCGSRYNAYCCPGWKTLPGGNQCIVPICRHSCGDGFCSRPNMCTCPSGQIAPSCGSRSIQHCNIRCMNGGSCSDDHCLCQKGYIGTHCGQPVCESGCLNGGRCVAPNRCACTYGFTGPQCERDYRTGPCFTVISNQMCQGQLSGIVCTKTLCCATVGRAWGHPCEMCPAQPHPCRRGFIPNIRTGACQDVDECQAIPGLCQGGNCINTVGSFECKCPAGHKLNEVSQKCEDIDECSTIPGICEGGECTNTVSSYFCKCPPGFYTSPDGTRCIDVRPGYCYTALTNGRCSNQLPQSITKMQCCCDAGRCWSPGVTVAPEMCPIRATEDFNKLCSVPMVIPGRPEYPPPPLGPIPPVLPVPPGFPPGPQIPVPRPPVEYLYPSREPPRVLPVNVTDYCQLVRYLCQNGRCIPTPGSYRCECNKGFQLDLRGECIDVDECEKNPCAGGECINNQGSYTCQCRAGYQSTLTRTECRDIDECLQNGRICNNGRCINTDGSFHCVCNAGFHVTRDGKNCEDMDECSIRNMCLNGMCINEDGSFKCICKPGFQLASDGRYCKDINECETPGICMNGRCVNTDGSYRCECFPGLAVGLDGRVCVDTHMRSTCYGGYKRGQCIKPLFGAVTKSECCCASTEYAFGEPCQPCPAQNSAEYQALCSSGPGMTSAGSDINECALDPDICPNGICENLRGTYKCICNSGYEVDSTGKNCVDINECVLNSLLCDNGQCRNTPGSFVCTCPKGFIYKPDLKTCEDIDECESSPCINGVCKNSPGSFICECSSESTLDPTKTICIETIKGTCWQTVIDGRCEININGATLKSQCCSSLGAAWGSPCTLCQVDPICGKGYSRIKGTQCEDIDECEVFPGVCKNGLCVNTRGSFKCQCPSGMTLDATGRICLDIRLETCFLRYEDEECTLPIAGRHRMDACCCSVGAAWGTEECEECPMRNTPEYEELCPRGPGFATKEITNGKPFFKDINECKMIPSLCTHGKCRNTIGSFKCRCDSGFALDSEERNCTDIDECRISPDLCGRGQCVNTPGDFECKCDEGYESGFMMMKNCMDIDECQRDPLLCRGGVCHNTEGSYRCECPPGHQLSPNISACIDINECELSAHLCPNGRCVNLIGKYQCACNPGYHSTPDRLFCVDIDECSIMNGGCETFCTNSEGSYECSCQPGFALMPDQRSCTDIDECEDNPNICDGGQCTNIPGEYRCLCYDGFMASEDMKTCVDVNECDLNPNICLSGTCENTKGSFICHCDMGYSGKKGKTGCTDINECEIGAHNCGKHAVCTNTAGSFKCSCSPGWIGDGIKCTDLDECSNGTHMCSQHADCKNTMGSYRCLCKEGYTGDGFTCTDLDECSENLNLCGNGQCLNAPGGYRCECDMGFVPSADGKACEDIDECSLPNICVFGTCHNLPGLFRCECEIGYELDRSGGNCTDVNECLDPTTCISGNCVNTPGSYICDCPPDFELNPTRVGCVDTRSGNCYLDIRPRGDNGDTACSNEIGVGVSKASCCCSLGKAWGTPCEMCPAVNTSEYKILCPGGEGFRPNPITVILEDIDECQELPGLCQGGKCINTFGSFQCRCPTGYYLNEDTRVCDDVNECETPGICGPGTCYNTVGNYTCICPPDYMQVNGGNNCMDMRRSLCYRNYYADNQTCDGELLFNMTKKMCCCSYNIGRAWNKPCEQCPIPSTDEFATLCGSQRPGFVIDIYTGLPVDIDECREIPGVCENGVCINMVGSFRCECPVGFFYNDKLLVCEDIDECQNGPVCQRNAECINTAGSYRCDCKPGYRFTSTGQCNDRNECQEIPNICSHGQCIDTVGSFYCLCHTGFKTNDDQTMCLDINECERDACGNGTCRNTIGSFNCRCNHGFILSHNNDCIDVDECASGNGNLCRNGQCINTVGSFQCQCNEGYEVAPDGRTCVDINECLLEPRKCAPGTCQNLDGSYRCICPPGYSLQNEKCEDIDECVEEPEICALGTCSNTEGSFKCLCPEGFSLSSSGRRCQDLRMSYCYAKFEGGKCSSPKSRNHSKQECCCALKGEGWGDPCELCPTEPDEAFRQICPYGSGIIVGPDDSAVDMDECKEPDVCKHGQCINTDGSYRCECPFGYILAGNECVDTDECSVGNPCGNGTCKNVIGGFECTCEEGFEPGPMMTCEDINECAQNPLLCAFRCVNTYGSYECKCPVGYVLREDRRMCKDEDECEEGKHDCTEKQMECKNLIGTYMCICGPGYQRRPDGEGCVDENECQTKPGICENGRCLNTRGSYTCECNDGFTASPNQDECLDNREGYCFTEVLQNMCQIGSSNRNPVTKSECCCDGGRGWGPHCEICPFQGTVAFKKLCPHGRGFMTNGADIDECKVIHDVCRNGECVNDRGSYHCICKTGYTPDITGTSCVDLNECNQAPKPCNFICKNTEGSYQCSCPKGYILQEDGRSCKDLDECATKQHNCQFLCVNTIGGFTCKCPPGFTQHHTSCIDNNECTSDINLCGSKGICQNTPGSFTCECQRGFSLDQTGSSCEDVDECEGNHRCQHGCQNIIGGYRCSCPQGYLQHYQWNQCVDENECLSAHICGGASCHNTLGSYKCMCPAGFQYEQFSGGCQDINECGSAQAPCSYGCSNTEGGYLCGCPPGYFRIGQGHCVSGMGMGRGNPEPPVSGEMDDNSLSPEACYECKINGYPKRGRKRRSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 63 - protein, 2912 aa, Homo sapiens
MGRRRRLCLQLYFLWLGCVVLWAQGTAGQPQPPPPKPPRPQPPPQQVRSATAGSEGGFLAPEYREEGAAVASRVRRRGQQDVLRGPNVCGSRFHSYCCPGWKTLPGGNQCIVPICRNSCGDGFCSRPNMCTCSSGQISSTCGSKSIQQCSVRCMNGGTCADDHCQCQKGYIGTYCGQPVCENGCQNGGRCIGPNRCACVYGFTGPQCERDYRTGPCFTQVNNQMCQGQLTGIVCTKTLCCATIGRAWGHPCEMCPAQPQPCRRGFIPNIRTGACQDVDECQAIPGICQGGNCINTVGSFECRCPAGHKQSETTQKCEDIDECSIIPGICETGECSNTVGSYFCVCPRGYVTSTDGSRCIDQRTGMCFSGLVNGRCAQELPGRMTKMQCCCEPGRCWGIGTIPEACPVRGSEEYRRLCMDGLPMGGIPGSAGSRPGGTGGNGFAPSGNGNGYGPGGTGFIPIPGGNGFSPGVGGAGVGAGGQGPIITGLTILNQTIDICKHHANLCLNGRCIPTVSSYRCECNMGYKQDANGDCIDVDECTSNPCTNGDCVNTPGSYYCKCHAGFQRTPTKQACIDIDECIQNGVLCKNGRCVNTDGSFQCICNAGFELTTDGKNCVDHDECTTTNMCLNGMCINEDGSFKCICKPGFVLAPNGRYCTDVDECQTPGICMNGHCINSEGSFRCDCPPGLAVGMDGRVCVDTHMRSTCYGGIKKGVCVRPFPGAVTKSECCCANPDYGFGEPCQPCPAKNSAEFHGLCSSGVGITVDGRDINECALDPDICANGICENLRGSYRCNCNSGYEPDASGRNCIDIDECLVNRLLCDNGLCRNTPGSYSCTCPPGYVFRTETETCEDINECESNPCVNGACRNNLGSFNCECSPGSKLSSTGLICIDSLKGTCWLNIQDSRCEVNINGATLKSECCATLGAAWGSPCERCELDTACPRGLARIKGVTCEDVNECEVFPGVCPNGRCVNSKGSFHCECPEGLTLDGTGRVCLDIRMEQCYLKWDEDECIHPVPGKFRMDACCCAVGAAWGTECEECPKPGTKEYETLCPRGAGFANRGDVLTGRPFYKDINECKAFPGMCTYGKCRNTIGSFKCRCNSGFALDMEERNCTDIDECRISPDLCGSGICVNTPGSFECECFEGYESGFMMMKNCMDIDECERNPLLCRGGTCVNTEGSFQCDCPLGHELSPSREDCVDINECSLSDNLCRNGKCVNMIGTYQCSCNPGYQATPDRQGCTDIDECMIMNGGCDTQCTNSEGSYECSCSEGYALMPDGRSCADIDECENNPDICDGGQCTNIPGEYRCLCYDGFMASMDMKTCIDVNECDLNSNICMFGECENTKGSFICHCQLGYSVKKGTTGCTDVDECEIGAHNCDMHASCLNIPGSFKCSCREGWIGNGIKCIDLDECSNGTHQCSINAQCVNTPGSYRCACSEGFTGDGFTCSDVDECAENINLCENGQCLNVPGAYRCECEMGFTPASDSRSCQDIDECSFQNICVFGTCNNLPGMFHCICDDGYELDRTGGNCTDIDECADPINCVNGLCVNTPGRYECNCPPDFQLNPTGVGCVDNRVGNCYLKFGPRGDGSLSCNTEIGVGVSRSSCCCSLGKAWGNPCETCPPVNSTEYYTLCPGGEGFRPNPITIILEDIDECQELPGLCQGGNCINTFGSFQCECPQGYYLSEDTRICEDIDECFAHPGVCGPGTCYNTLGNYTCICPPEYMQVNGGHNCMDMRKSFCYRSYNGTTCENELPFNVTKRMCCCTYNVGKAWNKPCEPCPTPGTADFKTICGNIPGFTFDIHTGKAVDIDECKEIPGICANGVCINQIGSFRCECPTGFSYNDLLLVCEDIDECSNGDNLCQRNADCINSPGSYRCECAAGFKLSPNGACVDRNECLEIPNVCSHGLCVDLQGSYQCICHNGFKASQDQTMCMDVDECERHPCGNGTCKNTVGSYNCLCYPGFELTHNNDCLDIDECSSFFGQVCRNGRCFNEIGSFKCLCNEGYELTPDGKNCIDTNECVALPGSCSPGTCQNLEGSFRCICPPGYEVKSENCIDINECDEDPNICLFGSCTNTPGGFQCLCPPGFVLSDNGRRCFDTRQSFCFTNFENGKCSVPKAFNTTKAKCCCSKMPGEGWGDPCELCPKDDEVAFQDLCPYGHGTVPSLHDTREDVNECLESPGICSNGQCINTDGSFRCECPMGYNLDYTGVRCVDTDECSIGNPCGNGTCTNVIGSFECNCNEGFEPGPMMNCEDINECAQNPLLCAFRCMNTFGSYECTCPIGYALREDQKMCKDLDECAEGLHDCESRGMMCKNLIGTFMCICPPGMARRPDGEGCVDENECRTKPGICENGRCVNIIGSYRCECNEGFQSSSSGTECLDNRQGLCFAEVLQTICQMASSSRNLVTKSECCCDGGRGWGHQCELCPLPGTAQYKKICPHGPGYTTDGRDIDECKVMPNLCTNGQCINTMGSFRCFCKVGYTTDISGTSCIDLDECSQSPKPCNYICKNTEGSYQCSCPRGYVLQEDGKTCKDLDECQTKQHNCQFLCVNTLGGFTCKCPPGFTQHHTACIDNNECGSQPSLCGAKGICQNTPGSFSCECQRGFSLDATGLNCEDVDECDGNHRCQHGCQNILGGYRCGCPQGYIQHYQWNQCVDENECSNPNACGSASCYNTLGSYKCACPSGFSFDQFSSACHDVNECSSSKNPCNYGCSNTEGGYLCGCPPGYYRVGQGHCVSGMGFNKGQYLSLDTEVDEENALSPEACYECKINGYSKKDSRQKRSIHEPDPTAVEQISLESVDMDSPVNMKFNLSHLGSKEHILELRPAIQPLNNHIRYVISQGNDDSVFRIHQRNGLSYLHTAKKKLMPGTYTLEITSIPLYKKKELKKLEESNEDDYLLGELGEALRMRLQIQLY

SEQ ID NO: 64 - protein, 2809 aa, Homo sapiens
MTLEGLYLARGPLARLLLAWSALLCMAGGQGRWDGALEAAGPGRVRRRGSPGILQGPNVCGSRFHAYCCPGWRTFPGRSQCVVPICRRACGEGFCSQPNLCTCADGTLAPSCGVSRGSGCSVSCMNGGTCRGASCLCQKGYTGTVCGQPICDRGCHNGGRCIGPNRCACVYGFMGPQCERDYRTGPCFGQVGPEGCQHQLTGLVCTKALCCATVGRAWGLPCELCPAQPHPCRRGFIPNIHTGACQDVDECQAVPGLCQGGSCVNMVGSFHCRCPVGHRLSDSSAACEDYRAGACFSVLFGGRCAGDLAGHYTRRQCCCDRGRCWAAGPVPELCPPRGSNEFQQLCAQRLPLLPGHPGLFPGLLGFGSNGMGPPLGPARLNPHGSDARGIPSLGPGNSNIGTATLNQTIDICRHFTNLCLNGRCLPTPSSYRCECNVGYTQDVRGECIDVDECTSSPCHHGDCVNIPGTYHCRCYPGFQATPTRQACVDVDECIVSGGLCHLGRCVNTEGSFQCVCNAGFELSPDGKNCVDHNECATSTMCVNGVCLNEDGSFSCLCKPGFLLAPGGHYCMDIDECQTPGICVNGHCTNTEGSFRCQCLGGLAVGTDGRVCVDTHVRSTCYGAIEKGSCARPFPGTVTKSECCCANPDHGFGEPCQLCPAKDSAEFQALCSSGLGITTDGRDINECALDPEVCANGVCENLRGSYRCVCNLGYEAGASGKDCTDVDECALNSLLCDNGWCQNSPGSYSCSCPPGFHFWQDTEICKDVDECLSSPCVSGVCRNLAGSYTCKCGPGSRLDPSGTFCLDSTKGTCWLKIQESRCEVNLQGASLRSECCATLGAAWGSPCERCEIDPACARGFARMTGVTCDDVNECESFPGVCPNGRCVNTAGSFRCECPEGLMLDASGRLCVDVRLEPCFLRWDEDECGVTLPGKYRMDVCCCSIGAVWGVECEACPDPESLEFASLCPRGLGFASRDFLSGRPFYKDVNECKVFPGLCTHGTCRNTVGSFHCACAGGFALDAQERNCTDIDECRISPDLCGQGTCVNTPGSFECECFPGYESGFMLMKNCMDVDECARDPLLCRGGTCTNTDGSYKCQCPPGHELTAKGTACEDIDECSLSDGLCPHGQCVNVIGAFQCSCHAGFQSTPDRQGCVDINECRVQNGGCDVHCINTEGSYRCSCGQGYSLMPDGRACADVDECEENPRVCDQGHCTNMPGGHRCLCYDGFMATPDMRTCVDVDECDLNPHICLHGDCENTKGSFVCHCQLGYMVRKGATGCSDVDECEVGGHNCDSHASCLNIPGSFSCRCLPGWVGDGFECHDLDECVSQEHRCSPRGDCLNVPGSYRCTCRQGFAGDGFFCEDRDECAENVDLCDNGQCLNAPGGYRCECEMGFDPTEDHRACQDVDECAQGNLCAFGSCENLPGMFRCICNGGYELDRGGGNCTDINECADPVNCINGVCINTPGSYLCSCPQDFELNPSGVGCVDTRAGNCFLETHDRGDSGISCSAEIGVGVTRASCCCSLGRAWGNPCELCPMANTTEYRTLCPGGEGFQPNRITVILEDIDECQELPGLCQGGDCVNTFGSFQCECPPGYHLSEHTRICEDIDECSTHSGICGPGTCYNTLGNYTCVCPAEYLQVNGGNNCMDMRKSVCFRHYNGTCQNELAFNVTRKMCCCSYNIGQAWNRPCEACPTPISPDYQILCGNQAPGFLTDIHTGKPLDIDECGEIPAICANGICINQIGSFRCECPAGFNYNSILLACEDVDECGSRESPCQQNADCINIPGSYRCKCTRGYKLSPGGACVGRNECREIPNVCSHGDCMDTEGSYMCLCHRGFQASADQTLCMDIDECDRQPCGNGTCKNIIGSYNCLCFPGFVVTHNGDCVDFDECTTLVGQVCRFGHCLNTAGSFHCLCQDGFELTADGKNCVDTNECLSLAGTCLPGTCQNLEGSFRCICPPGFQVQSDHCIDIDECSEEPNLCLFGTCTNSPGSFQCLCPPGFVLSDNGHRCFDTRQSFCFTRFEAGKCSVPKAFNTTKTRCCCSKRPGEGWGDPCELCPQEGSAAFQELCPFGHGAVPGPDDSREDVNECAENPGVCTNGVCVNTDGSFRCECPFGYSLDFTGINCVDTDECSVGHPCGQGTCTNVIGGFECACADGFEPGLMMTCEDIDECSLNPLLCAFRCHNTEGSYLCTCPAGYTLREDGAMCRDVDECADGQQDCHARGMECKNLIGTFACVCPPGMRPLPGSGEGCTDDNECHAQPDLCVNGRCVNTAGSFRCDCDEGFQPSPTLTECHDIRQGPCFAEVLQTMCRSLSSSSEAVTRAECCCGGGRGWGPRCELCPLPGTSAYRKLCPHGSGYTAEGRDVDECRMLAHLCAHGECINSLGSFRCHCQAGYTPDATATTCLDMDECSQVPKPCTFLCKNTKGSFLCSCPRGYLLEEDGRTCKDLDECTSRQHNCQFLCVNTVGAFTCRCPPGFTQHHQACFDNDECSAQPGPCGAHGHCHNTPGSFRCECHQGFTLVSSGHGCEDVNECDGPHRCQHGCQNQLGGYRCSCPQGFTQHSQWAQCVDENECALSPPTCGSASCRNTLGGFRCVCPSGFDFDQALGGCQEVDECAGRRGPCSYSCANTPGGFLCGCPQGYFRAGQGHCVSGLGFSPGPQDTPDKEELLSSEACYECKINGLSPRDRPRRSAHRDHQVNLATLDSEALLTLGLNLSHLGRAERILELRPALEGLEGRIRYVIVRGNEQGFFRMHHLRGVSSLQLGRRRPGPGTYRLEVVSHMAGPWGVQPEGQPGPWGQALRLKVQLQLL